(19) United States
(12) Patent Application Publication
(10) Pub. No.: US 2004/0006026 A1
(43) Pub. Date: Jan. 8, 2004

(54) Avilamycin derivatives

(75) Inventors: Gabriele Weitnauer, Freiburg (DE); Agnes Muhlenweg, Berlin (DE); Axel Trefzer, Eichstatt (DE); Andreas Bechthold, Schallstadt-Mengen (DE)

Correspondence Address: HESLIN ROTHENBERG FARLEY & MESITI PC, 5 COLUMBIA CIRCLE, ALBANY, NY, 12203, US

(73) Assignee: Combinature Biopharm AG, Berlin (DE)

(21) Appl. No.: 10/084,846

(22) Filed: Feb. 25, 2002

(30) Foreign Application Priority Data
Aug. 24, 2001 (WO) PCT/EP01/09815
Feb. 25, 2001 (DE) 10109166.4

(51) Int. Cl. A61K 31/704; A61K 31/715; C08B 35/02

(52) U.S. Cl. 514/25; 514/61; 536/108

(57) ABSTRACT

Avilamycin derivatives, gene technology biosynthesis processes for their production, medications containing these compounds, as well as the use of these compounds for the production of a active agent against infectious diseases, as well as nucleic acids, proteins, and gene clusters and corresponding cells that are connected to the production of these avilamycin derivatives are disclosed.

### CLAIM FOR FOREIGN PRIORITY

This application claims priority from DE 10109166.4 filed Feb. 25, 2001 and International Application PCT/EPO1/09815 filed Aug. 24, 2001. The entire contents of each prior application is incorporated herein by reference.

### FIELD OF THE INVENTION

The invention relates to avilamycin derivatives (also referred to as gavibamycins hereinafter), gene technology biosynthesis processes for their production, medications containing these compounds, as well as the use of these compounds for the production of a medication, for example one against infectious diseases, as well as nucleic acids, proteins, and gene clusters and corresponding cells that are connected to the production of these avilamycin derivatives.

### BACKGROUND OF THE INVENTION

The occurrence of pathogenic bacteria that are multiresistant to antibiotics represents a growing threat to human health and has intensified the search for new drugs. During the past two decades, fewer and fewer new drugs have been found in target-specific drug screenings, so that researchers have begun to utilize new technologies for the production of new compounds, in addition to the search for new antibiotic drugs. A promising new technology is denoted as recombinant biosynthesis and utilizes biosynthetic genes as the means for the production of new active agents.

An interesting class of compounds in this context, among others, is the orthosomycins. They are a known class of antibiotics that are produced from various actinomycetes. Members of this class act on a broad spectrum of gram-positive pathogenic bacteria, including glycopeptide-resistant enterococci, methicillin-resistant staphylococci, and penicillin-resistant streptococci.

Prominent examples of orthosomycins are avilamycins and evernimycins that are produced from Streptomyces viridochromogenes Tü57 and Micromonospora carbonacea, respectively. These antibiotics consist of a heptasaccharide side chain and a dichloroisoeverninic acid derived from the polyketide (acetate units) as the aglycon, where the sugar moieties are partly linked with one another via ortho ester bonds. This bond gives the entire class of orthosomycins their name. The precise mode of action of the orthosomycins is unknown. While a cell membrane effect was discussed for a specific orthosomycin (Ziracin) (Walker, 1976; Langer, 1987), an interaction with the ribosomal protein LI 6 is mentioned in more recent publications (Foster and Rybak, 1999). For another orthosomycin, avilamycin A, an inhibition of protein biosynthesis is assumed, and an inhibition of the translation-initiation complex is suggested (Wolf, 1973).

The known avilamycins were isolated in 1959 from culture filtrates of Streptomyces viridochromogenes Tü57 (Buzzetti et al., 1968; Mertz et al., 1986). As already indicated above, avilamycin A, one of the main components, is made up of sugars. Individual components are D-olivose, 2-deoxy-D-evalose, 4-O-methyl-D-fucose, 2,6-di-O-methyl-D-mannose, L-lyxose, and methyl eurekanate. In studies, avilamycin A demonstrated excellent activity against multiresistant Staphylococcus aureus strains (Zähner, 1999). In addition to the ortho esters, the terminal dichloroisoeverninic acid unit is supposed to be essential for its efficacy (Wright, 1979). DE 1116864 describes the substance class of avilamycins, including a general reference to derivatives, as well as the production and efficacy of avilamycins, as does U.S. Pat. No. 3,131,126.

Ziracin is also a member of the group of orthosomycins. Ziracin (SCH27899) is an evernimycin and has already been clinically tested.

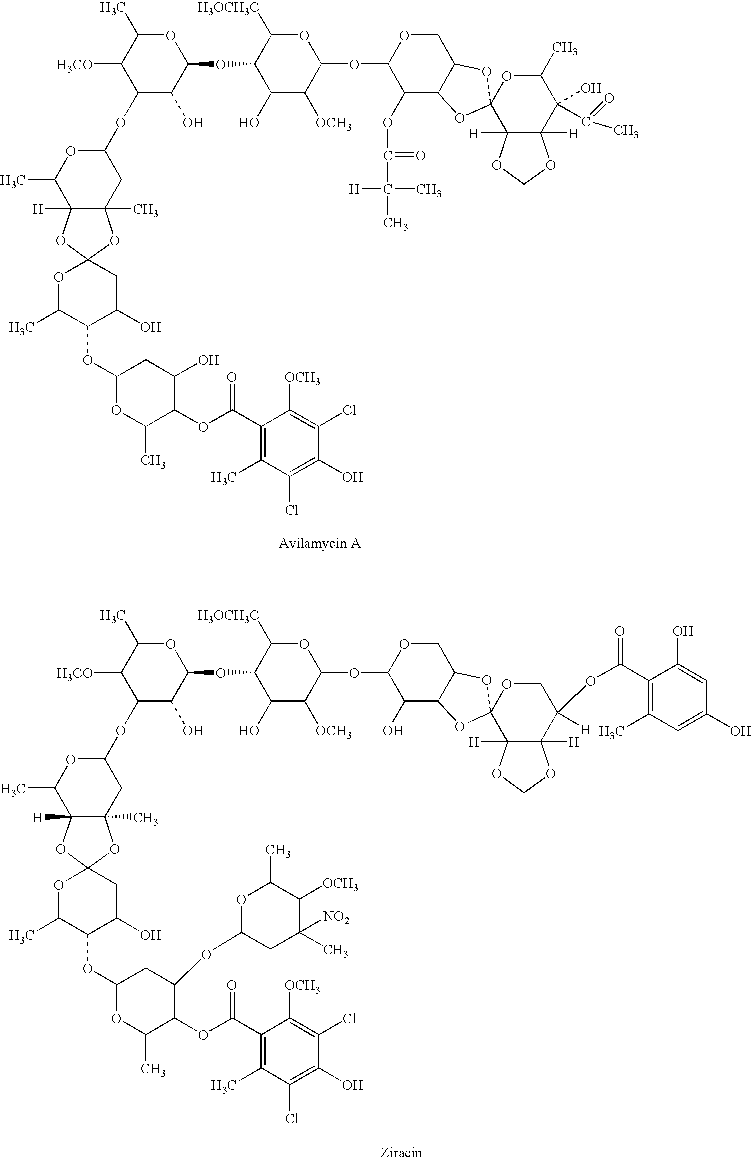

However, for both avilamycin A and Ziracin, practical experience has shown that therapeutic use appears to be limited due to an insufficient degree of hydrophilia.

Specifically for the class of orthosomycins, and particularly for the avilamycins, molecular cloning and characterization of the enzymes that determine the biosynthesis of avilamycin A should be of great interest, since this information could point the direction for the development of new (antimicrobial) antibiotics. The genes are an interesting system for studying the formation and linking of unusual deoxy sugars, and therefore might be of great value for combinatorial synthesis.

Earlier work on the biosynthetic gene cluster of avilamycins resulted in decoding the sequence of an NDP-glucose-synthase gene (avid [consecutive number 53 in accordance with Table 1]), an NDP-glucose-4,6-dehydratase gene (aviE1 [consecutive number 54 in accordance with Table 1]), and a polyketide synthase gene (aviM [consecutive number 52 in accordance with Table 1]). These presumably have a function as part of an interactive Type I polyketide synthase for the formation of orsellinic acid, an intermediate product in the biosynthesis of dichloroisoeverninic acid. The expression of aviM in S. lividans resulted in the formation of orsellinic acid [Gaisser, S., Trefzer, A., Stockert, S., Kirschning, A., and Bechthold, A. (1997), J. Bacteriol. 179, 6271-6278].

In addition to finding and identifying suitable enzyme systems and synthesis paths, it was therefore a task of the invention to make new antibiotics available, particularly including antibiotics that demonstrate improved hydrophilia.

### SUMMARY OF THE INVENTION

Surprisingly, it turned out that certain avilamycin derivatives (also referred to as gavibamycins according to the invention), not previously described in the state of the art, particularly those with a substitution pattern that is modified as compared with avilamycin A in decisive regions, are able to accomplish this task and demonstrate both an antibiotic effect and improved hydrophilia. An object of the invention is therefore an avilamycin derivative according to the general Formula I, also in the form of its diastereomers or enantiomers, its racemic mixtures or other mixtures or pure diastereomers and/or enantiomers,

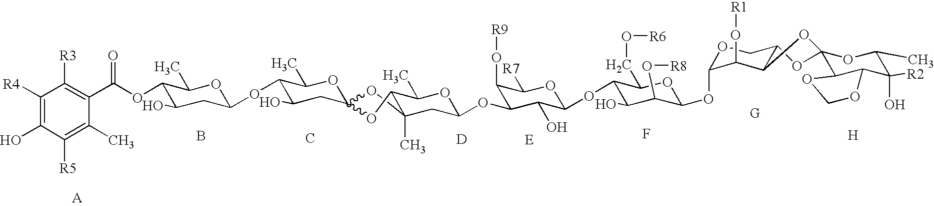

wherein, independently of one another, with the following exception,

R1 is selected from H, COCH₃, COC₄H₉, COCH(CH₃)₂ or COCH₂CH₃,

R2 is selected from H, CHO, COCH₃ or CH(OH)CH₃,

R3 corresponds to OCH₃,

R4 corresponds to Cl,

R5 corresponds to Cl,

R6 corresponds to CH₃,

R7 corresponds to H, CH₃ or CH₂OH,

R8 corresponds to CH₃, and

R9 corresponds to CH₃, and wherein the following applies with reference to at least one of the residues R3-R6, R8, or R9 in Formula I, in deviation from the above definition:

R3 is to be replaced by OH,

R4 is to be replaced by H,

R5 is to be replaced by H,

R6 is to be replaced by H,

R8 is to be replaced by H, and/or

R9 is to be replaced by H,

with the proviso that R1-R9 cannot simultaneously take on the meanings in accordance with the combination, in each instance, in one of the compounds 1-4:

| No. | R1 | R2 | R3 | R4 | R5 | R6 | R7 | R8 | R9 |
|---|---|---|---|---|---|---|---|---|---|
| 1 | COCH(CH₃)₂ | COCH₃ | OH | H | CI | CH₃ | CH₃ | CH₃ | CH₃ |
| 2 | COCH(CH₃)₂ | COCH₃ | OCH₃ | CI | H | CH₃ | CH₃ | CH₃ | CH₃ |
| 3 | COCH(CH₃)₂ | COCH₃ | OCH₃ | CI | CI | H | CH₃ | CH₃ | CH₃ |
| 4 | COCH(CH₃)₂ | COCH₃ | OCH₃ | CI | CI | CH₃ | CH₃ | H | CH₃ |

In this context, the expression “with the following exception” is to be understood to mean that there are exceptions from the general definitions of the moieties R1-R9 directly following this expression, which are introduced with the phrase “where the following applies with reference to at least one of the moieties R3-R6, R8, or R9 in Formula I, in deviation from the above definition.”

The avilamycin derivatives according to the invention are characterized, as a whole, not only by their surprisingly strong antibiotic activity against Staphylococcus aureus, but also by clearly improved hydrophilia, as compared with the known orthosomycins such as avilamycin A or C as well as evernimycin (=everninomycin=evernimicin). It is specifically this increased hydrophilia, however, that makes these compounds attractive, particularly antibiotic drugs, since increased hydrophilia is very desirable in specific therapeutic uses. In addition, for this avilamycin derivative, as for all the others, also those described below, it holds true that it exhibits a structure that can be developed only with difficulty using traditional organic synthesis. The use of gene technology biosynthesis for the production of the avilamycin derivatives according to the invention, which is a fundamental factor here, is thereby finding modified, new, and previously inaccessible active agents, particularly antibiotics.

### DETAILED DESCRIPTION OF THE INVENTION

Within the scope of the invention, an avilamycin derivative is preferred in which at least R3 is to be replaced by OH, with the proviso that R1-R9 cannot simultaneously take on the meanings in accordance with the combination in the compound 1:

| No. | R1 | R2 | R3 | R4 | R5 | R6 | R7 | R8 | R9 |
|---|---|---|---|---|---|---|---|---|---|
| 1 | COCH(CH₃)₂ | COCH₃ | OH | H | Cl | CH₃ | CH₃ | CH₃ | CH₃ |

Likewise, an avilamycin derivative according to the invention in which at least R4 and R5 in Formula I are to be replaced by H is preferred.

Likewise, an avilamycin derivative in which at least R6, R8 and/or R9 is/are to be replaced by H, with the proviso that R1-R9 cannot simultaneously take on the meanings in accordance with the combination in the compound 3 or cannot simultaneously take on the meanings in accordance with the combination in the compound 4:

| No. | R1 | R2 | R3 | R4 | R5 | R6 | R7 | R8 | R9 |
|---|---|---|---|---|---|---|---|---|---|
| 3 | COCH(CH₃)₂ | COCH₃ | OCH₃ | Cl | Cl | H | CH₃ | CH₃ | CH₃ |
| 4 | COCH(CH₃)₂ | COCH₃ | OCH₃ | Cl | Cl | CH₃ | CH₃ | H | CH₃ |

It is furthermore preferred to combine these characteristics, resulting in an avilamycin derivative according to the invention in which at least R3 is to be replaced by OH, for one thing, and at least R4 and R5 are to be replaced by H, for another thing, or at least R6, R8 and/or R9 is/are to be replaced by H.

In this context, an especially preferred object of the invention, which accomplishes the task in a particularly advantageous manner, is an avilamycin derivative according to the general Formula I that is selected from among compounds in which R1-R9 have the meaning as indicated in the following table, in each instance, and are combined as follows:

| No. | R1 | R2 | R3 | R4 | R5 | R6 | R7 | R8 | R9 |
|---|---|---|---|---|---|---|---|---|---|
| 5 | COCH(CH₃)₂ | COCH₃ | OH | Cl | Cl | CH₃ | CH₃ | CH₃ | CH₃ |
| 6 | COCH₂CH₃ | H | OH | Cl | Cl | CH₃ | CH₃ | CH₃ | CH₃ |
| 7 | COCH₃ | COCH₃ | OH | Cl | Cl | CH₃ | CH₃ | CH₃ | CH₃ |
| 8 | COCH(CH₃)₂ | CH(OH)CH₃ | OH | Cl | Cl | CH₃ | CH₃ | CH₃ | CH₃ |
| 9 | H | COCH₃ | OH | Cl | Cl | CH₃ | CH₃ | CH₃ | CH₃ |
| 10 | COCH₃ | CH(OH)CH₃ | OH | Cl | Cl | CH₃ | CH₃ | CH₃ | CH₃ |
| 11 | H | CH(OH)CH₃ | OH | Cl | Cl | CH₃ | CH₃ | CH₃ | CH₃ |
| 12 | COC₄H₉ | COCH₃ | OH | Cl | Cl | CH₃ | CH₃ | CH₃ | CH₃ |
| 13 | COCH(CH₃)₂ | COCH₃ | OH | Cl | H | CH₃ | CH₃ | CH₃ | CH₃ |
| 14 | COCH₂CH₃ | COCH₃ | OH | Cl | Cl | CH₃ | CH₃ | CH₃ | CH₃ |
| 15 | COCH(CH₃)₂ | COCH₃ | OH | Cl | Cl | H | CH₃ | CH₃ | CH₃ |
| 16 | COCH(CH₃)₂ | COCH₃ | OH | Cl | Cl | CH₃ | CH₂OH | CH₃ | CH₃ |
| 17 | COCH(CH₃)₂ | CHO | OH | Cl | Cl | CH₃ | CH₃ | CH₃ | CH₃ |
| 18 | COCH(CH₃)₂ | COCH₃ | OH | Cl | Cl | CH₃ | H | CH₃ | CH₃ |
| 19 | COCH(CH₃)₂ | COCH₃ | OH | Cl | Cl | CH₃ | CH₃ | H | CH₃ |
| 20 | COCH(CH₃)₂ | COCH₃ | OH | H | H | CH₃ | CH₃ | CH₃ | CH₃ |
| 21 | COCH₂CH₃ | H | OH | H | H | CH₃ | CH₃ | CH₃ | CH₃ |
| 22 | COCH₃ | COCH₃ | OH | H | H | CH₃ | CH₃ | CH₃ | CH₃ |
| 23 | COCH(CH₃)2 | CH(OH)CH₃ | OH | H | H | CH₃ | CH₃ | CH₃ | CH₃ |
| 24 | H | COCH₃ | OH | H | H | CH₃ | CH₃ | CH₃ | CH₃ |
| 25 | COCH₃ | CH(OH)CH₃ | OH | H | H | CH₃ | CH₃ | CH₃ | CH₃ |
| 26 | H | CH(OH)CH₃ | OH | H | H | CH₃ | CH₃ | CH₃ | CH₃ |
| 27 | COCH(CH₃)₂ | COCH₃ | OH | H | H | CH₃ | CH₃ | CH₃ | CH₃ |
| 28 | COC₄H₉ | COCH₃ | OH | H | H | CH₃ | CH₃ | CH₃ | CH₃ |
| 29 | COCH(CH₃)₂ | COCH₃ | OH | H | H | CH₃ | CH₃ | CH₃ | CH₃ |
| 30 | COCH₂CH₃ | COCH₃ | OH | H | H | CH₃ | CH₃ | CH₃ | CH₃ |
| 31 | COCH(CH₃)₂ | COCH₃ | OH | H | H | H | CH₃ | CH₃ | CH₃ |
| 32 | COCH(CH₃)₂ | COCH₃ | OH | H | H | CH₃ | CH₂OH | CH₃ | CH₃ |
| 33 | COCH(CH₃)₂ | CHO | OH | H | H | CH₃ | CH₃ | CH₃ | CH₃ |
| 34 | COCH(CH₃)₂ | COCH₃ | OH | H | H | CH₃ | H | CH₃ | CH₃ |
| 35 | COCH(CH₃)₂ | COCH₃ | OH | H | H | CH₃ | CH₃ | H | CH₃ |
| 36 | COCH(CH₃)₂ | COCH₃ | OH | Cl | Cl | H | CH₃ | CH₃ | CH₃ |
| 37 | COCH₂CH₃ | H | OH | Cl | Cl | H | CH₃ | CH₃ | CH₃ |
| 38 | COCH₃ | COCH₃ | OH | Cl | Cl | H | CH₃ | CH₃ | CH₃ |
| 39 | COCH(CH₃)₂ | CH(OH)CH₃ | OH | Cl | Cl | H | CH₃ | CH₃ | CH₃ |
| 40 | H | COCH₃ | OH | Cl | Cl | H | CH₃ | CH₃ | CH₃ |
| 41 | COCH₃ | CH(OH)CH₃ | OH | Cl | Cl | H | CH₃ | CH₃ | CH₃ |
| 42 | H | CH(OH)CH₃ | OH | Cl | Cl | H | CH₃ | CH₃ | CH₃ |
| 43 | COCH(CH₃)₂ | COCH₃ | OH | H | Cl | H | CH₃ | CH₃ | CH₃ |
| 44 | COC₄H₉ | COCH₃ | OH | Cl | Cl | H | CH₃ | CH₃ | CH₃ |
| 45 | COCH(CH₃)₂ | COCH₃ | OH | Cl | H | H | CH₃ | CH₃ | CH₃ |
| 46 | COCH₂CH₃ | COCH₃ | OH | Cl | Cl | H | CH₃ | CH₃ | CH₃ |
| 47 | COCH(CH₃)₂ | COCH₃ | OH | Cl | Cl | H | CH₃ | CH₃ | CH₃ |
| 48 | COCH(CH₃)₂ | COCH₃ | OH | Cl | Cl | H | CH₂OH | CH₃ | CH₃ |
| 49 | COCH(CH₃)₂ | CHO | OH | Cl | Cl | H | CH₃ | CH₃ | CH₃ |
| 50 | COCH(CH₃)₂ | COCH₃ | OH | Cl | Cl | H | H | CH₃ | CH₃ |
| 51 | COCH(CH₃)₂ | COCH₃ | OH | Cl | Cl | H | CH₃ | H | CH₃ |
| 52 | COCH(CH₃)₂ | COCH₃ | OH | Cl | Cl | CH₃ | CH₃ | H | CH₃ |
| 53 | COCH₂CH₃ | H | OH | Cl | Cl | CH₃ | CH₃ | H | CH₃ |
| 54 | COCH₃ | COCH₃ | OH | Cl | Cl | CH₃ | CH₃ | H | CH₃ |
| 55 | COCH(CH₃)₂ | CH(OH)CH₃ | OH | Cl | Cl | CH₃ | CH₃ | H | CH₃ |
| 56 | H | COCH₃ | OH | Cl | Cl | CH₃ | CH₃ | H | CH₃ |
| 57 | COCH₃ | CH(OH)CH₃ | OH | Cl | Cl | CH₃ | CH₃ | H | CH₃ |
| 58 | H | CH(OH)CH₃ | OH | Cl | Cl | CH₃ | CH₃ | H | CH₃ |
| 59 | COCH(CH₃)₂ | COCH₃ | OH | H | Cl | CH₃ | CH₃ | H | CH₃ |
| 60 | COC₄H₉ | COCH₃ | OH | Cl | Cl | CH₃ | CH₃ | H | CH₃ |
| 61 | COCH(CH₃)₂ | COCH₃ | OH | Cl | H | CH₃ | CH₃ | H | CH₃ |
| 62 | COCH₂CH₃ | COCH₃ | OH | Cl | Cl | CH₃ | CH₃ | H | CH₃ |
| 63 | COCH(CH₃)₂ | COCH₃ | OH | Cl | Cl | H | CH₃ | H | CH₃ |
| 64 | COCH(CH₃)₂ | COCH₃ | OH | Cl | Cl | CH₃ | CH₂OH | H | CH₃ |
| 65 | COCH(CH₃)₂ | CHO | OH | Cl | Cl | CH₃ | CH₃ | H | CH₃ |
| 66 | COCH(CH₃)₂ | COCH₃ | OH | Cl | Cl | CH₃ | H | H | CH₃ |
| 67 | COCH(CH₃)₂ | COCH₃ | OH | Cl | Cl | CH₃ | CH₃ | H | CH₃ |
| 68 | COCH(CH₃)₂ | COCH₃ | OH | Cl | Cl | CH₃ | CH₃ | CH₃ | H |
| 69 | COCH₃CH₃ | H | OH | Cl | Cl | CH₃ | CH₃ | CH₃ | H |
| 70 | COCH₃ | COCH₃ | OH | Cl | Cl | CH₃ | CH₃ | CH₃ | H |
| 71 | COCH(CH₃)₂ | CH(OH)CH₃ | OH | Cl | Cl | CH₃ | CH₃ | CH₃ | H |
| 72 | H | COCH₃ | OH | Cl | Cl | CH₃ | CH₃ | CH₃ | H |
| 73 | COCH₃ | CH(OH)CH₃ | OH | Cl | Cl | CH₃ | CH₃ | CH₃ | H |
| 74 | H | CH(OH)CH₃ | OH | Cl | Cl | CH₃ | CH₃ | CH₃ | H |
| 75 | COCH(CH₃)₂ | COCH₃ | OH | H | Cl | CH₃ | CH₃ | CH₃ | H |
| 76 | COC₄H₉ | COCH₃ | OH | Cl | Cl | CH₃ | CH₃ | CH₃ | H |
| 77 | COCH(CH₃)₂ | COCH₃ | OH | Cl | H | CH₃ | CH₃ | CH₃ | H |
| 78 | COCH₂CH₃ | COCH₃ | OH | Cl | Cl | CH₃ | CH₃ | CH₃ | H |
| 79 | COCH(CH₃)₂ | COCH₃ | OH | Cl | Cl | H | CH₃ | CH₃ | H |
| 80 | COCH(CH₃)₂ | COCH₃ | OH | Cl | Cl | CH₃ | CH₂OH | CH₃ | H |
| 81 | COCH(CH₃)₂ | CHO | OH | Cl | Cl | CH₃ | CH₃ | CH₃ | H |
| 82 | COCH(CH₃)₂ | COCH₃ | OH | Cl | Cl | CH₃ | H | CH₃ | H |
| 83 | COCH(CH₃)₂ | COCH₃ | OH | Cl | Cl | CH₃ | CH₃ | H | H |
| 84 | COCH(CH₃)₂ | COCH₃ | OH | Cl | Cl | H | CH₃ | H | H |
| 85 | COCH₂CH₃ | H | OH | Cl | Cl | H | CH₃ | H | H |
| 86 | COCH₃ | COCH₃ | OH | Cl | Cl | H | CH₃ | H | H |
| 87 | COCH(CH₃)₂ | CH(OH)CH₃ | OH | Cl | Cl | H | CH₃ | H | H |
| 88 | H | COCH₃ | OH | Cl | Cl | H | CH₃ | H | H |
| 89 | COCH₃ | CH(OH)CH₃ | OH | Cl | Cl | H | CH₃ | H | H |
| 90 | H | CH(OH)CH₃ | OH | Cl | Cl | H | CH₃ | H | H |
| 91 | COCH(CH₃)₂ | COCH₃ | OH | H | Cl | H | CH₃ | H | H |
| 92 | COC₄H₉ | COCH₃ | OH | Cl | Cl | H | CH₃ | H | H |
| 93 | COCH(CH₃)₂ | COCH₃ | OH | Cl | H | H | CH₃ | H | H |
| 94 | COCH₂CH₃ | COCH₃ | OH | Cl | Cl | H | CH₃ | H | H |
| 95 | COCH(CH₃)₂ | COCH₃ | OH | Cl | Cl | H | CH₃ | H | H |
| 96 | COCH(CH₃)₂ | COCH₃ | OH | Cl | Cl | H | CH₂OH | H | H |
| 97 | COCH(CH₃)₂ | CHO | OH | Cl | Cl | H | CH₃ | H | H |
| 98 | COCH(CH₃)₂ | COCH₃ | OH | Cl | Cl | H | H | H | H |
| 99 | COCH(CH₃)₂ | COCH₃ | OH | Cl | Cl | H | CH₃ | H | H |

preferably,

| R1 | R2 | R3 | R4 | R5 | R6 | R7 | R8 | R9 |
|---|---|---|---|---|---|---|---|---|
| COCH(CH₃)₂ | COCH₃ | OH | Cl | Cl | CH₃ | CH₃ | CH₃ | CH₃ |
| COCH(CH₃)₂ | CH(OH)CH₃ | OH | Cl | Cl | CH₃ | CH₃ | CH₃ | CH₃ |
| COCH(CH₃)₂ | COCH₃ | OH | H | H | CH₃ | CH₃ | CH₃ | CH₃ |
| COCH(CH₃)₂ | CH(OH)CH₃ | OH | H | H | CH₃ | CH₃ | CH₃ | CH₃ |
| COCH(CH₃)₂ | COCH₃ | OH | Cl | Cl | H | CH₃ | CH₃ | CH₃ |
| COCH(CH₃)₂ | CH(OH)CH₃ | OH | Cl | Cl | H | CH₃ | CH₃ | CH₃ |
| COCH(CH₃)₂ | COCH₃ | OH | Cl | Cl | CH₃ | CH₃ | H | CH₃ |
| COCH(CH₃)₂ | CH(OH)CH₃ | OH | Cl | Cl | CH₃ | CH₃ | H | CH₃ |
| COCH(CH₃)₂ | COCH₃ | OH | Cl | Cl | CH₃ | CH₃ | CH₃ | H |
| COCH(CH₃)₂ | CH(OH)CH₃ | OH | Cl | Cl | CH₃ | CH₃ | CH₃ | H |

The task is also accomplished by avilamycin derivatives that can be produced by means of a special process that involves gene technology manipulations and biosynthesis. Another object of the invention is therefore an avilamycin derivative that can be obtained in a cell that can be cultivated that exhibits the necessary genes and/or enzymes for the synthesis of an orthosomycin basic body consisting of:

a) a terminal dichloroisoeverninic acid moiety (A in Formula I); and

b) a heptasaccharide esterified with it, linked via normal ester bonds and ortho ester bonds (B to H in Formula I), comprised of:

(i) two D-olivose moieties (B and C);

(ii) a 2-deoxy-D-evalose moiety (D);

(iii) a D-fucose moiety (E);

(iv) a D-mannose moiety (F);

(v) an L-lyxose moiety (G); and

(vi) a (methyl) eurekanate moiety (H),

by modifying with gene technology, deleting, and/or not expressing at least one nucleic acid, the sequence of which corresponds by at least 95%, preferably 97%, and particularly precisely to the nucleic acid sequence in accordance with one of FIGS. 1 to 54, by cultivating the cell in this way, by receiving and processing the top portion of the culture, by purifying and isolating the avilamycin derivative(s), and, if applicable, by separating different derivatives from one another, with the proviso that R1-R9 cannot simultaneously take on the meanings in accordance with the combination as shown below:

| R1 | R2 | R3 | R4 | R5 | R6 | R7 | R8 | R9 |
|---|---|---|---|---|---|---|---|---|
| COCH(CH₃)₂ | COCH₃ | OH | H | Cl | CH₃ | CH₃ | CH₃ | CH₃ |
| COCH(CH₃)₂ | COCH₃ | OCH₃ | Cl | H | CH₃ | CH₃ | CH₃ | CH₃ |
| COCH(CH₃)₂ | COCH₃ | OCH₃ | Cl | Cl | H | CH₃ | CH₃ | CH₃ |
| COCH(CH₃)₂ | COCH₃ | OCH₃ | Cl | Cl | CH₃ | CH₃ | H | CH₃ |
| COCH(CH₃)₂ | CHO | OCH₃ | Cl | Cl | CH₃ | CH₃ | CH₃ | CH₃ |
| COCH(CH₃)₂ | COCH₃ | OCH₃ | Cl | Cl | CH₃ | H | CH₃ | CH₃ |
| COCH(CH₃)₂ | COCH₃ | OCH₃ | Cl | Cl | CH₃ | CH₃ | CH₃ | CH₃ |
| COCH₂CH₃ | H | OCH₃ | Cl | Cl | CH₃ | CH₃ | CH₃ | CH₃ |
| COCH₃ | COCH₃ | OCH₃ | Cl | Cl | CH₃ | CH₃ | CH₃ | CH₃ |
| COCH(CH₃)₂ | CH(OH)CH₃ | OCH₃ | Cl | Cl | CH₃ | CH₃ | CH₃ | CH₃ |
| H | COCH₃ | OCH₃ | Cl | Cl | CH₃ | CH₃ | CH₃ | CH₃ |
| COCH₃ | CH(OH)CH₃ | OCH₃ | Cl | Cl | CH₃ | CH₃ | CH₃ | CH₃ |
| H | CH(OH)CH₃ | OCH₃ | Cl | Cl | CH₃ | CH₃ | CH₃ | CH₃ |
| COC₄H₉ | COCH₃ | OCH₃ | Cl | Cl | CH₃ | CH₃ | CH₃ | CH₃ |
| COCH₂CH₃ | COCH₃ | OCH₃ | Cl | Cl | CH₃ | CH₃ | CH₃ | CH₃ |
| COCH(CH)₂ | COCH₃ | OCH₃ | Cl | CL | CH₃ | CH₂OH | CH₃ | CH₃ |

In this context, in the sense of the invention, one understands the statement that “the cell exhibits the necessary genes and/or enzymes for the synthesis of an orthosomycin basic body” to mean that the genes that code for the necessary enzymes and/or the functional enzymes themselves, which are necessary for the synthesis of an “orthosomycin basic body” from the precursor stages that are usually present are present in the cell. Examples would be the gene cluster according to the invention in accordance with FIG. 109 or the “Open Reading Frames” (ORF), and/or genes in accordance with consecutive number 1-54 in accordance with Table 1 in combination with FIG. 1, and/or the related enzymes and/or proteins in accordance with consecutive number 55-108 in accordance with Table 1 in combination with FIG. 1.

The definition of the “orthosomycin basic body” has already been given, where the arrangement of the ortho ester bond and the normal ester bond can be derived from Formula I. Avilamycins and evernimycins, among others, as well as avilamycin derivatives according to the invention, exhibit such a basic body (see Formula I).

Furthermore, in the sense of this invention, gene is understood to mean a segment of DNA, from which an individual mRNA molecule (which is then translated into an individual polypeptide or protein) or a functional RNA molecule (rRNA, tRNA) is transcribed.

Furthermore, in the sense of this invention, an “Open Reading Frame” (ORF) is understood to mean a DNA segment that begins with a start codon, ends with a stop codon, and contains an uninterrupted sequence of codons for amino acids. Here, the term “Open Reading Frame” (ORF) is used to describe a cloned and sequenced DNA segment that corresponds to a gene.

Codon is understood to mean the coding genetic basic unit. It consists of a triplet of three consecutive nucleotides that code either for an amino acid or for the start or end of a polypeptide chain.

Furthermore, in the sense of this invention, cells that can be cultivated are understood to mean cells that grow and reproduce in vitro in a solid or liquid medium, nourished by a liquid or solidified nutrient solution, the culture medium. In the narrower sense, these are specifically cells of microorganisms or easily transfectable cells in which the corresponding genes can be expressed. For example, they can be cells of gram-positive and gram-negative bacteria, such as Streptomyces cells (e.g., Streptomyces viridochromogenes Tü57), but also systems such as mammalian cells, e.g., CHO cells (Chinese hamster ovary), or immortalized cell lines, such as HeLa or HEK cells, but also insect, fish, amphibian, fungus, or yeast cells, etc.

In the sense of this invention, a nucleic acid is understood to mean the basic unit of DNA and RNA and therefore particularly also the basic unit of a gene and an ORF. Analogously, a nucleic acid can comprise a gene or an ORF, and a specific nucleic acid sequence (the sequence of bases on the phosphate-sugar backbone of a nucleic acid) can define a gene or an ORF. The term nucleic acid is also understood to mean sequences that contain additional sequence regions, other than the coding regions, particularly at the 5′ or the 3′ end of the coding region. These sequences can be without function or can be promoter or enhancer signals, preferably bacterial signals or signals corresponding to the host cell system used for expression. In addition to the sequence regions that code for the proteins according to the invention, those nucleotide sequences that code for so-called “tags” (e.g., His or Flag Tag) are very especially preferred, so that the proteins according to the invention that are expressed in the host cells can be easily purified, for example by way of affinity chromatography. In this way, any desired sequences that code for amino acid sequences and contain a tag (for example an antigen) for binding to an antibody, for example on a column, can be appended to coding nucleotide sequences according to the invention, preferably at the 5′ or the 3′ end. Therefore amino acid sequences that result from the combination of coding nucleic acids according to the invention with other nucleotide sequences are also disclosed.

Gene technology, in the sense of the invention, is understood to mean the use of various techniques by means of which DNA is introduced into a host cell or that the DNA of a cell is specifically changed. This includes the use of cloning techniques, vectors, restriction enzymes, etc.

Analogously, modified by gene technology means that an intervention has changed the base sequence, the sequence of the nucleic acid, particularly that it has shortened the base sequence (going as far as deletion), or that mutations were built in, in most cases with the consequence that the nucleic acid (the gene) cannot be transcribed into an mRNA, or can be transcribed only in modified form.

In this case, deleted means that a nucleic acid that in most cases comprises a gene or an ORF here has been removed from the DNA, entirely or at least to a great extent, so that the nucleic acid (the gene) cannot be transcribed into an mRNA, or can be transcribed only in modified form. Analogously, not expressed means that the nucleic acid was modified in such a way that the nucleic acid (gene) cannot be transcribed into an mRNA, or can be transcribed only in modified form, and therefore, the polypeptide or protein for which the nucleic acid (the gene or the ORF) originally coded is no longer formed by translation.

Moderately stringent hybridization conditions are understood to mean different standard conditions, depending on the nucleic acid sequence being used (oligonucleotide, longer fragment, or complete sequence), and also depending on what type of nucleic acid (DNA or RNA) is being used for hybridization. For example, the melting temperatures for DNA:DNA hybrids are approximately 10° C. lower than those for DNA:RNA hybrids with the same length. Standard conditions are understood to mean, for example, depending on the nucleic acid, temperatures between 42 and 58° C. in an aqueous buffer solution with a concentration from 0.1 to 5×SSC (1×SSC=0.15 M NaCl, 15 mM sodium citrate, pH 7.2) or additionally in the presence of 50% formamide, for example 42° C. in 5×SSC, 50% formamide. It is advantageous if the hybridization conditions for DNA:DNA hybrids are at 0.1×SSC and the temperatures between approximately 20° C. to 45° C., preferably between approximately 30° C. to 45° C. For DNA:RNA hybrids, the hybridization conditions are advantageously 0.1×SSC and temperature between approximately 30° C. to 55° C., preferably between approximately 45° C. to 55° C. These temperatures as indicated for hybridization are melting temperature values calculated as an example for a nucleic acid with a length of approximately 100 nucleotides and a G+C content of 50% in the absence of formamide. The experimental conditions for DNA hybridization are described in relevant textbooks about genetics, such as Sambrook et al. (“Molecular Cloning,” Cold Spring Harbor Laboratory, 1989), and can be calculated according to formulas known to a person skilled in the art, for example as a function of the length of the nucleic acids, the type of hybrids, or the G+C content. Additional information concerning hybridization can be obtained by a person skilled in the art from the following textbooks: Ausübel et al. (eds.), Current Protocols in Molecular Biology, John Wiley & Sons, New York (1985); Hames and Higgins (eds.), Nucleic Acids Hybridization: A Practical Approach, IRL Press at Oxford University Press, Oxford (1985); Brown (ed.), Essential Molecular Biology: A Practical Approach, IRL Press at Oxford University Press, Oxford (1991).

Cultivation in the sense of this invention is understood to mean in vitro cultivation of cells that can be cultivated, where these grow and reproduce in a solid or liquid medium, nourished by a liquid or solidified nutrient solution.

In this context, top culture portion is understood to mean the liquid culture medium that contains not only the nutrients for the cells that can be cultivated but also the metabolites and substances given off by them to the outside, into the medium (e.g. avilamycin derivatives). This top culture portion can be recovered and processed, which is understood specifically to mean suctioning off and/or filtering the top portion to separate the solids left over from cultivation and from the cells.

The top culture portion, which in most cases contains avilamycin derivatives according to the invention, can be purified after processing; this is generally understood to mean separation by chromatography and/or separation by way of liquid phases, or a combination of these methods of procedure. Examples for this are solid phase extraction with a methanol-in-water gradient or ethyl acetate extraction. In this context, the fraction that contains the avilamycin derivatives is separated, to a great extent, from the other fractions that contain other components of the top portion, and thereby the avilamycin derivatives are isolated to a great extent. Other alternative separation and/or purification processes are the method of salting-out or recrystallization or crystallizing-out. If necessary, this can be followed by isolation and separation of the individual derivatives, where chromatography methods, in particular, are used to do so. HPLC methods or affinity chromatography methods are especially preferred.

It is preferred for the production method by way of which the avilamycin derivative according to the invention is defined if the cell that can be cultivated is selected from a cell of the type Streptomyces viridochromogenes or a cell that, with the exception of the nucleic acid(s) modified by gene technology, deleted, or not expressed, contains the nucleic acids in accordance with consecutive number 1-54 in accordance with Table 1 in combination with FIG. 1, and/or nucleic acids that are homologous to it by at least 95%, preferably 97%, or hybridizes with one of these sequences under moderately stringent conditions, or contains the gene cluster in accordance with FIG. 109. The second point of selection is particularly understood to mean cells in which the enzymes necessary for avilamycin derivative synthesis are expressed using gene technology methods, where one of the nucleic acids that codes for the enzyme that occurs endogenously in Streptomyces viridochromogenes Tü 57 is modified by gene technology or deleted or not expressed, in particular that the nucleic acid/DNA is not introduced into the host cell by gene technology in the first place. However, it is especially preferred if the cell is selected from a cell of the type Streptomyces viridochromogenes, particularly a cell of the type Streptomyces viridochromogenes Tü 57 or A 23575.

In every case, it is preferred if the modified (e.g., deleted or not introduced into the host cell) nucleic acid(s) coded for a methyl transferase and/or for a halogenase.

Alternatively, the production can also take place outside an in vivo process, as an in vitro synthesis. In this context, the enzymes and/or enzyme systems required for synthesis are presented in at least one experimental batch, where preferably the reaction steps necessary for synthesis are carried out in several consecutive experimental batches, by catalysis, by the required enzymes and enzymes according to the invention. If necessary, separation and/or purification steps can be inserted between the individual reactions, which are carried out in a suitable sequence, to purify the desired intermediate products, in each instance.

Methyl transferases are understood to be enzymes that can transfer a methyl group to an organic molecule. In the sense of the invention, these are particularly enzymes that transfer a methyl group either to the orsellinic acid or to the sugars, preferably after the formation of a heptasaccharide, particularly the ORFs aviG2, aviG3, aviG5, aviG6, aviG1, aviG4, aviRa, and aviRb, particularly aviG4.

Halogenases are understood to be enzymes that can enzymatically transfer halogens to organic molecules. In the sense of the invention, these are particularly enzymes that transfer one, preferably two Cl atoms onto orsellinic acid, at the positions R4 and/or R5, particularly the ORF aviH.

Analogously, it is a particularly preferred object of the invention if, with regard to the production process indicated above, the sequence(s) of the modified nucleic acid(s) before being modified correspond(s) by at least 95%, preferably 97%, and particularly precisely to the nucleic acid sequence(s) of at least one of the sequences in accordance with consecutive number 1 or 2-7 in accordance with Table 1 in combination with FIG. 1, preferably one of the sequences with consecutive number 1, 2, 4, or 6 (Table 1 in combination with FIG. 1), particularly the sequence with consecutive number 2 or the sequences with consecutive numbers 2 and 1, numbers 2 and 4, or numbers 2 and 6 (in accordance with Table 1 in combination with FIG. 1), or if it hybridizes with one of these sequences under moderately stringent conditions.

In this context, “before modification” in the sense of this invention means that the modified nucleic acid demonstrates the stated nucleic acid sequence before it is manipulated by gene technology; before any deletion or modification, particularly shortening or mutation in the base sequence, but also before the step of not even introducing this nucleic acid/DNA into the host cell by means of gene technology.

Furthermore, it is preferred if the modification of the nucleic acid(s) in the production process that defines the avilamycin derivatives has the result that the protein(s) or polypeptide(s) coded by the nucleic acid(s) modified by gene technology is/are no longer synthesized after the modification by gene technology.

In this context, polypeptide is understood to mean a peptide with between 10≦ and <100 amino acid residues and a protein is understood to mean a macromolecule with more than 10 amino acid residues linked via peptide bonds. In this context, the proteins in connection with this invention are preferably enzymes. Of course, other proteins in the sense of this invention are covered by this term.

The avilamycin derivatives according to the invention that have been described so far predominantly or all have the advantage, as compared with the related orthosomycins described in the state of the art, particularly as compared with avilamycin A, of being more hydrophilic, and this offers significant advantages therapeutically. This is particularly true for a comparison with avilamycin A or C or also with the evernimycin ziracin.

It was also a task of the invention—in addition to making new antibiotics available—to clarify the synthesis of avilamycin A, in order to develop new antimicrobial substances based on it, and new methods for their production. A key point in this context was molecular cloning and characterization of the genes involved in avilamycin biosynthesis. A piece with a size of about 60 kb around the known genes avid, aviE1 and aviM was sequenced. In this context, it turned out that the genes involved were arranged in the immediate vicinity of one another in a cluster. The sequence of the individual ORFs as well as their arrangement on the central gene cluster (consecutive numbers 1 to 54) are shown in FIG. 1 in combination with Table 1, as in FIG. 109. As already explained, the sequence of an NDP-glucose-synthase gene (avid [consecutive number 53 in accordance with Table 1 in combination with FIG. 1]), an NDP-glucose-4,6-dehydratase gene (aviE1 [consecutive number 54 in accordance with Table 1 in combination with FIG. 1]), and a polyketide synthase gene (aviM [consecutive number 52 in accordance with Table 1 in combination with FIG. 1]) were known, as was their presumed function as part of an interactive Type I polyketide synthase for the formation of orsellinic acid, an intermediate product in the biosynthesis of dichloroisoeverninic acid [Gaisser, S., Trefzer, A., Stockert, S., Kirschning, A., and Bechthold, A., J. Bacteriol., 179:6271-6278 (1997)].

The sequences of the other ORFs involved in the synthesis of avilamycins, which were discovered as a result of extensive cloning, can also be seen in FIG. 1, as can their relative arrangement on the gene cluster of FIG. 109. Here, giving the consecutive number from Table 1 makes it possible to assign the designation of the ORFs by name. Under the designation by name, the sequences can be derived from FIG. 1, specifically in the manner shown in the description of FIG. 1. The precise cloning strategy as well as further details regarding sequencing are presented in the examples, as are the functional analysis and characterization of the genes found (ORFs). The assignment of ORF abbreviations to function and sequence (including derived protein sequence) can be derived from Table 1, which follows the figure description.

Another important object of the invention is therefore one (or several) nucleic acid(s) that correspond(s) by at least 95%, preferably 97%, and particularly precisely to the nucleic acid sequence in accordance with one of the sequences of consecutive number 1 to 51 in accordance with Table 1 in combination with FIG. 1, or hybridizes with one of these sequences under moderately stringent conditions. In particular, sequences with the consecutive numbers 48 and 49 (in accordance with Table 1 and sequence presentation in FIG. 1) with a function as rRNA-methyl transferases (aviRa and avirb) and also the sequences with the consecutive numbers 50 and 51 (in accordance with Table 1 in combination with FIG. 1) with a function as ABC transporter genes (aviABC1 and aviABC2), which impart resistance to avilamycins and/or sequences that correspond by at least 95% with these sequences with the aforementioned consecutive numbers, or hybridize with one of these sequences under moderately stringent conditions, are described in the present invention. For the remainder, even mixtures of nucleic acids that represent any subcombination of the nucleic acids presented in accordance with FIG. 1, with the consecutive numbers 1 to 51 from Table 1, for example mixtures of two, three, four, . . . , 50 nucleic acids in any combination, are also disclosed according to the invention, if applicable also as a combination on one nucleic acid strand or on different strands.

In this context, (a) nucleic acid(s) that correspond(s) by at least 95%, preferably 97%, and particularly precisely with the nucleic acid sequence in accordance with one of the sequences with the consecutive number 1 to 32 in accordance with Table 1 (in combination with FIG. 1), preferably 1 to 7, particularly 1, 2, 4, or 6, or one of the sequences with the consecutive number 48 to 51 or 43, 44, or 46 in accordance with Table 1 (in combination with FIG. 1) or that hybridize(s) with one of the sequences under moderately stringent conditions is/are particularly preferred.

Analogously, gene clusters that contain “Open reading frames,” preferably 54, which correspond in their nucleic acid sequence by at least 95%, preferably 97%, and particularly precisely to the nucleic acid sequences according to the sequences with the consecutive numbers 1 to 54 (Table 1 in combination with FIG. 1) or hybridize with one of these sequences under moderately stringent conditions and that are arranged on a nucleic acid strand or in any combination on one of the other strands, preferably in accordance with FIG. 109, are another object of the invention. The genes in a gene cluster according to the invention can contain 2, three, four, . . . , 54 genes according to the invention, in any strand distribution and subcombination, if applicable in combination with the genes already known, and in particular, the segments located between the ORFs can be any nucleotide sequence.

This particularly relates to a gene cluster in accordance with FIG. 109, but also to gene clusters that contain corresponding nucleic acids, possibly also in another arrangement, where it is preferred, but not necessary, that all the ORFs can be found in the gene cluster in accordance with the consecutive numbers 1-54 (Table 1 in combination with FIG. 1).

The term gene cluster in the sense of this invention is understood to mean a segment of a DNA on which several genes are located in close spatial proximity. Such gene clusters according to the invention can be present in a vector, for example a BAC or a YAC, a cosmid or a plasmid. Vectors that contain at least one sequence according to the invention are therefore also an object of the present invention. Genes according to the invention can be combined in vectors according to the invention, with additional signal sequences or additional genes, in particular additional antibiotic resistance genes.

It was possible to derive protein and polypeptide sequences from the newly discovered sequences of the ORFs or genes. Accordingly, another object of the invention is a protein or polypeptide that corresponds by at least 95%, preferably 97%, or particularly precisely to the amino acid sequence in accordance with one of the sequences with the consecutive numbers 55-101 (Table 1 in combination with FIG. 1).

In this context, it is preferred if the protein or polypeptide according to the invention corresponds by at least 95%, preferably 97%, and particularly precisely to the amino acid sequence in accordance with one of the sequences with the consecutive number 55 to 86 or 97, 98 or 100 or 102 to 105 (Table 1 in combination with FIG. 1), preferably 55 to 61, particularly 55, 56, 58 or 60.

Accordingly, another object is also a protein or polypeptide that is coded by a nucleic acid in accordance with one of claims 13 or 14. In this context, “coded” in the sense of this invention is understood to mean that the codons (see above) of the corresponding nucleic acid segment (gene or ORF) code for the corresponding amino acid sequence, in other words that a corresponding protein or polypeptide with this amino acid sequence is formed after transcription and translation.

In particular, the proteins according to the invention are enzymes, or part of a multienzyme complex. Of course they can also have other functions.

Since the avilamycin derivatives according to the invention are defined by way of a gene technology or biotechnology process, or are produced in this way, on the one hand, while on the other hand the newly discovered genes and proteins (enzymes) can be used in gene technology or biotechnology processes for the production of corresponding antibiotics, cells modified by gene technology almost necessarily have an important function within the scope of this invention.

Therefore cells modified by gene technology, containing at least one non-endogenous nucleic acid according to the invention, one non-endogenous gene cluster according to the invention, and/or one non-endogenous protein or polypeptide according to the invention, are another object of this invention.

Likewise, a cell that contains at least one nucleic acid modified by gene technology, the sequence of which, before modification, corresponded to the nucleic acid sequence in accordance with one of the sequences with the consecutive number 1 to 54 (Table 1 in combination with FIG. 1) by at least 95%, preferably 97%, and particularly precisely that hybridized with one of these sequences under moderately stringent conditions, is also an object of the invention.

An especially preferred object of the invention is a cell of the type Streptomyces viridochromogenes, preferably of the subtype Tü57, in which at least one of the nucleic acids with a sequence with one of the consecutive numbers 1-54 (Table 1 in combination with FIG. 1) was modified by gene technology or deleted. In this context, it is especially preferred if at least one of the nucleic acids with a sequence with the consecutive number 1 or 2-7 (Table 1 in combination with FIG. 1) preferably with one of the sequences with the consecutive number 1, 2, 4, or 6 (Table 1 in combination with FIG. 1), particularly with a sequence with the consecutive number 2 or with a sequence with consecutive numbers 2 and 1, 2 and 4, or 2 and 6 (in accordance with Table 1 in combination with FIG. 1) was modified by gene technology or deleted.

Likewise, it is especially preferred if the cell is of the mutant type Streptomyces viridochromogenes GW4, Streptomyces viridochromogenes GW4-AM1, Streptomyces viridochromogenes GW2 or Streptomyces viridochromogenes GW5, where avilamycin derivatives in which R3=OH are synthesized by the mutant type Streptomyces viridochromogenes GW4, while avilamycin derivatives in which R3=OH, R4=H, and R5=H are synthesized by the mutant type Streptomyces viridochromogenes GW4-AM1, and avilamycin derivatives in which R3=OH and R6=H are synthesized by the mutant type Streptomyces viridochromogenes GW2, and avilamycin derivatives in which R3=OH and R9=H are synthesized by the mutant type Streptomyces viridochromogenes GW5.

Accordingly, another object of the invention, in accordance with the foregoing explanations, is the use of a nucleic acid according to the invention, of a gene cluster according to the invention, of a protein or polypeptide according to the invention, and/or one of the cells according to the invention for the production of an avilamycin derivative, preferably an avilamycin derivative according to the invention.

Another object of the invention is a process for the production of avilamycin derivatives according to the invention with the following steps:

(1) in a cell that can be cultivated, which exhibits the necessary genes and/or enzymes for the synthesis of the orthosomycin basic body consisting of:

a) a terminal dichloroisoeverninic acid moiety (A in Formula I);

b) a heptasaccharide esterified with it, linked via normal ester bonds and ortho ester bonds (B to H in Formula I), comprised of:

(i) two D-olivose moieties (B and C);

(ii) a 2-deoxy-D-evalose moiety (D);

(iii) a D-fucose moiety (E);

(iv) a D-mannose moiety (F);

(v) an L-lyxose moiety (G); and

(vi) a (methyl) eurekanate moiety (H),

at least one nucleic acid, the sequence of which corresponds by at least 95%, preferably 97%, and particularly to the nucleic acid sequence with a consecutive number from 1 to 54 in accordance with Table 1 in combination with FIG. 1, or a nucleic acid that hybridizes with one of these sequences under moderately stringent conditions, is modified by gene technology, deleted and/or not expressed;

(2) the cell modified by gene technology in this way is cultivated;

(3) the top portion of the culture is recovered;

(4) the top portion of the culture is processed and the avilamycin derivative(s) formed in this way is/are purified and isolated; and

(5) if applicable, different derivatives are separated from one another.

It is preferred if, in this process, the cell that can be cultivated is selected from a cell of the type Streptomyces viridochromogenes or a cell that, with the exception of the nucleic acid modified by gene technology, deleted or not expressed contains the nucleic acids in accordance with consecutive number 1-54 in accordance with Table 1 in combination with FIG. 1, or nucleic acids homologous to them by at least 95%, preferably 97%, or sequences that hybridize with these sequences, or the gene cluster according to the invention. In the technical literature, the latter is referred to as heterologous expression. In this context, it is especially preferred if the cell is selected from a cell of the type Streptomyces viridochromogenes, Streptomyces lividans, Streptomyces albus or Streptomyces fradiae, especially a cell of the type Streptomyces viridochromogenes Tü 57 or A 23575.

An alternative method of conducting the process is also possible according to the invention. Here, after process steps (1) and (2) have been performed, the avilamycin derivative is not recovered from the top culture portion, instead it accumulates in the host cells. In accordance with the alternative process, the host cells are therefore harvested in step (3), then digested, and the avilamycin derivatives are separated from the other cell components and finally purified. The aforementioned methods and any methods familiar to a person skilled in the art can be used for separation and purification.

It is furthermore preferred if, in the process according to the invention, the modified nucleic acid(s) coded for a methyl transferase and/or for a halogenase. In this context, it is especially preferred if the sequence(s) of the modified nucleic acid(s) before modification correspond(s) by at least 95%, preferably 97%, and particularly precisely to the nucleic acid sequence(s) of at least one of the sequences with the consecutive numbers 1 or 2-7 in accordance with Table 1 in combination with FIG. 1, preferably one of the sequences with the consecutive number 1, 2, 4, or 6 (Table 1 in combination with FIG. 1), particularly the sequence with the consecutive number 2 or the sequences with the consecutive numbers 2 and 1, 2 and 4, or 2 and 6 (in accordance with Table 1 in combination with FIG. 1).

It is furthermore preferred if, in the process according to the invention, modification of the nucleic acid(s), particularly of the methyl transferases and/or halogenases according to the invention, has the result that the protein(s) or polypeptide(s) coded by the nucleic acid(s) modified by gene technology is/are no longer synthesized after the gene technology modification.

The avilamycin derivatives according to the invention are fundamentally unproblematic in terms of toxicology, so that they are suitable as a pharmaceutical active ingredient in medications. Medications containing at least one avilamycin derivative according to the invention, preferably at least two, particularly also mixtures of one or more avilamycin derivatives with at least one other antibiotic from the state of the art, for example vancomycin, penicillin, streptomycin, neomycin, kanamycin, sisomycin, amikacin and/or tobramycin, as well as suitable additives and/or ancillary substances, are therefore another object of the invention. Other bacteriostatic or bactericidal substances can also be combined with substances according to the invention, for example cephalosporins, choramphenicol, ethambutol, isonicotinamides, tetracyclines, sulfonamides, oxalactams, (for example flomoxef, clavulanic acid) and/or nitrofurans.

Such substances are also understood especially to be carrier materials, fillers, solvents, diluents, pigments and/or binders. The medications can be administered as liquid medications in the form of injection solutions, drops, or syrups, as semi-solid medications in the form of granulates, tablets, pellets, patches, capsules, adhesive bandages or aerosols. The selection of the ancillary materials, etc., as well as the amounts of them to be used, depend on whether the medication is to be applied by oral, peroral, parenteral, intravenous, intraperitoneal, intradermal, intramuscular, intranasal, buccal, rectal, or local administration, for example to the skin, the mucous membranes, or the eyes. Formulations in the form of tablets, coated tablets, capsules, granulates, drops, syrups, and liquors are suitable for oral administration, while solutions, suspensions, easily reconstituted dry formulations, as well as sprays are suitable for parenteral, topical, and inhalation administration.

Formulations that can be used orally or percutaneously can release the avilamycin derivatives according to the invention in time-release manner, and thereby achieve a more uniform plasma level. Fundamentally, additional active ingredients known to a person skilled in the art can be added to the medications according to the invention.

The amount of active substance to be administered to the patient varies as a function of the patient's weight, the method of administration, the indication and the degree of severity of the illness. Usually, 0.005 to 1,000 mg/kg, preferably 0.5 to 5 mg/kg of at least one avilamycin derivative are administered.

Since an antibiotic effect has been demonstrated for the avilamycin derivatives according to the invention, they are, of course, fundamentally suitable for the treatment of diseases, especially infectious diseases, and/or for the production of a medication for treatment of such diseases. Accordingly, the use of an avilamycin derivative according to the invention for the production of a medication with an antibiotic effect for treatment of infectious diseases, for example, is another object of the invention.

Infectious diseases are understood to mean diseases that are caused by an infection with a viral, bacterial, or protozoological pathogen. Therefore the antibiotics according to the present invention are also suitable for treatment of mycoses, particularly cutaneous and subcutaneous mycoses.

However, it is preferable for the avilamycin derivatives according to the invention to be used to combat bacterial infections. In particular, infections with the following pathogens should be mentioned: leprosy bacteria, mycobacteria, Neisseriae, tuberculosis bacteria, actinomycetes, Corynebacteriae, Listeriae, Clostridia, Bacilla, enterococci, streptococci, staphylococci, particularly also for treatment of infections with Staphylococcus aureus strains, Rickettsiae, chlamydia, Mycoplasmae, Borreliae, spirochetes, Brucellae, Bortedellae, pseudomonas, helicobacter, hemophilus, vibrions, shigellae, yersinia, salmonellae, and other representatives of the family of Enterobacteriaceae.

Accordingly, the substances according to the invention are used for treatment of all clinical disease profiles that are caused by the aforementioned bacteria strains. The following disease states are mentioned as examples: tuberculosis; pneumonia; typhus; syphilis; paratyphus; gastritis; gastroenteritis; dysentery; plague; enteritis; extraintestinal infections, peritonitis and appendicitis with E. coli as well as intestinal infections with EHEC, EPEC, ETEC, or EIEC; cholera, Legionnaires' disease, whooping cough, Brucellosis, Lyme borreliosis, leptospirosis, spotted fever, trachoma, gonorrhea, meningitis, septicemia, leprosy, etc.

The treatment of a human or animal that requires this treatment, with an avilamycin derivative according to the invention, preferably in the case of infectious diseases, particularly involving Staphylococcus aureus, is another object of the invention.

In the following sections, the invention will be further explained with examples and figures, without being limited to them.

### BRIEF DESCRIPTION OF THE FIGURES

FIG. 1 shows the sequence of the entire gene cluster with its 54 nucleic acid sequences of the ORFs of Streptomyces viridochromogenes Tü 57. FIG. 1 contains the abbreviations of the corresponding nucleic acid sequences, where these abbreviations (without the prefix “Avi”) are inserted in the lines that contain the start codon of the 54 sequences, in each instance. The amino acid that is coded by the start codon in each instance is circled. The arrow drawn in at these locations, in each instance, indicates the direction in which the gene should be read (backwards or forwards), with the start codon as the starting point.

FIG. 1 contains the nucleotide sequences of the two complementary DNA strands as well as the (partially imaginary) amino acid sequences for both strands in all three reading rasters, which makes a total of two nucleotide sequences and the six protein sequences potentially resulting from them (single letter code). The three protein sequences of the upper nucleotide strand are drawn in above the related nucleotide sequence, the three protein sequences of the lower complementary nucleotide sequence are drawn in below the related lower nucleotide strand. The 54 protein sequences in the gene cluster drawn in by name in FIG. 1 result from FIG. 1 in that a circled amino acid is selected as the starting point and then the amino acid sequence is read off in the direction indicated by the arrow, in other words from there either forwards or backwards, in this reading raster (in the corresponding line, for example 2^{nd} line below the lower nucleotide sequence). The sequence ends with the stop codon in the corresponding reading raster, where stop codons are marked by a “star” symbol in the corresponding line.

The nucleotide sequence belonging to the amino acid of an ORF results from the corresponding triplet located above or below (for the upper strand). In this context, the single-letter designation of the amino acid is arranged in such a way, in each instance, that it lies above or below the middle nucleotide of the codon coding for this amino acid.

In the subsequent table, the name designations of the 54 coding regions in the gene cluster are each assigned to consecutive numbers, where the consecutive numbers 1 to 54 indicate the nucleotide sequences and the consecutive numbers 55 to 108 correspond to the related amino acid sequences, in each instance, where specifically, the nucleotide sequence with the consecutive number 1 codes for the amino acid with the consecutive number 55, the nucleotide sequence with the consecutive number 2 codes for the amino acid with the consecutive number 56, etc.

FIG. 109 shows the relative arrangement of the ORFs found on the gene cluster.

FIG. 110 shows a Southern blot with the mutant S. viridochromogenes GW4.

FIG. 111 shows the mass spectrum of the products of the mutant S. viridochromogenes GW4.

FIG. 112 shows the mass spectrum of the hydrolyzed products of the mutant S. viridochromogenes GW4.

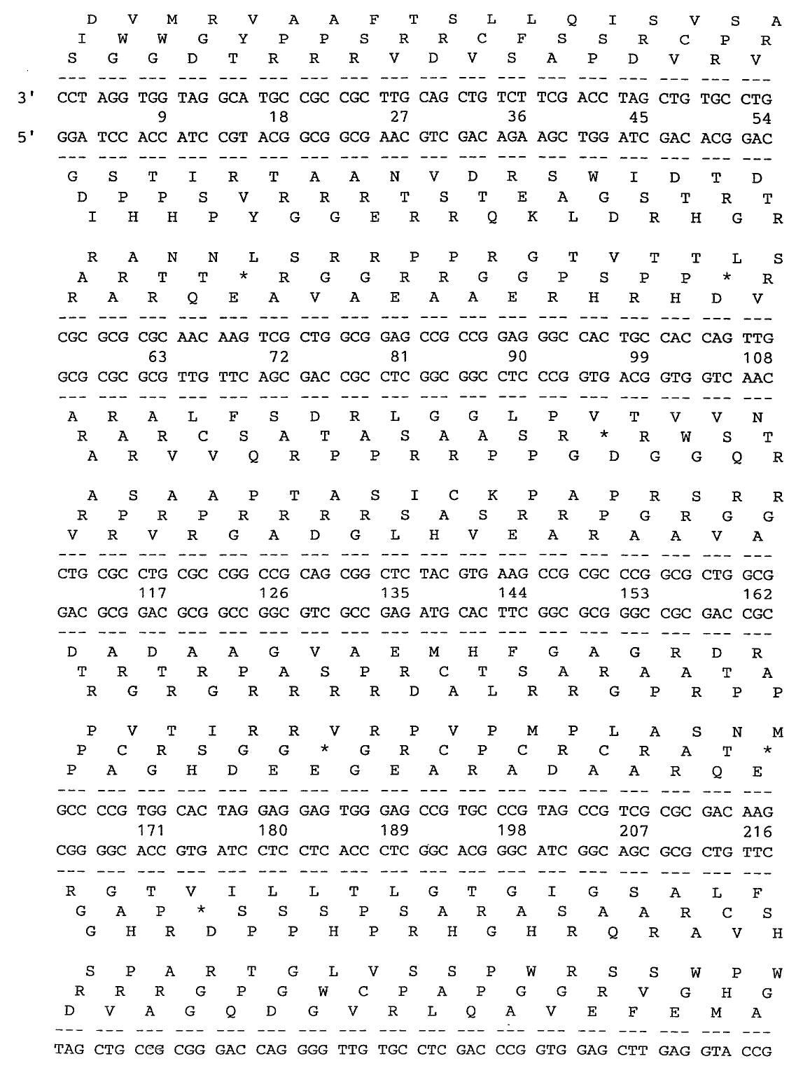

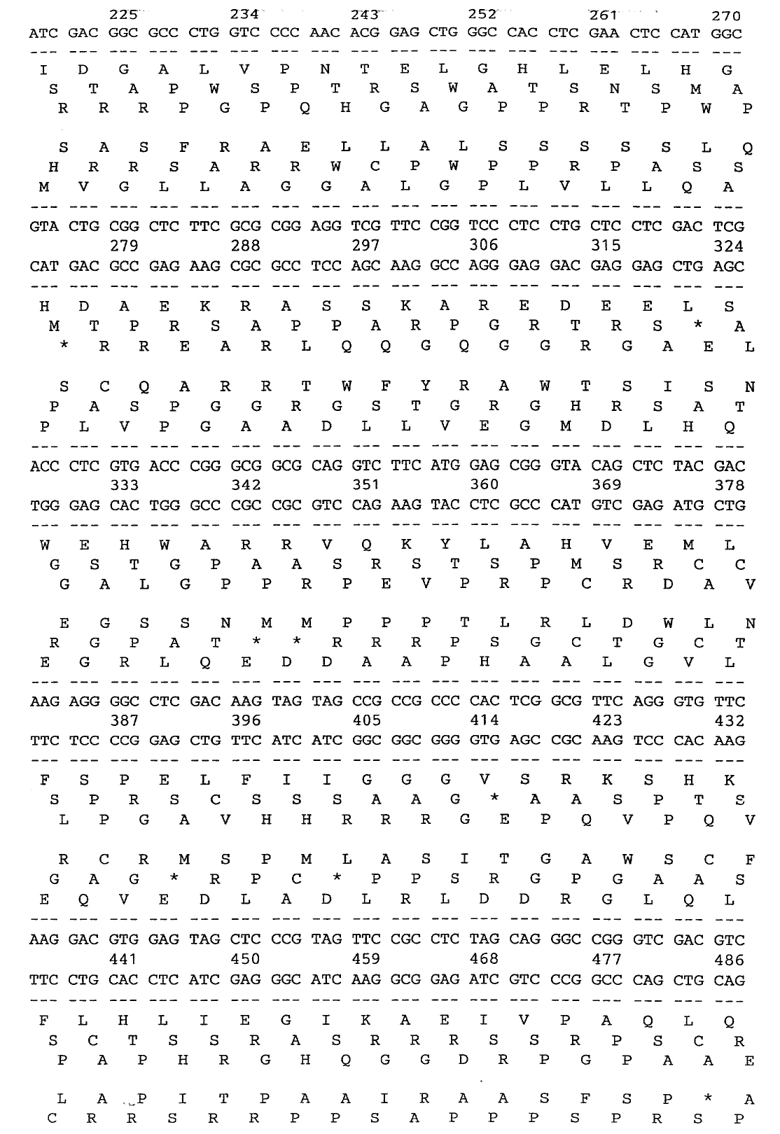

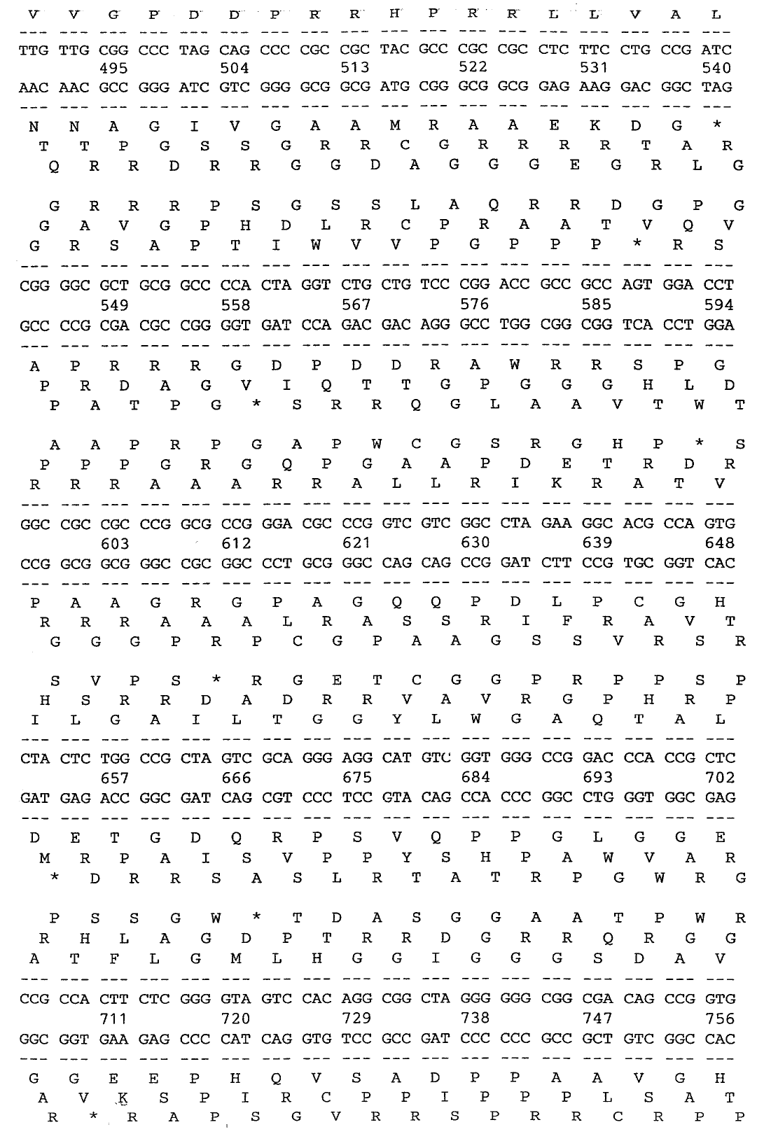

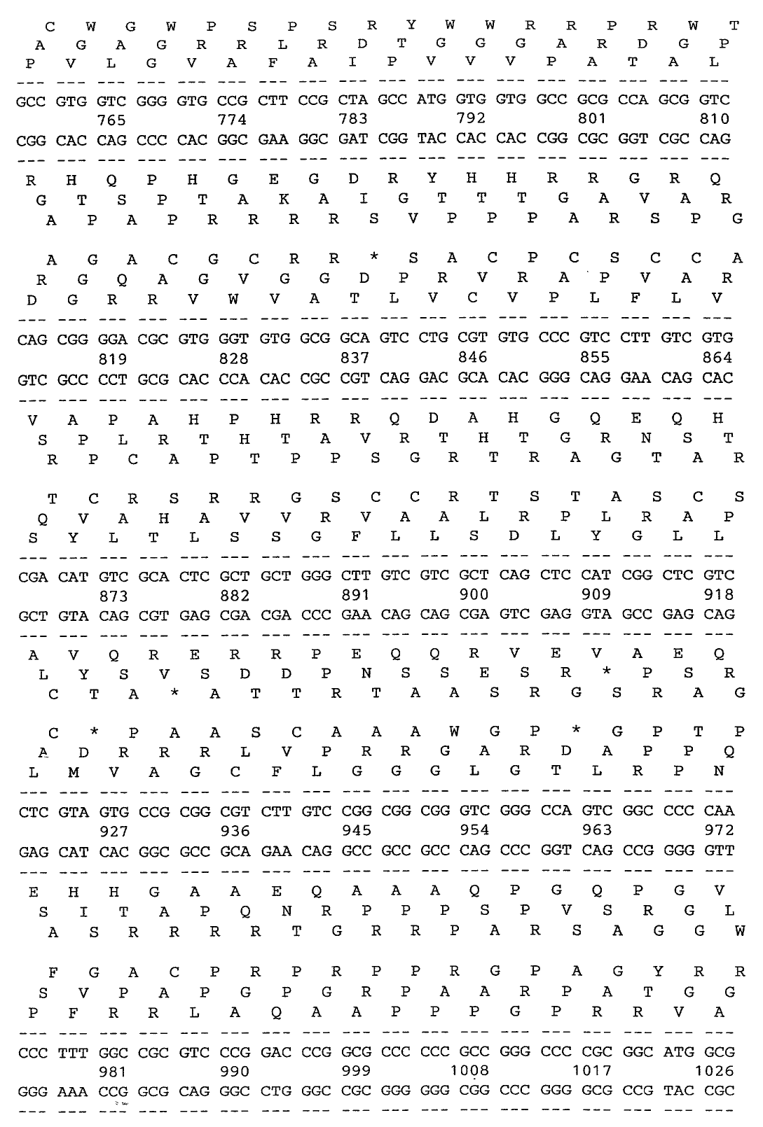

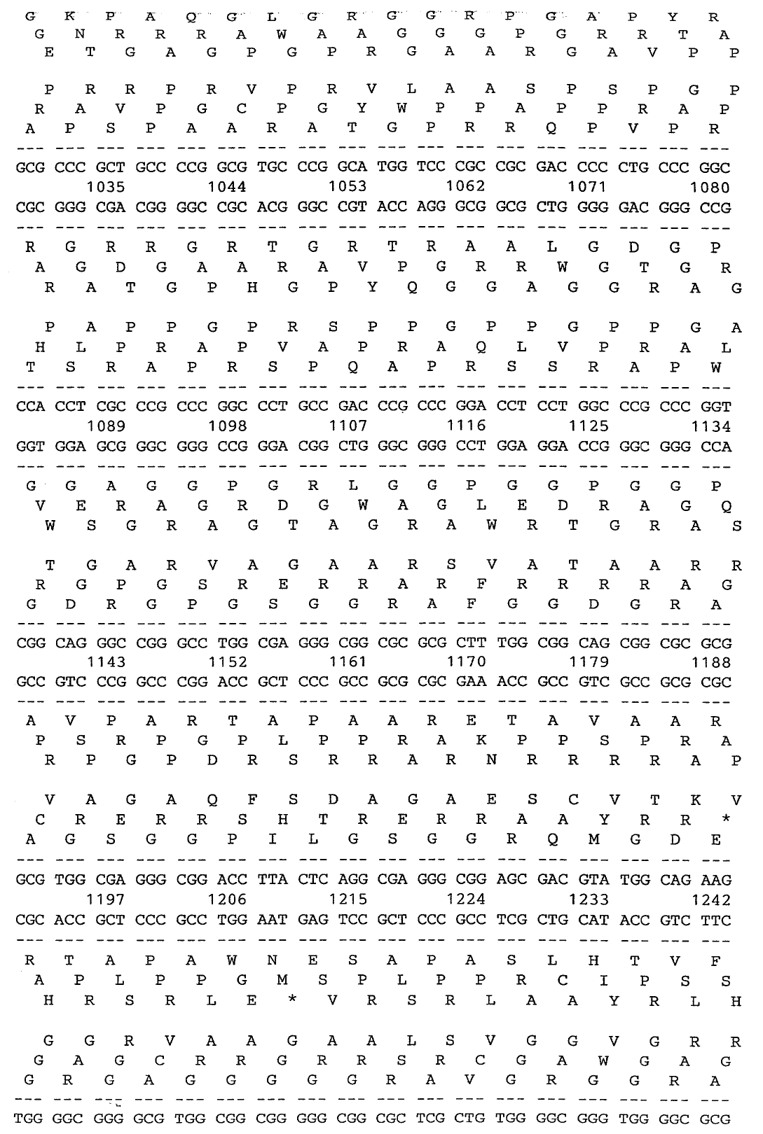

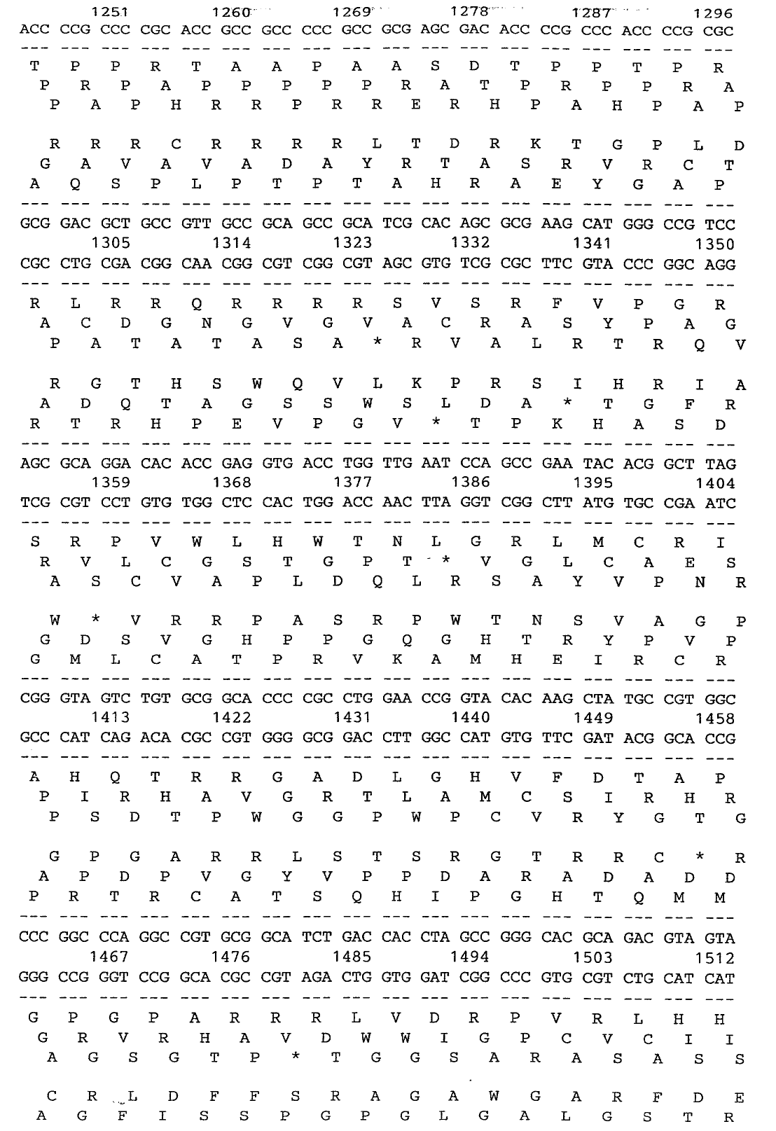

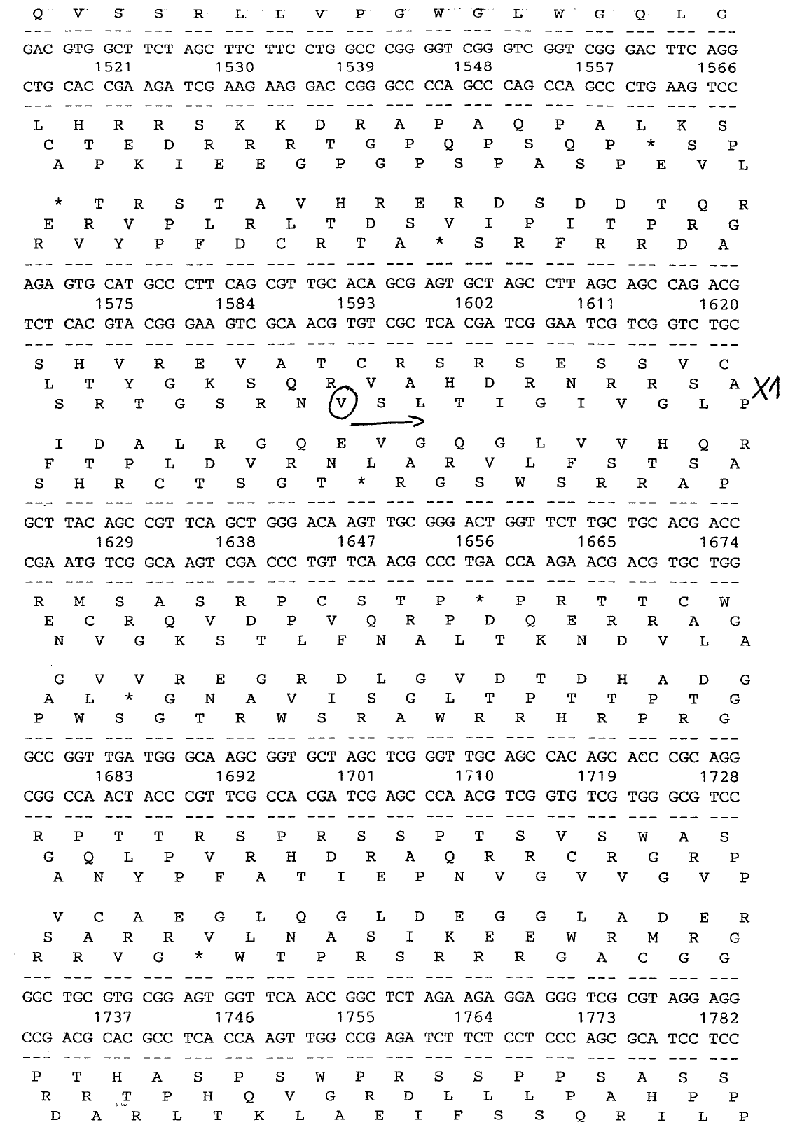

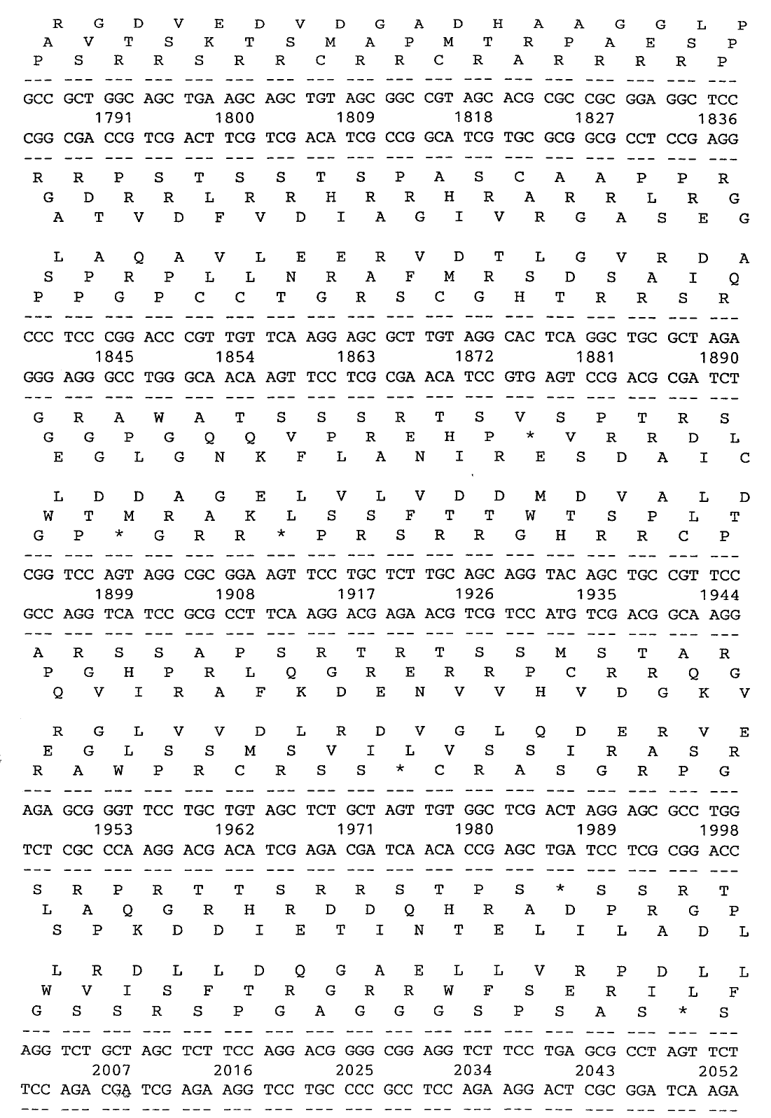

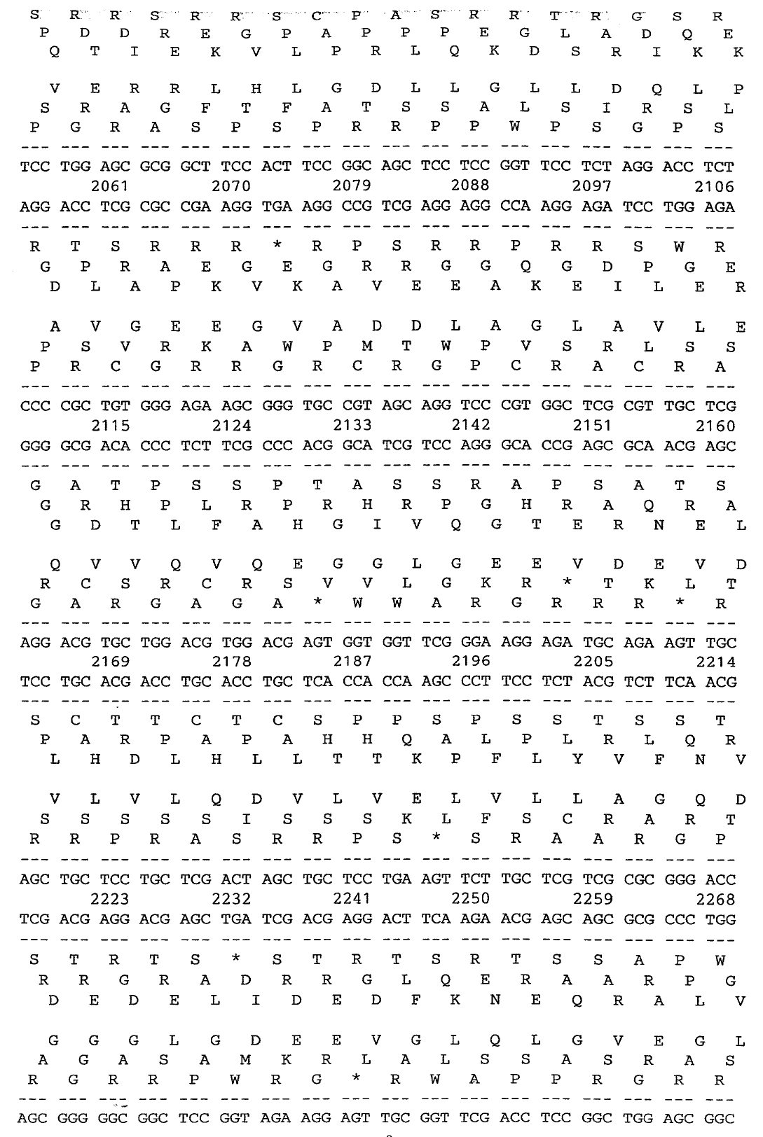

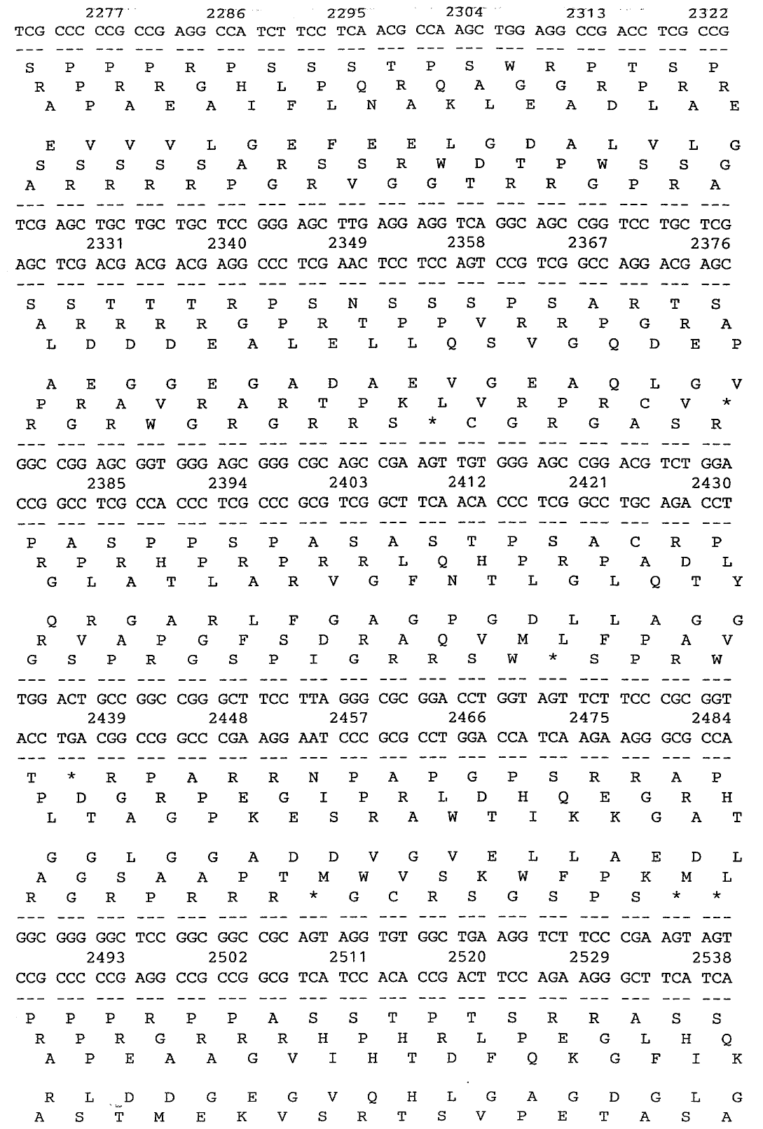

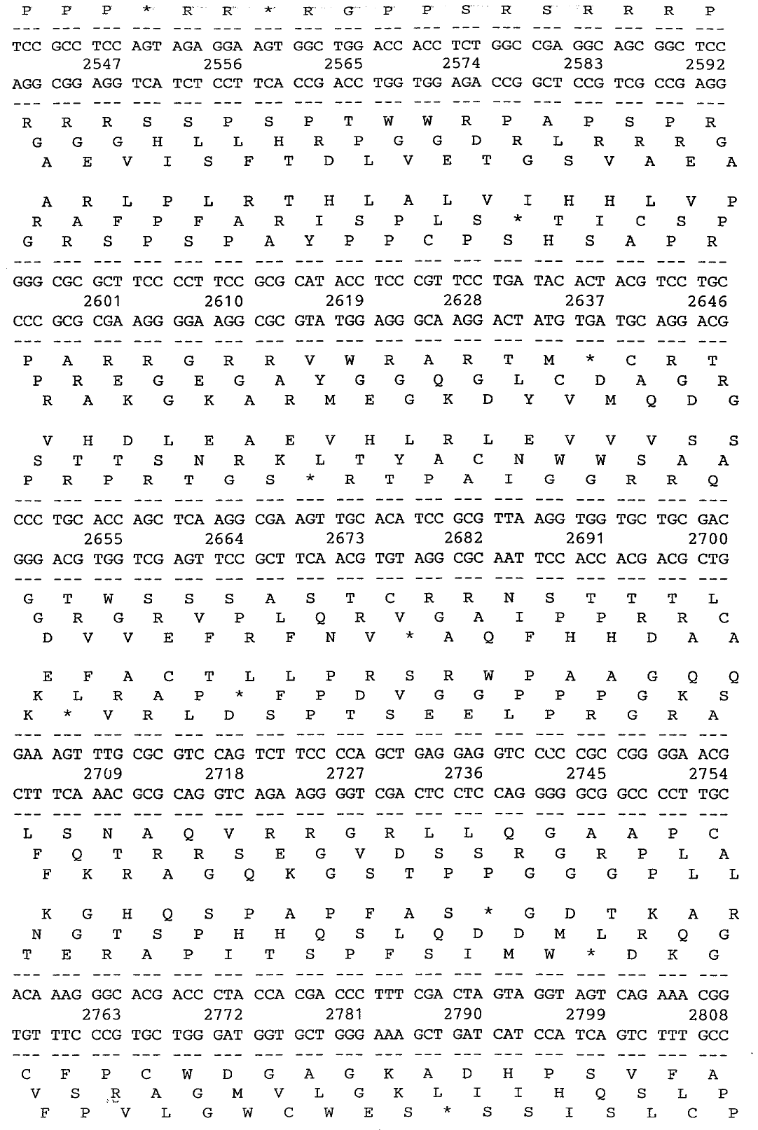

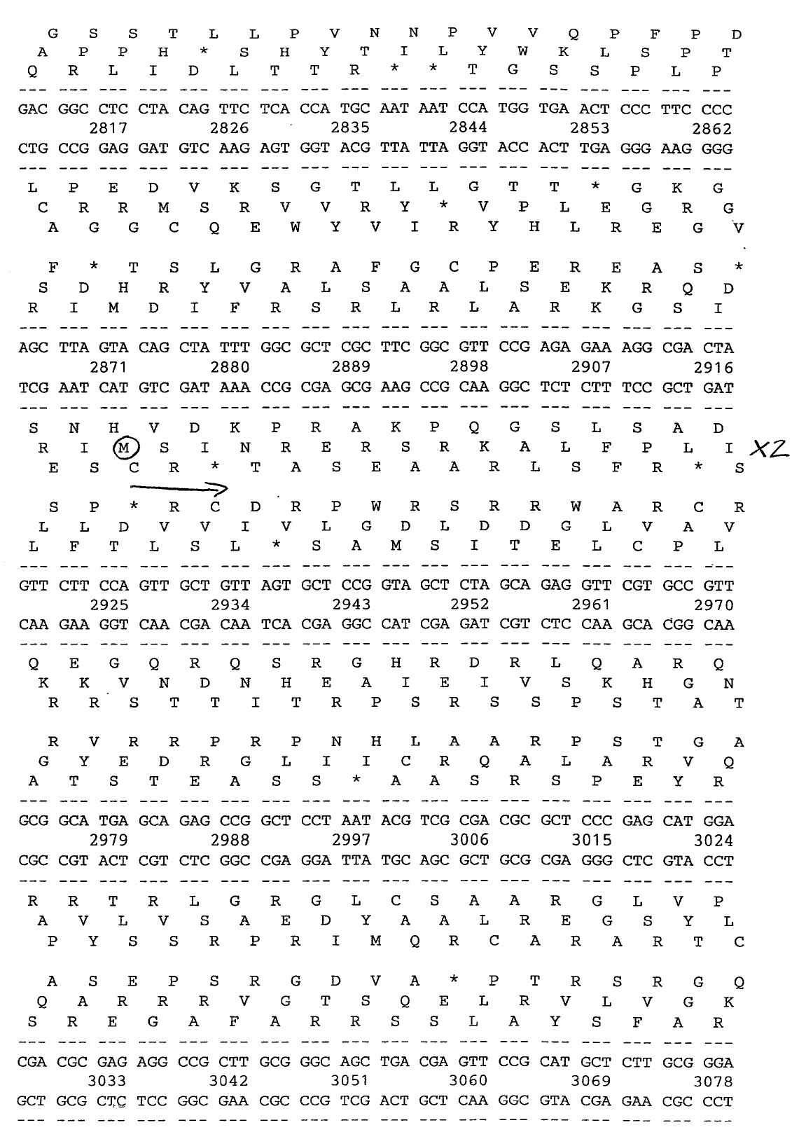

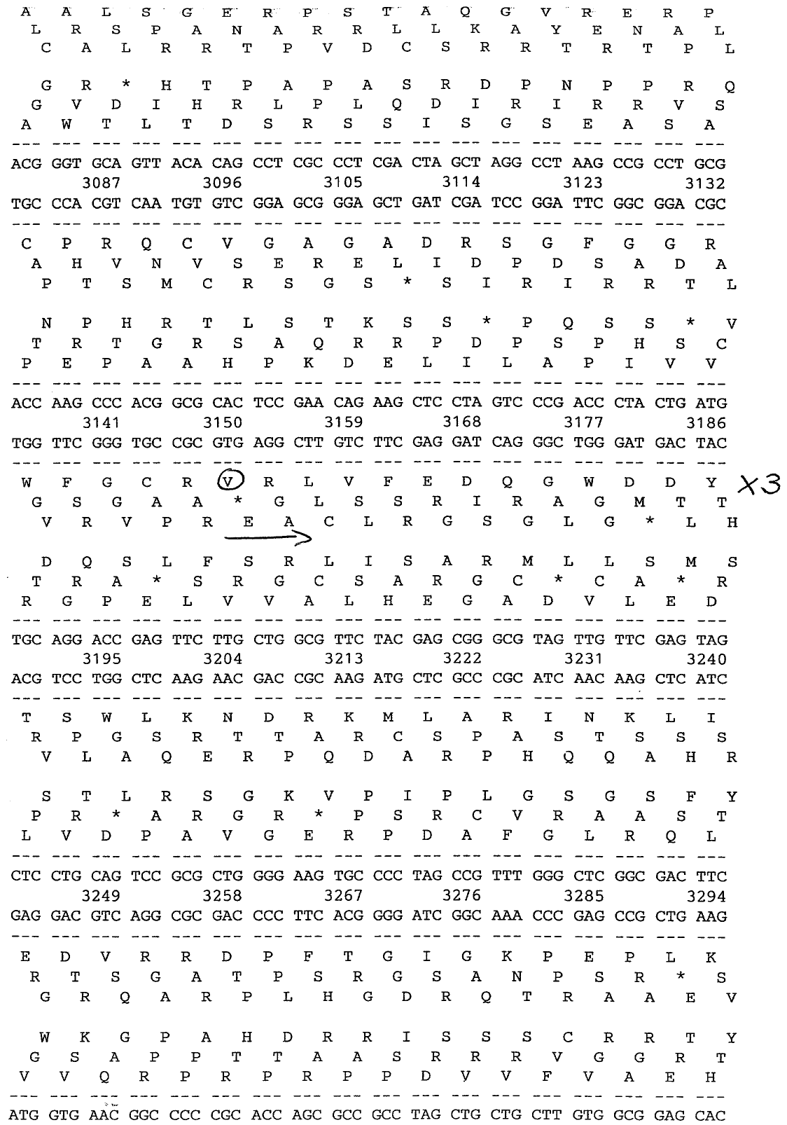

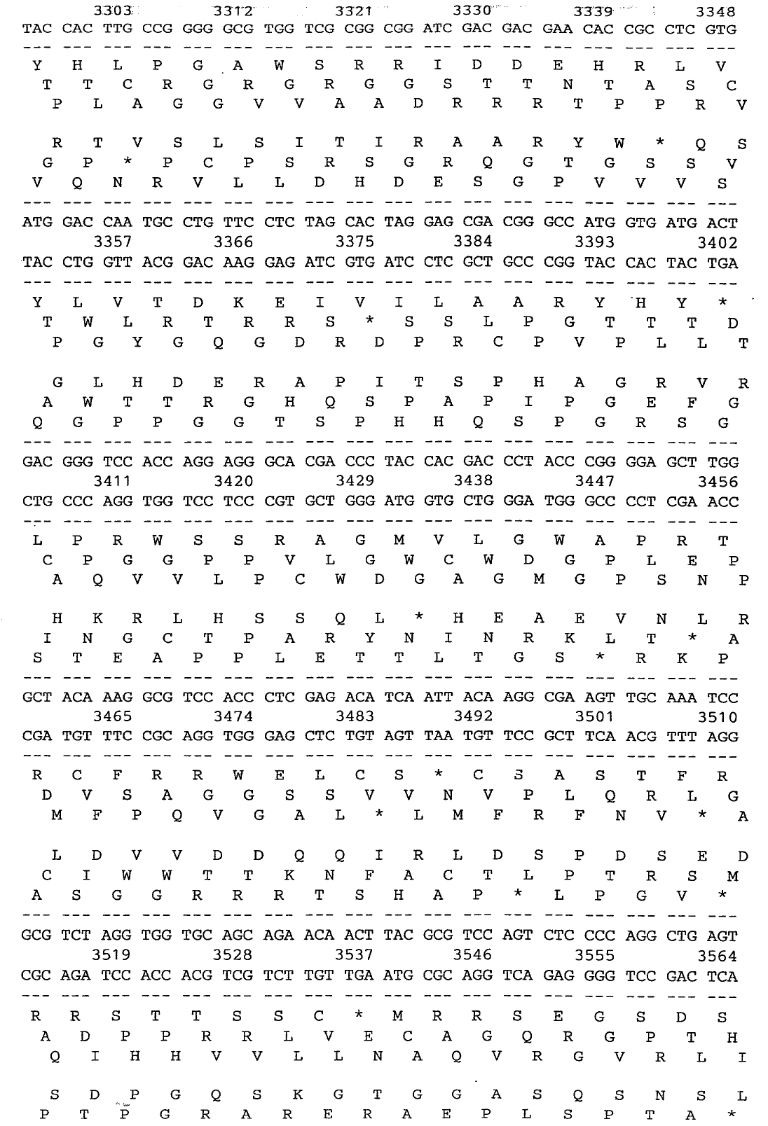

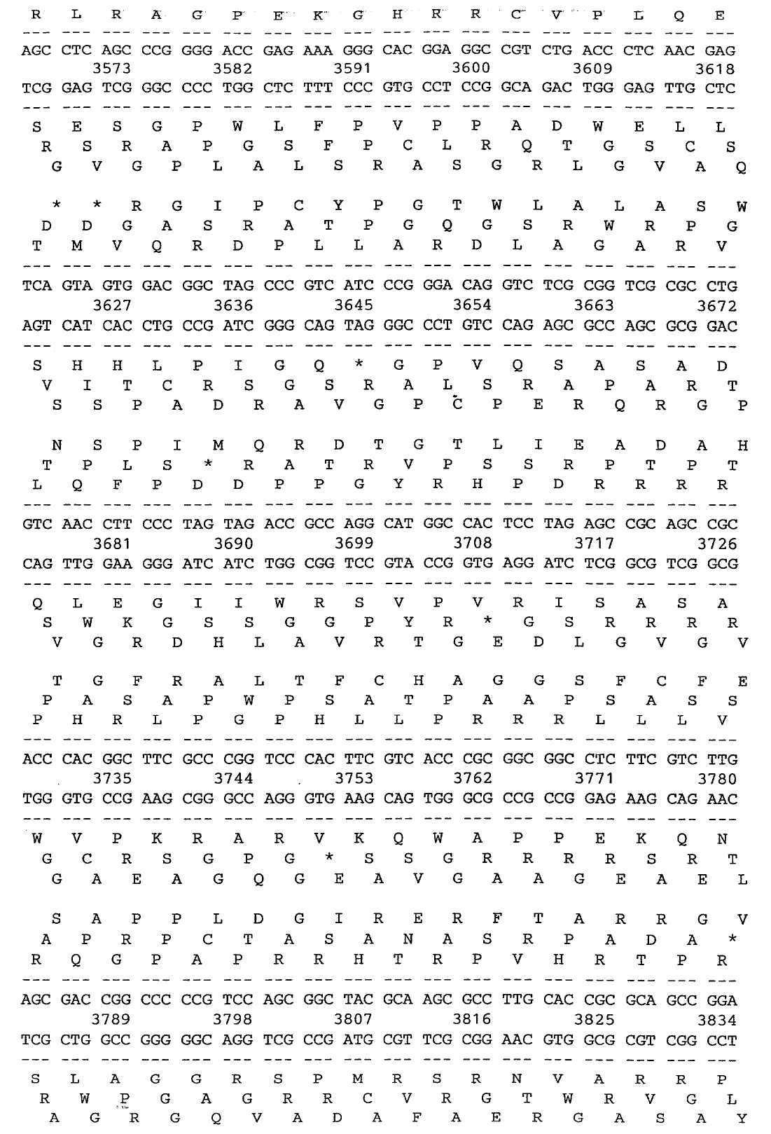

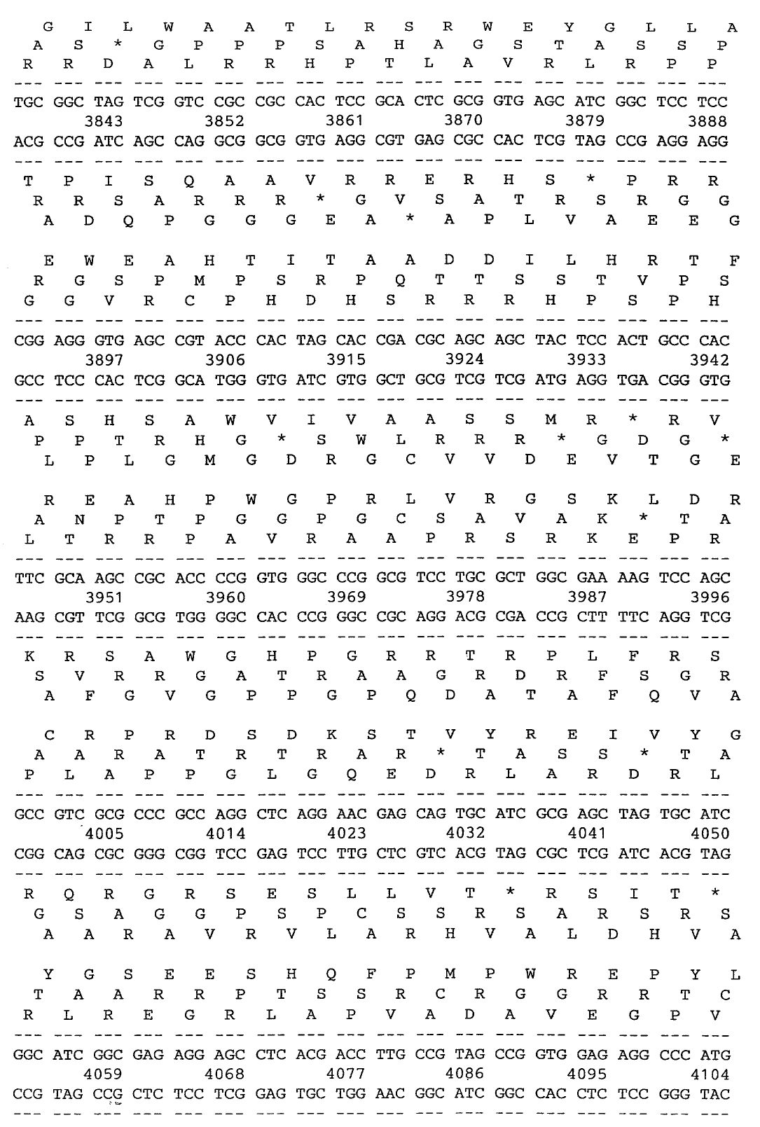

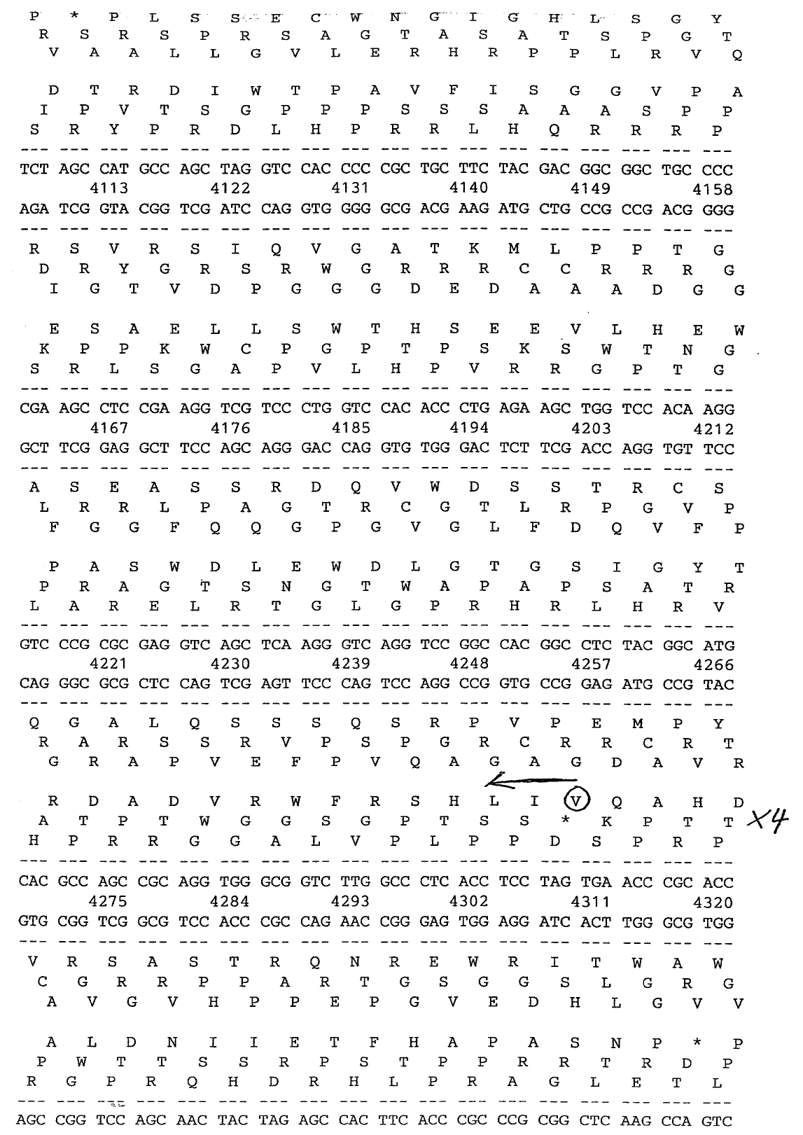

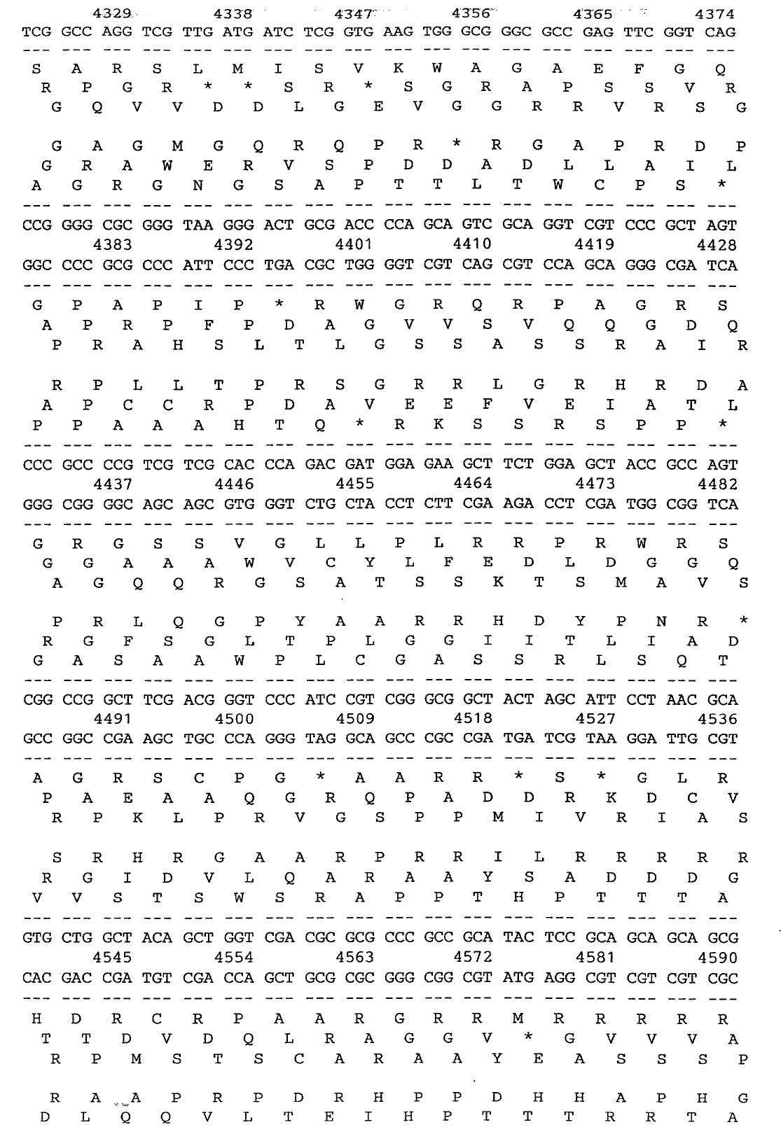

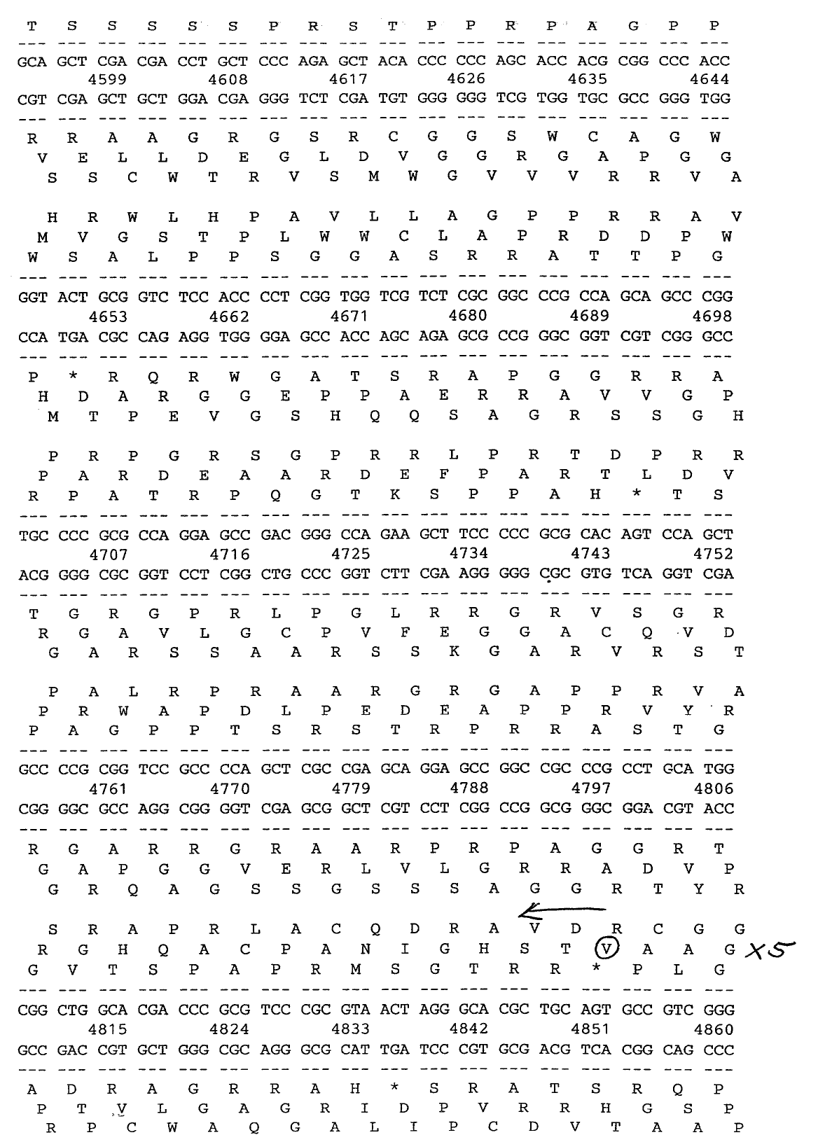

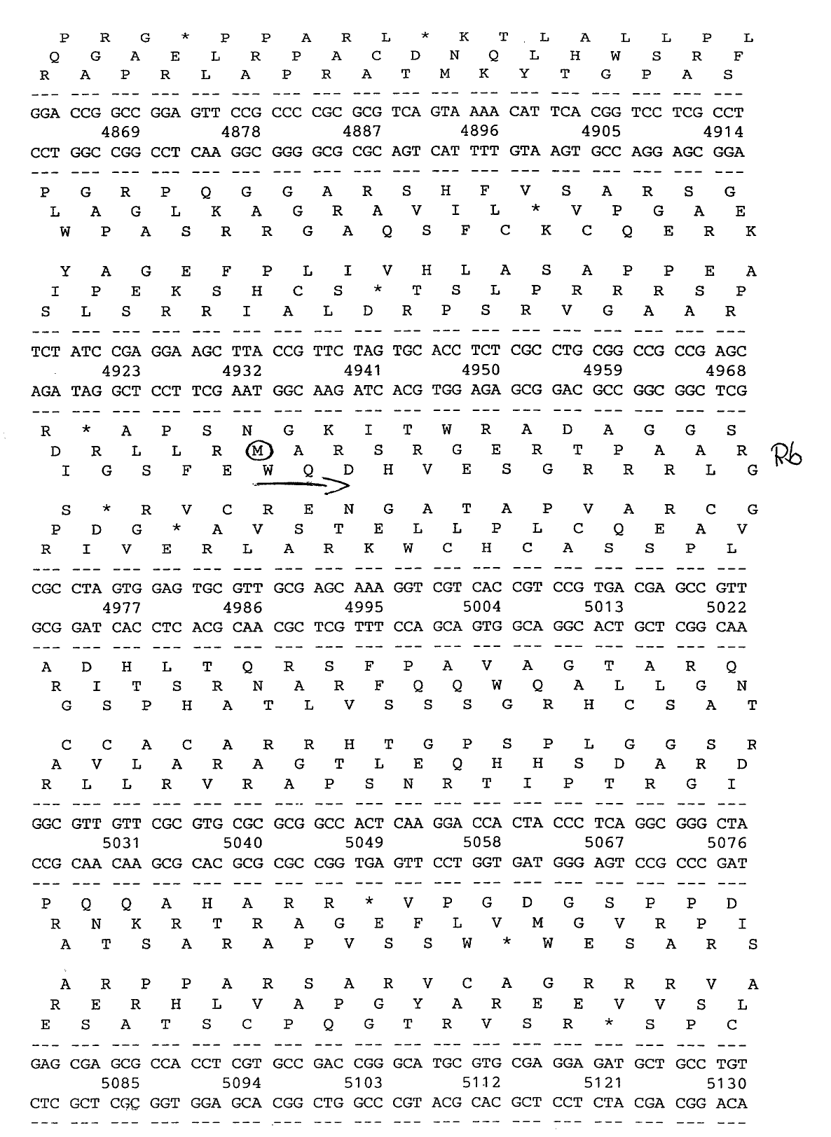

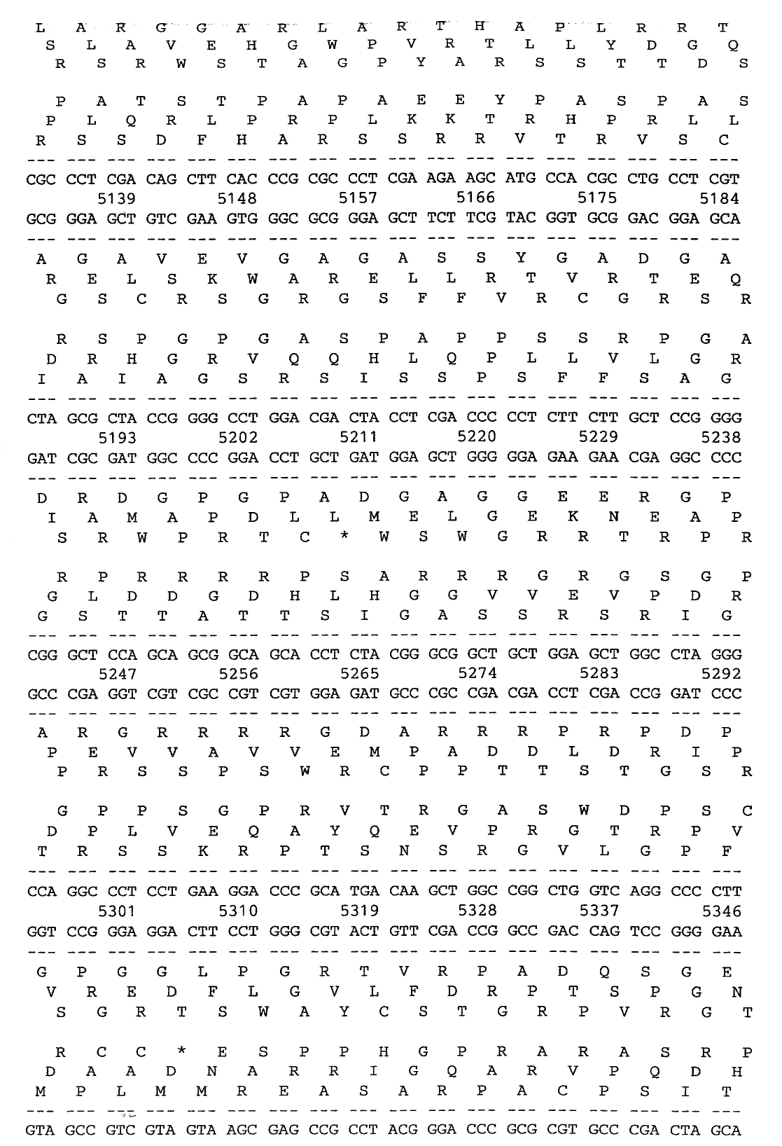

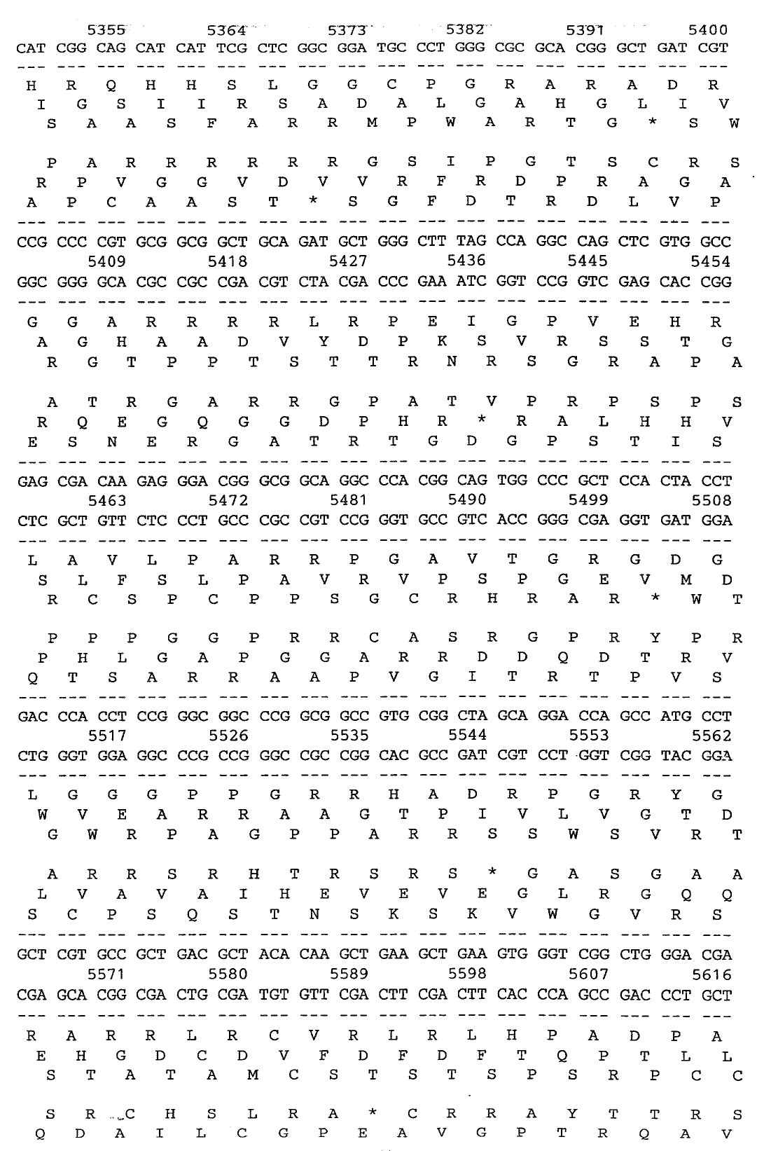

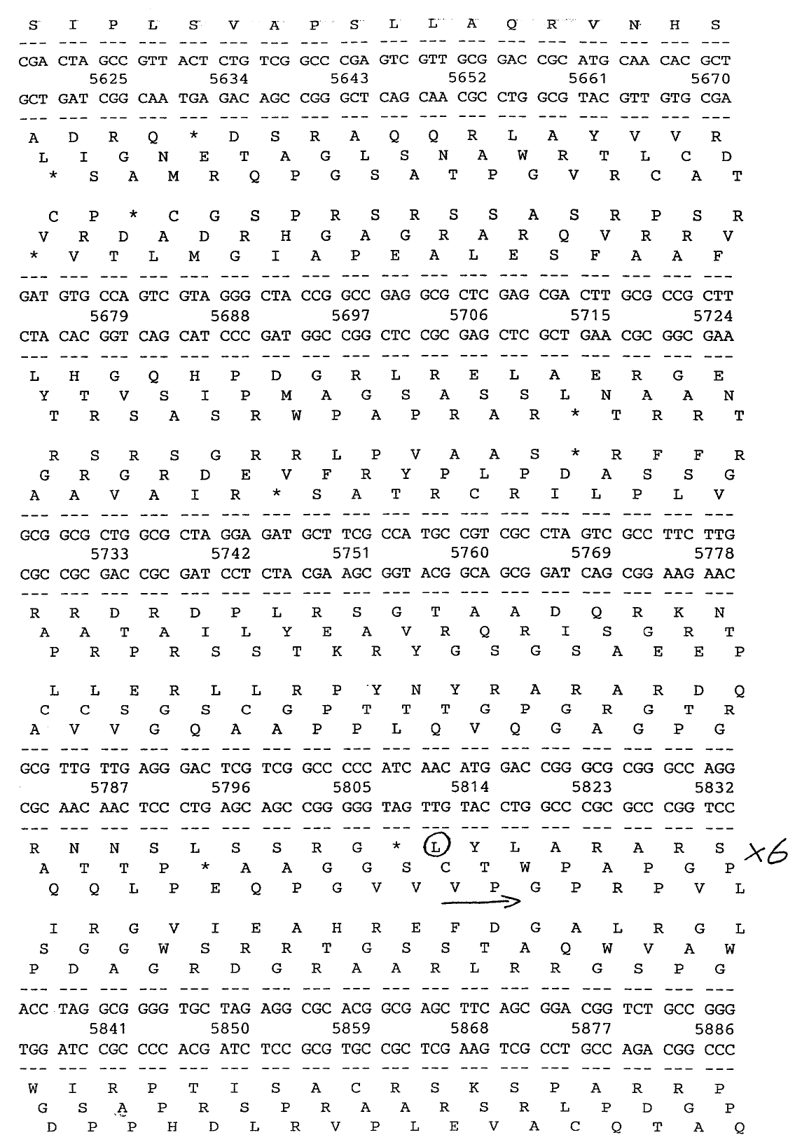

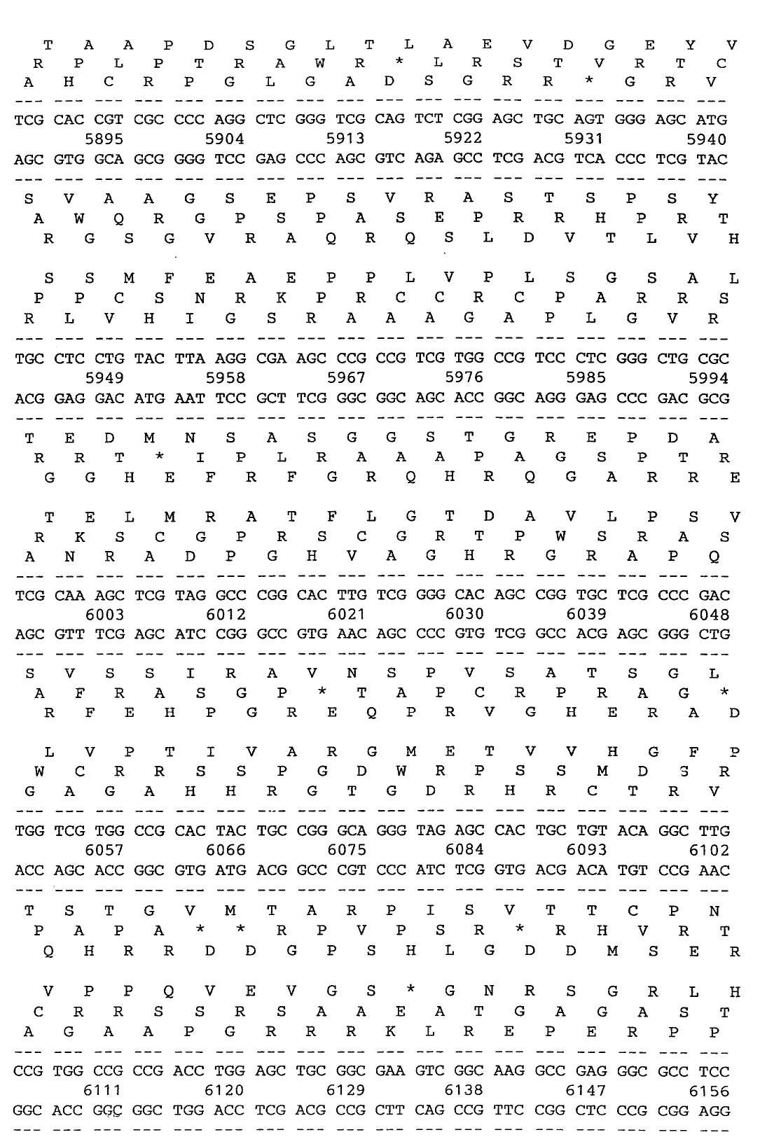

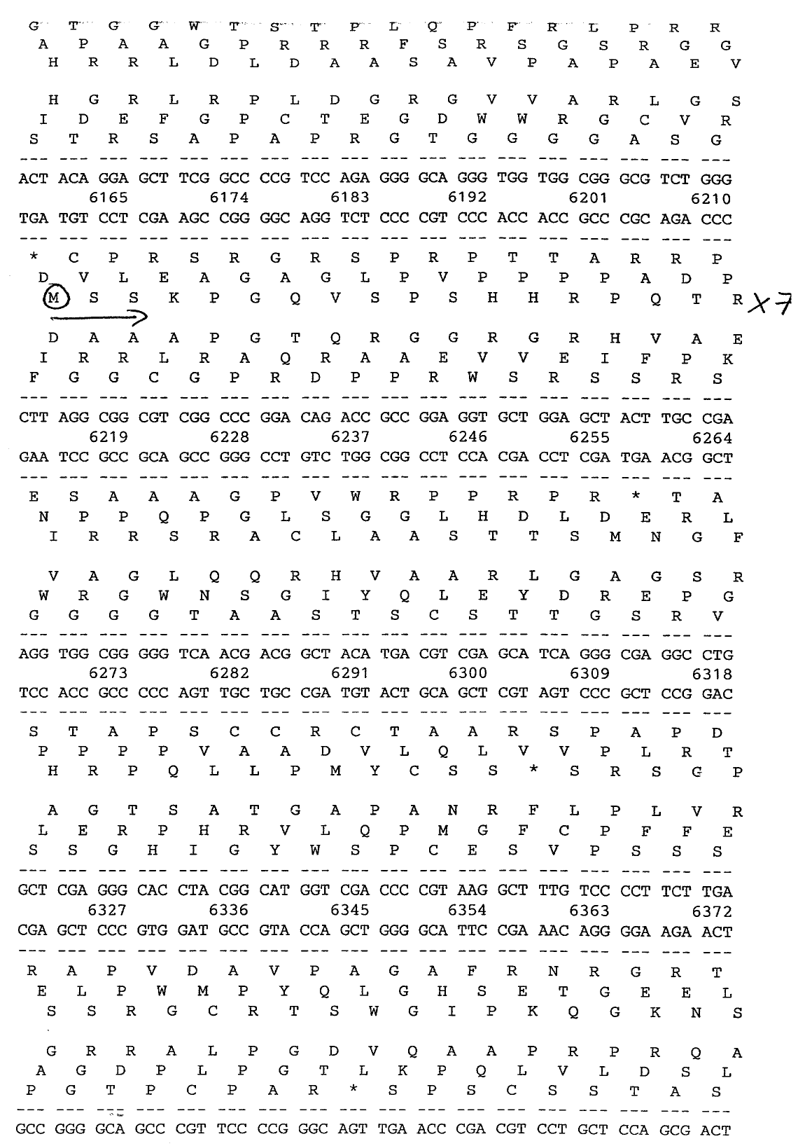

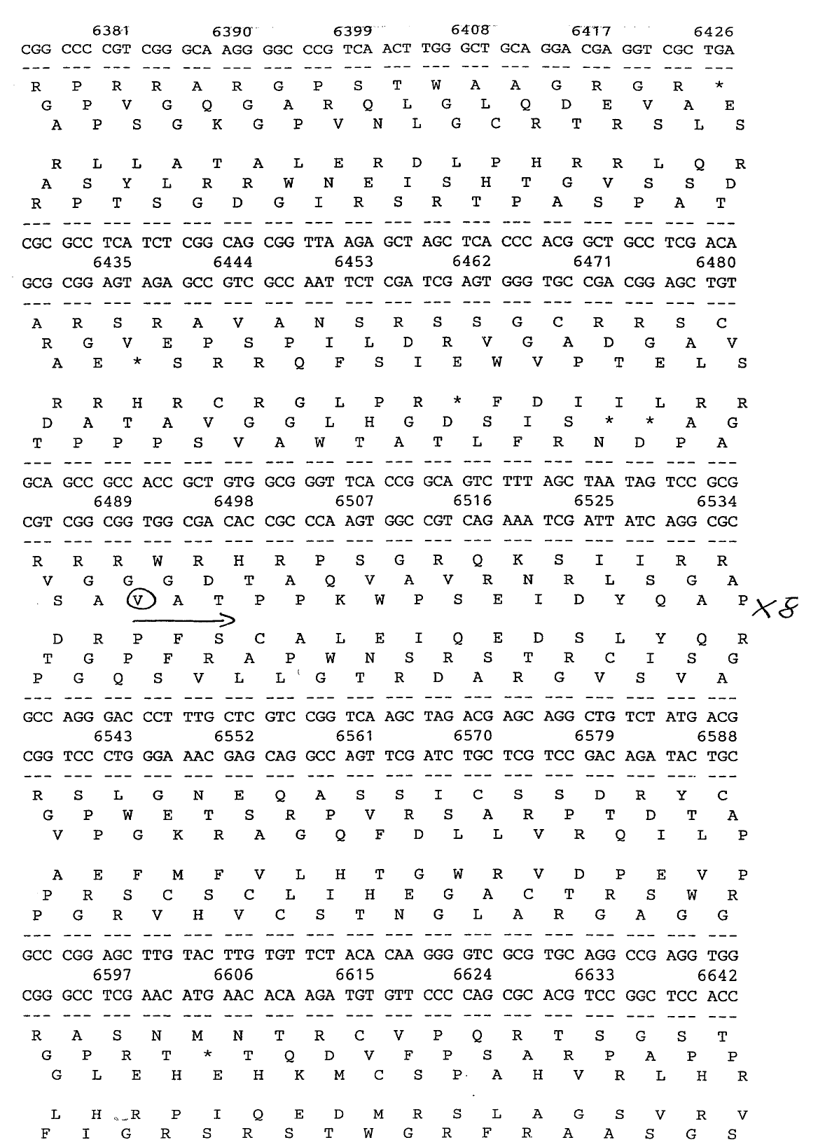

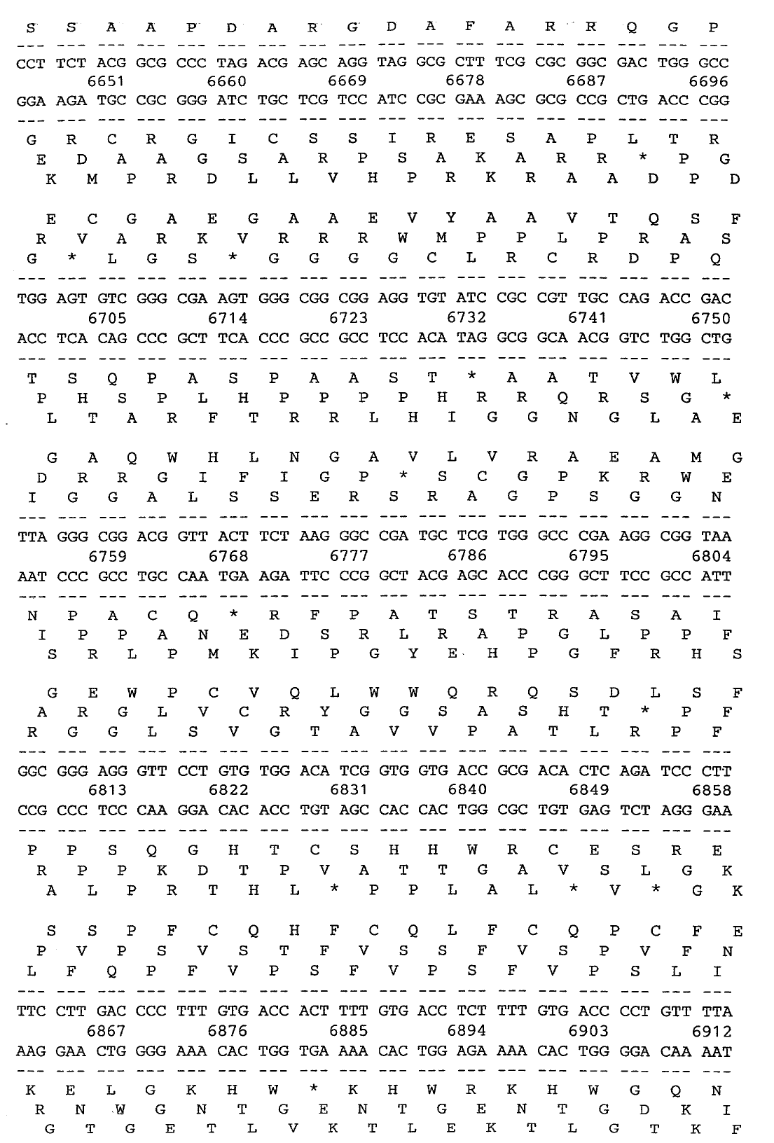

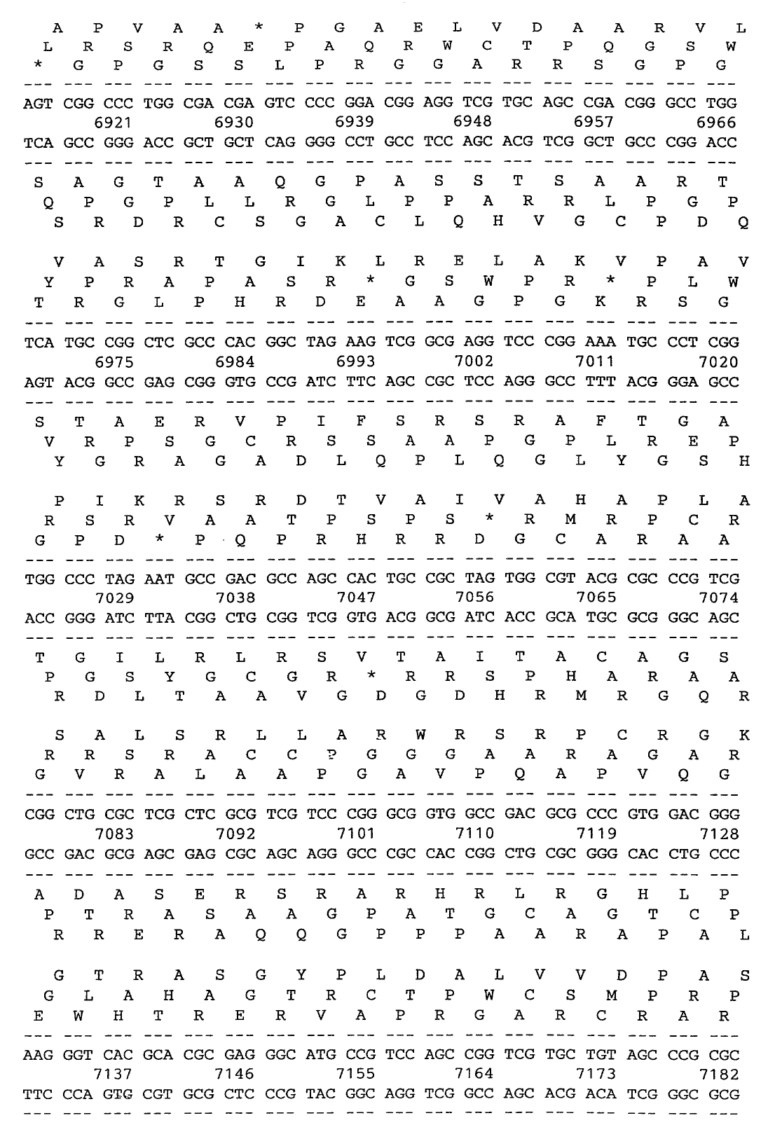

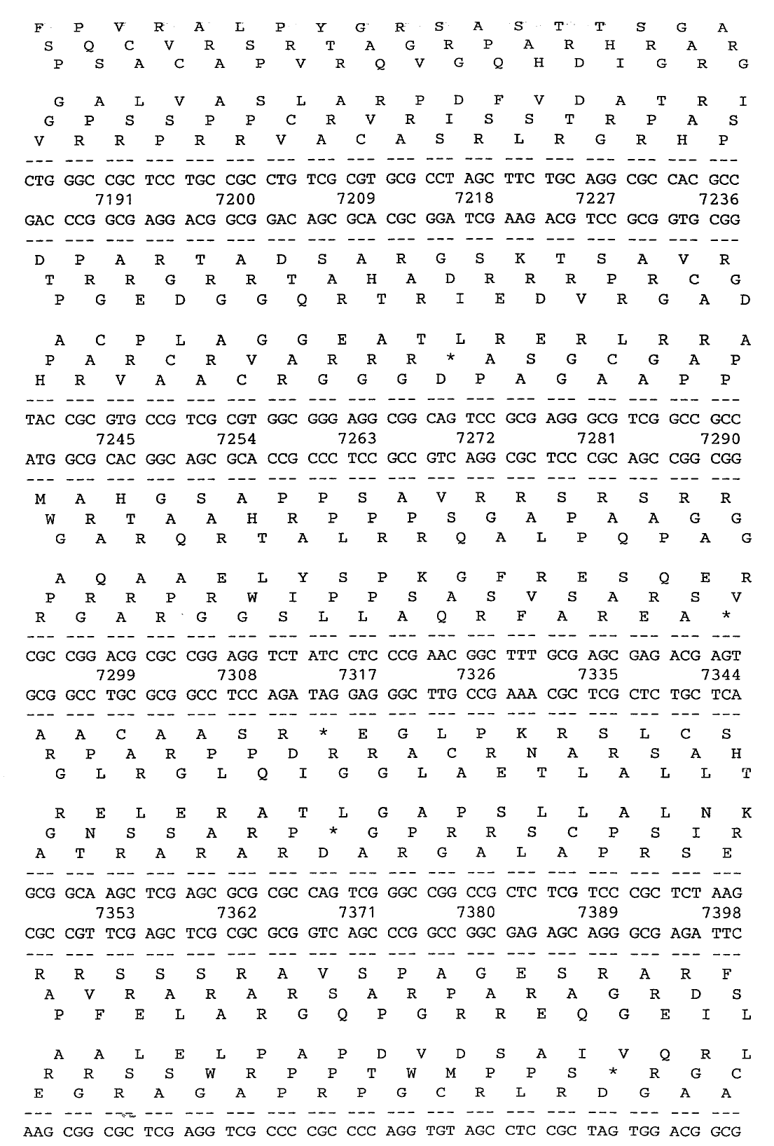

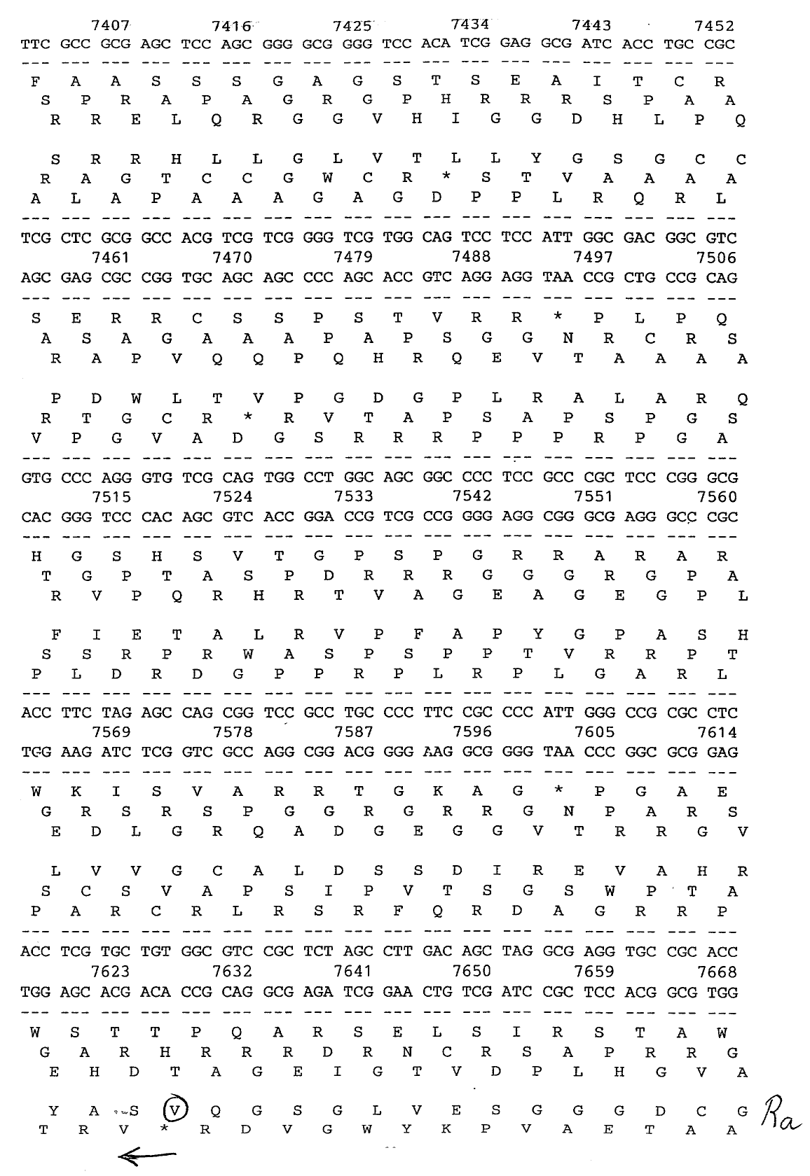

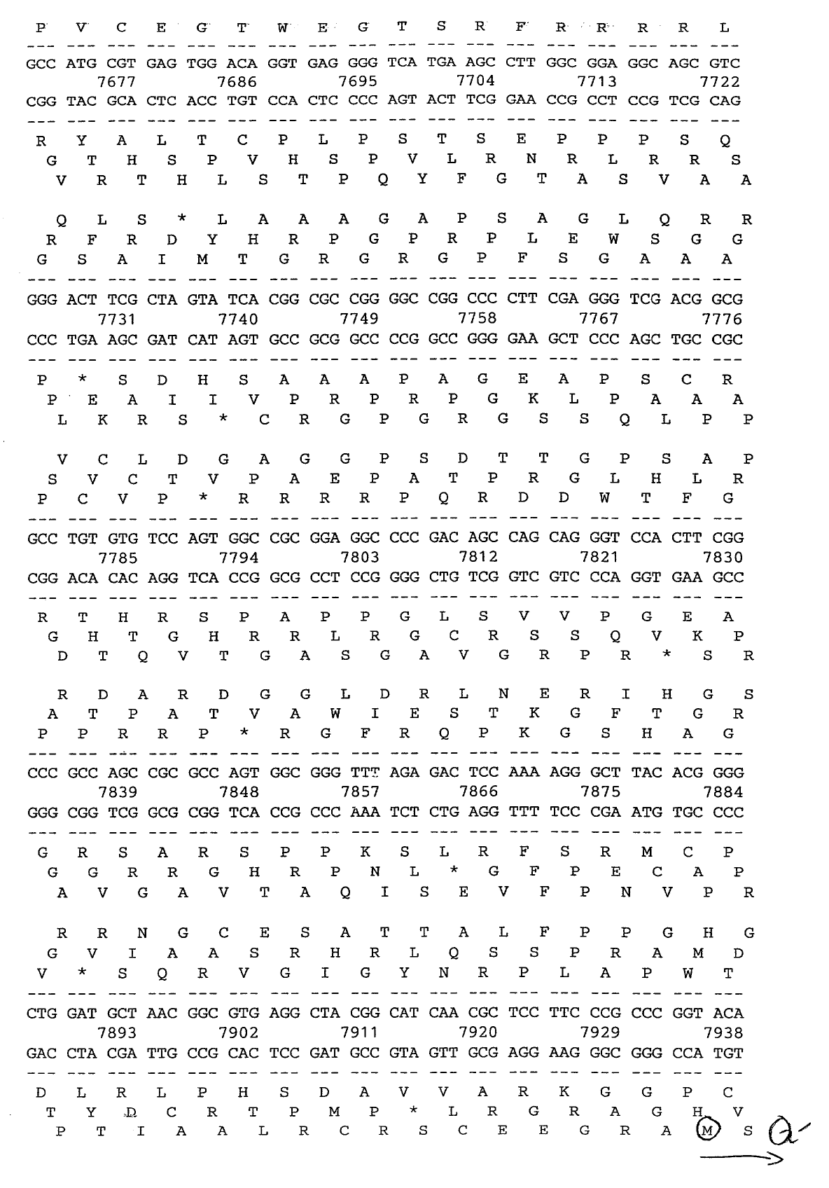

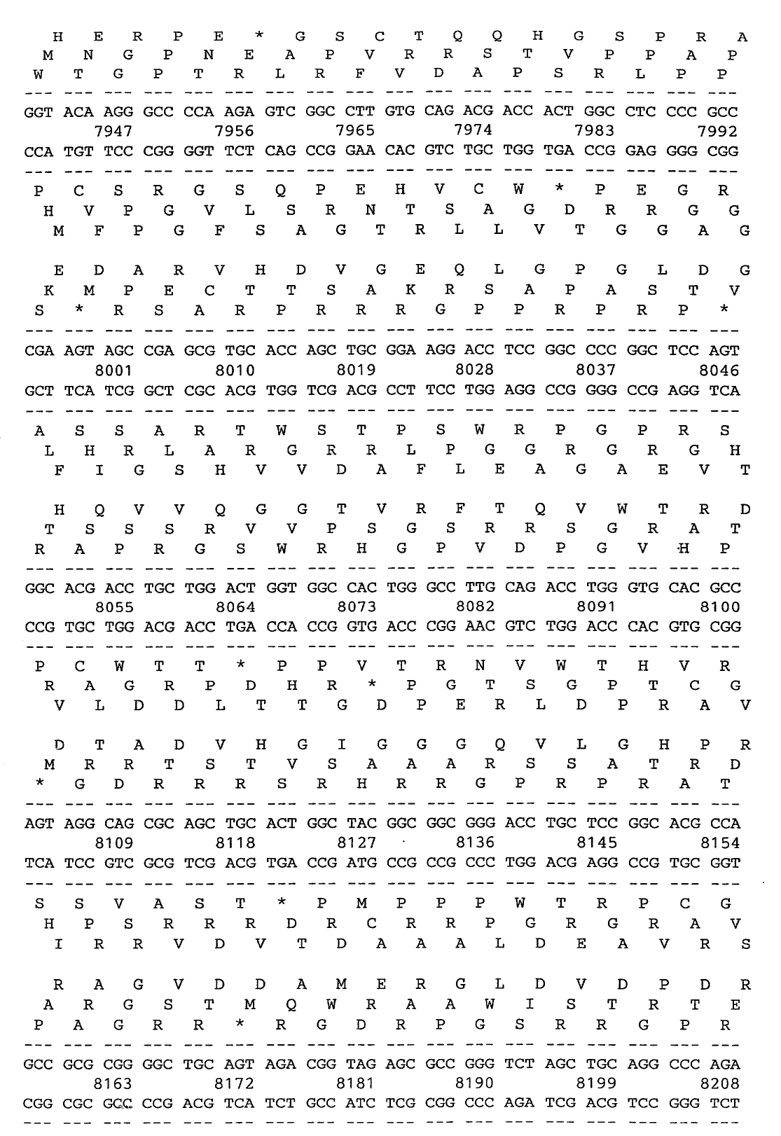

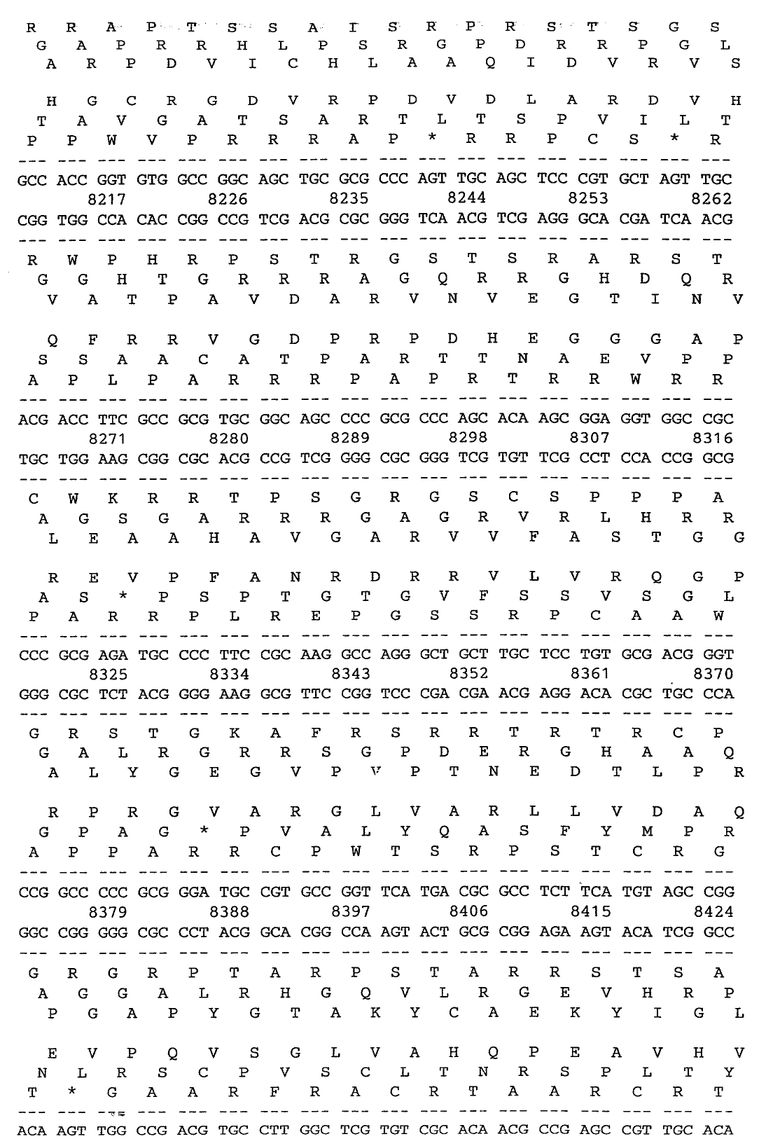

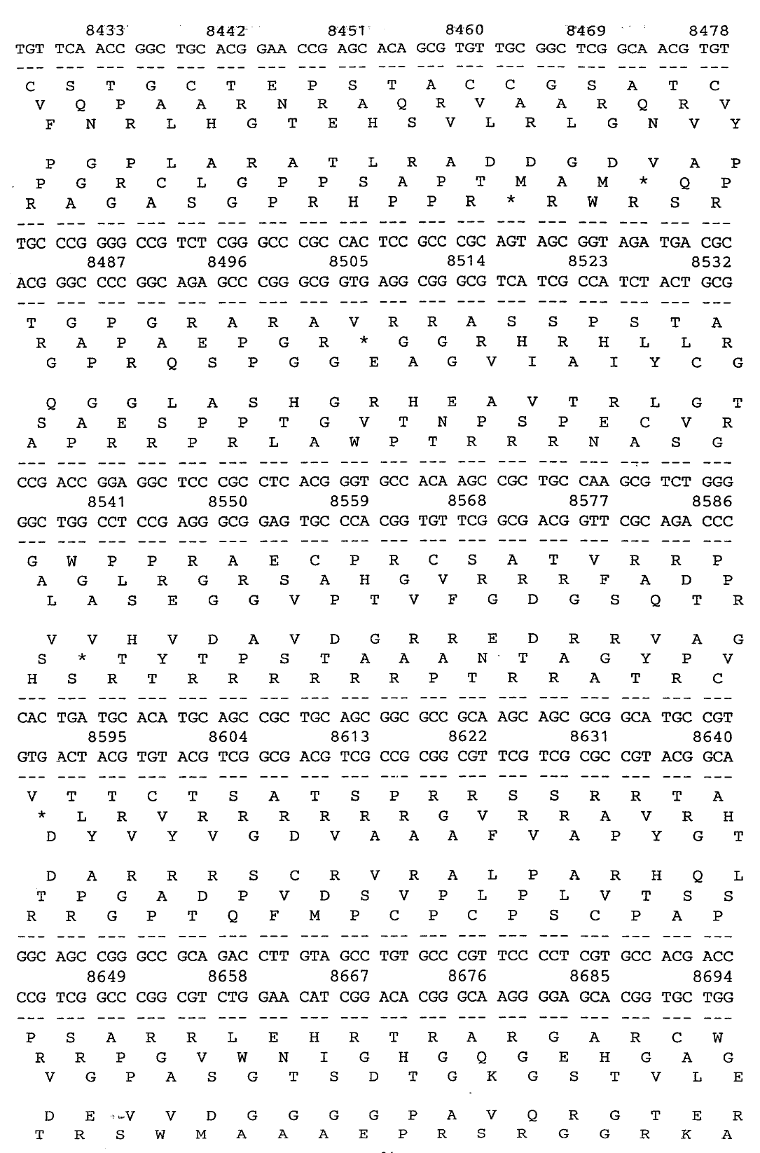

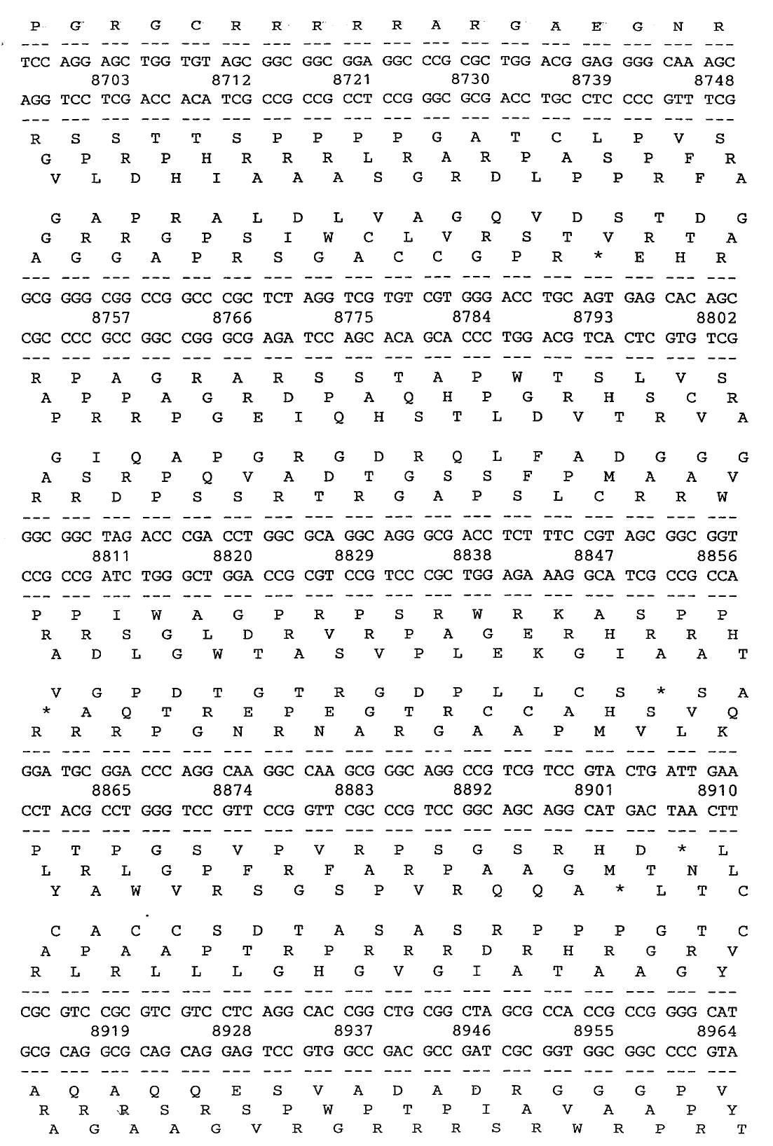

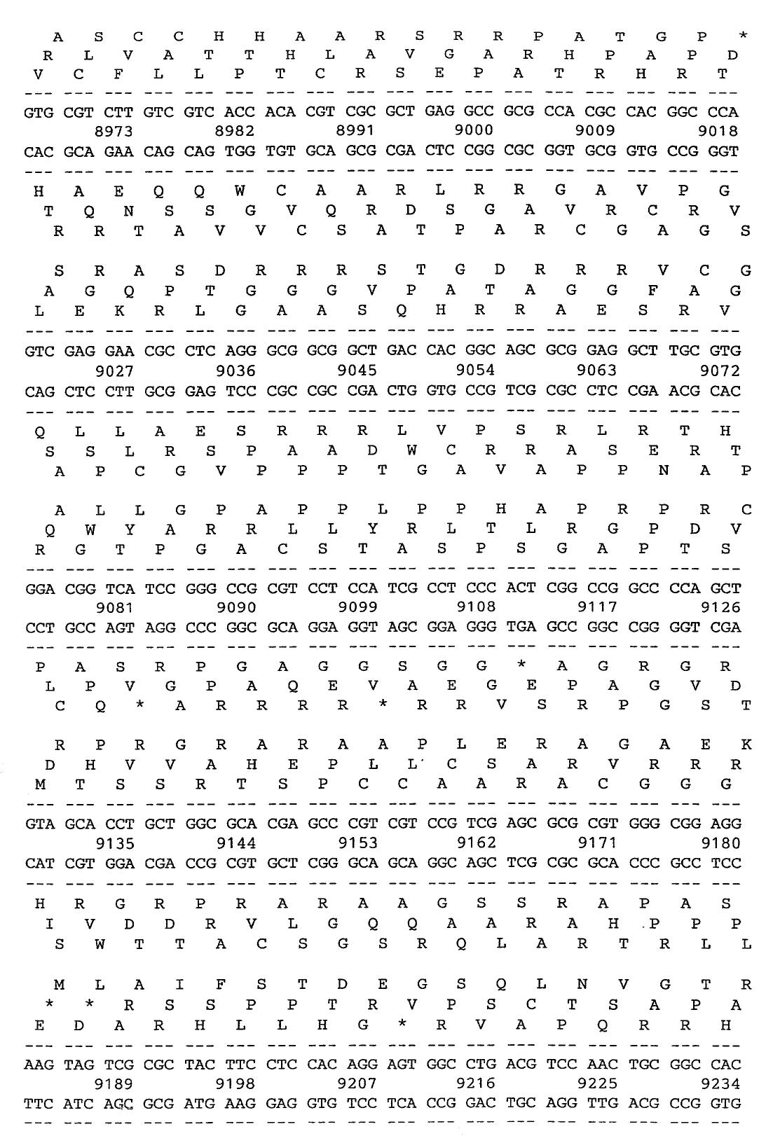

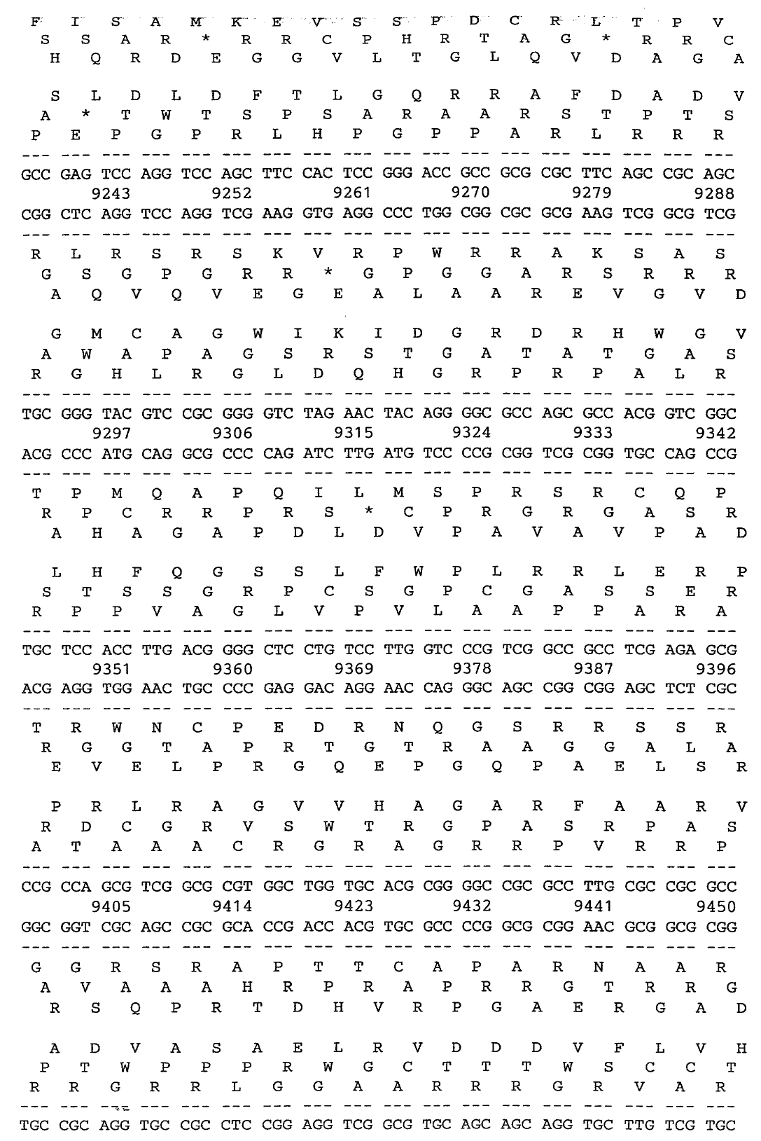

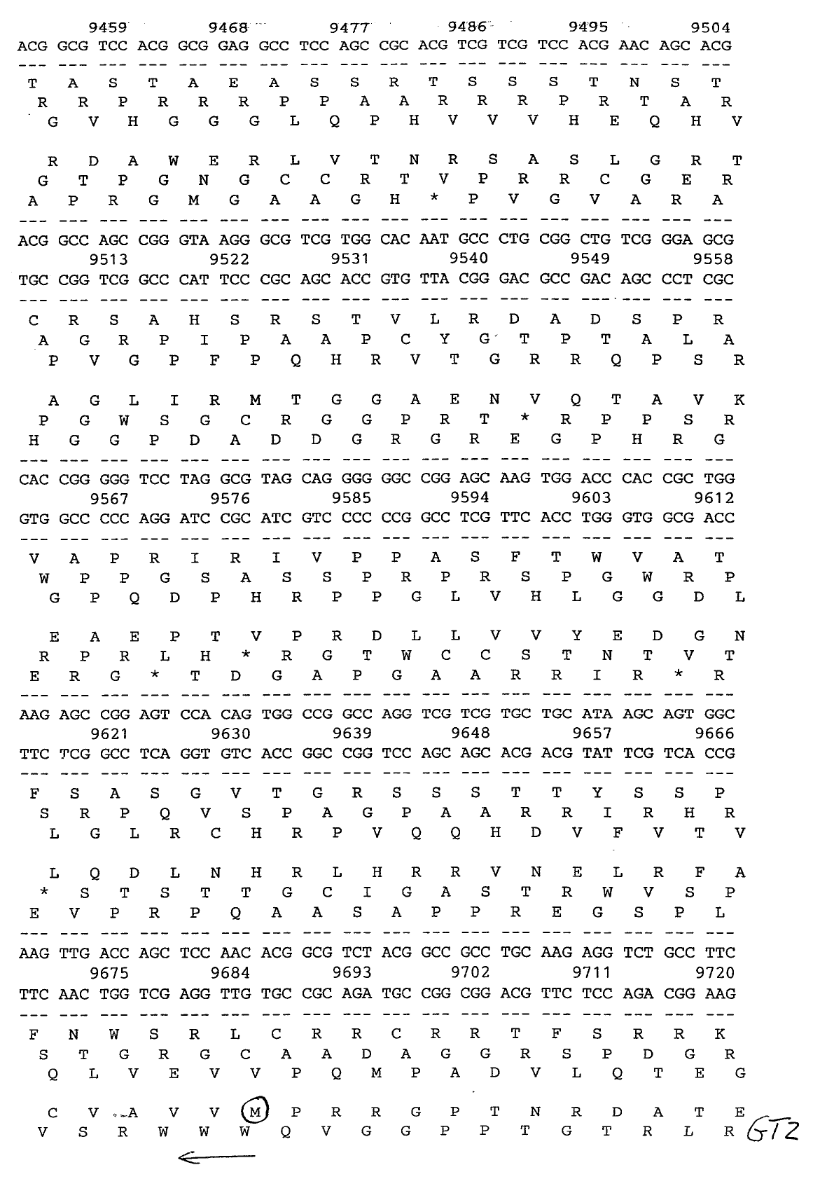

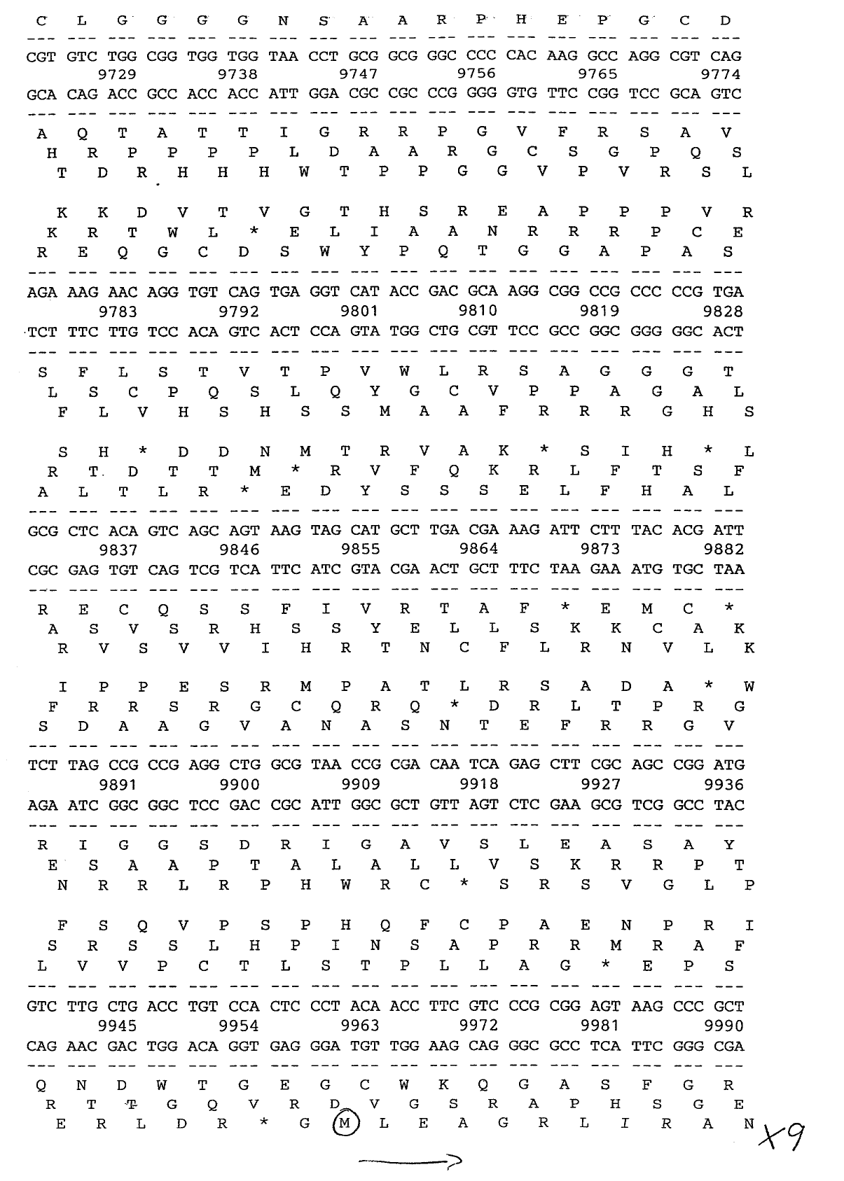

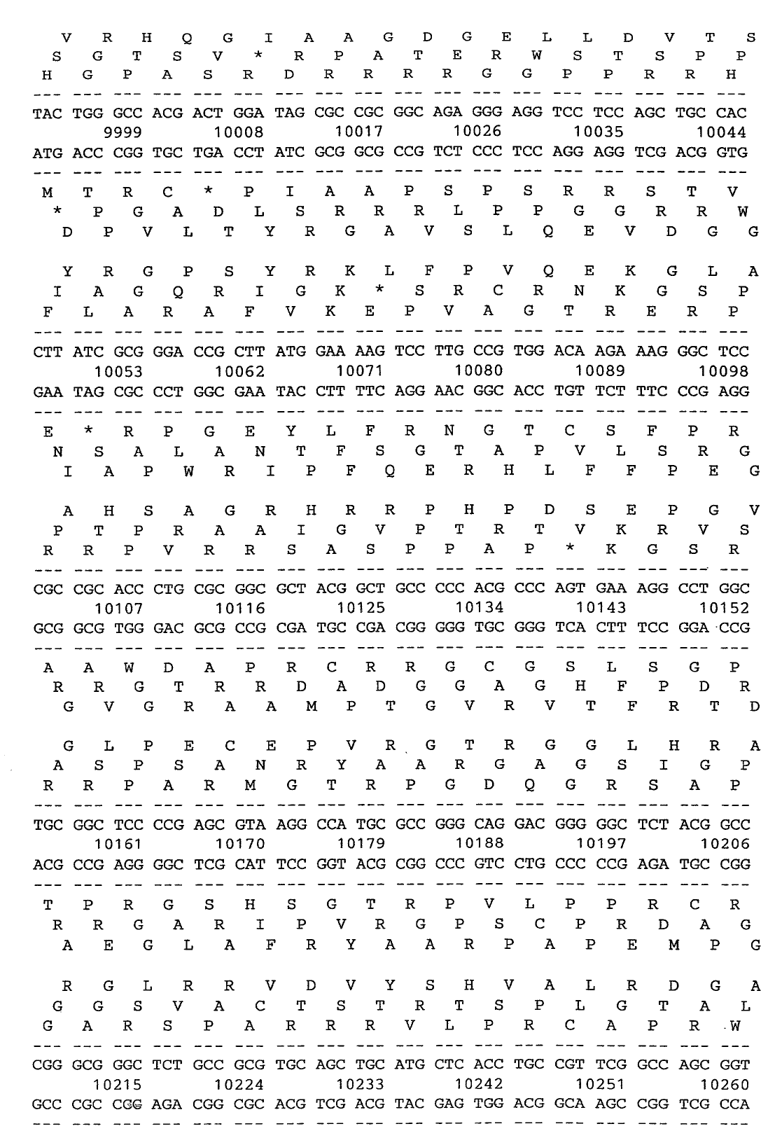

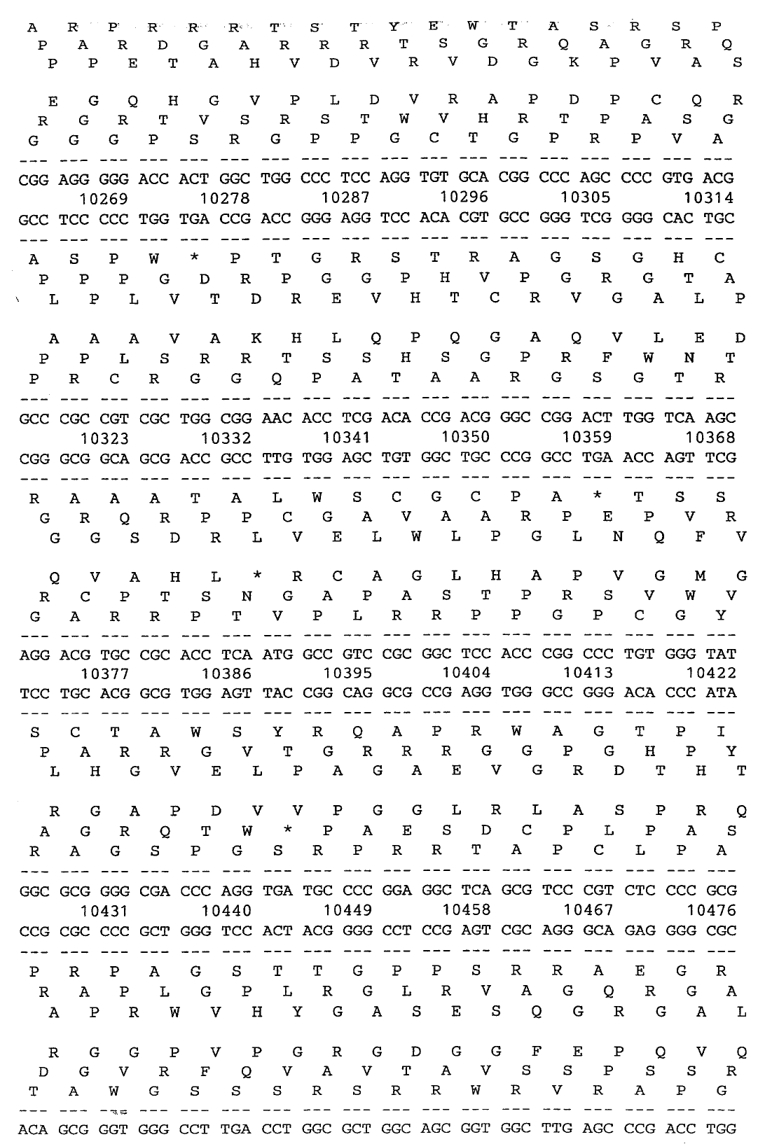

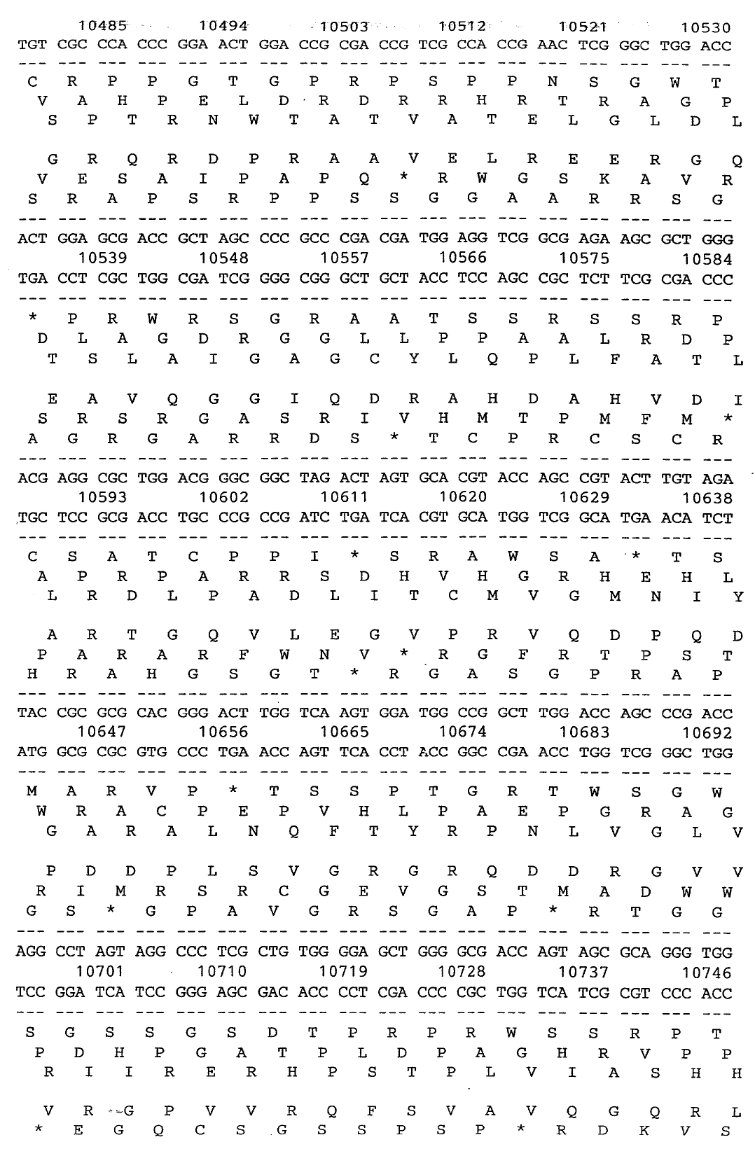

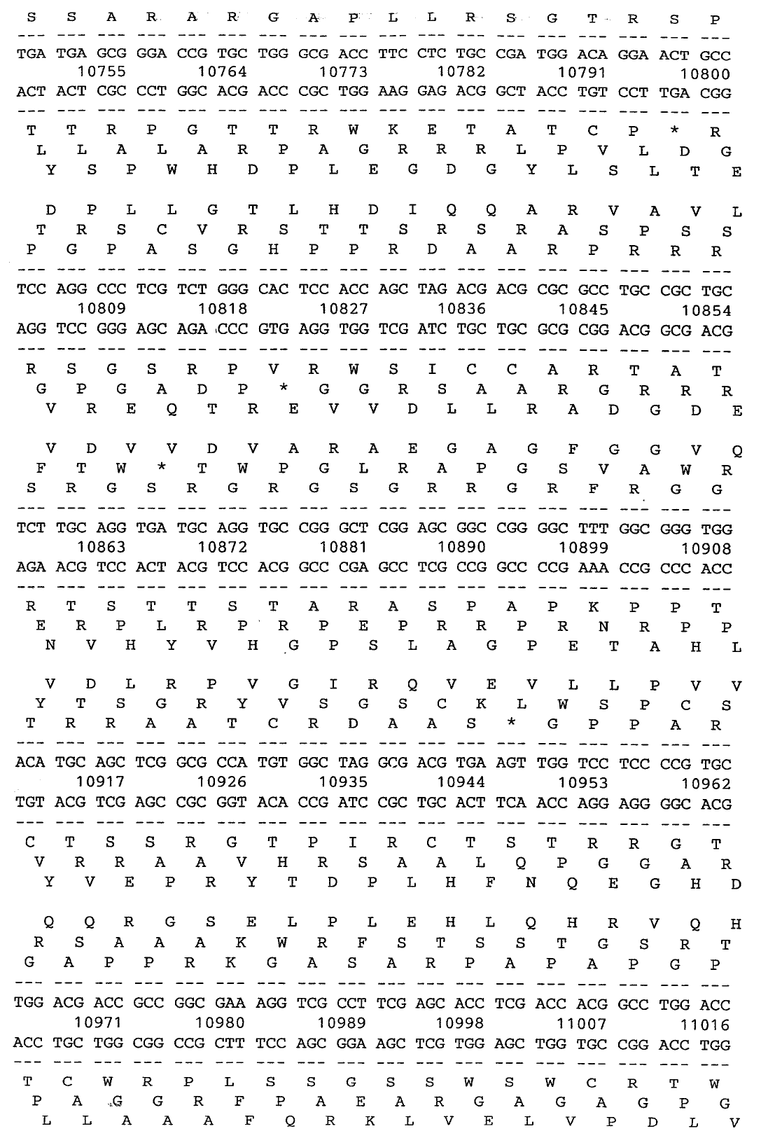

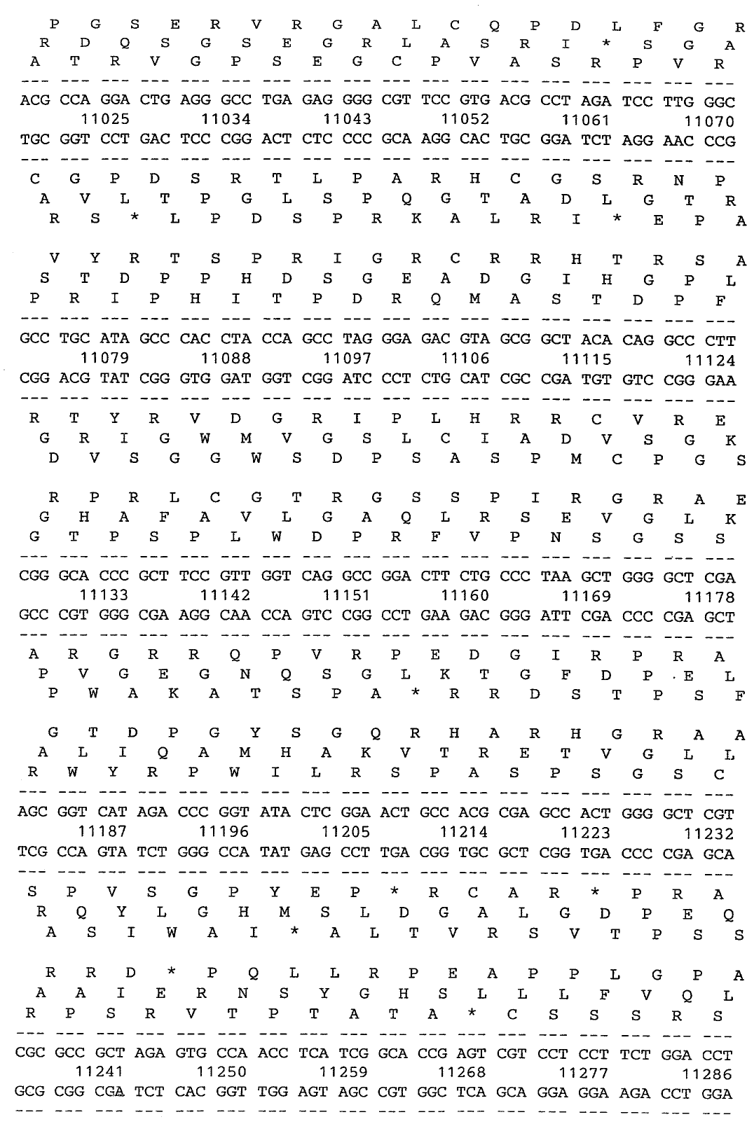

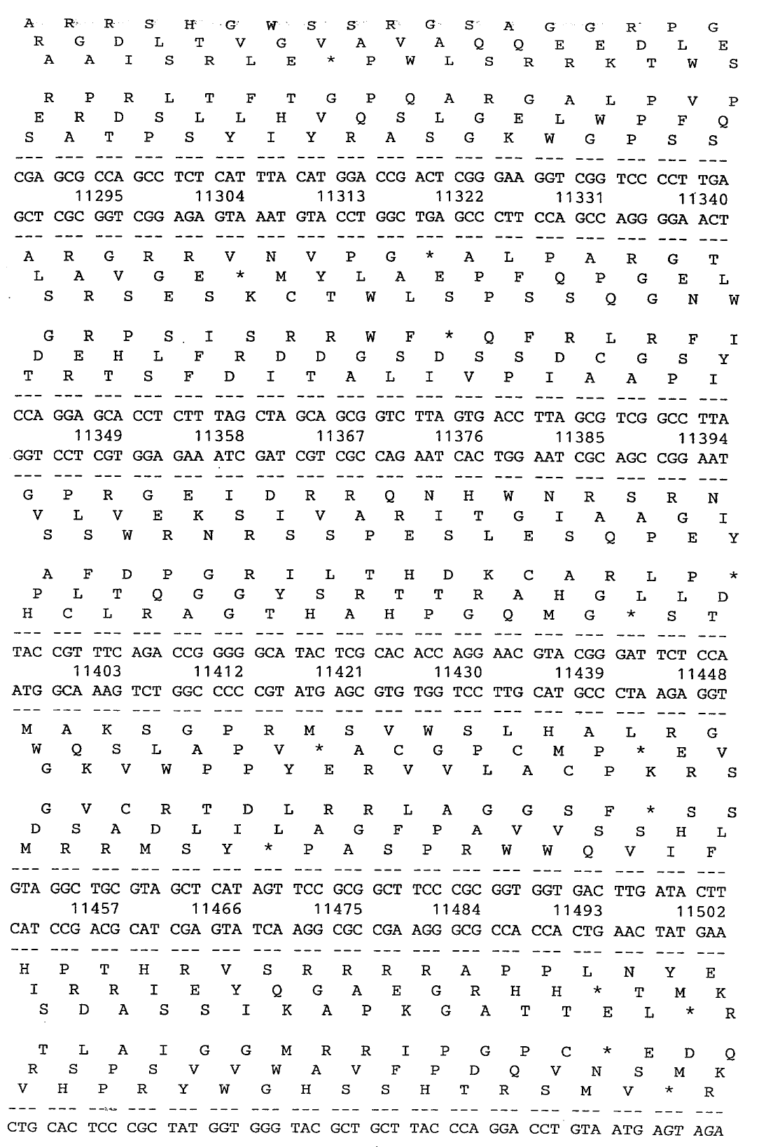

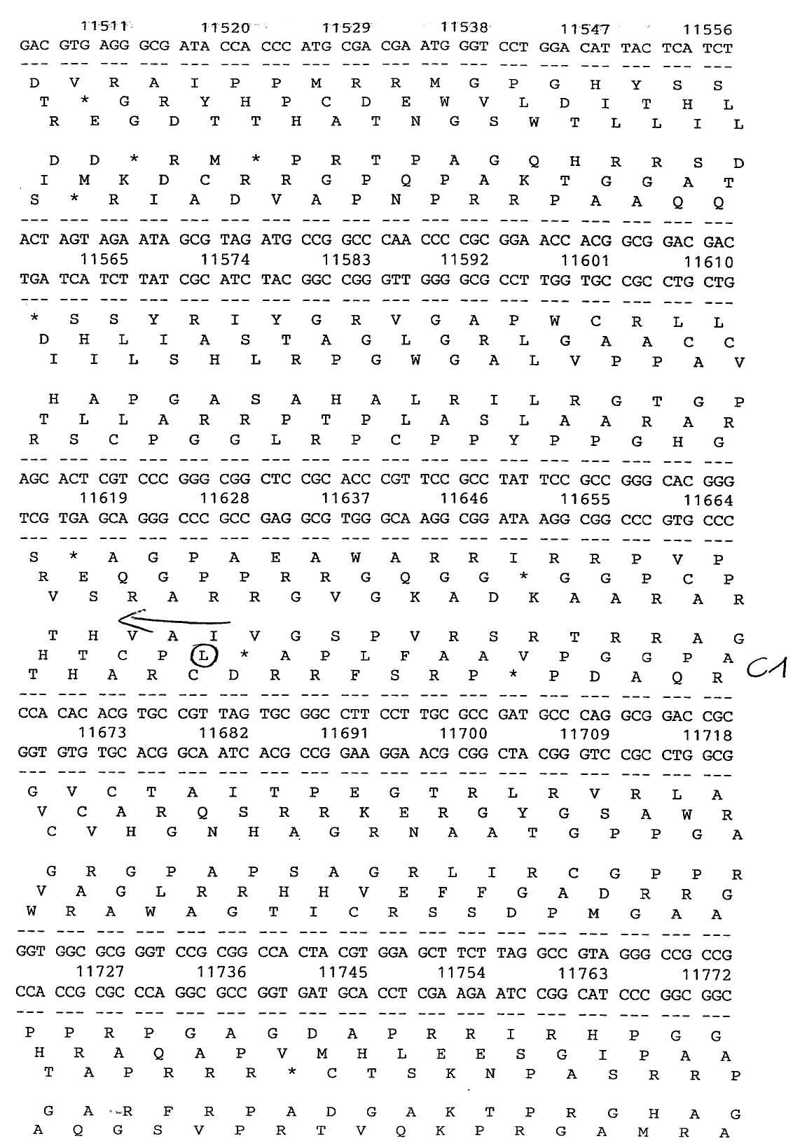

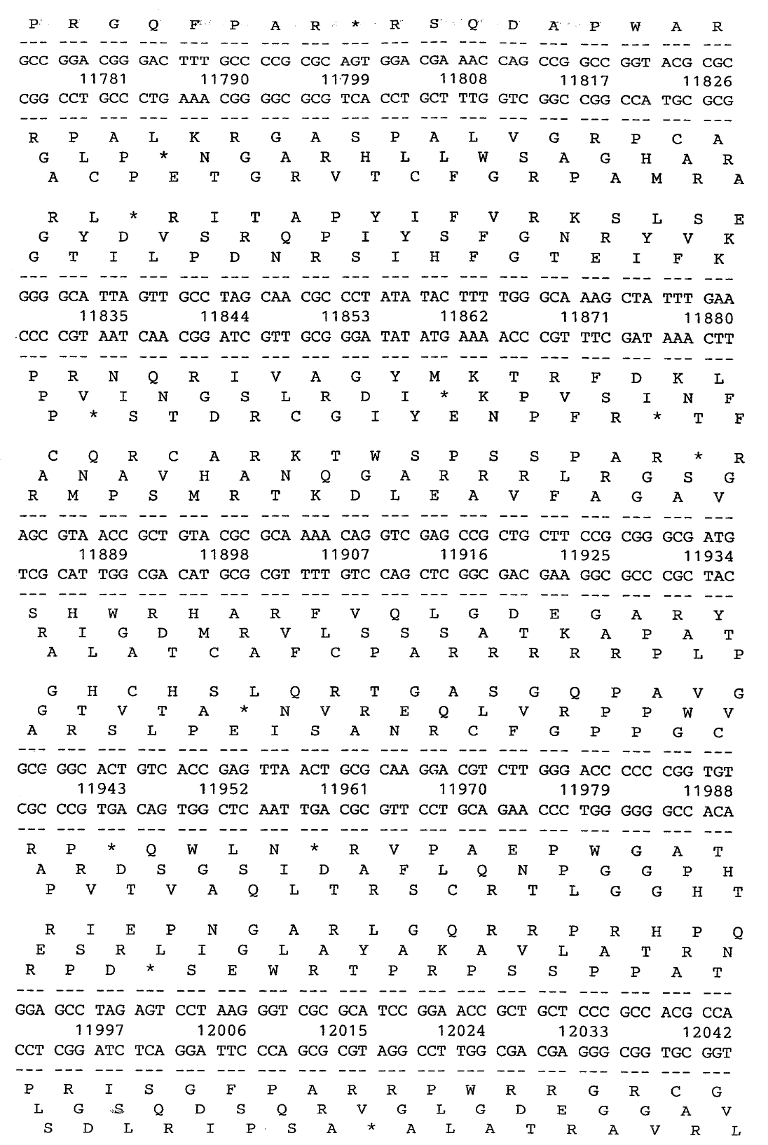

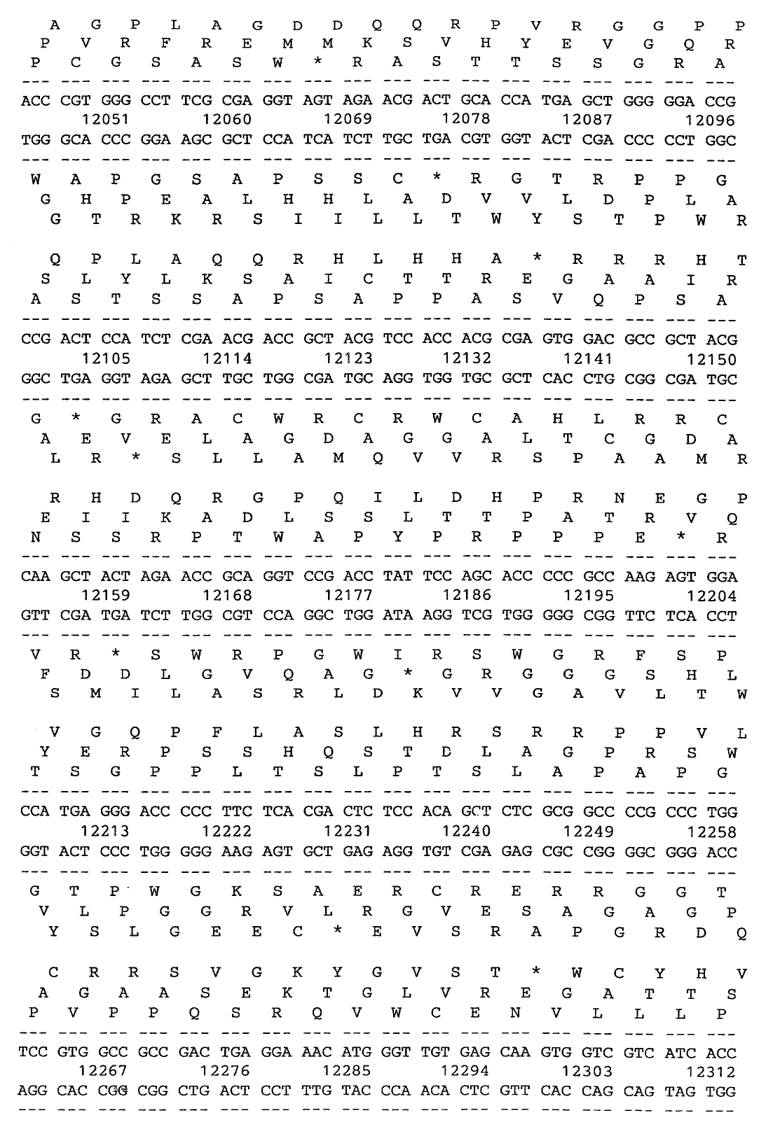

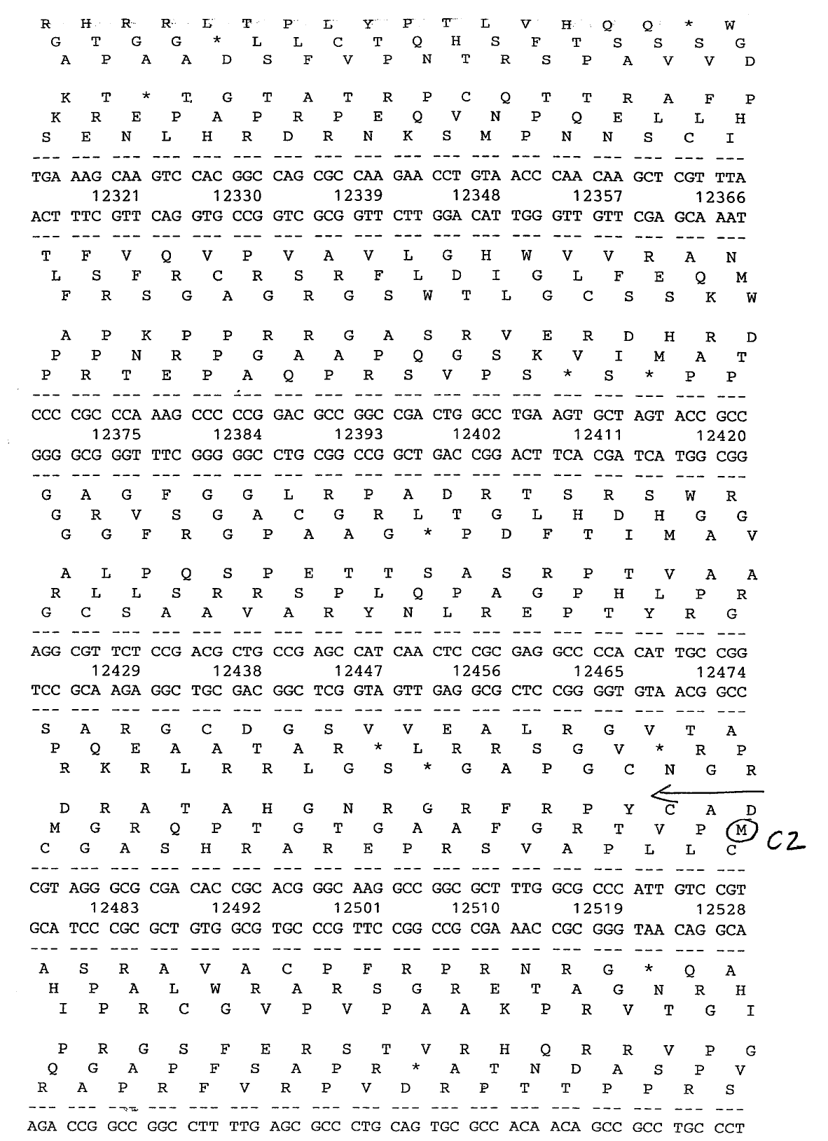

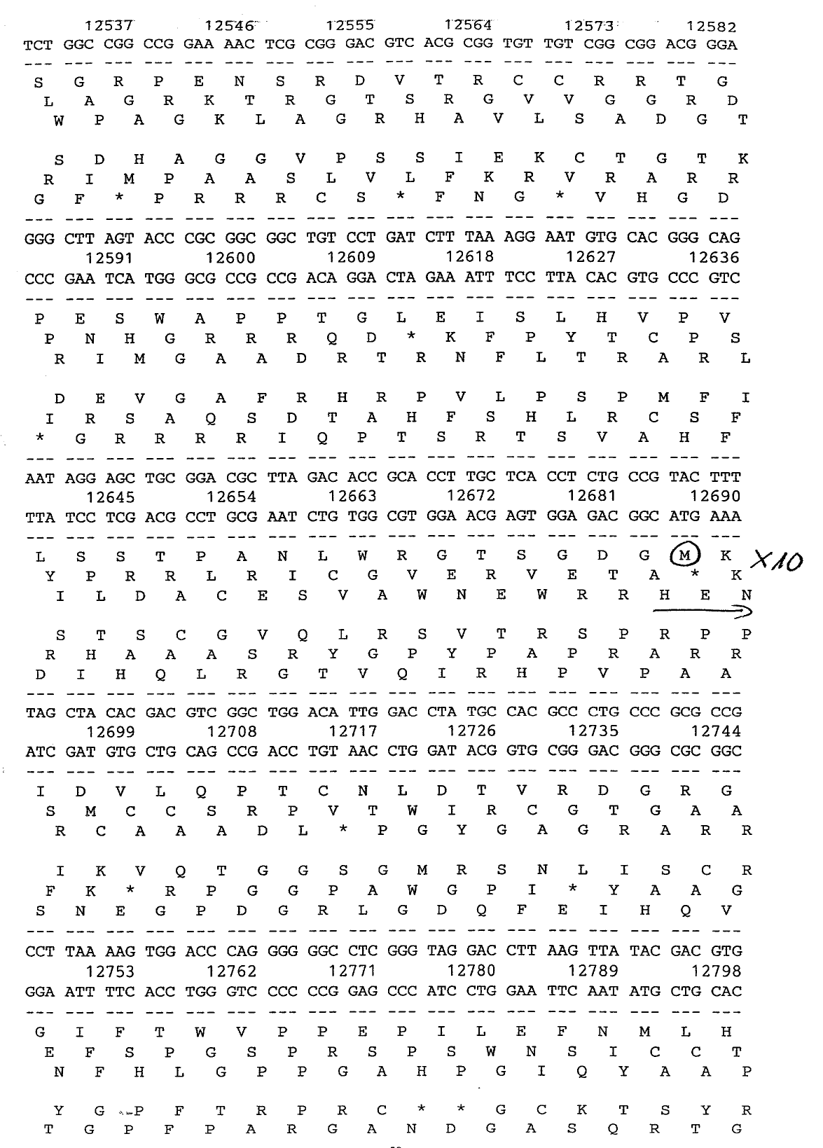

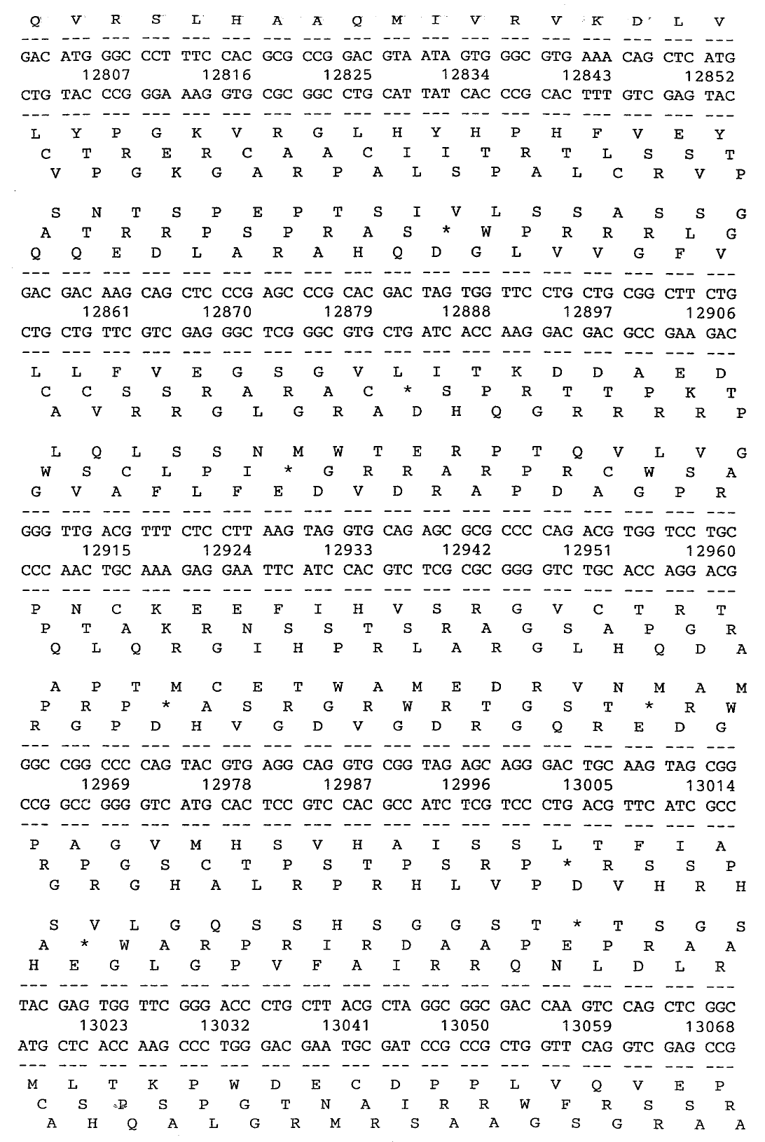

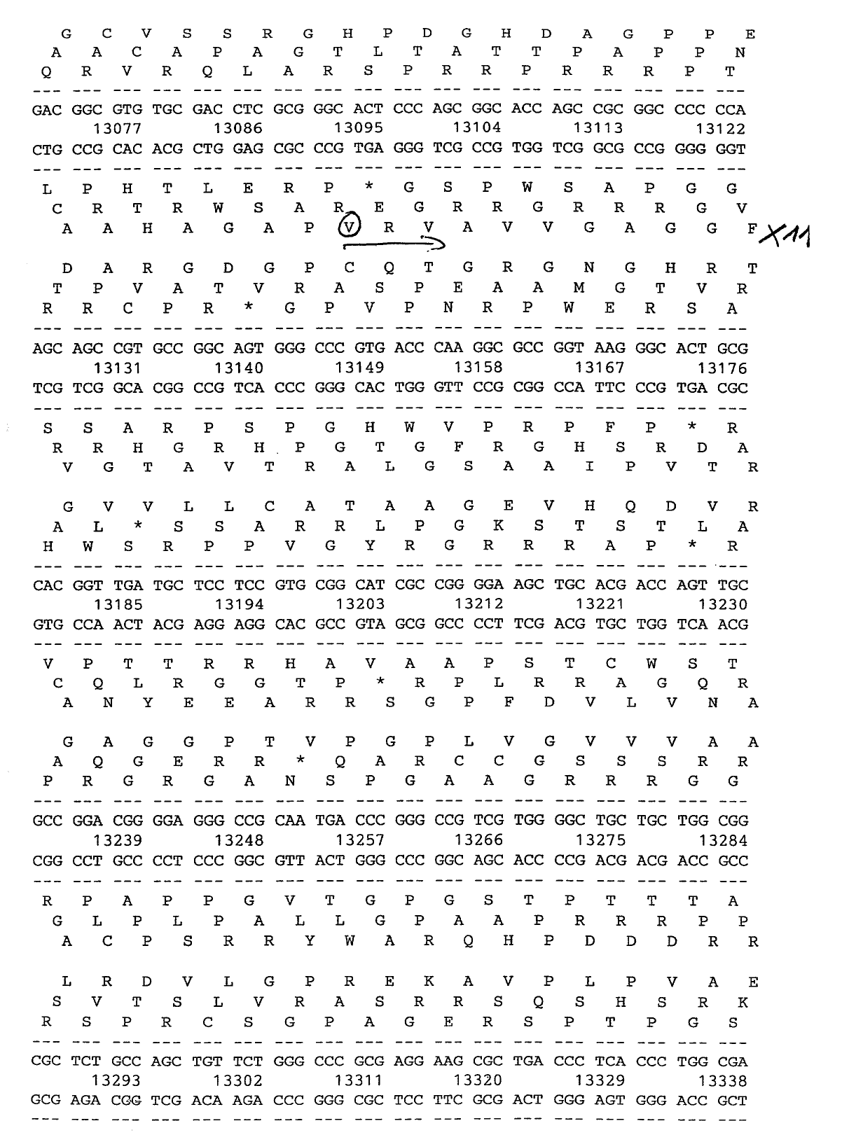

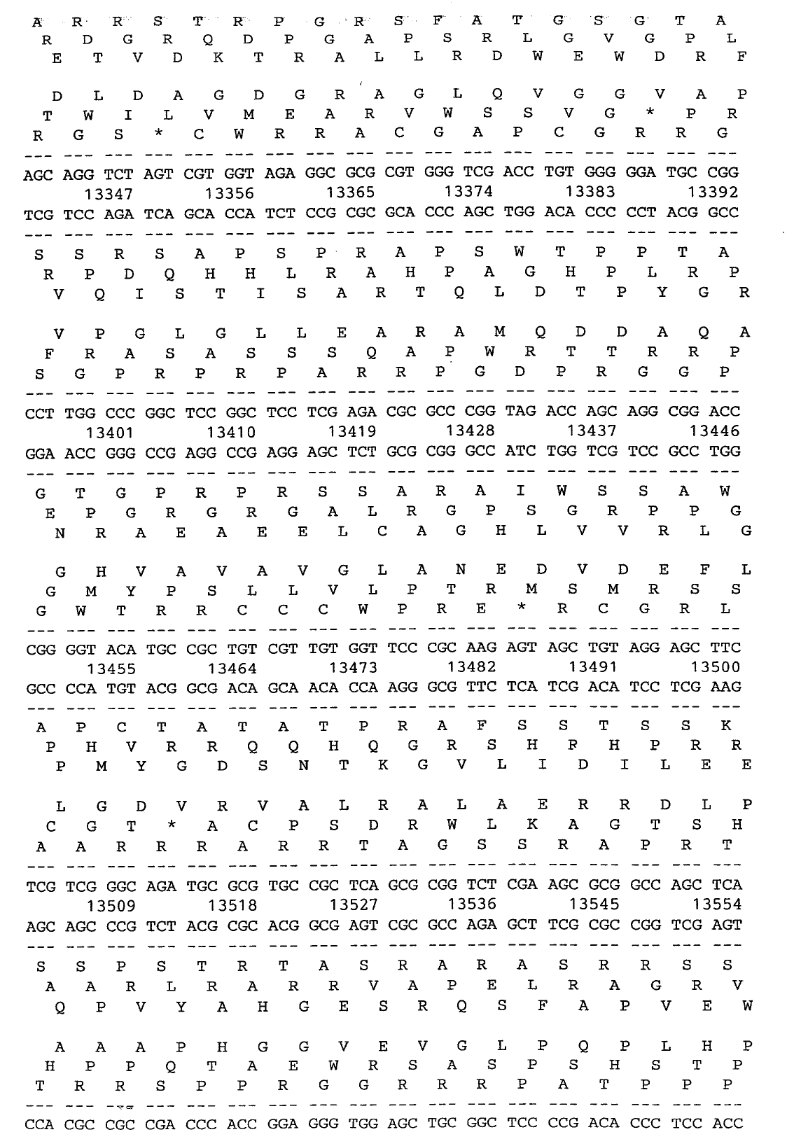

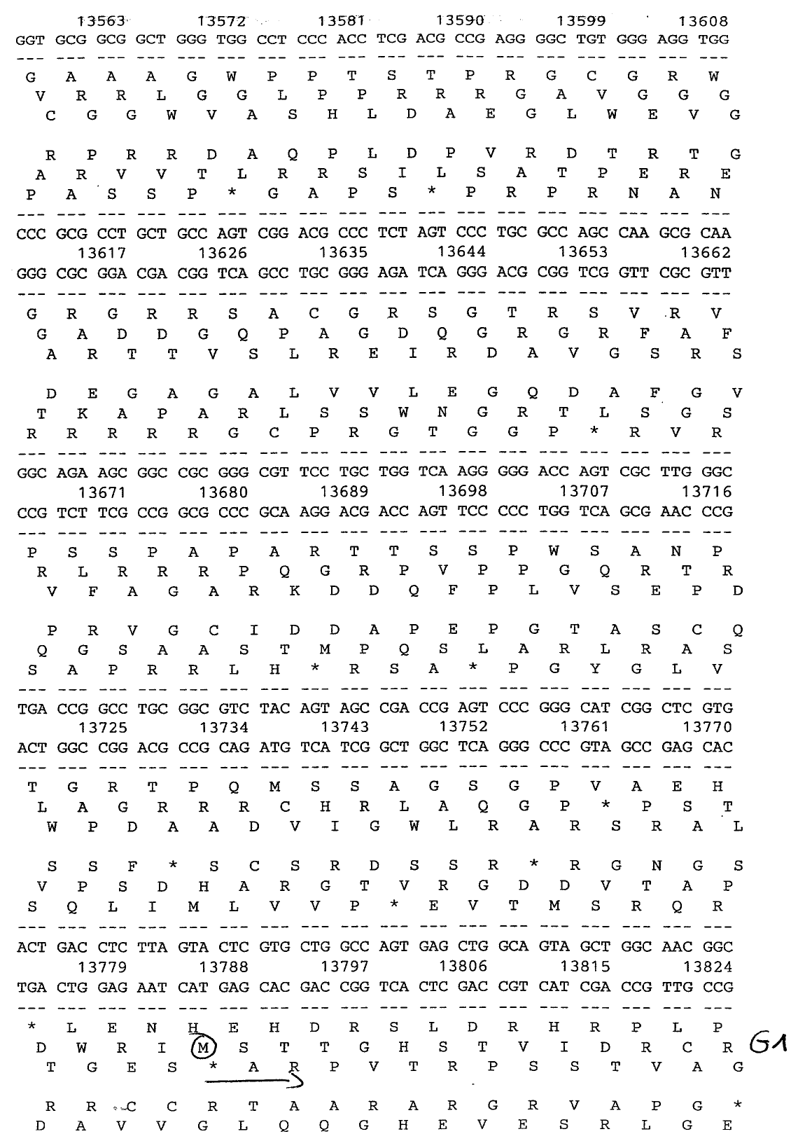

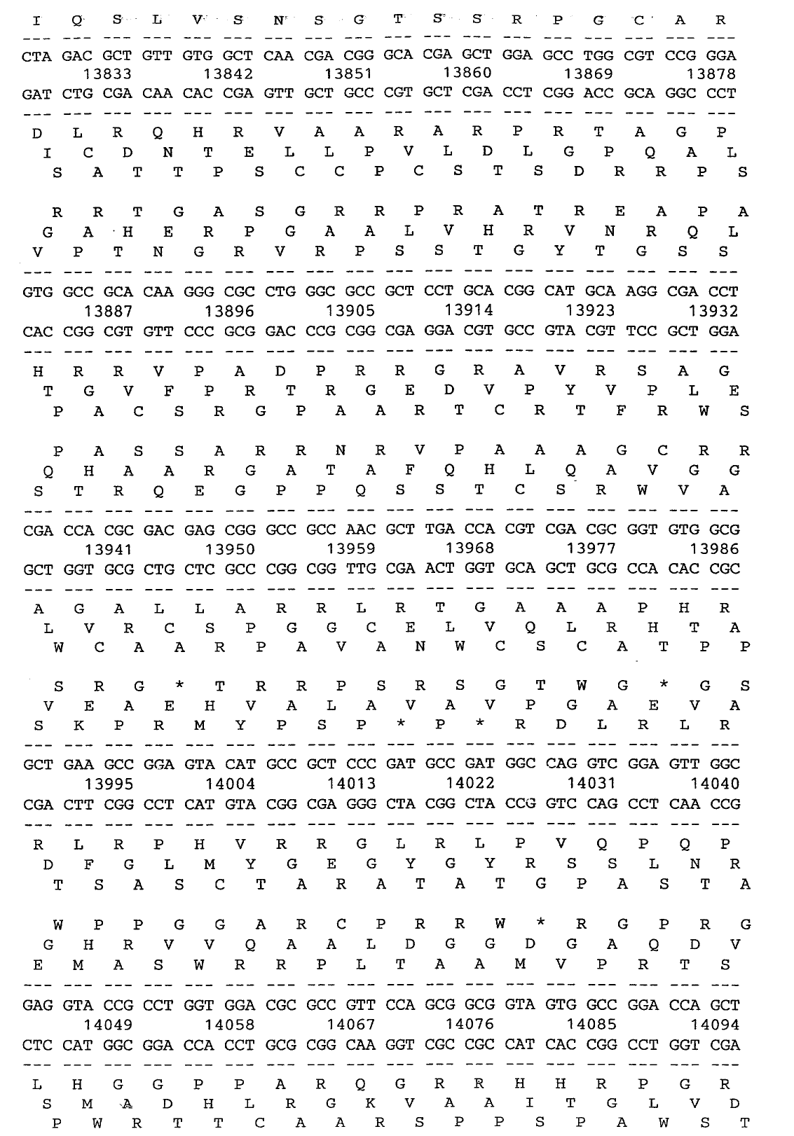

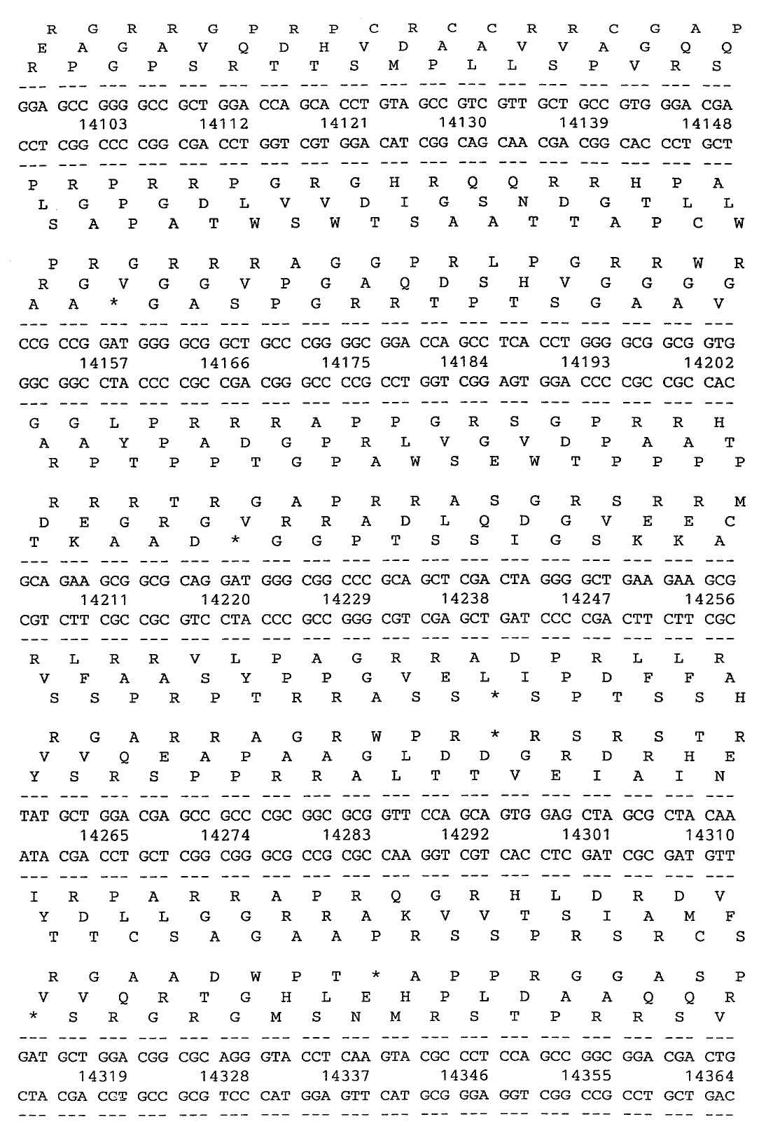

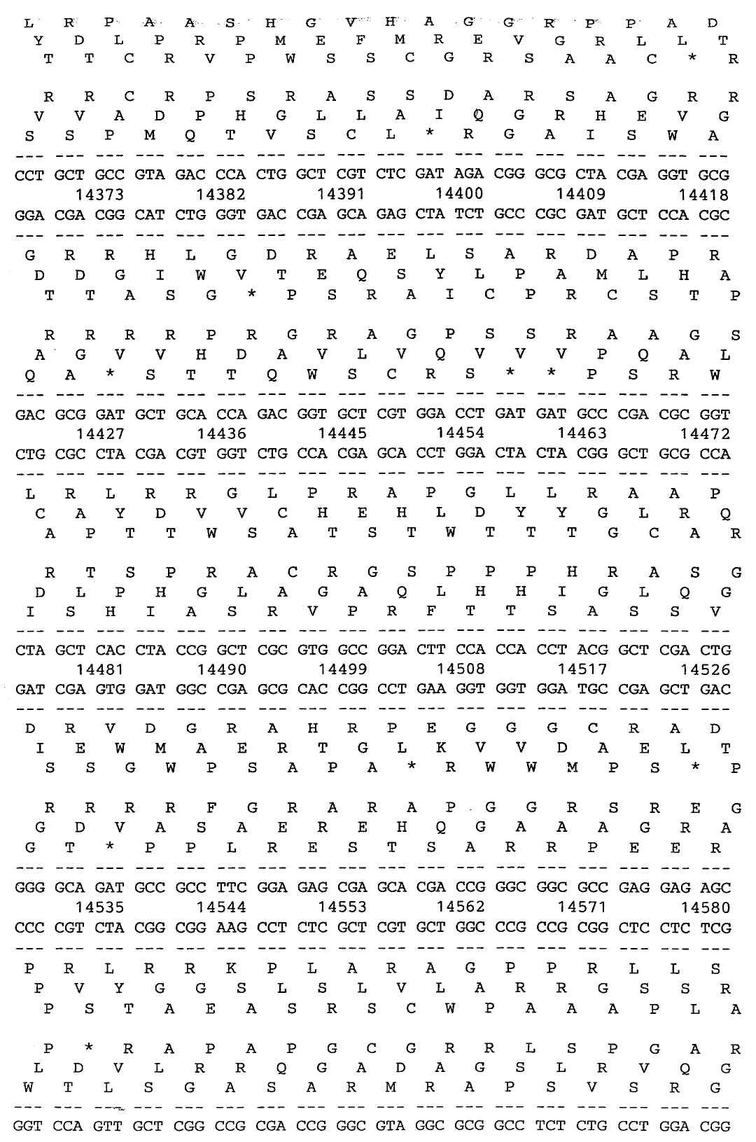

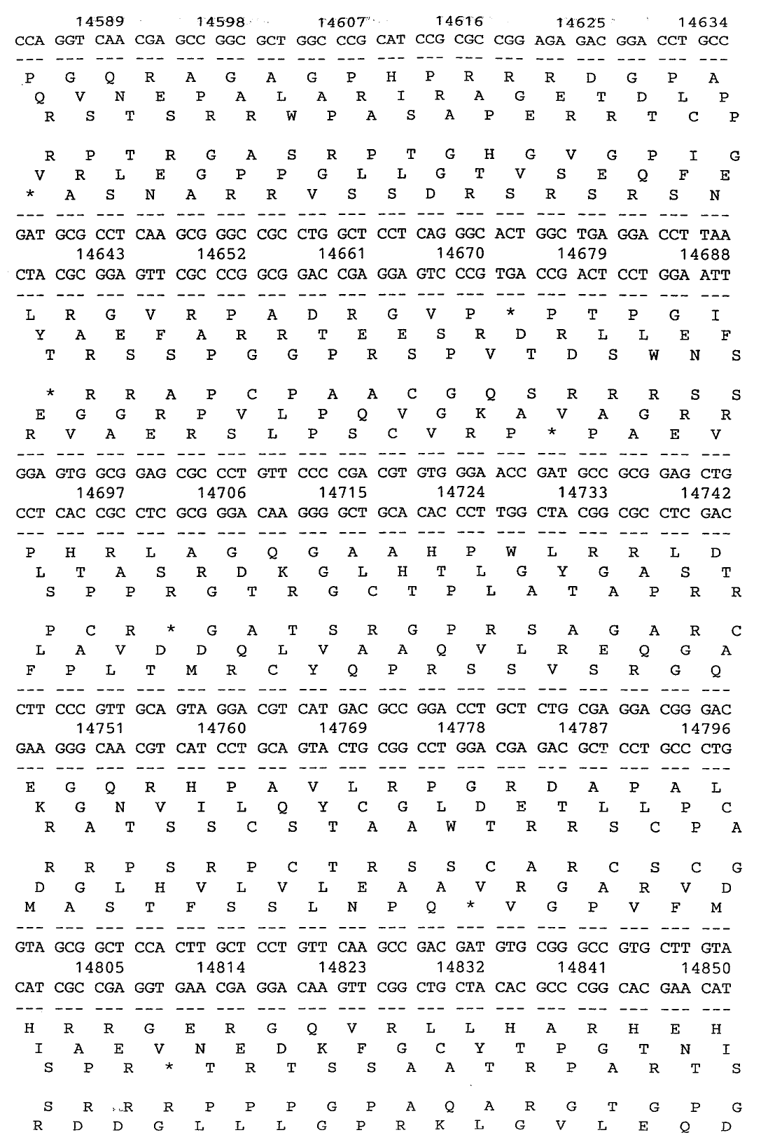

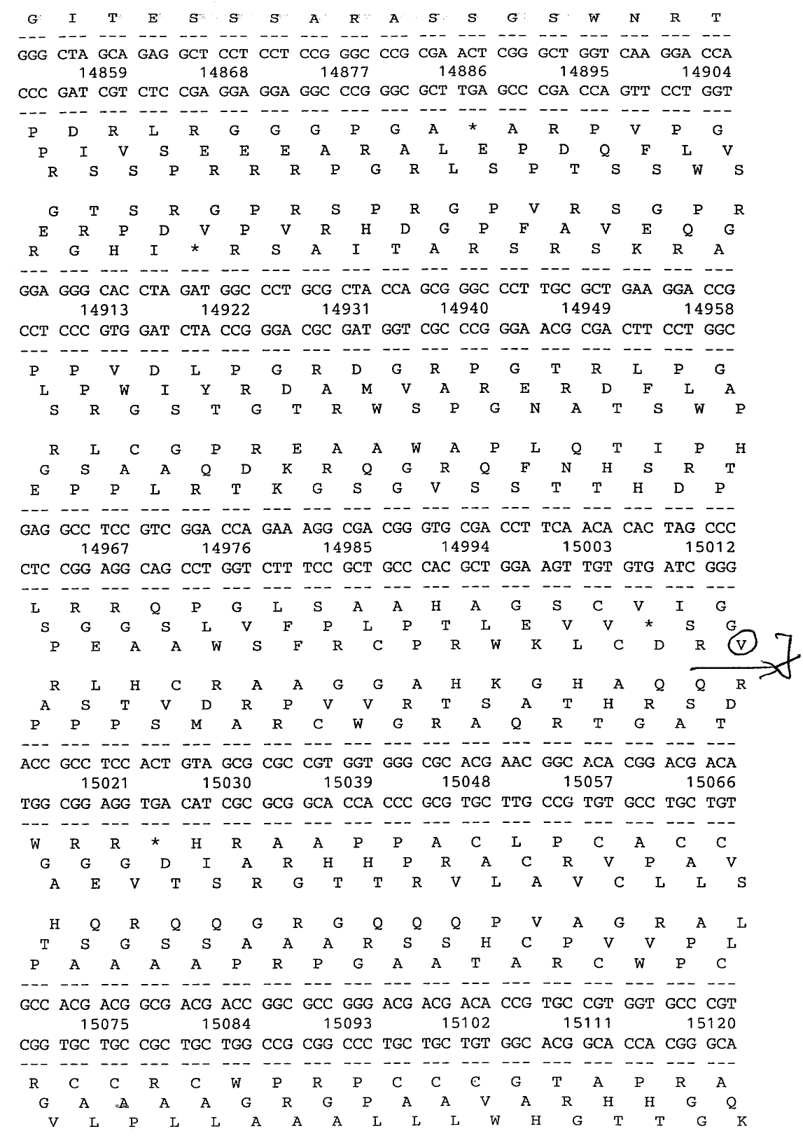

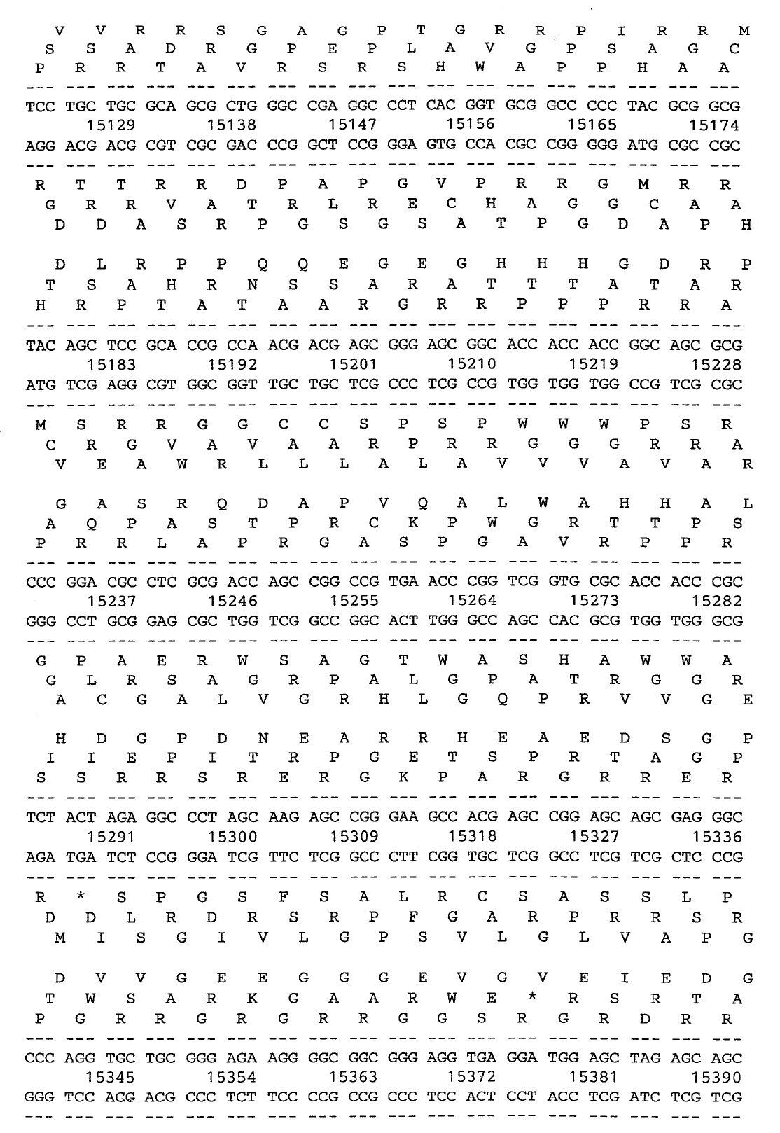

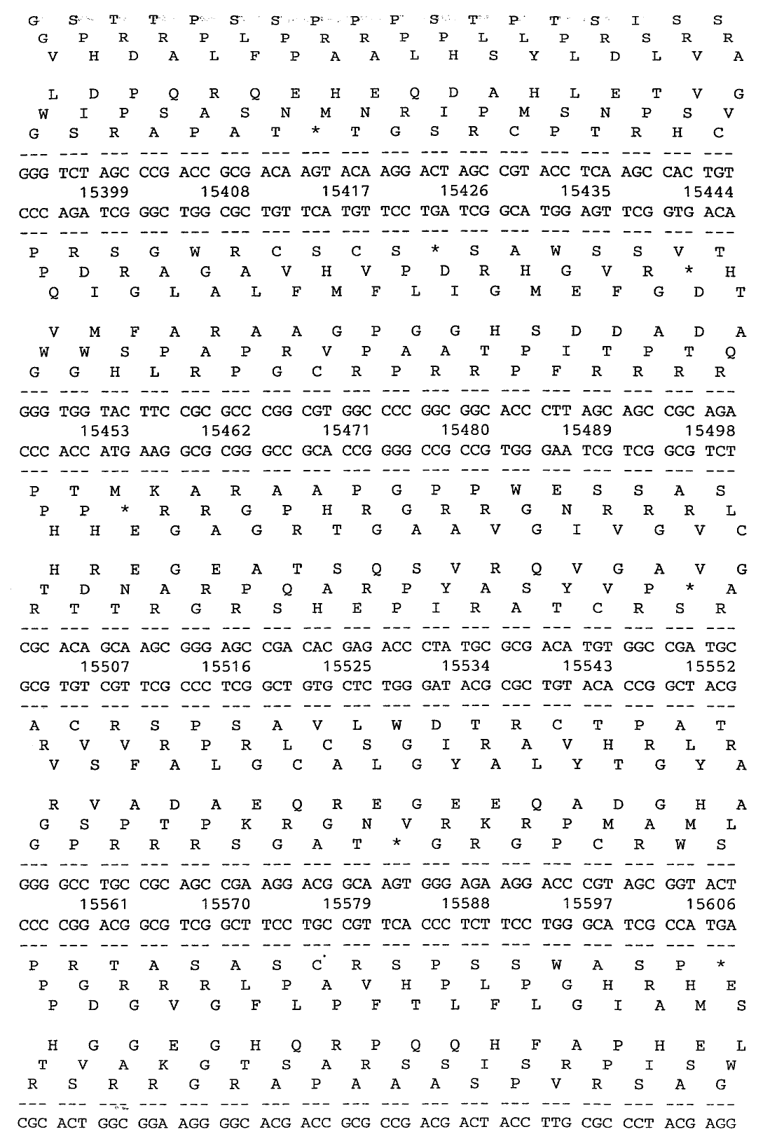

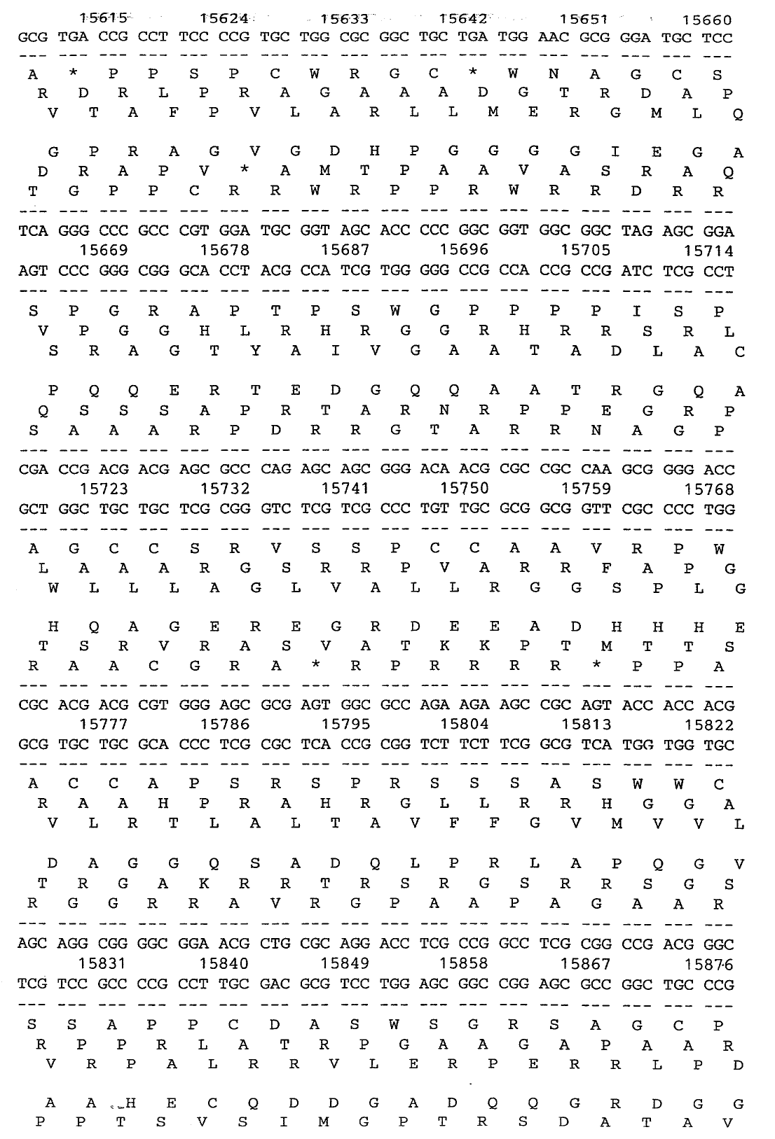

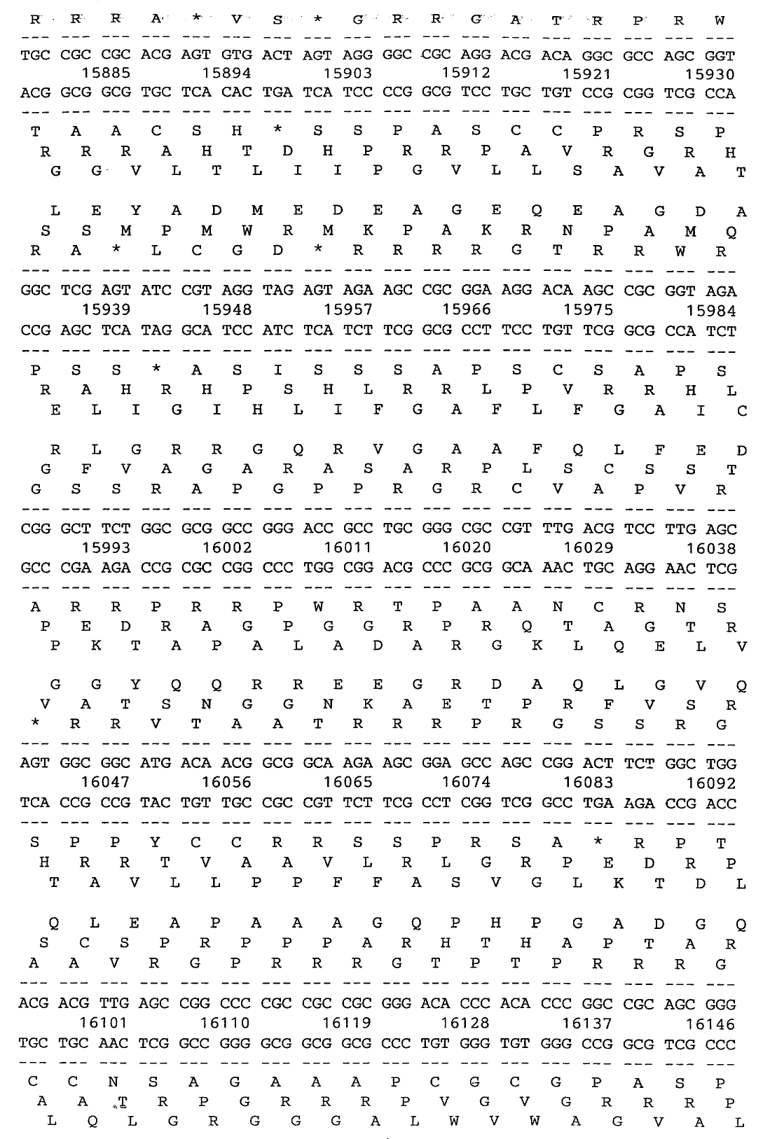

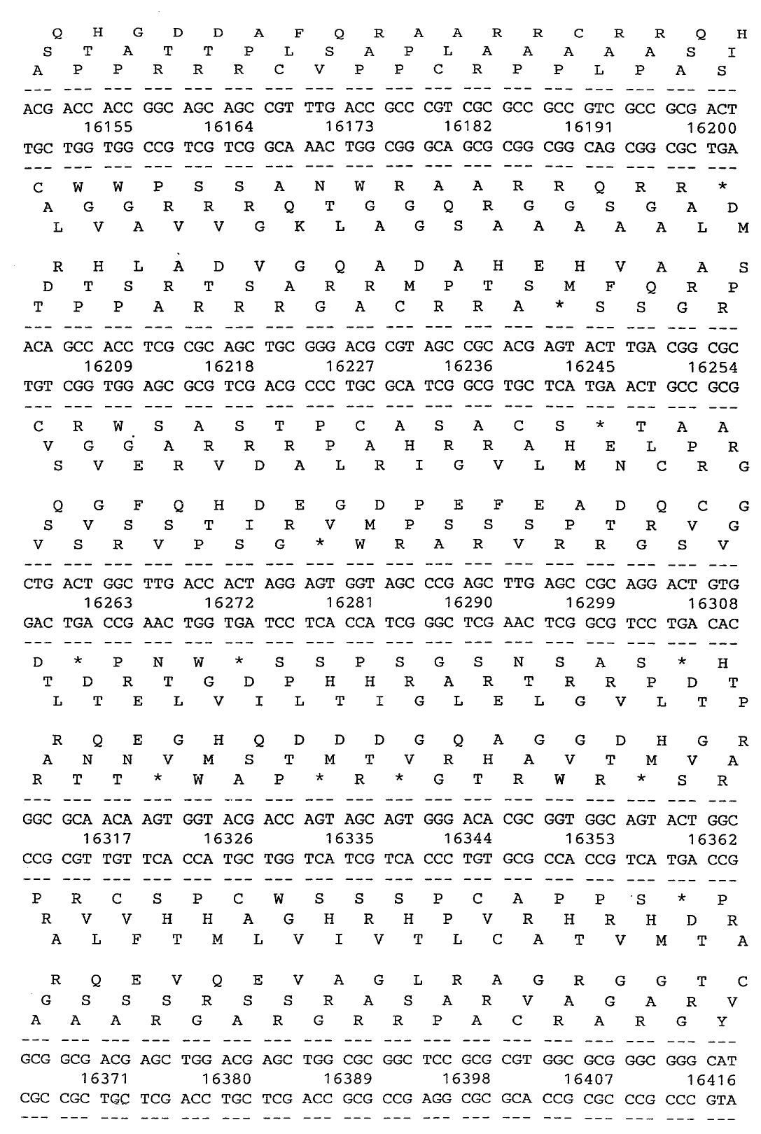

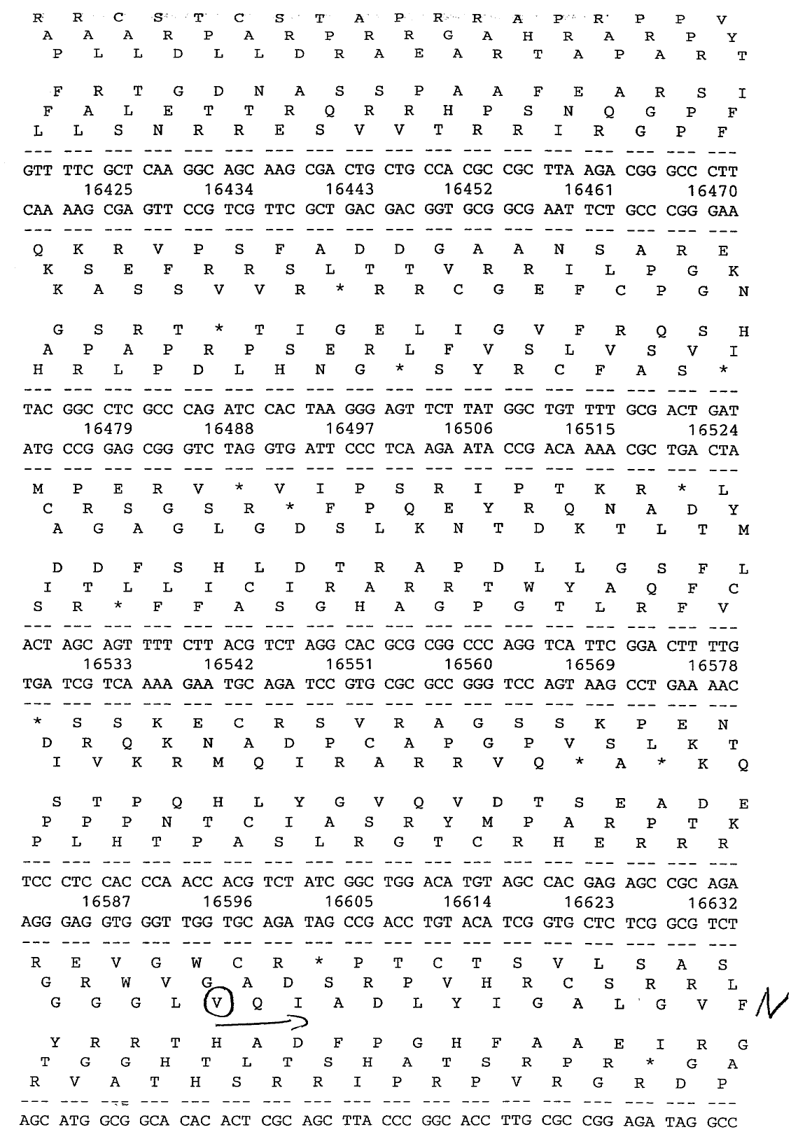

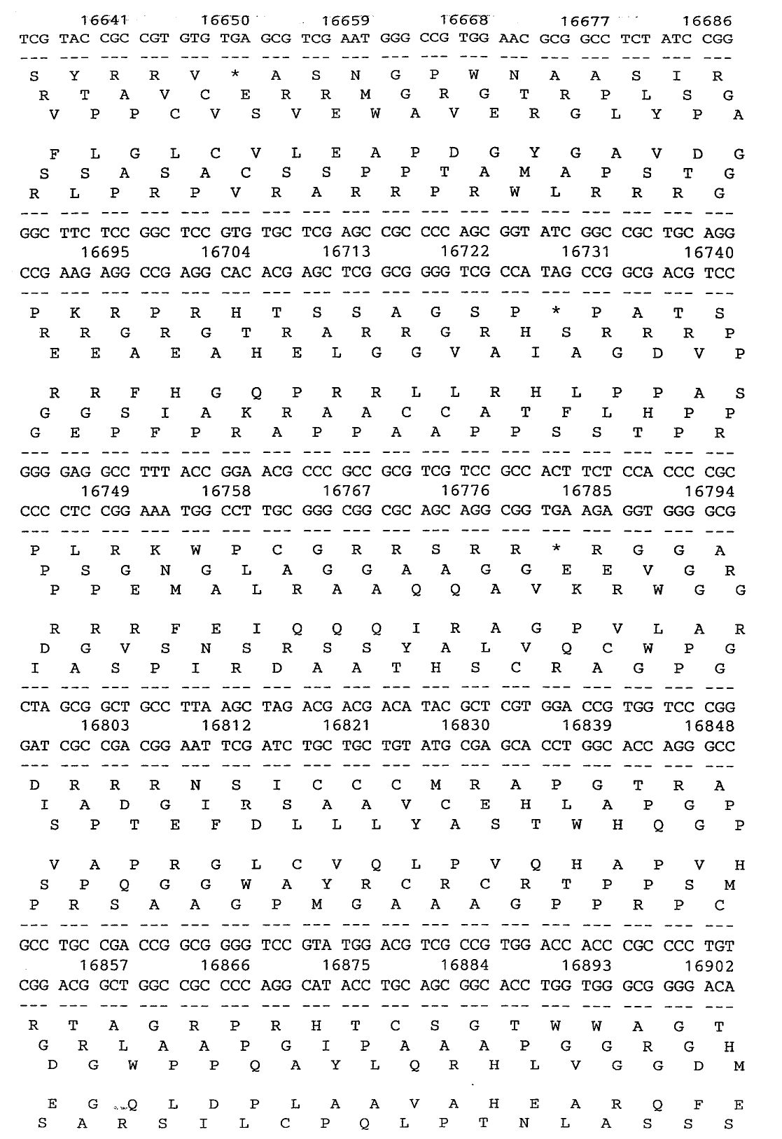

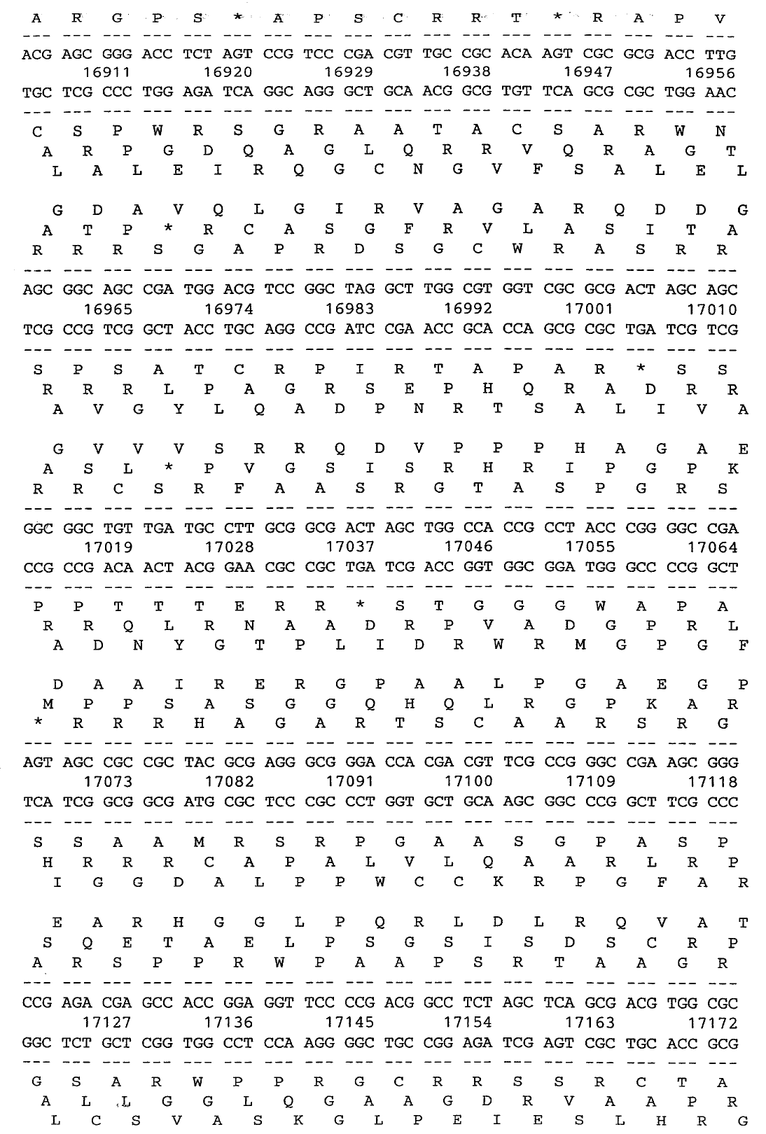

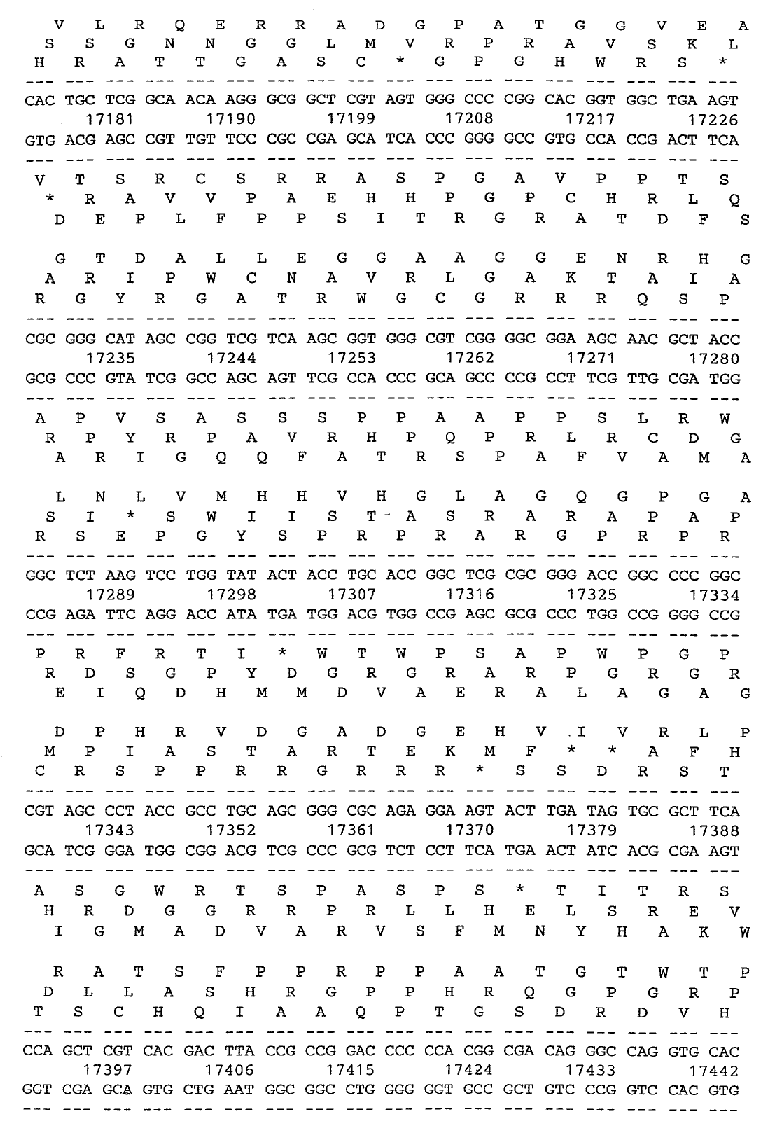

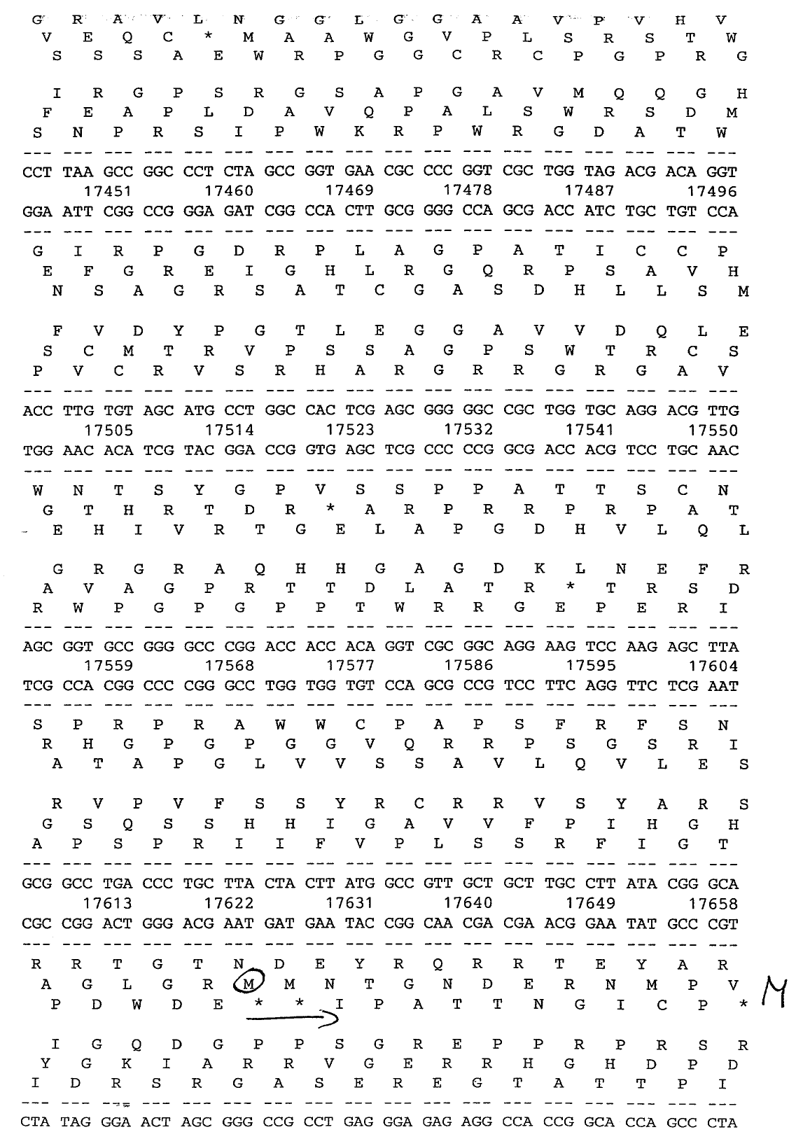

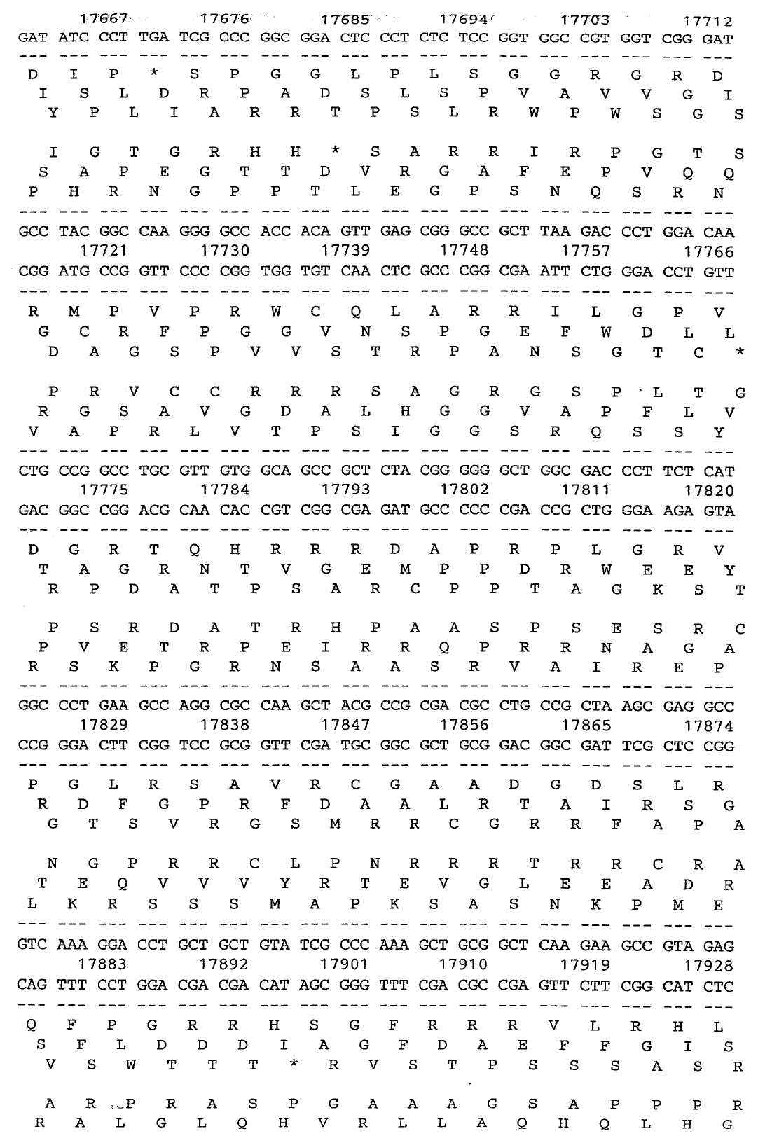

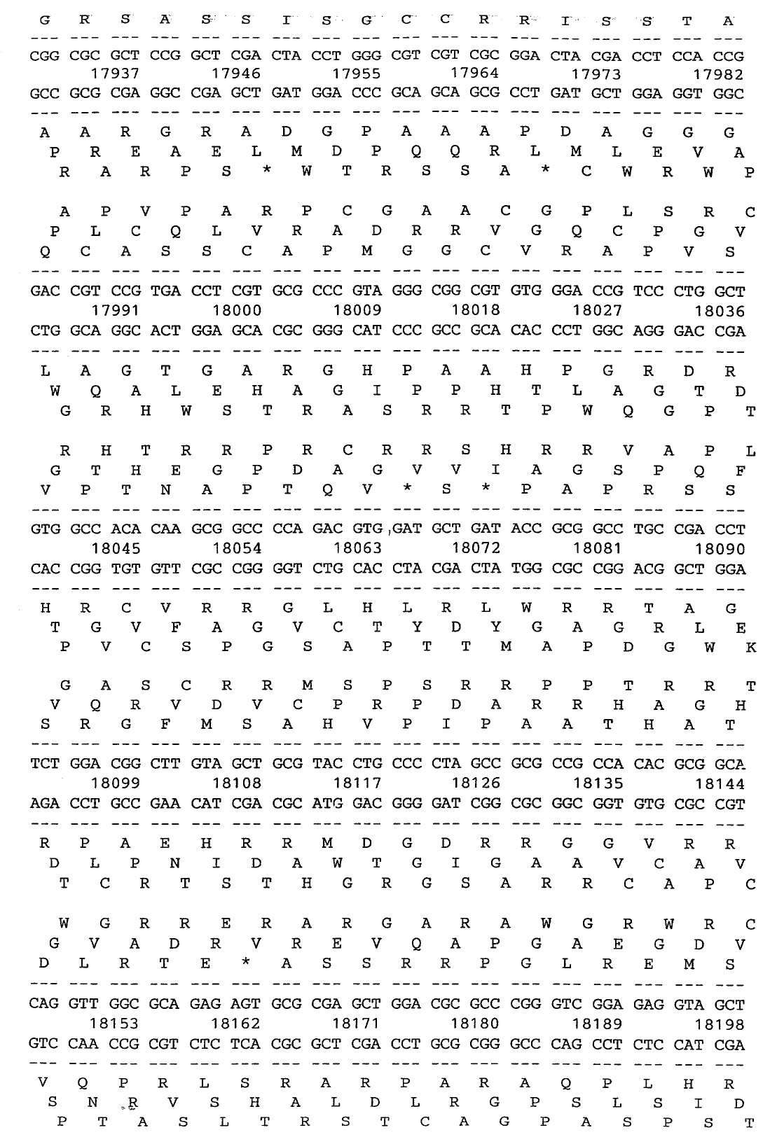

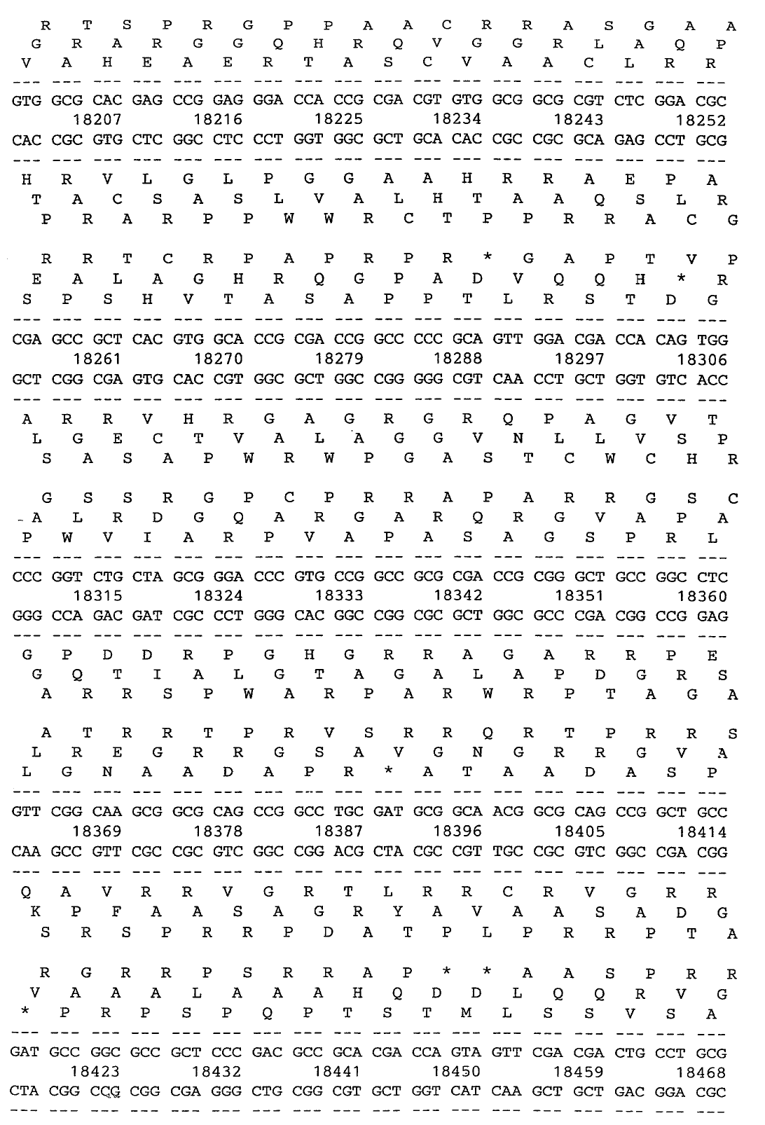

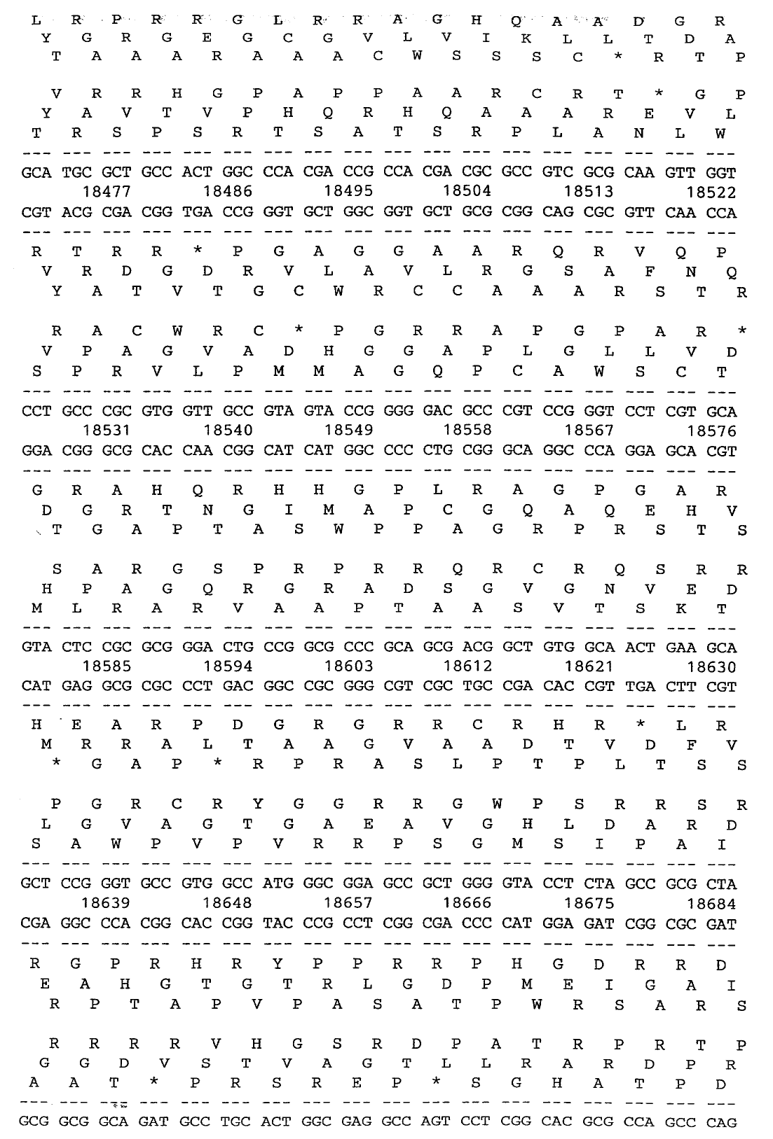

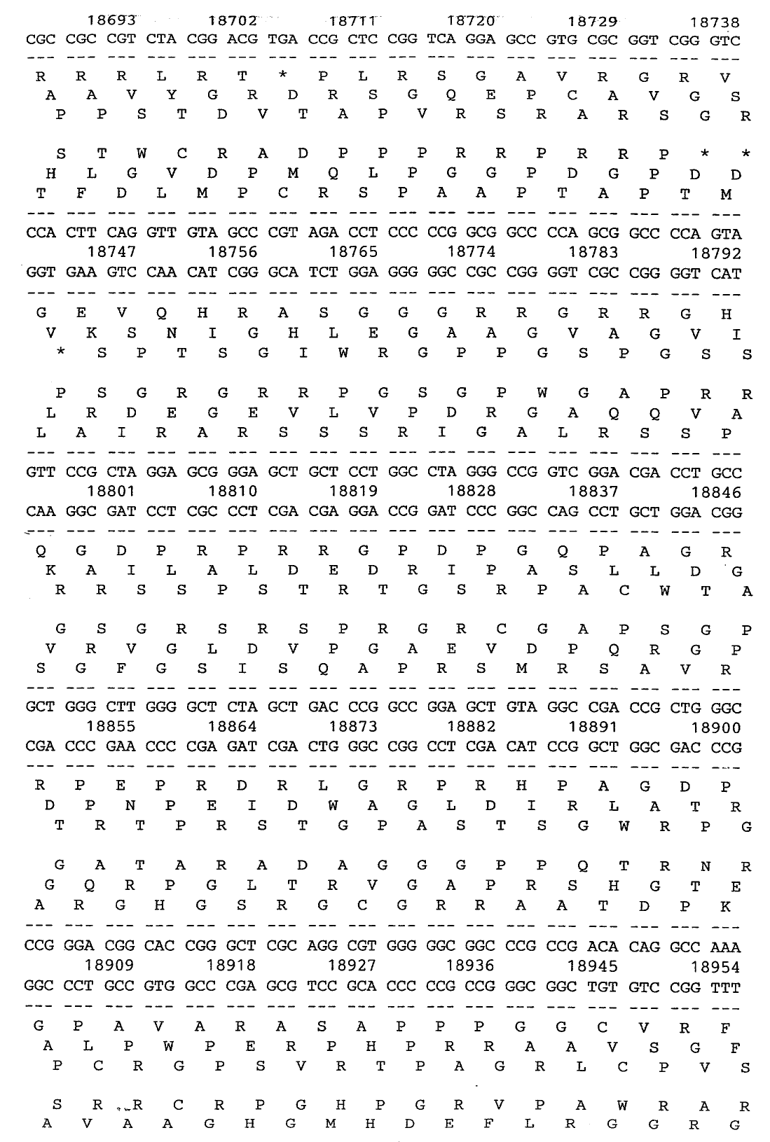

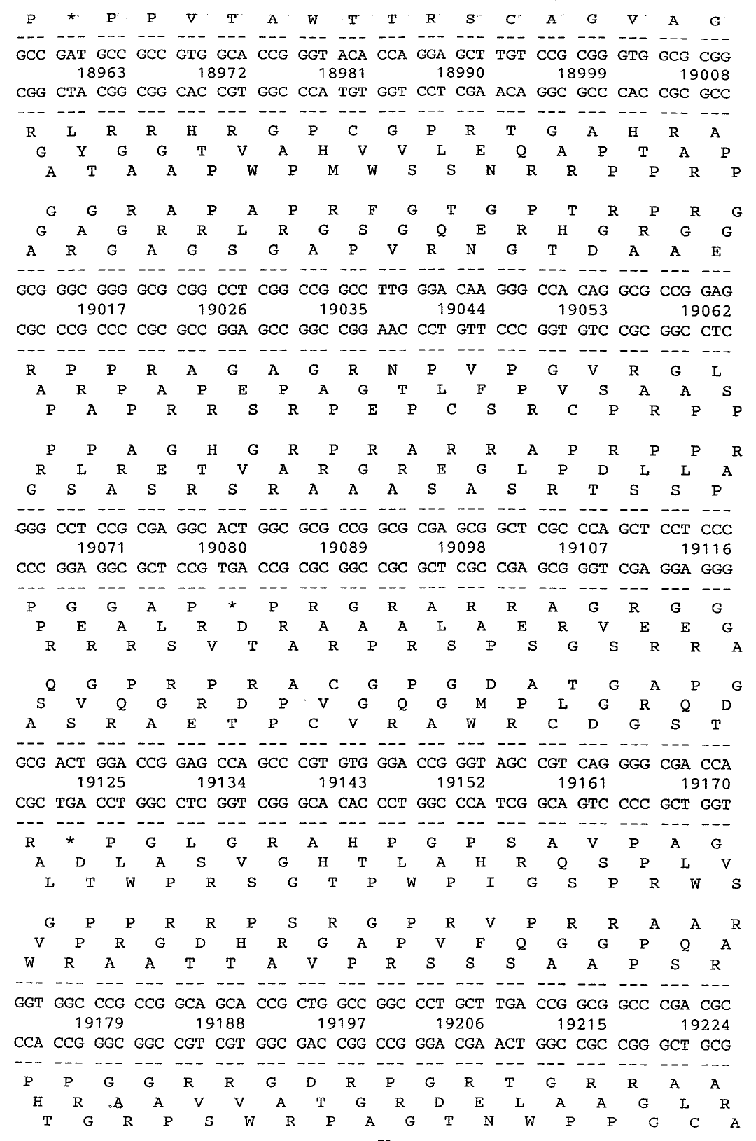

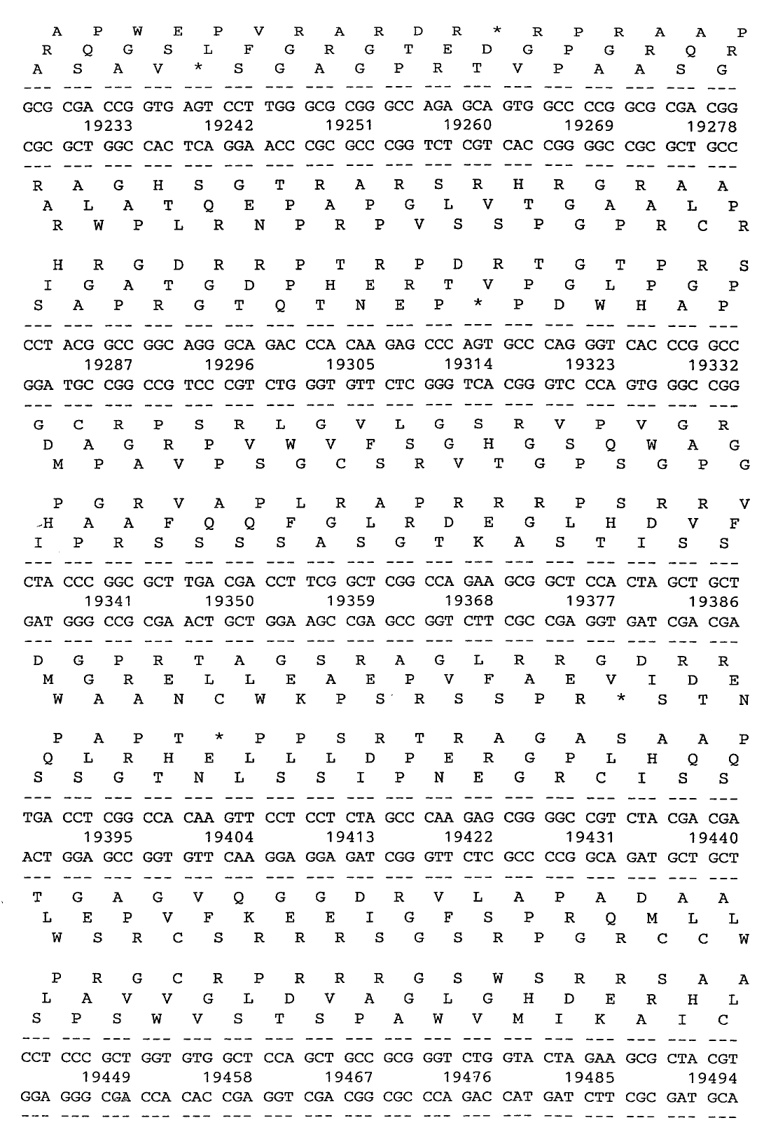

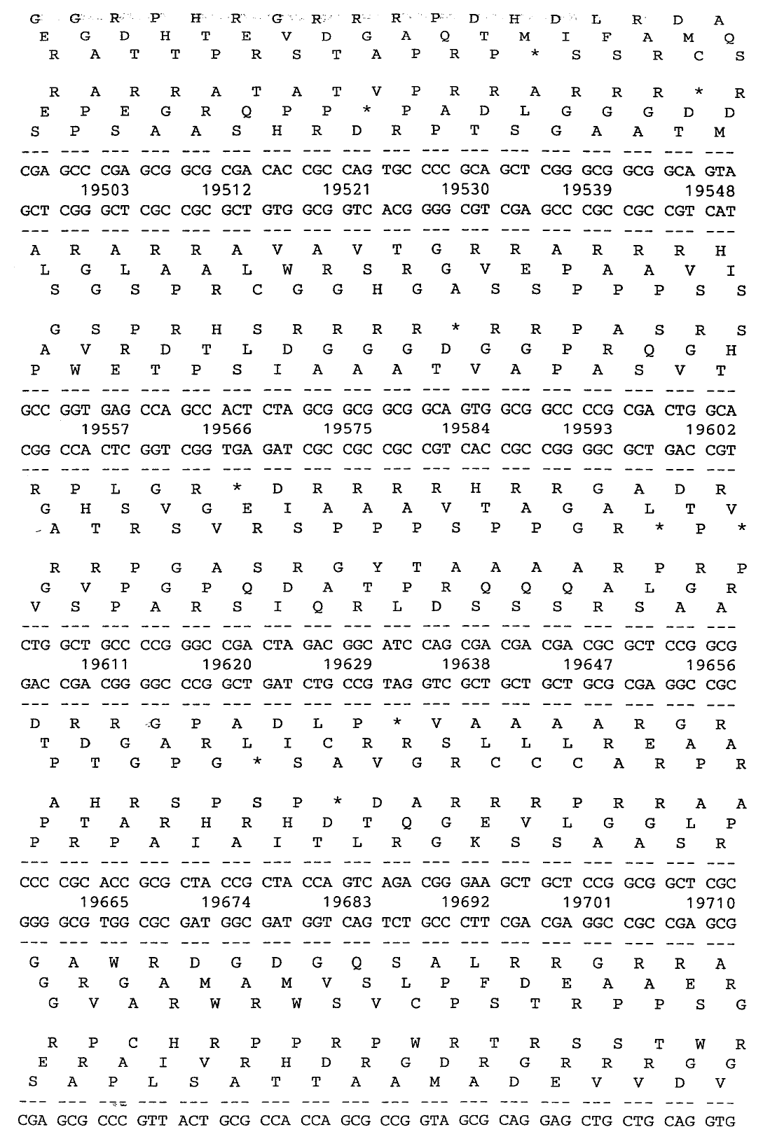

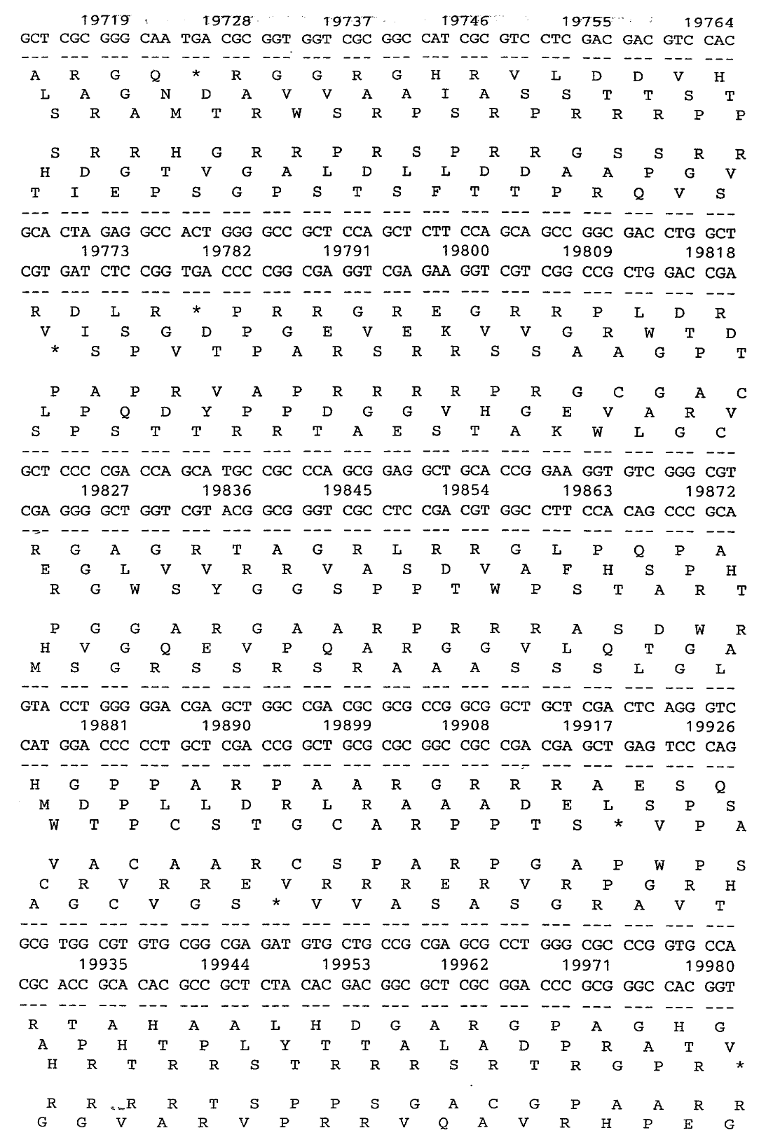

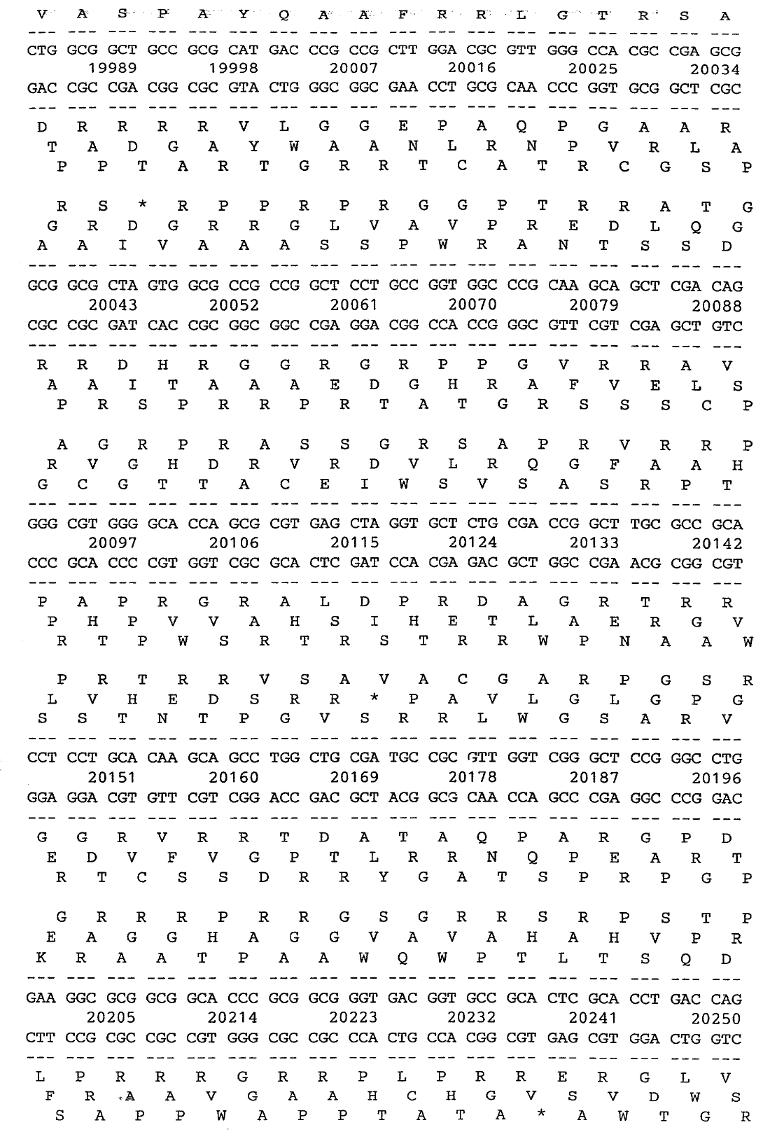

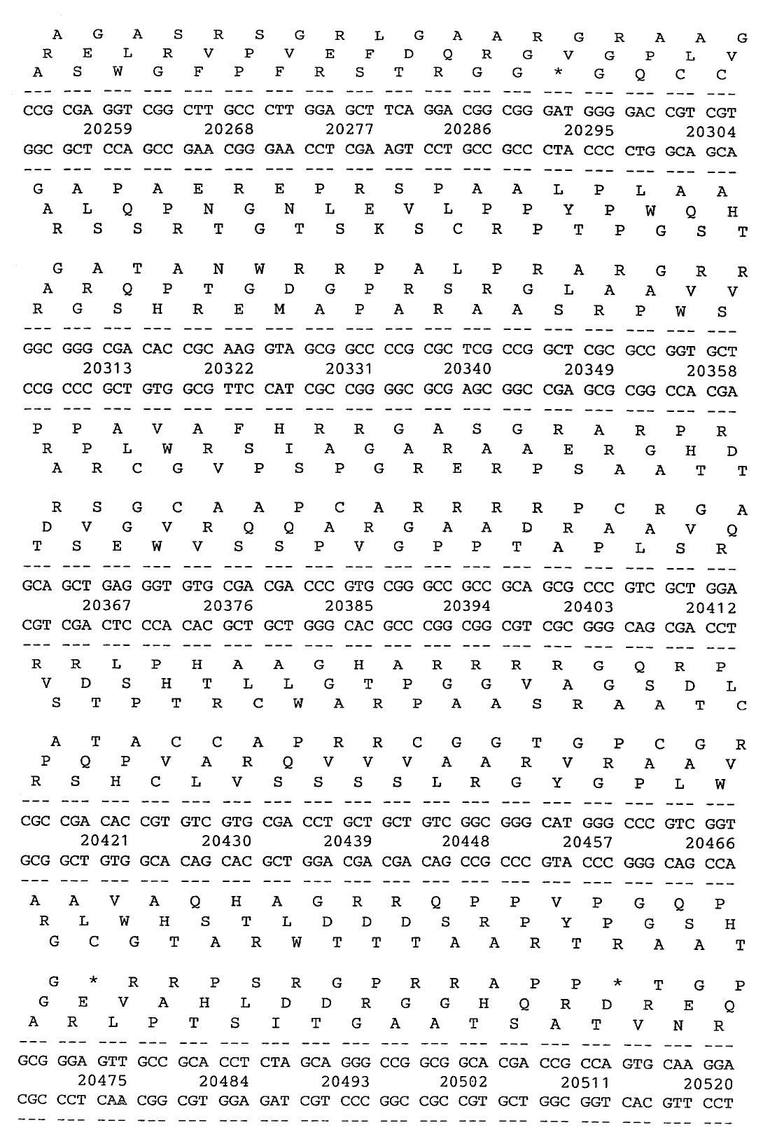

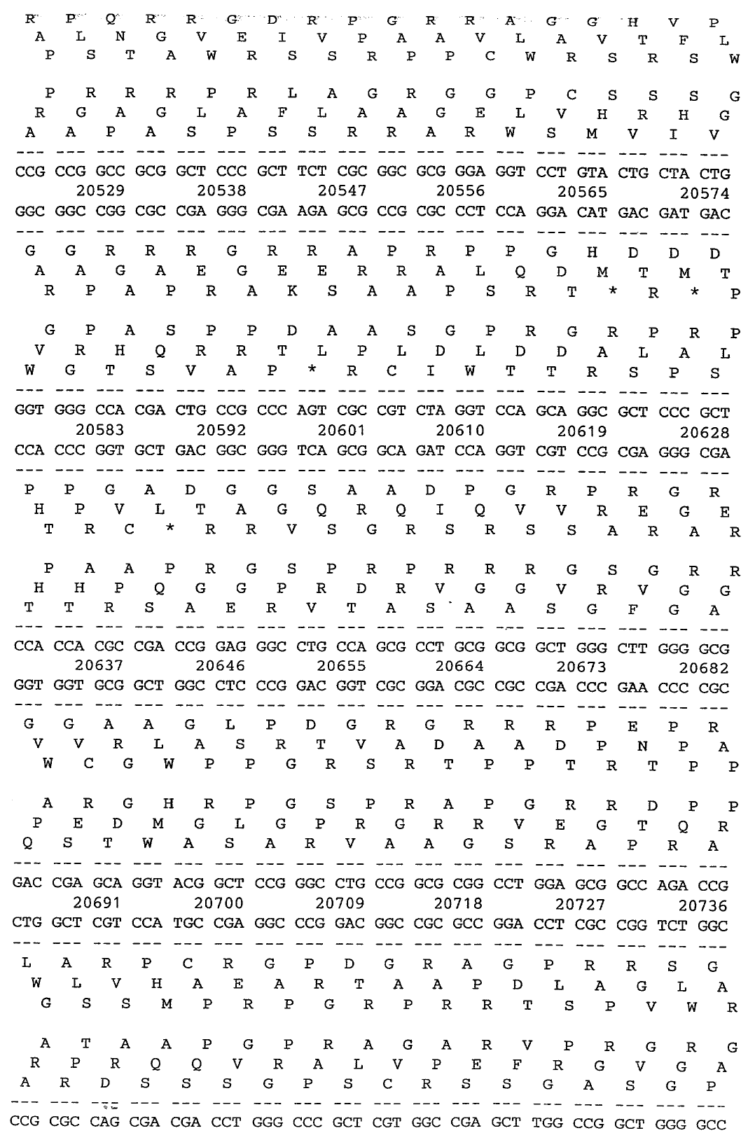

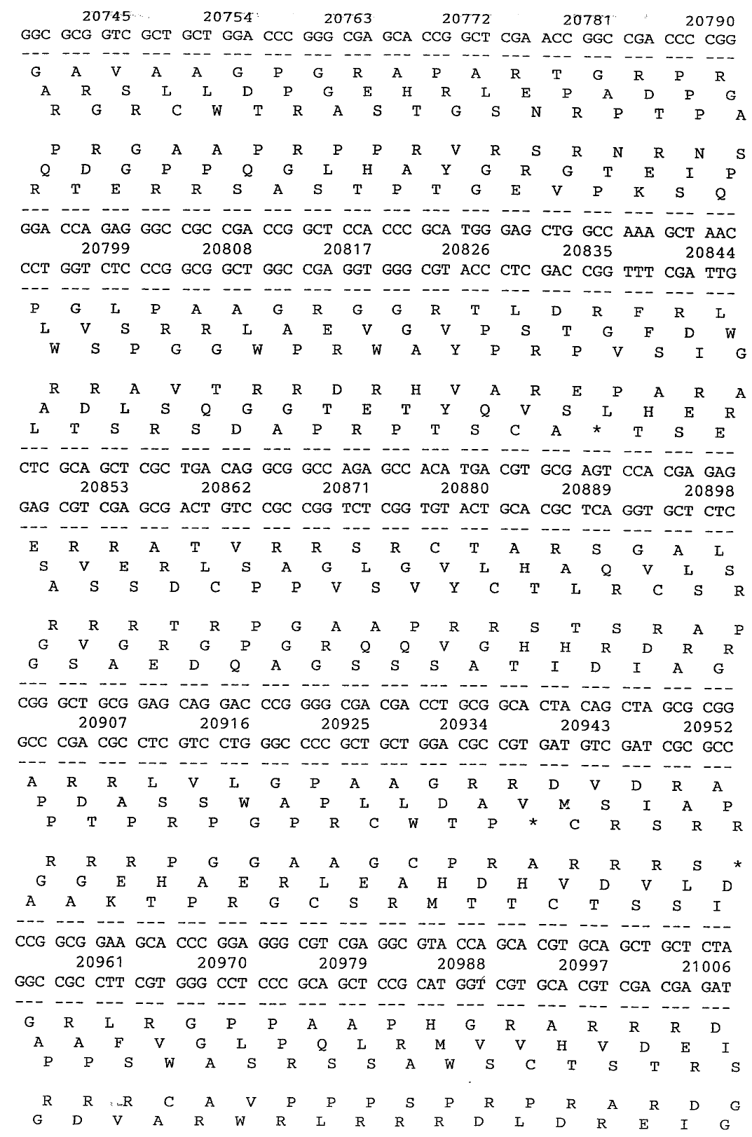

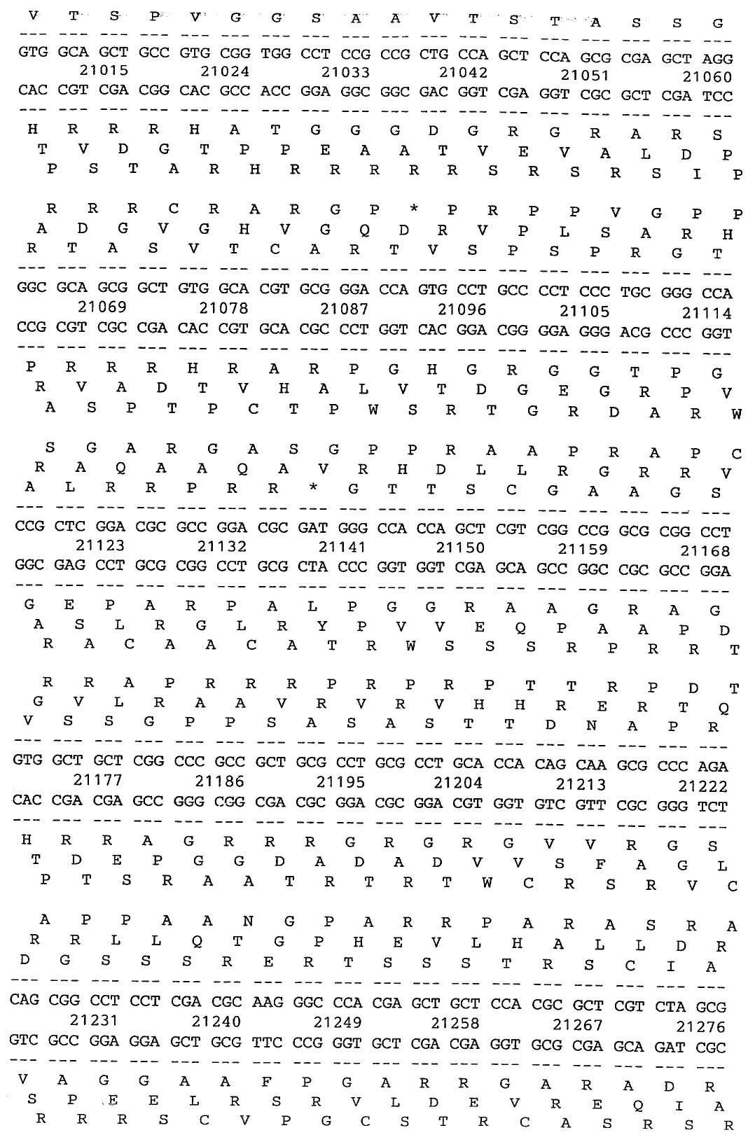

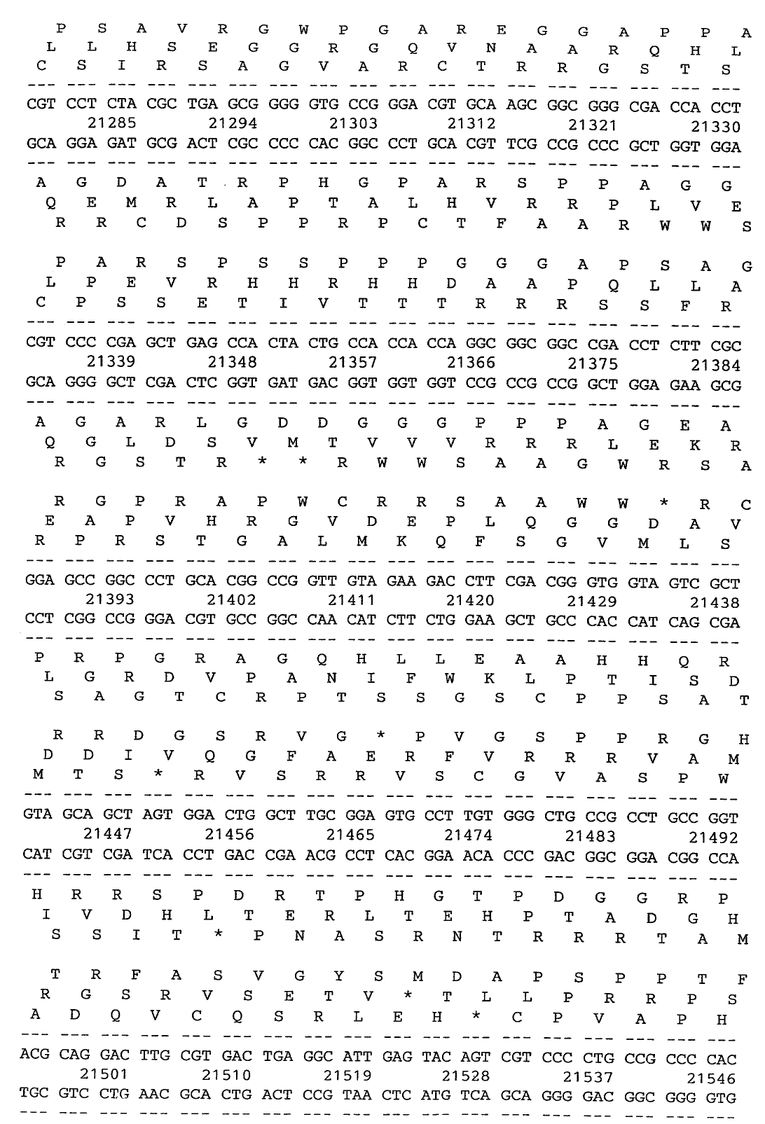

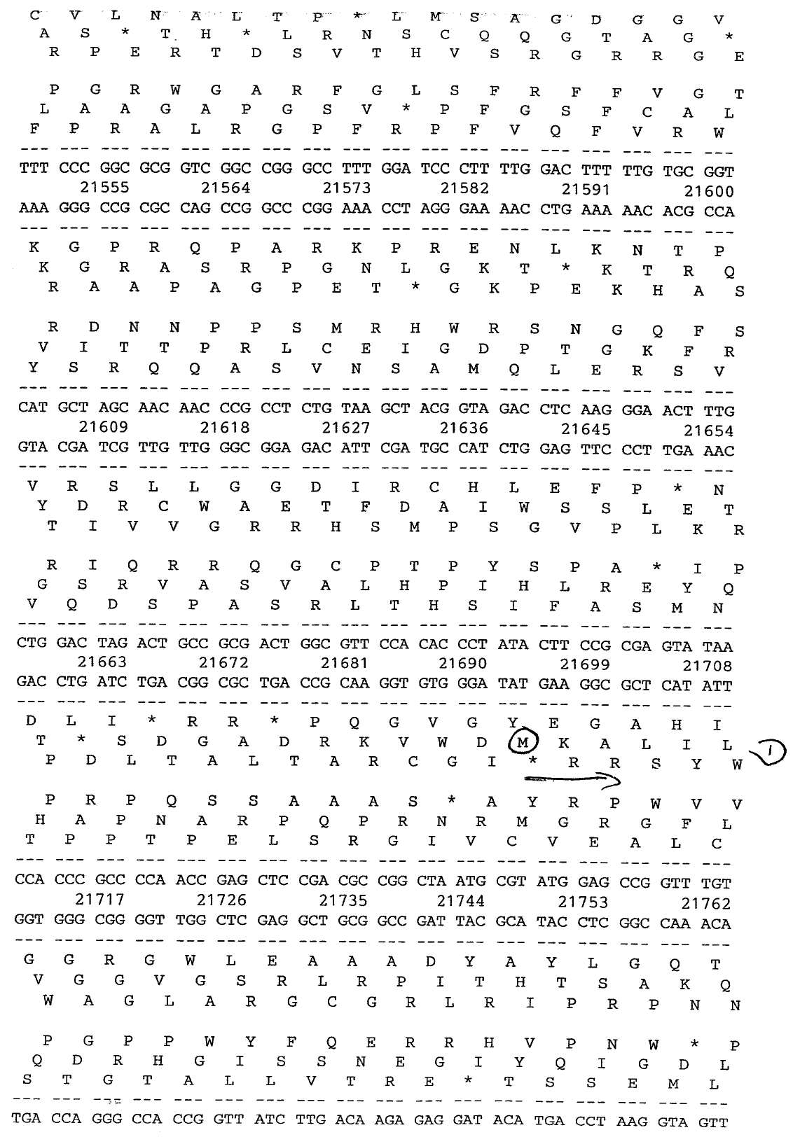

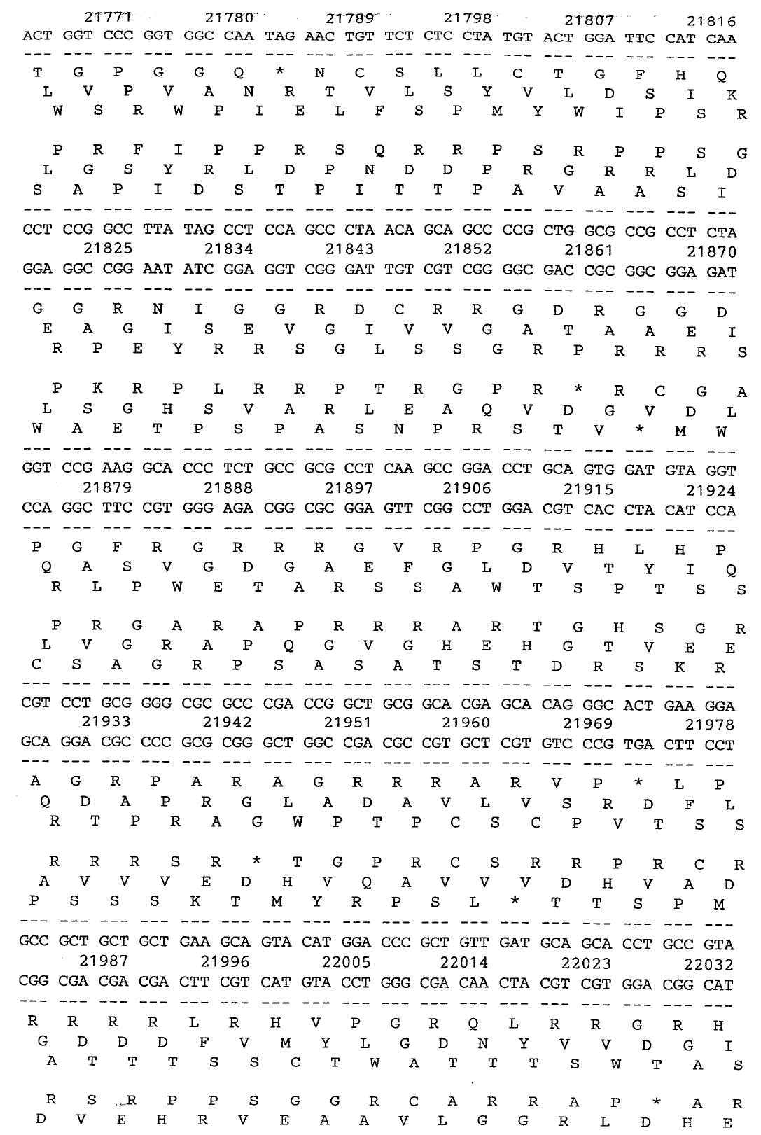

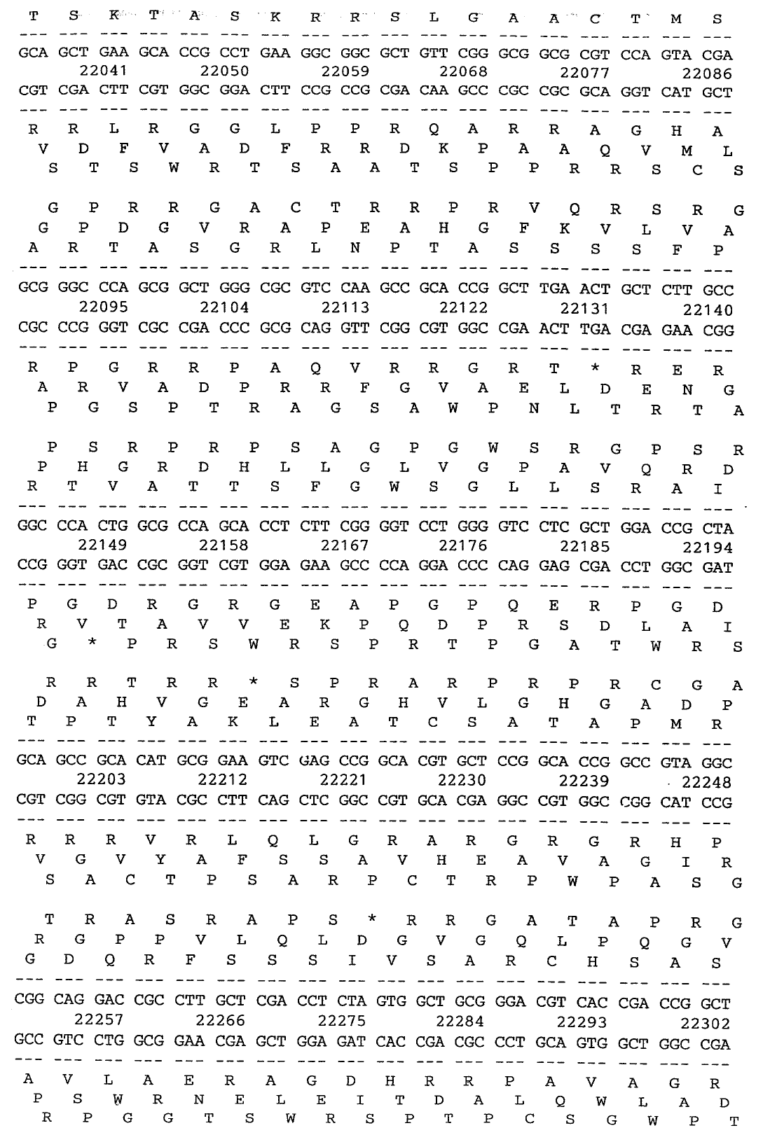

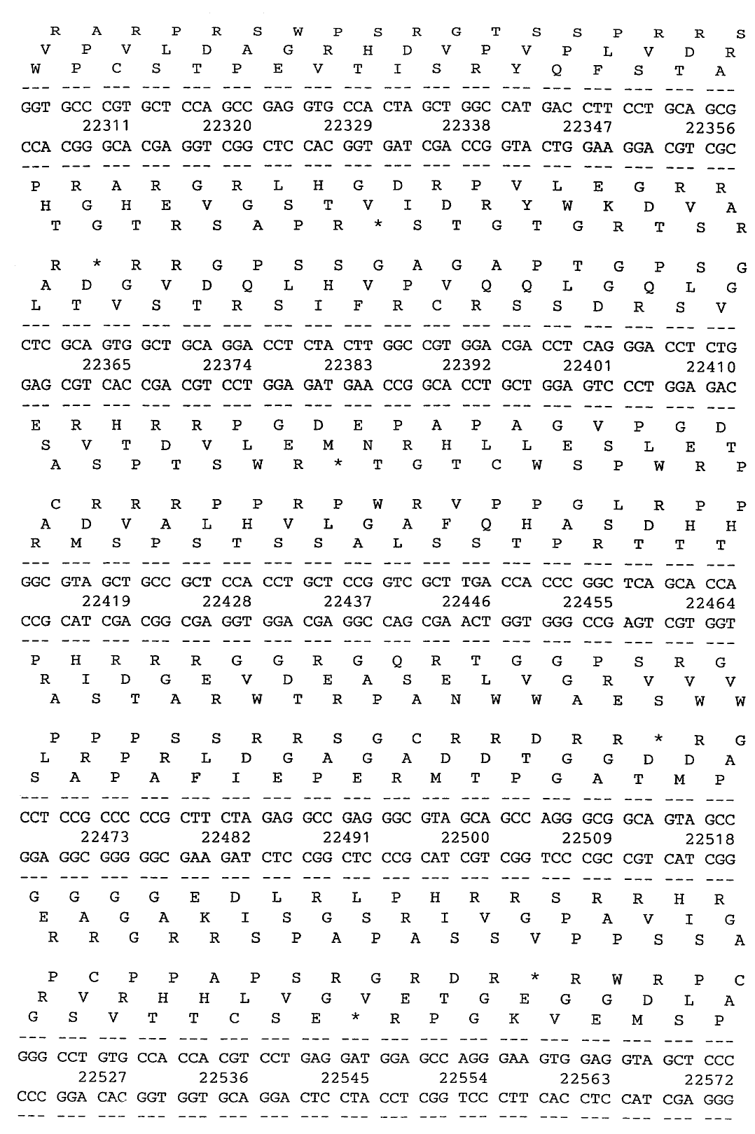

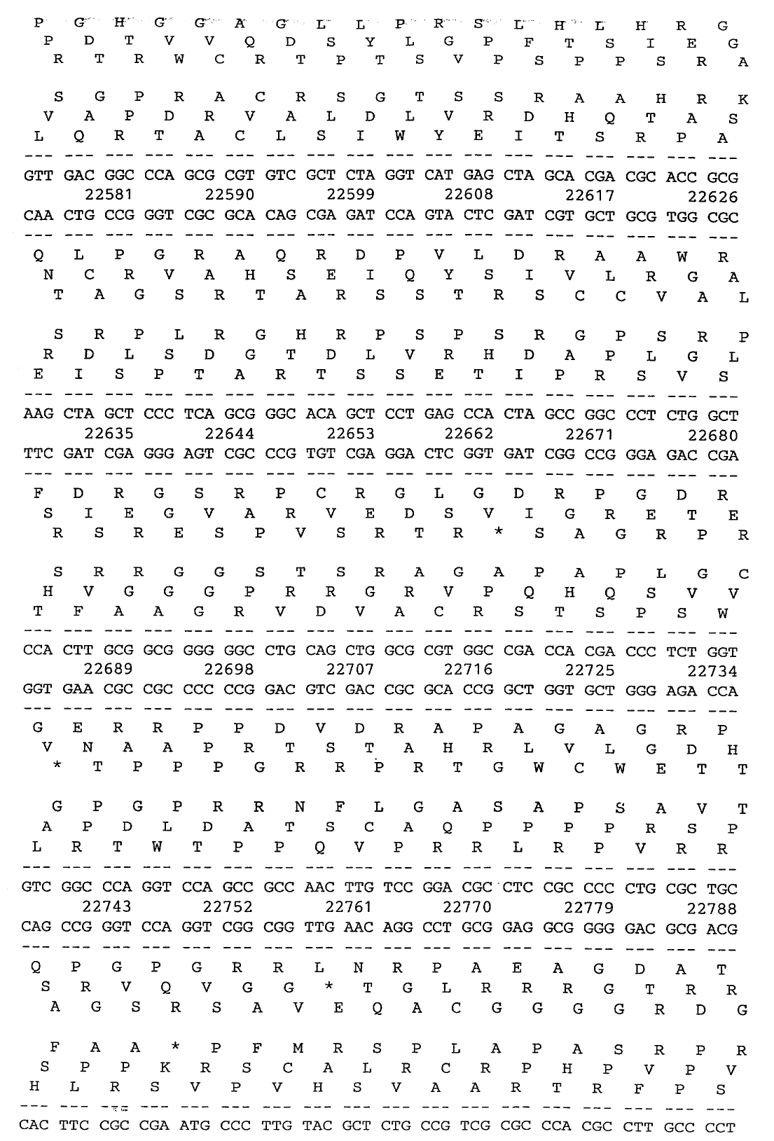

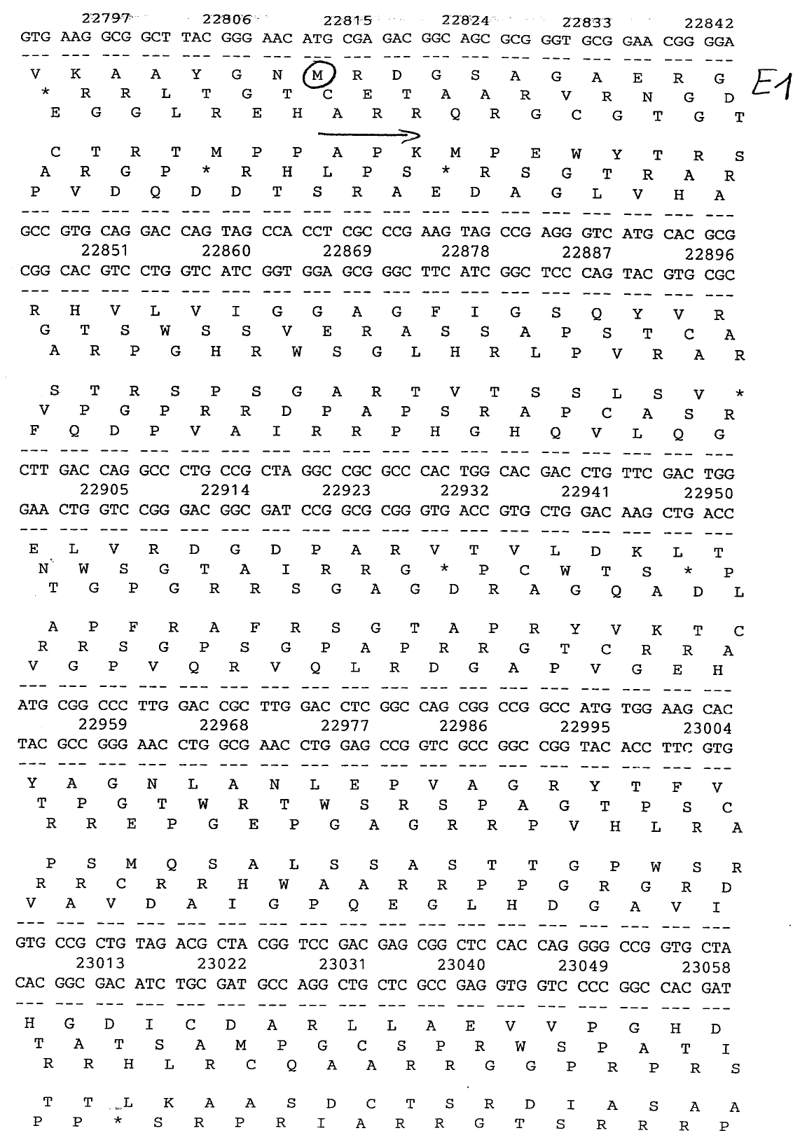

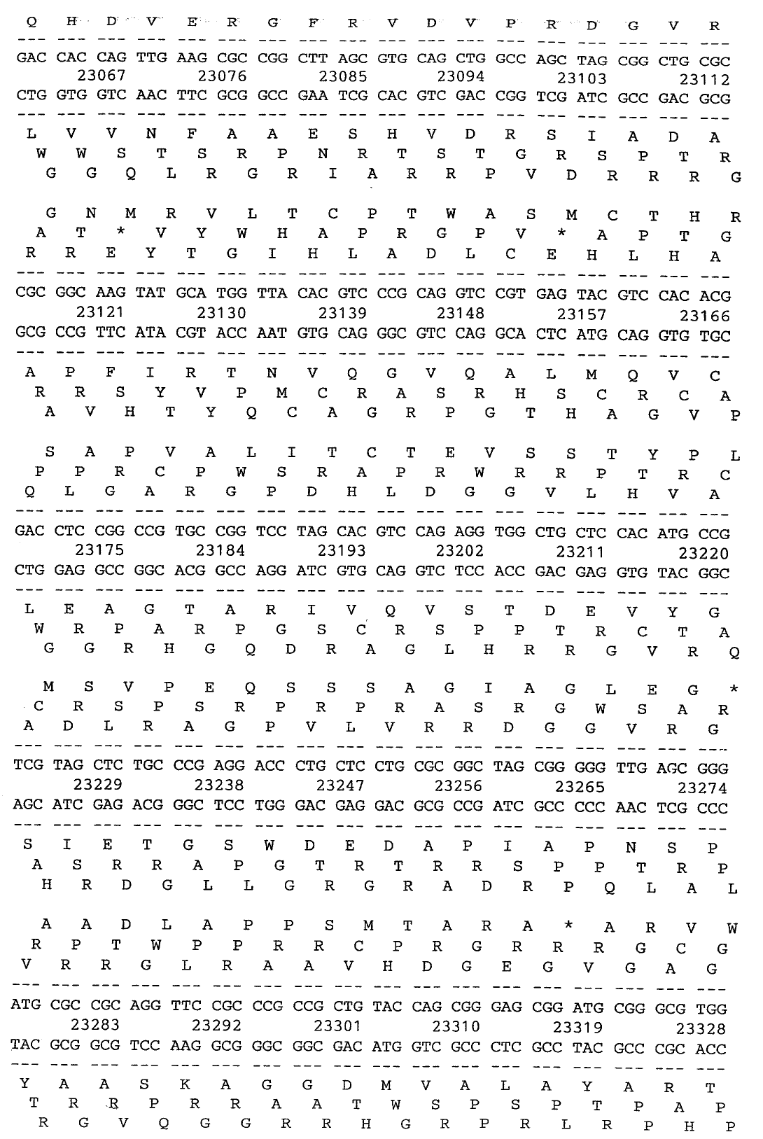

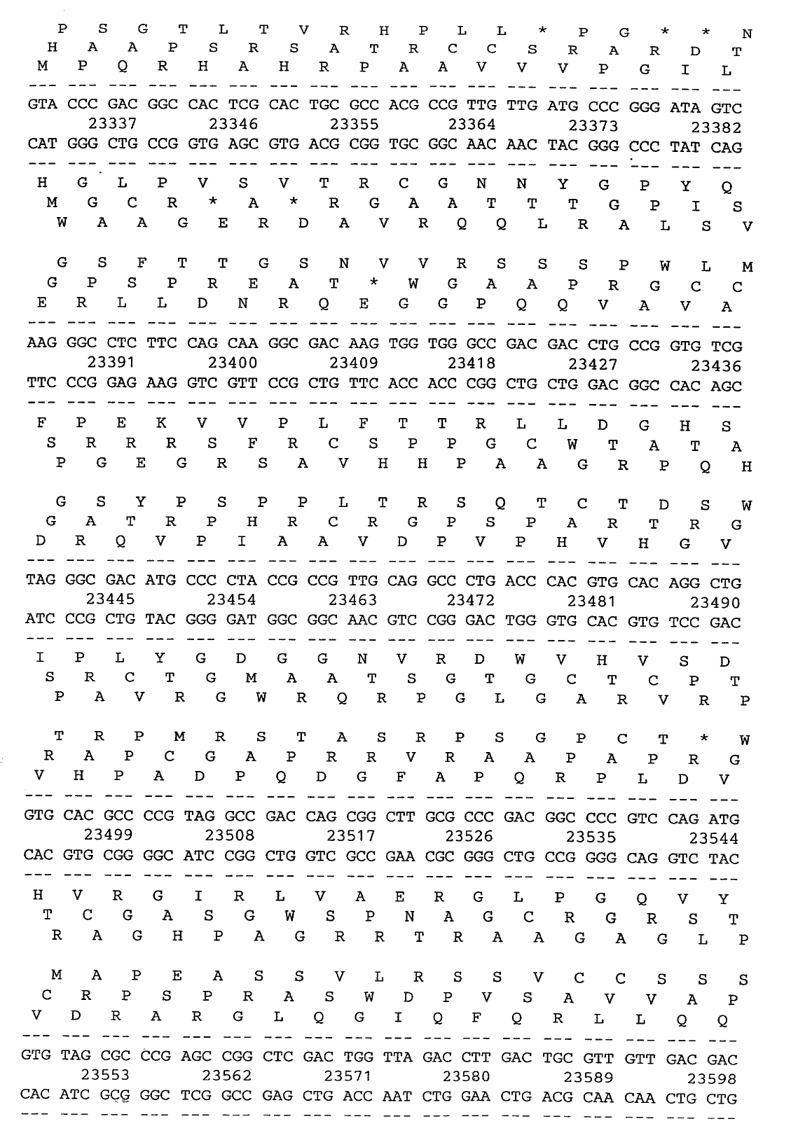

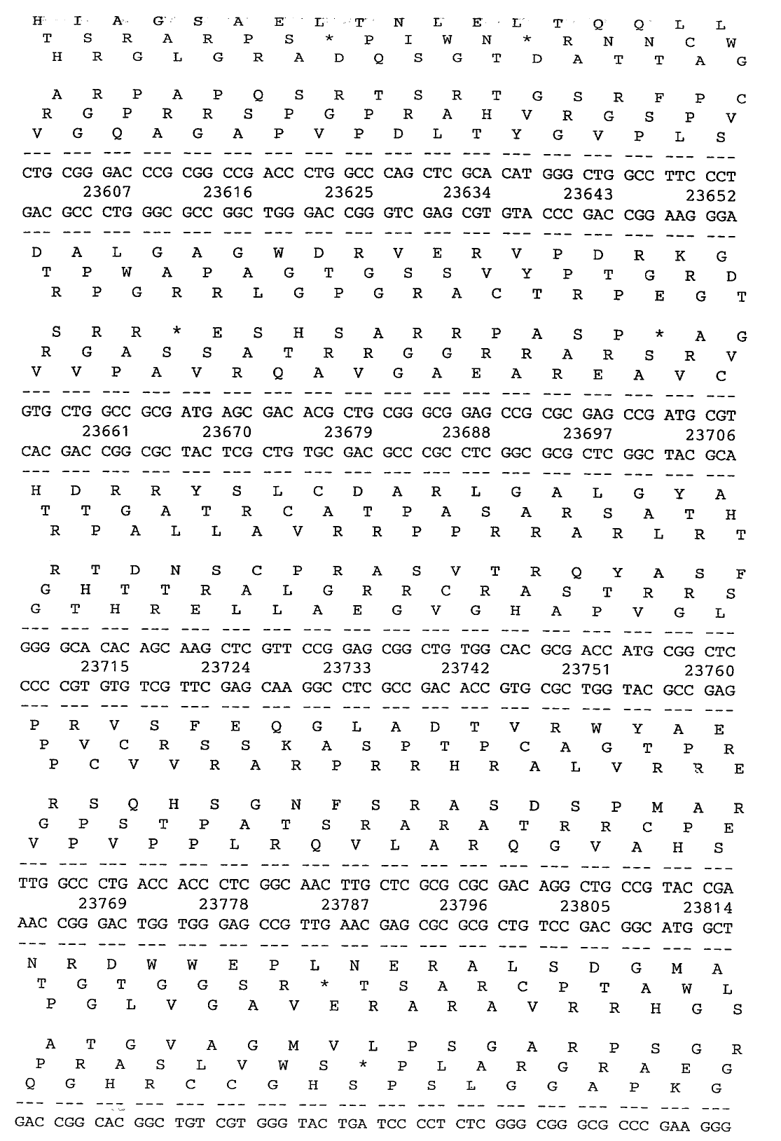

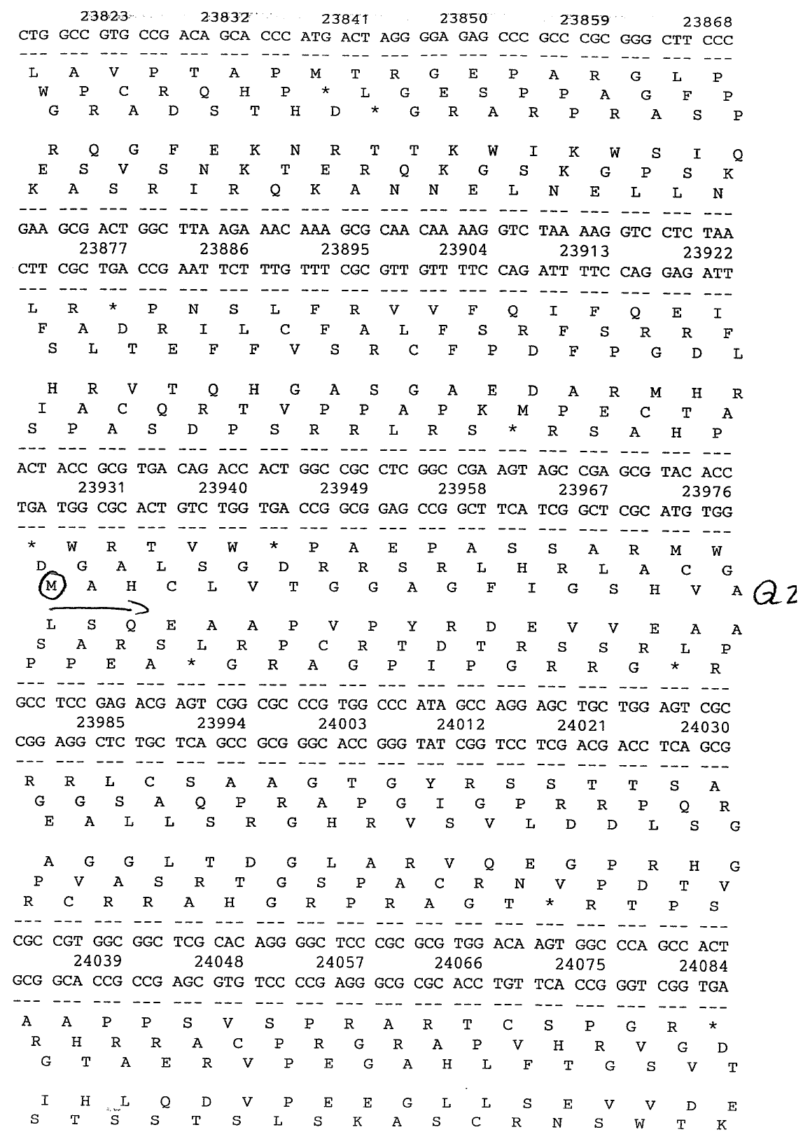

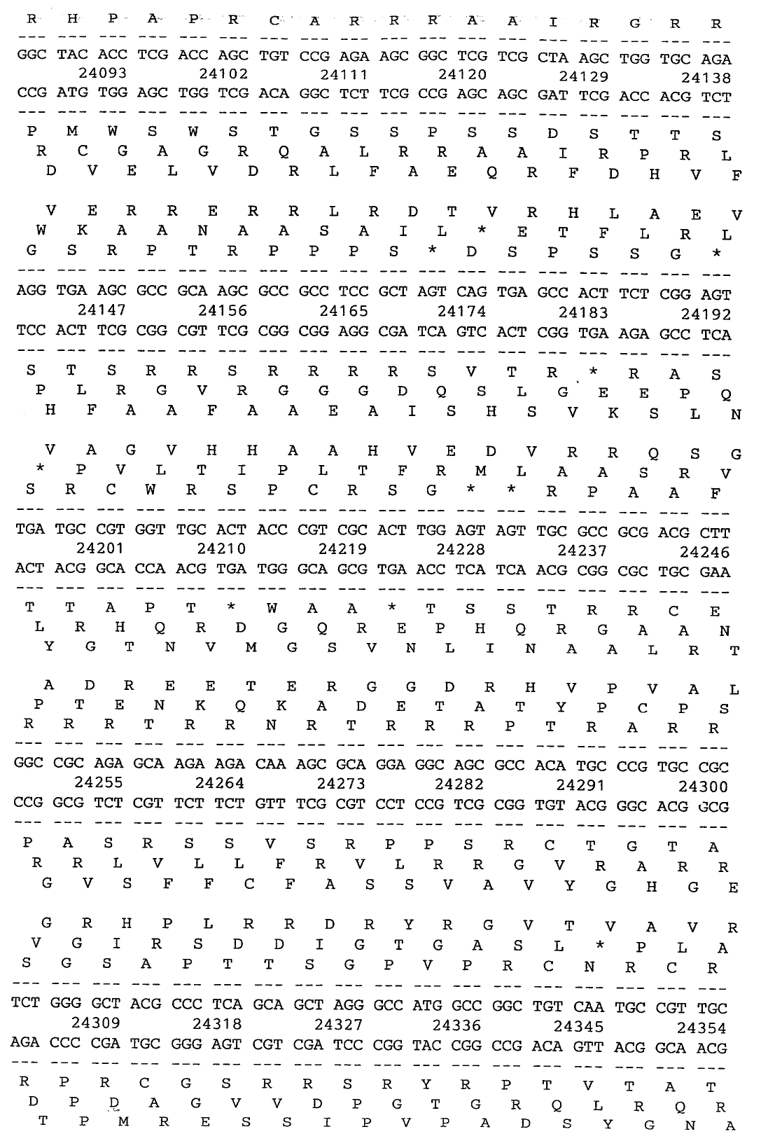

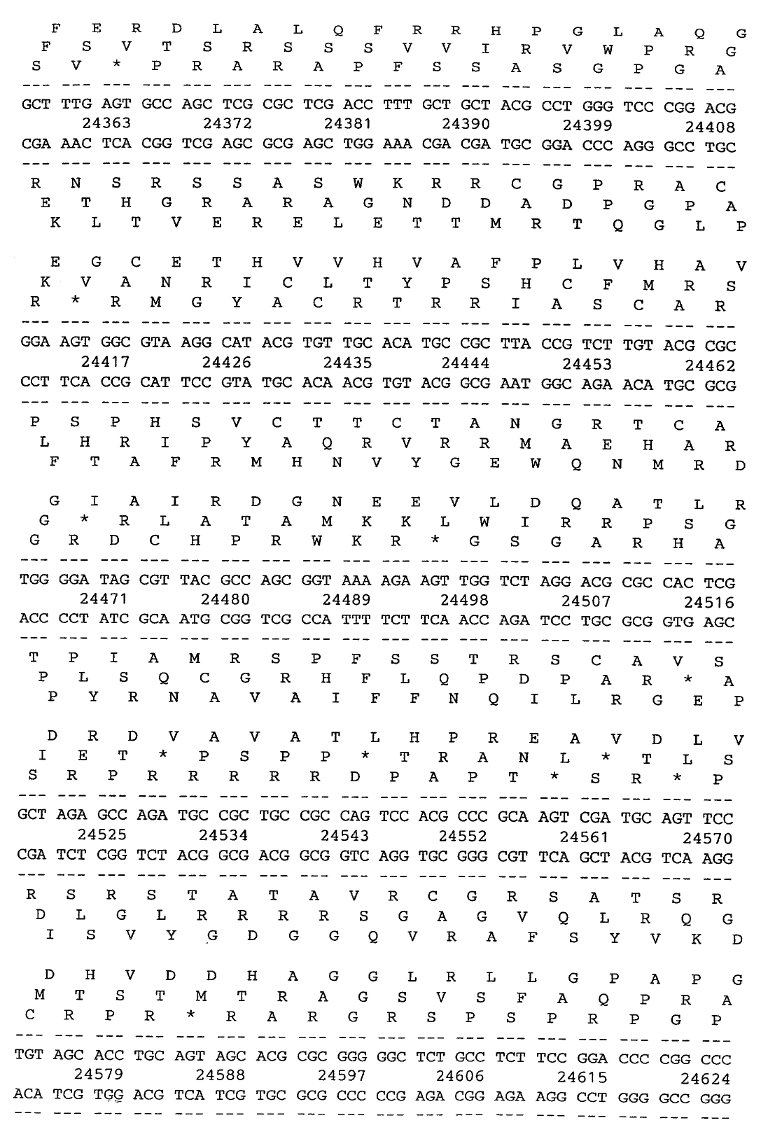

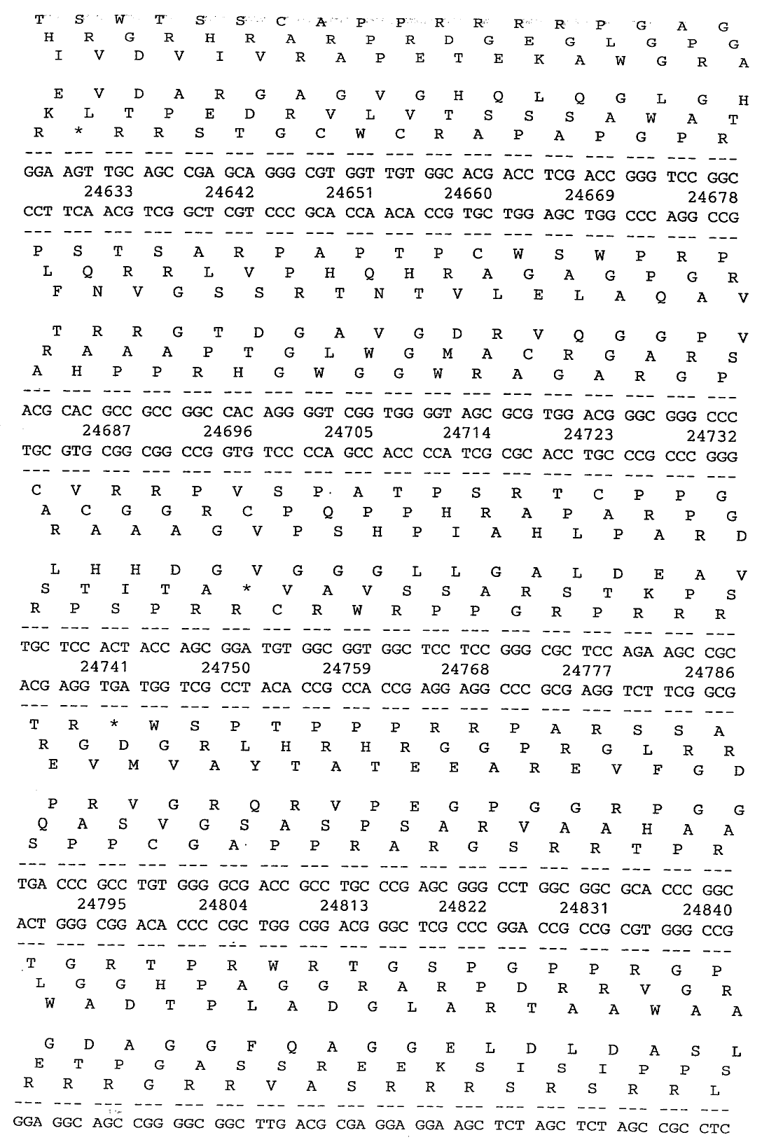

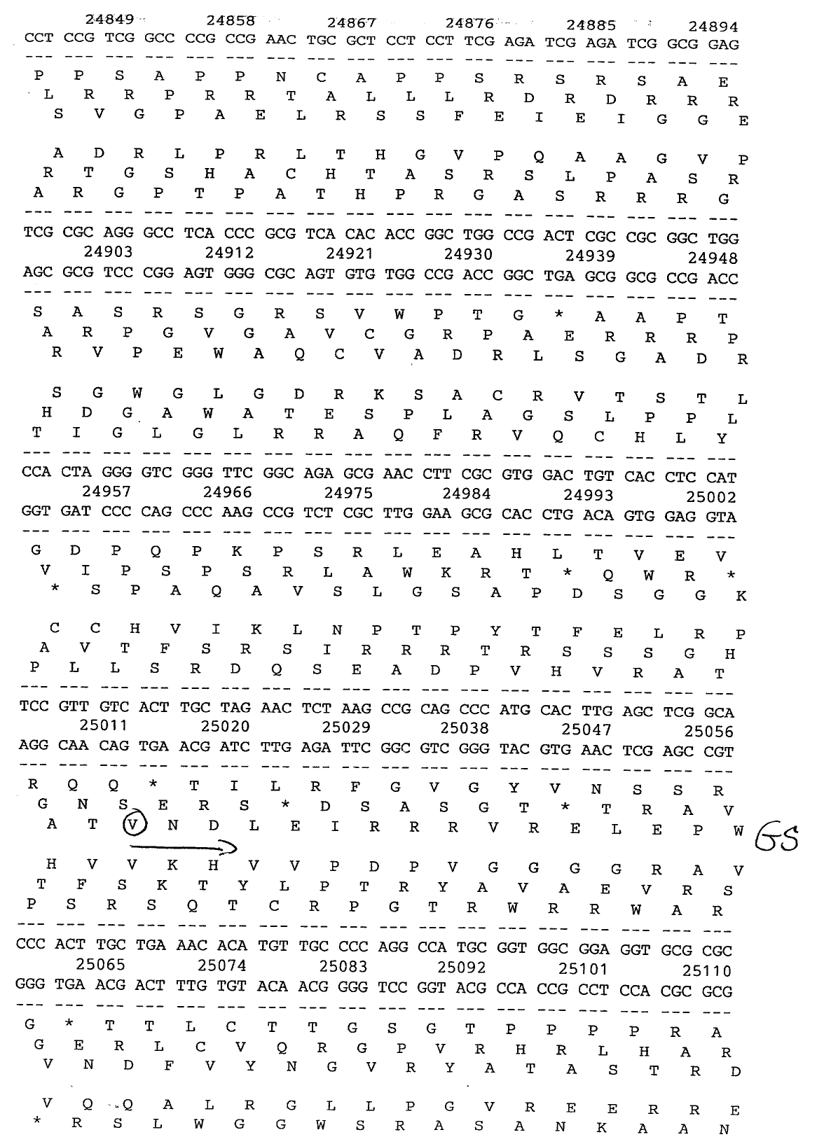

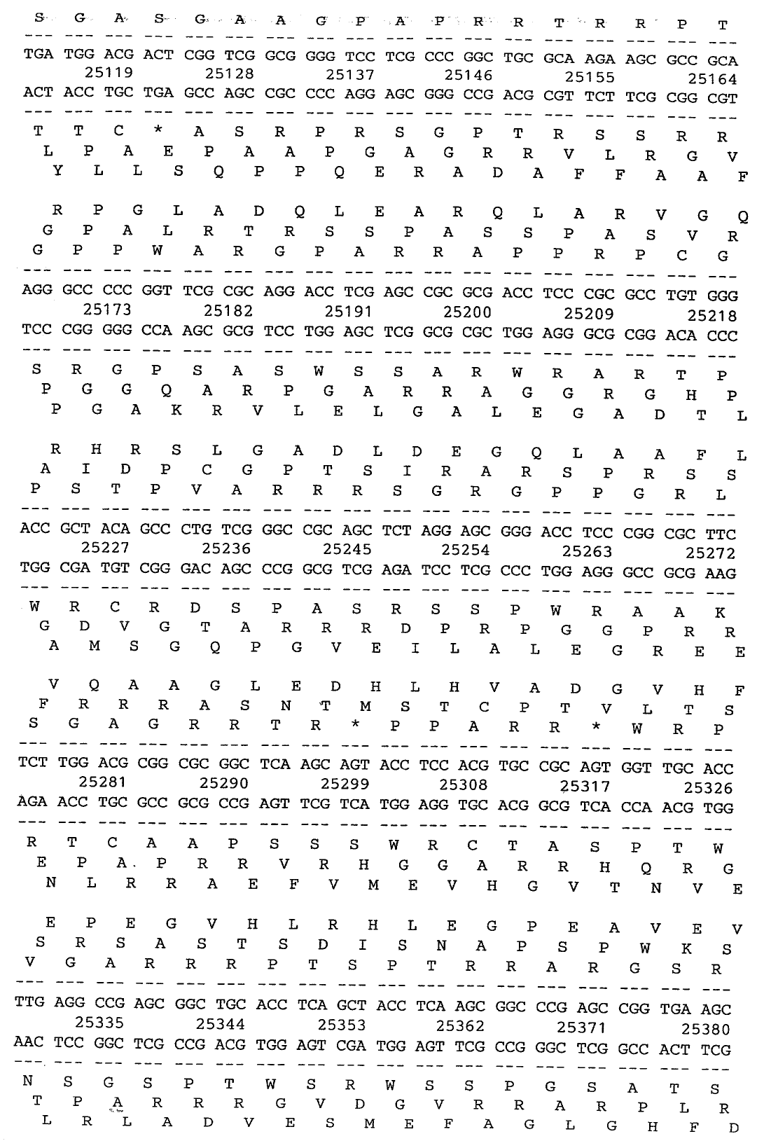

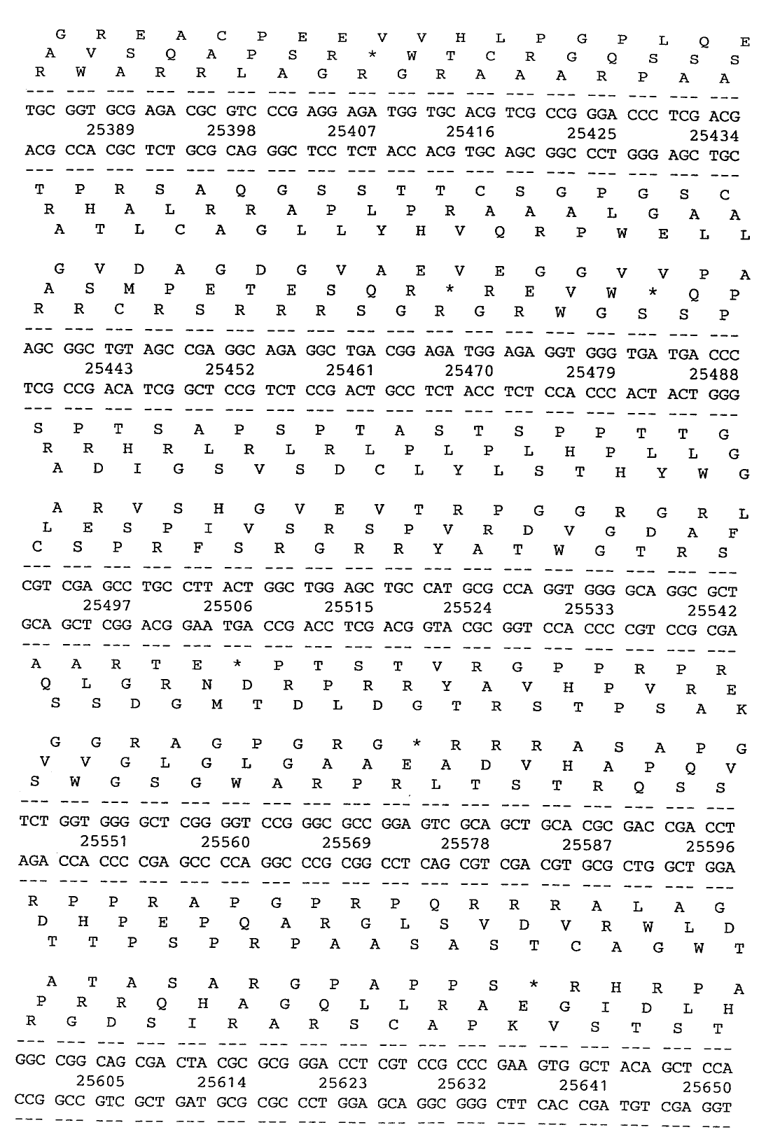

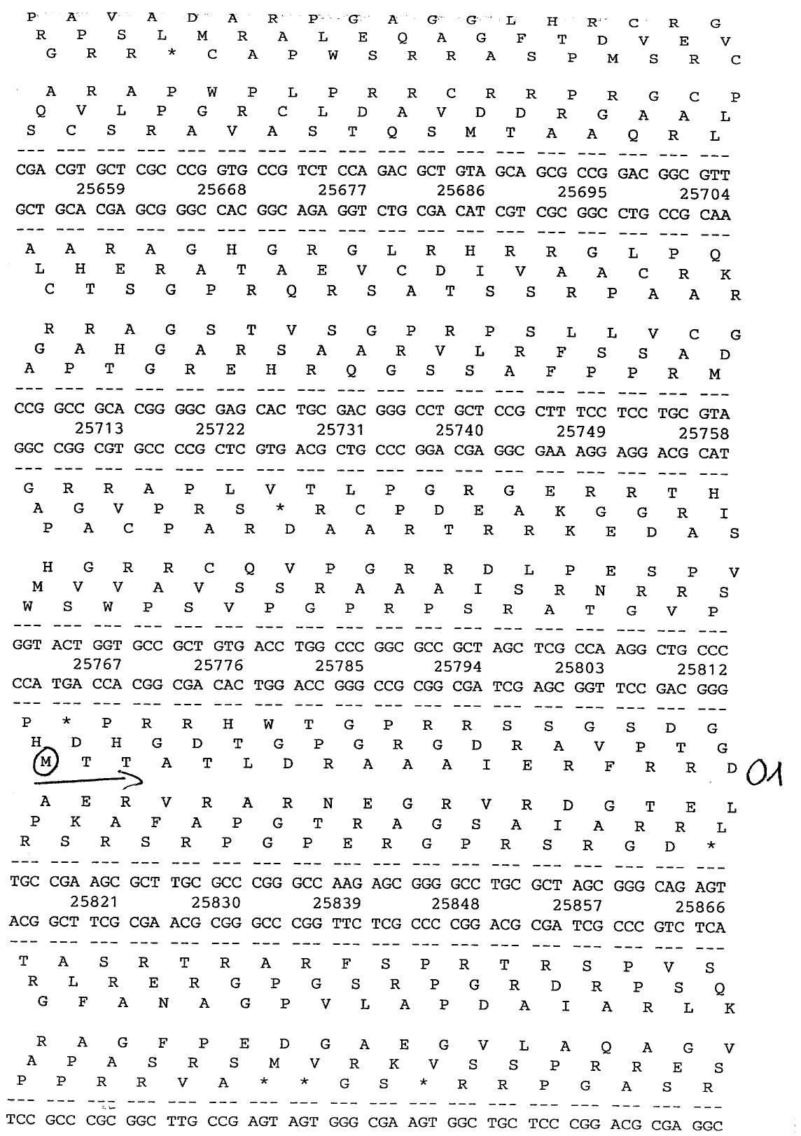

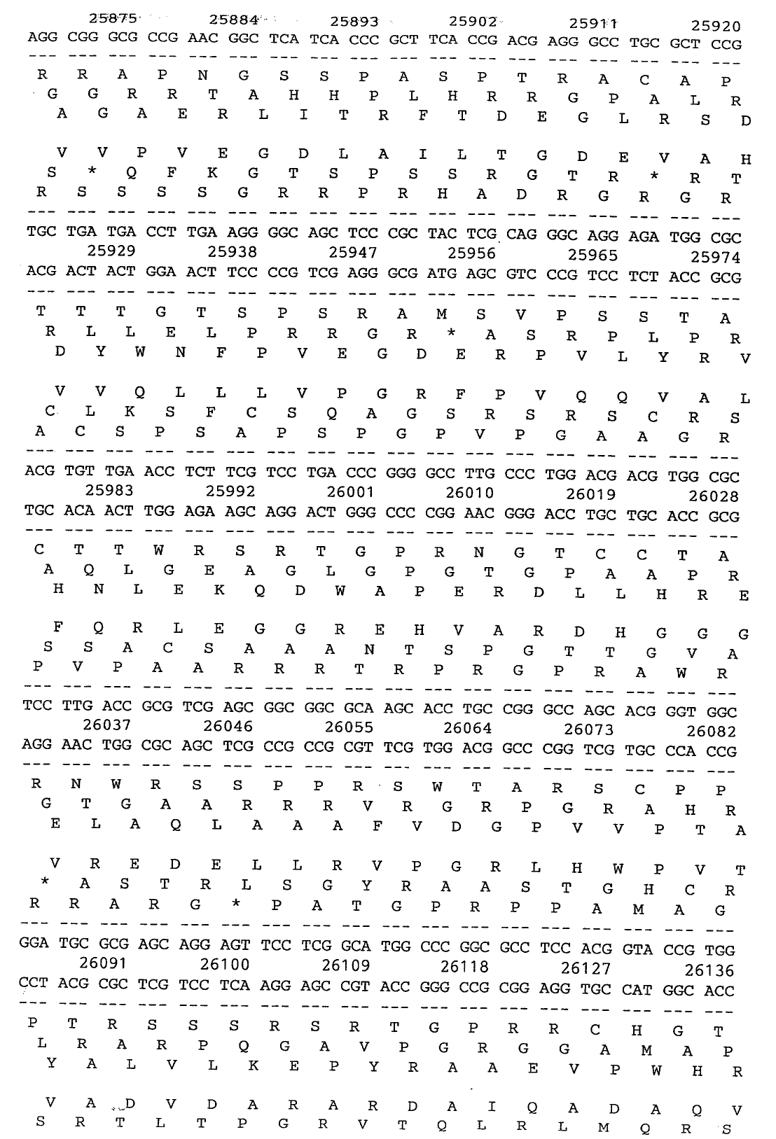

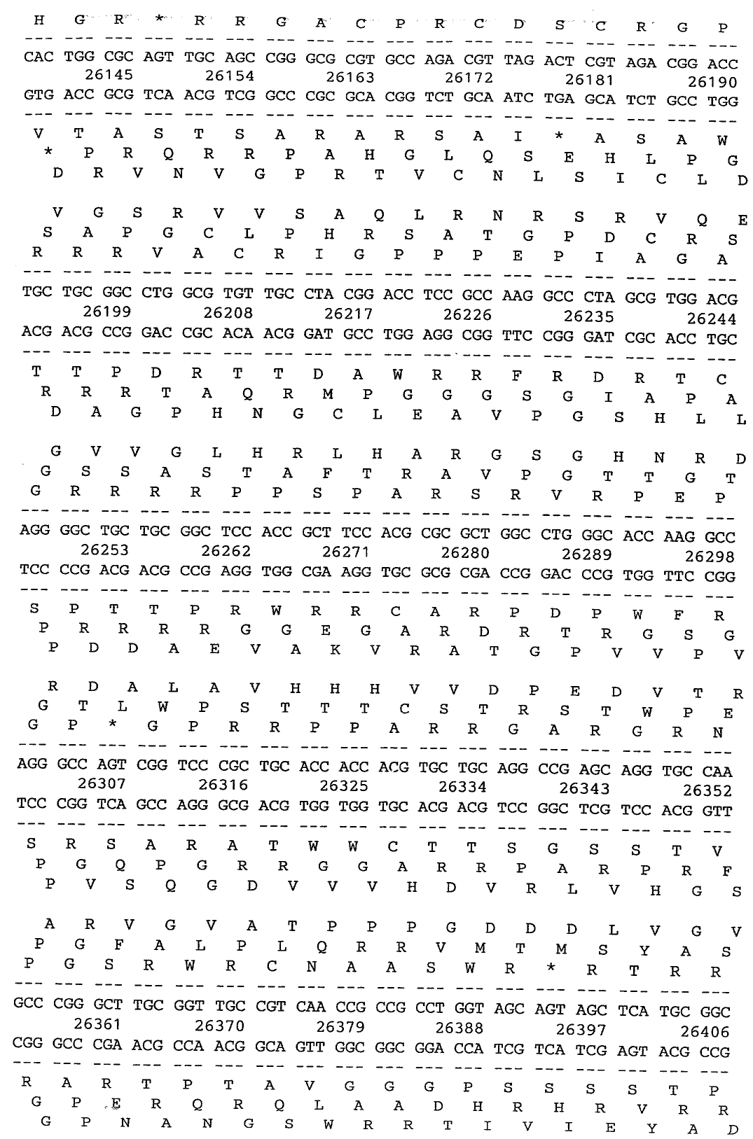

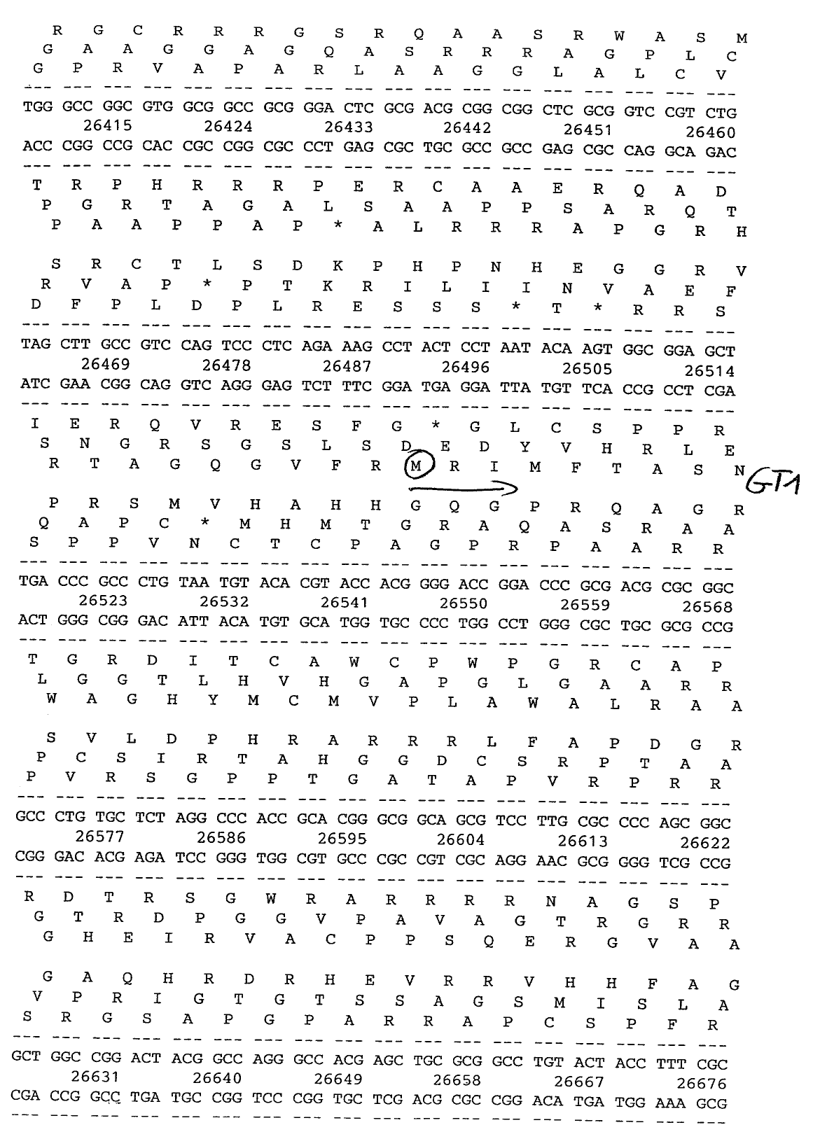

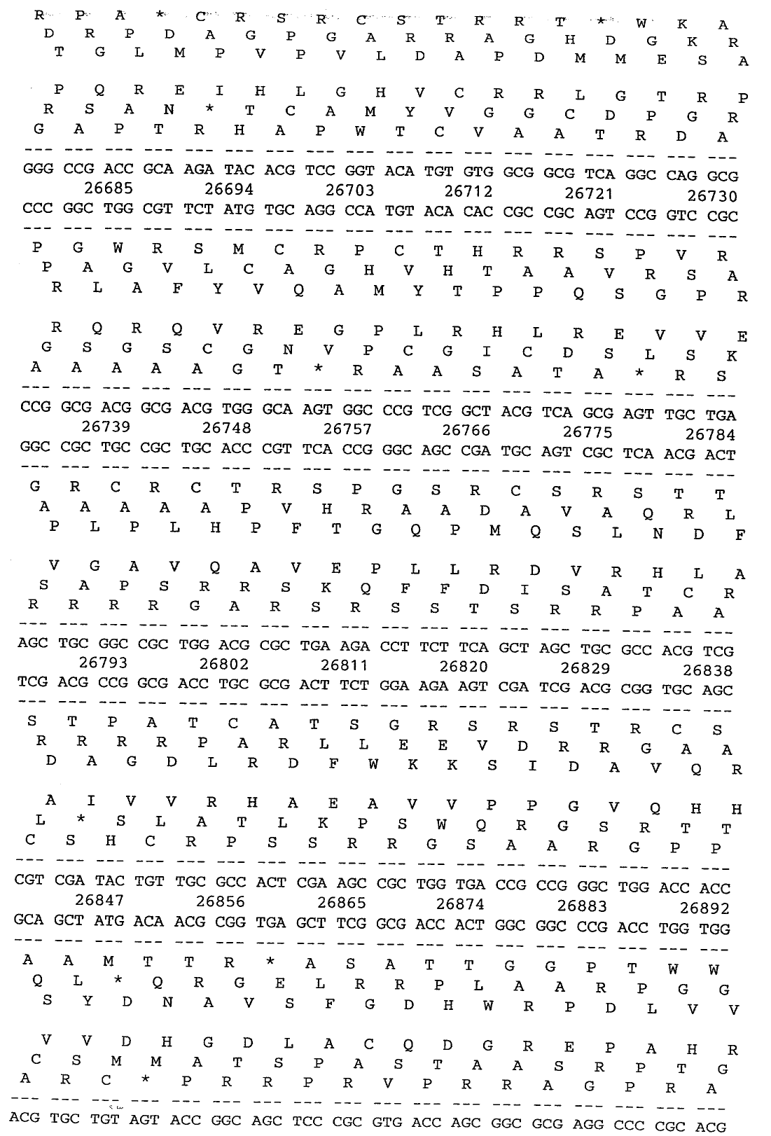

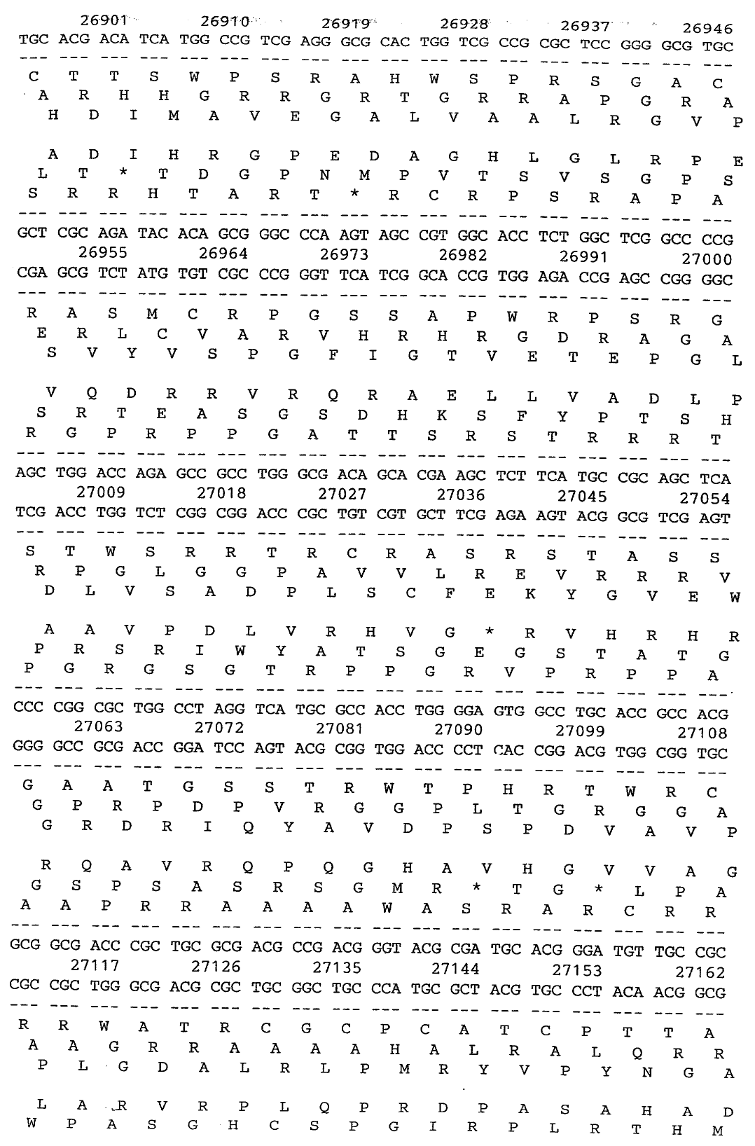

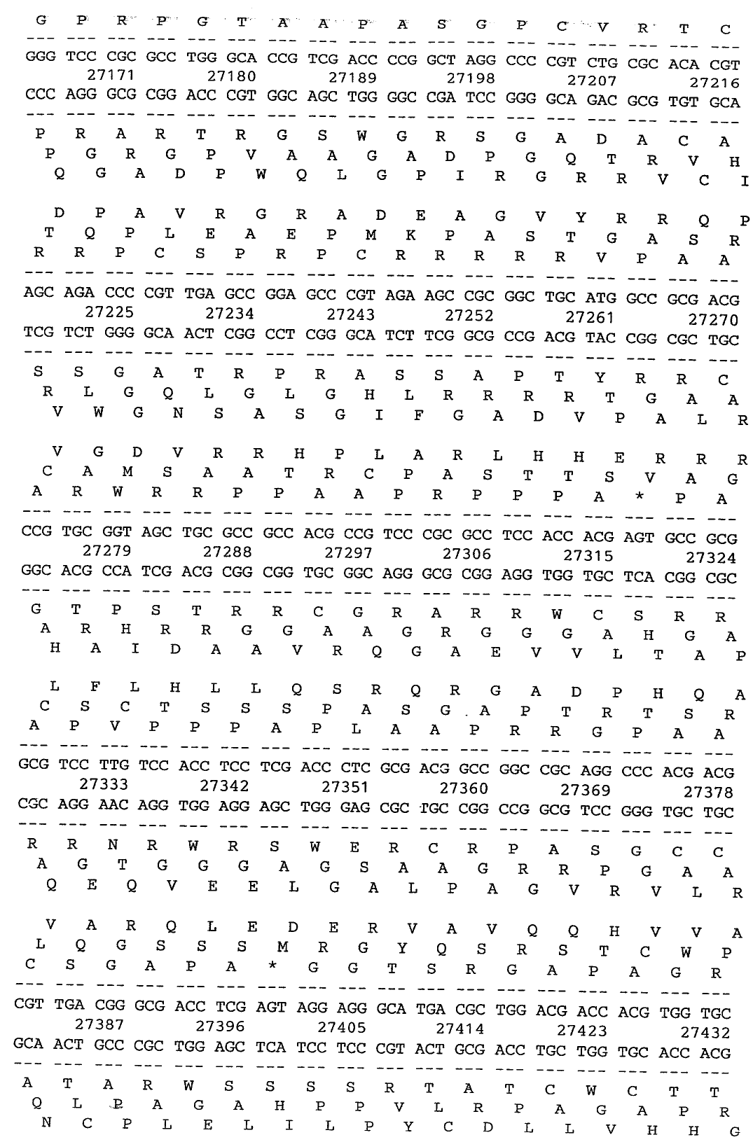

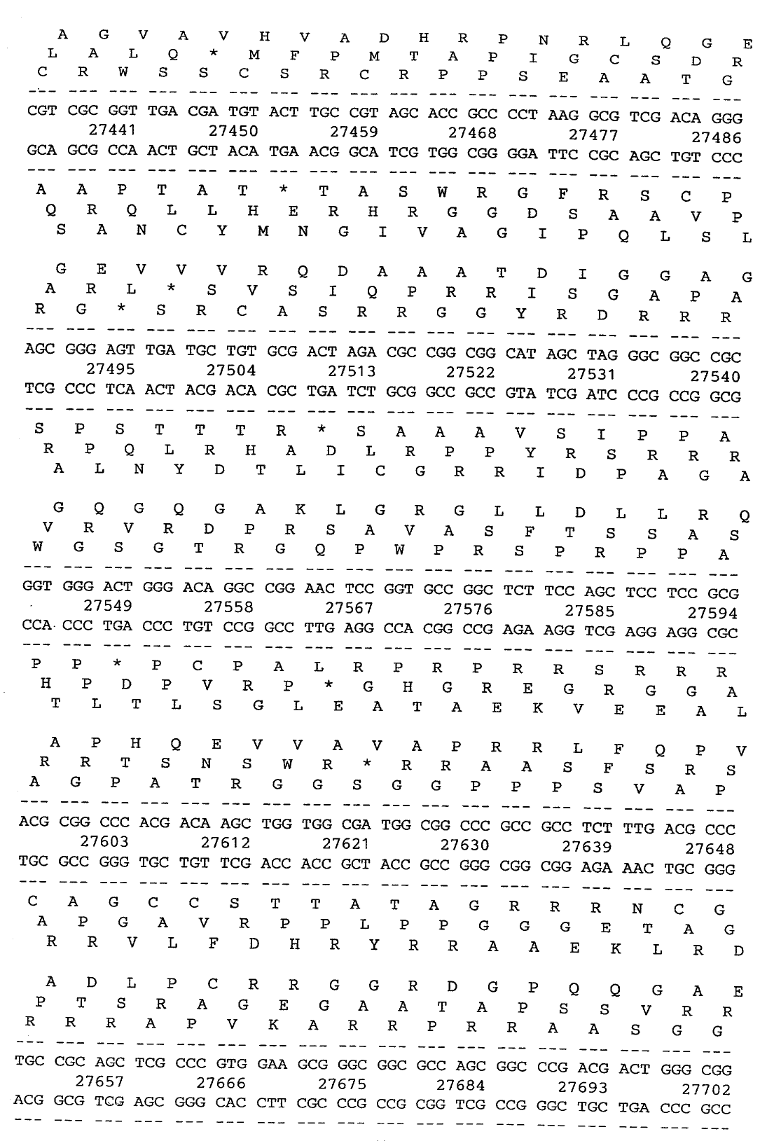

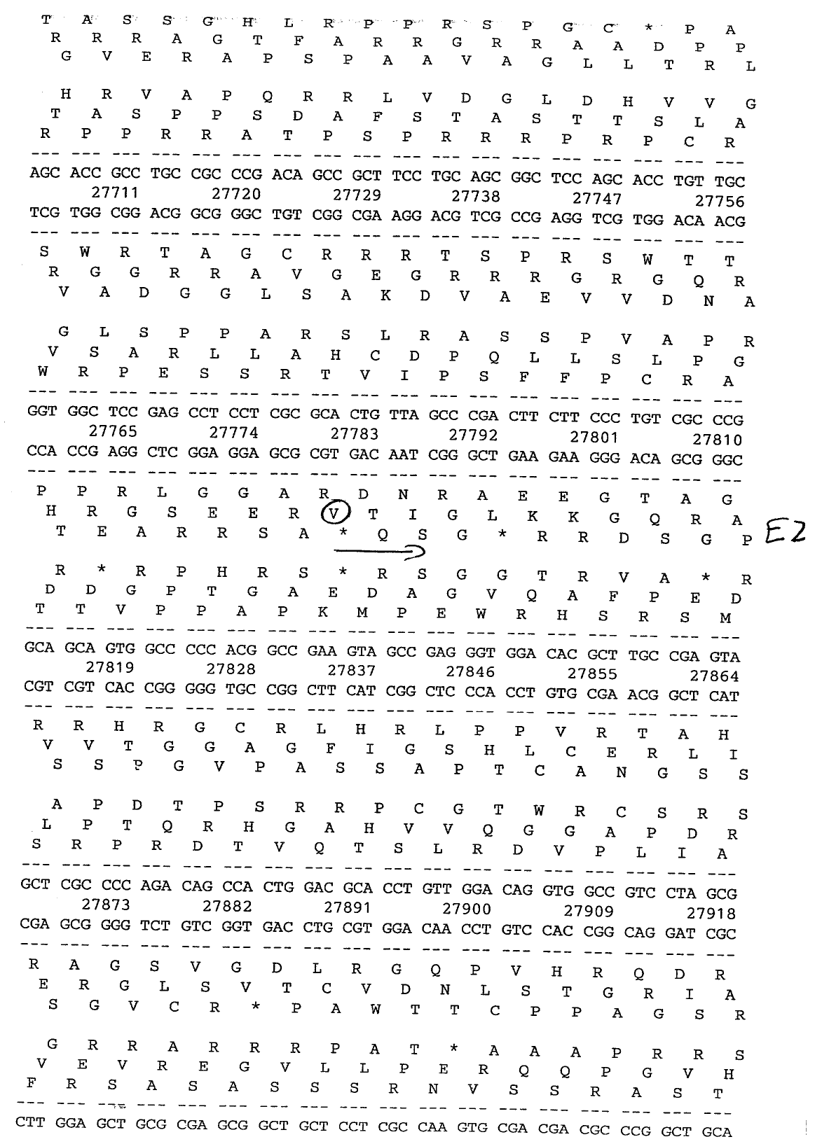

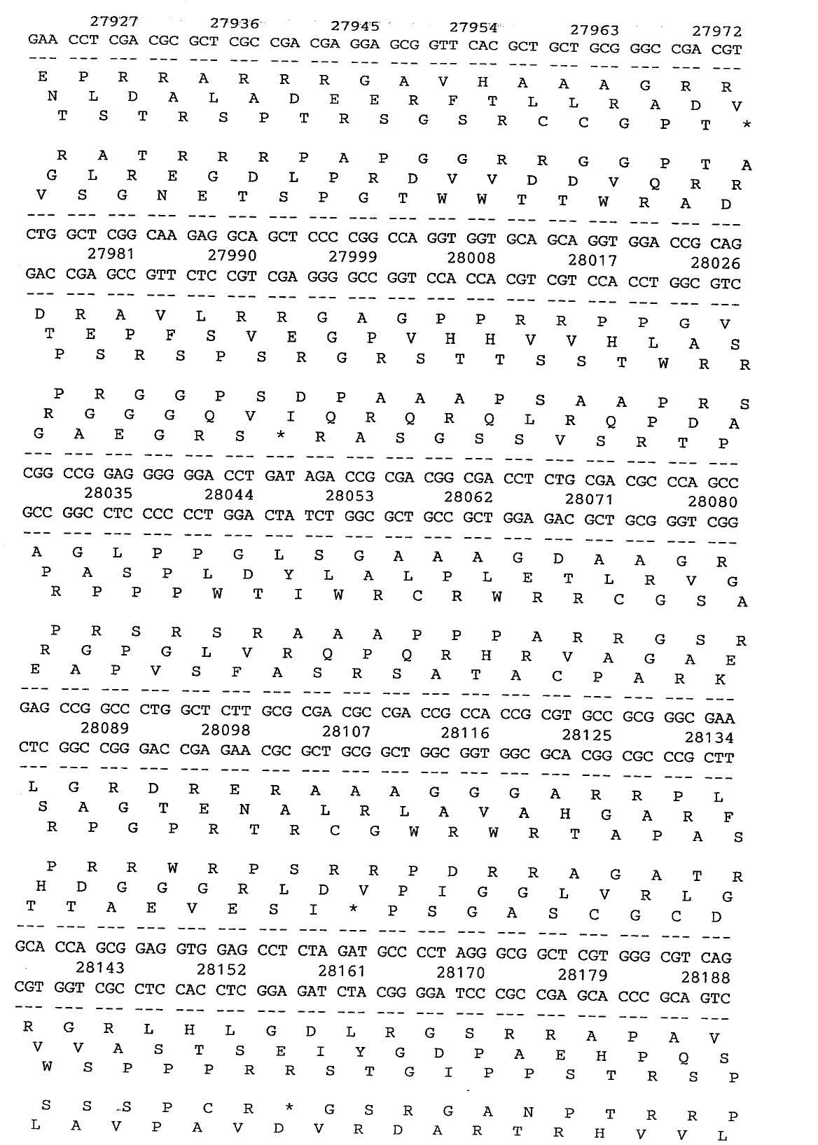

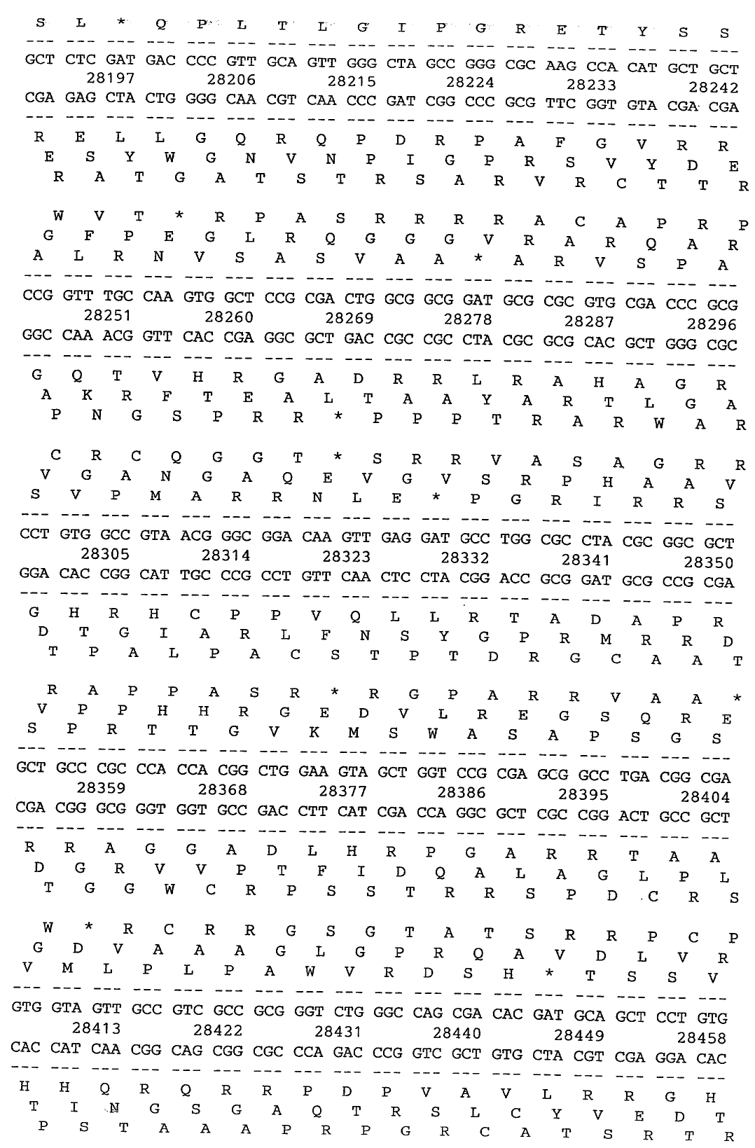

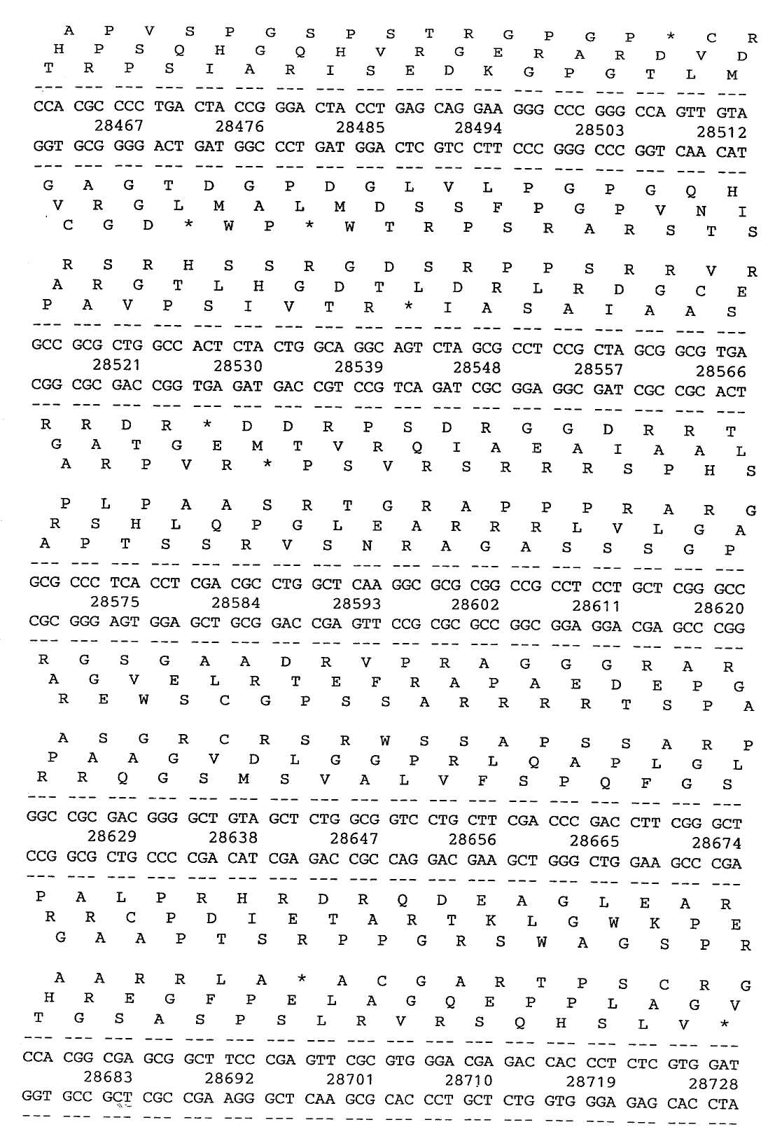

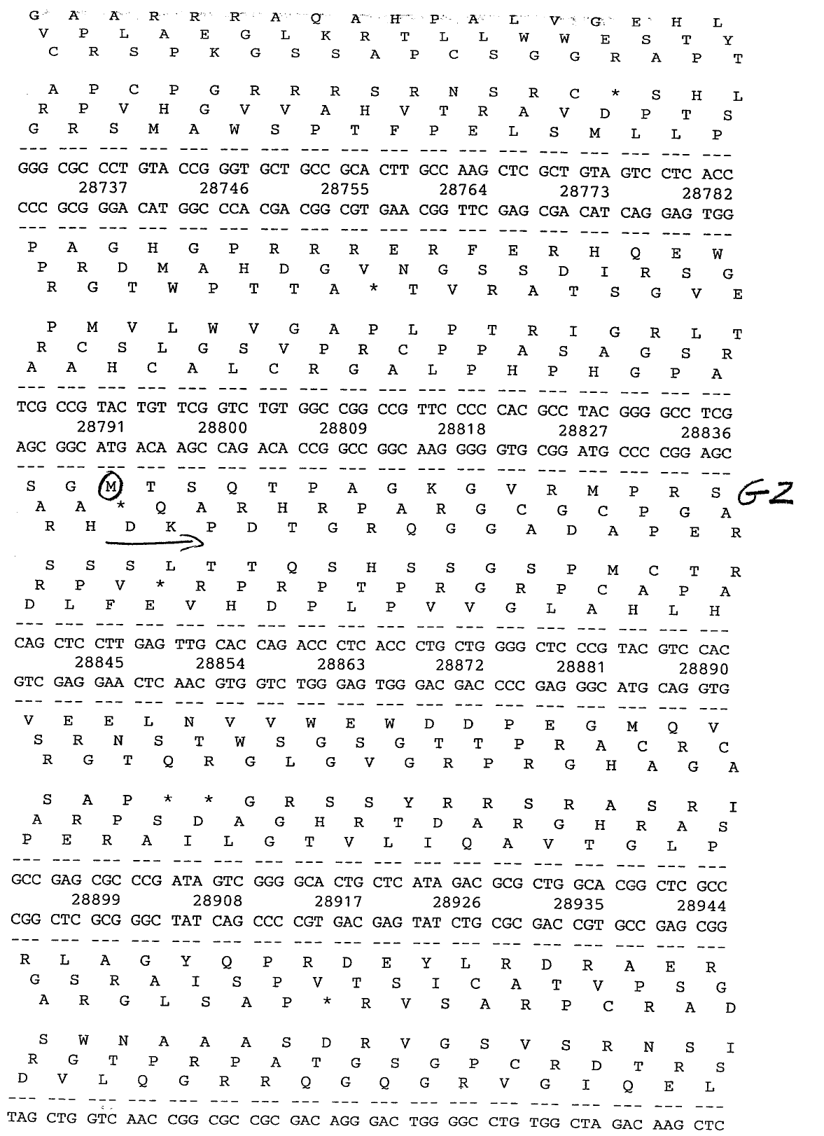

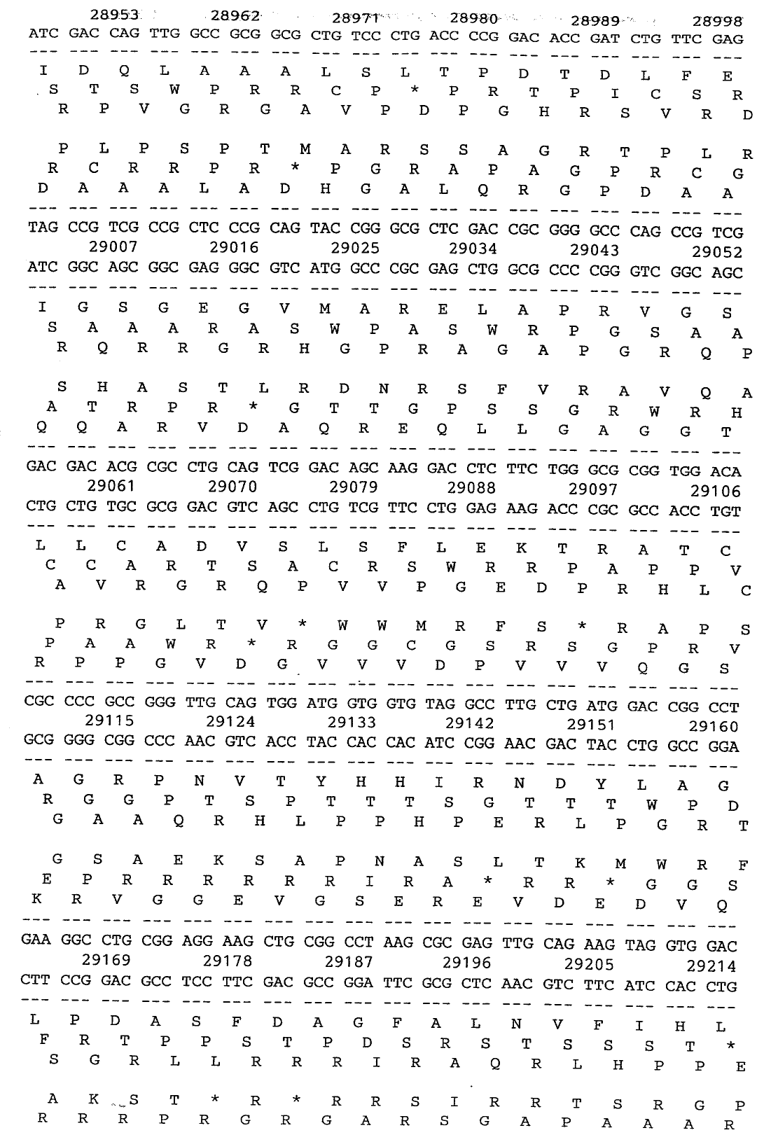

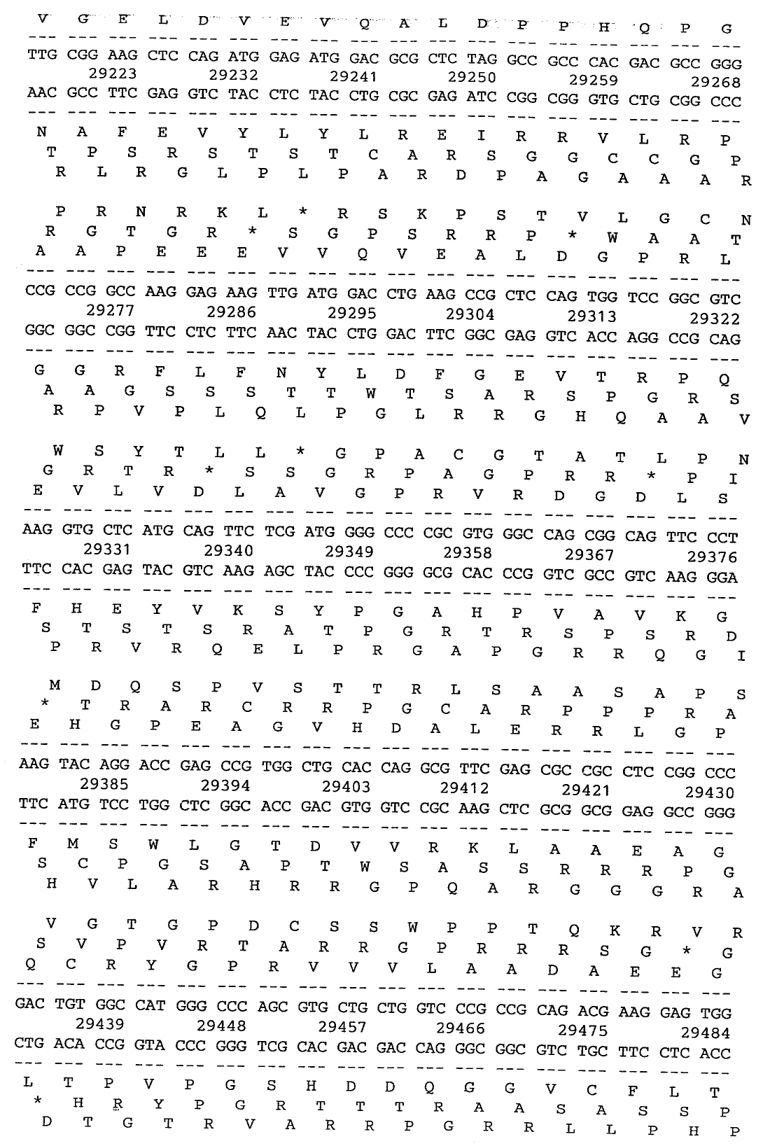

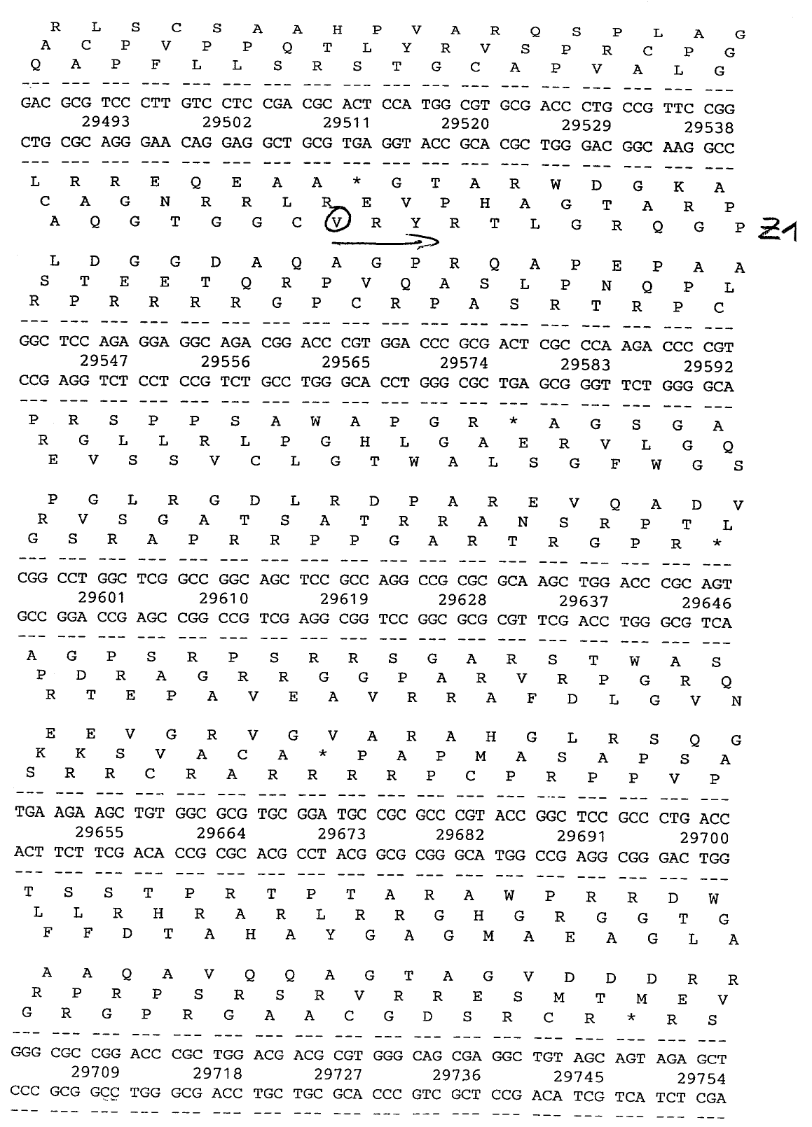

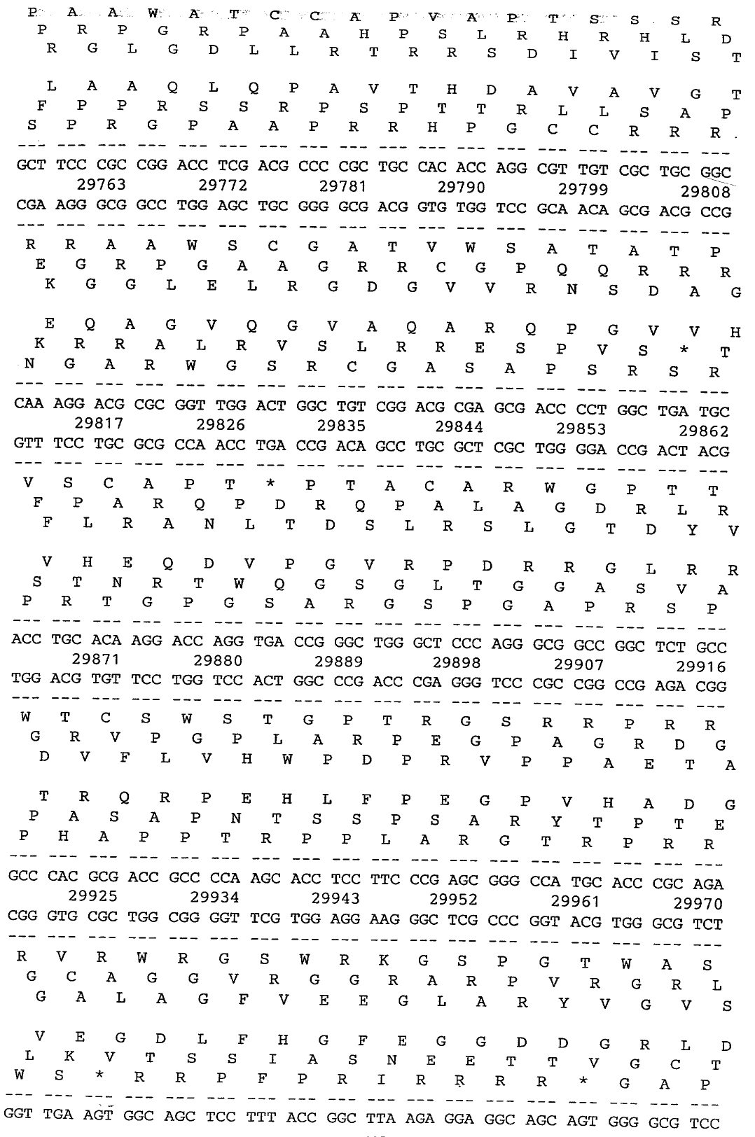

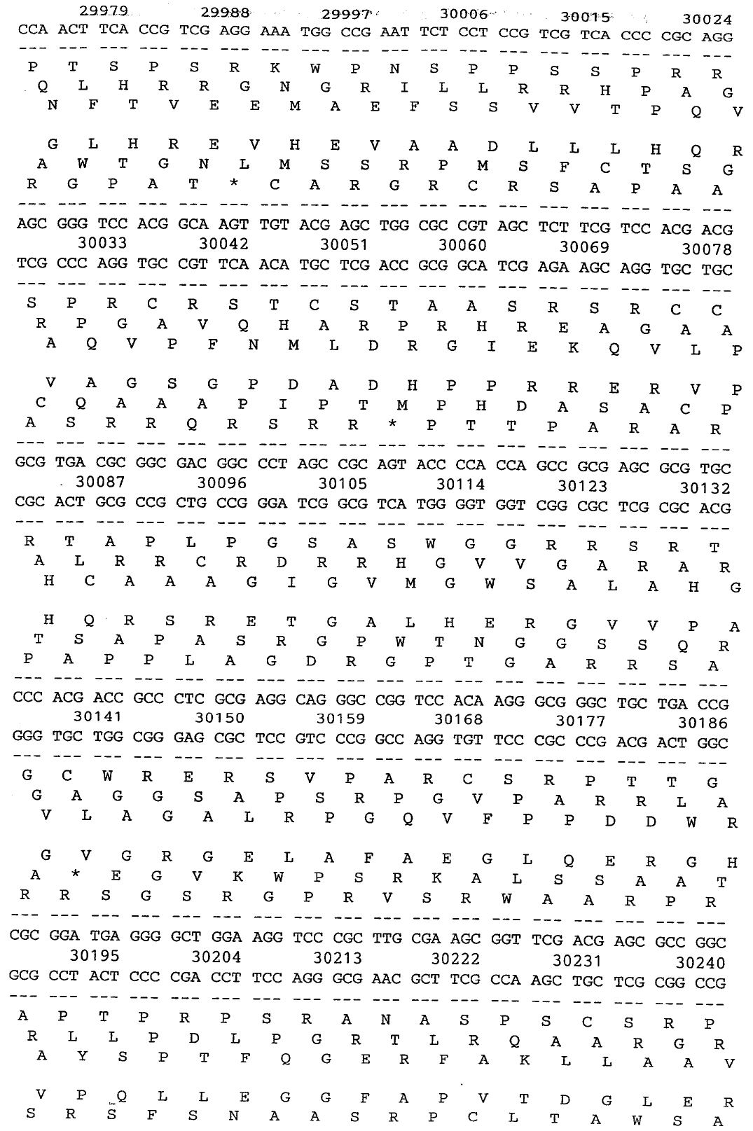

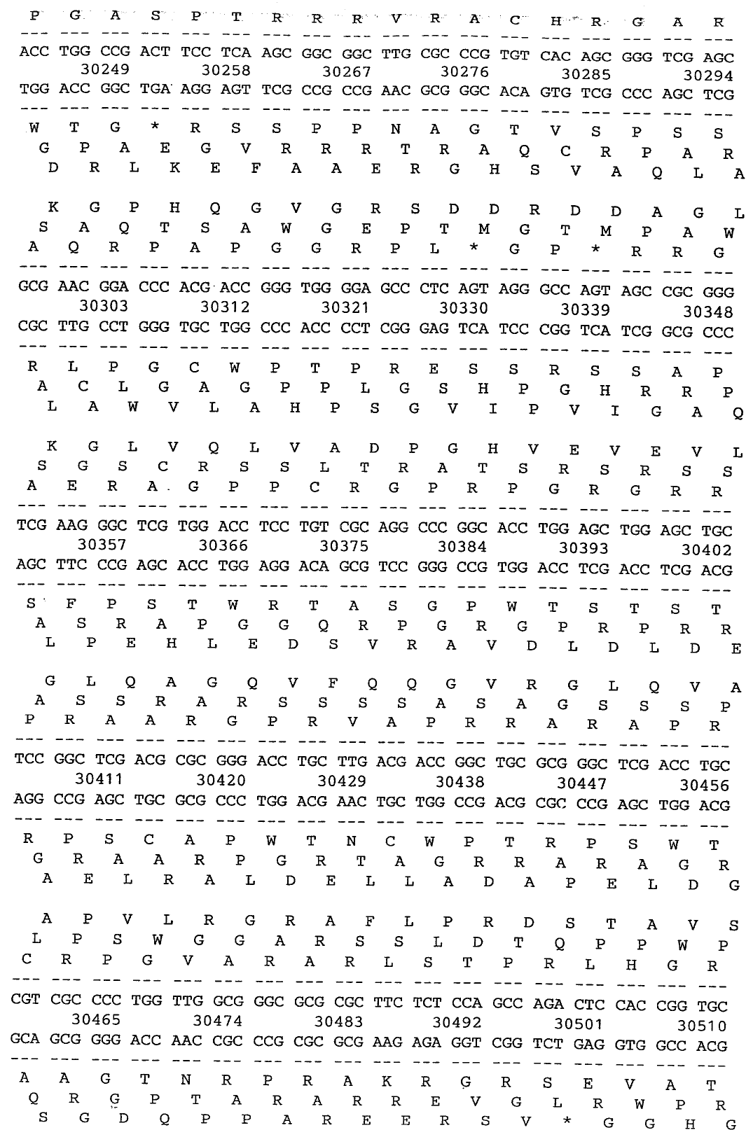

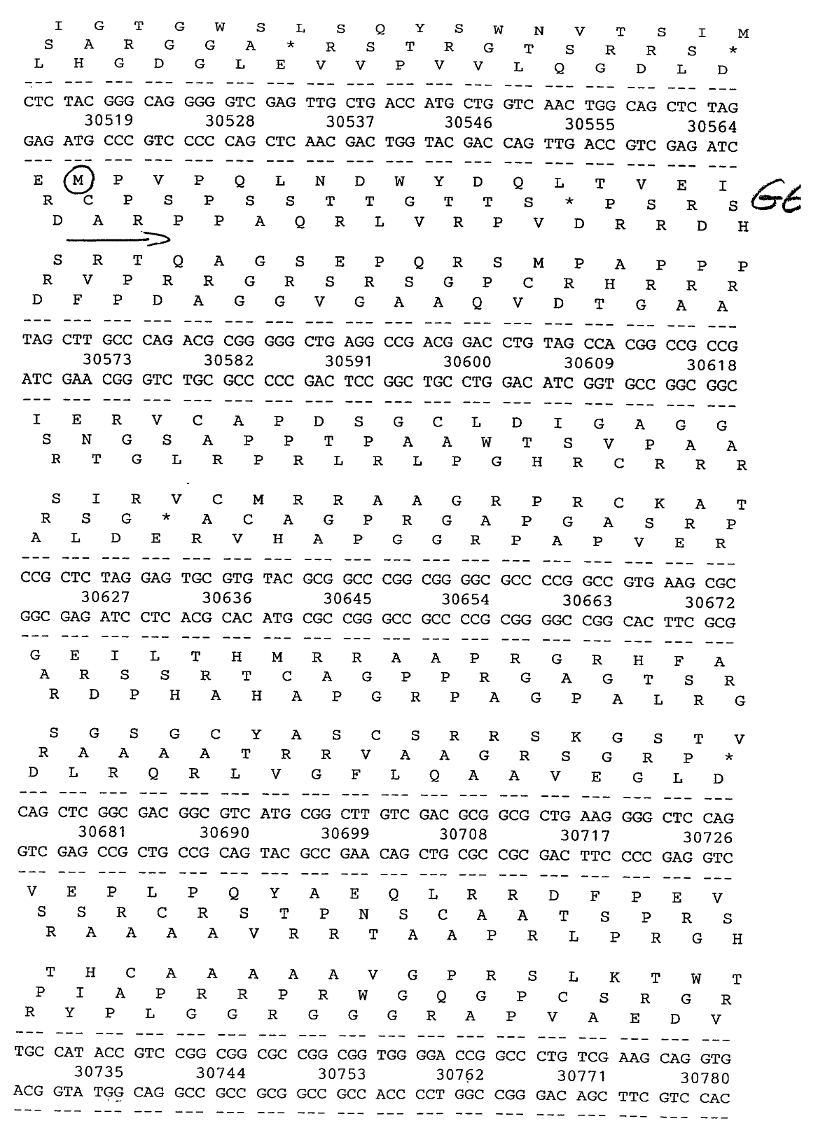

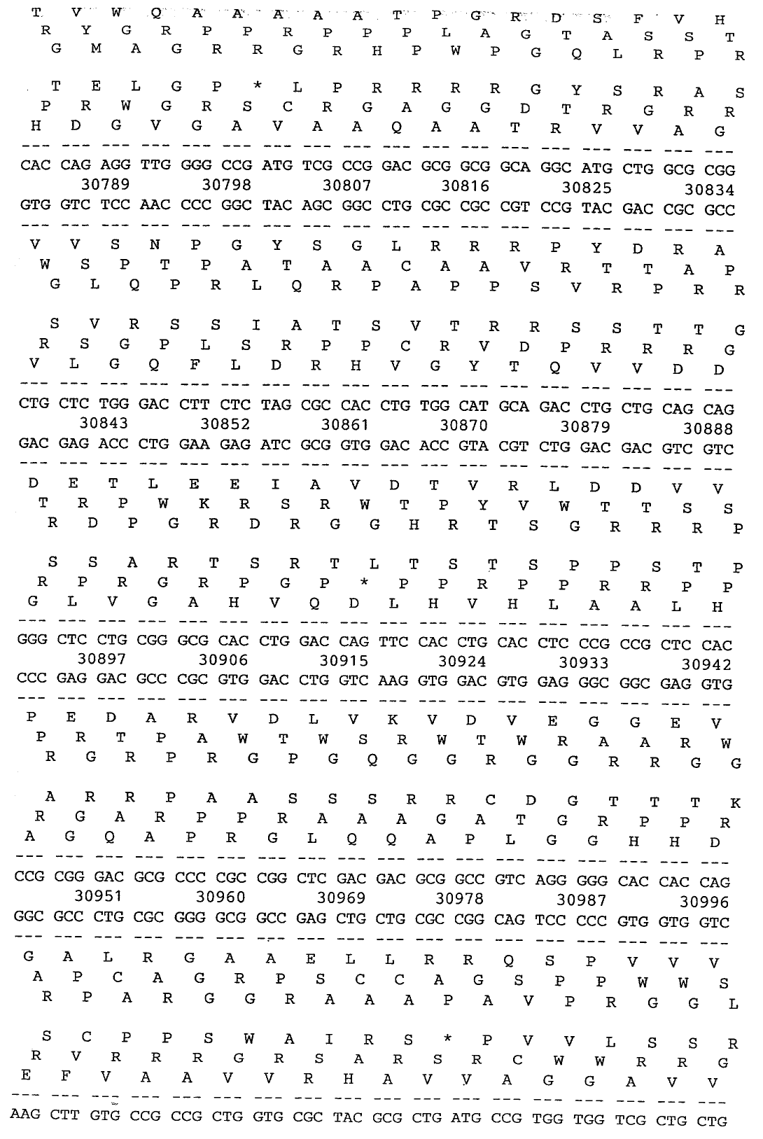

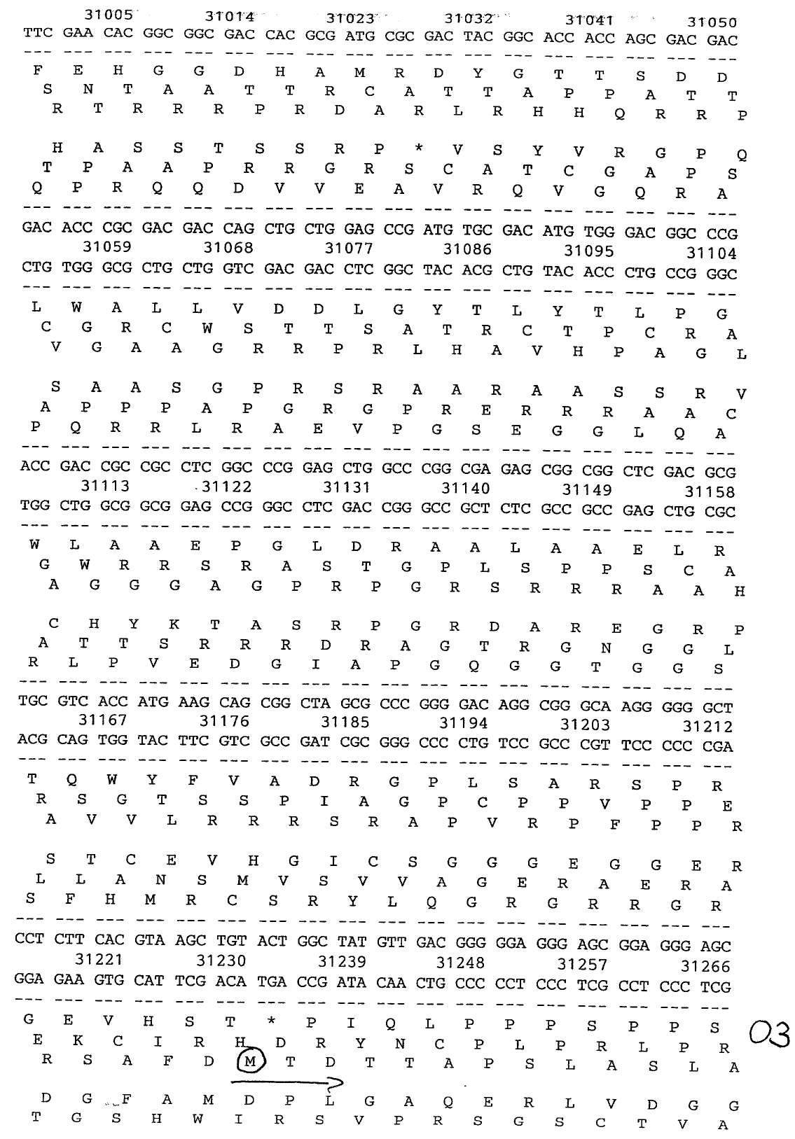

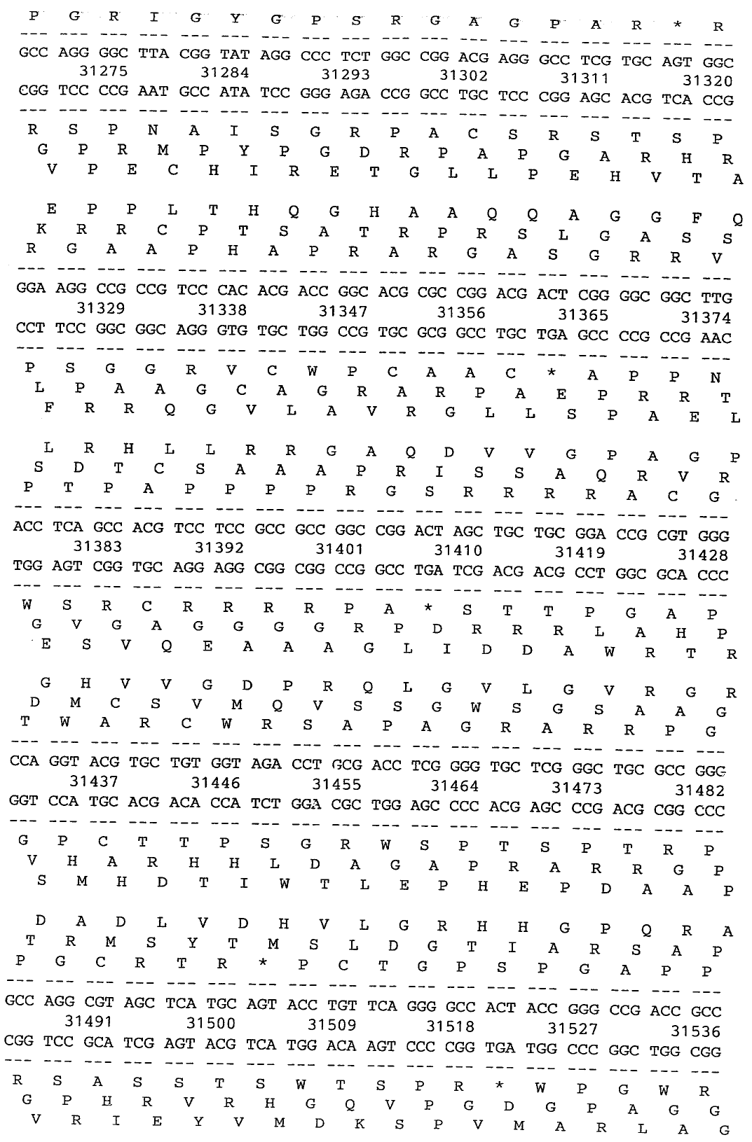

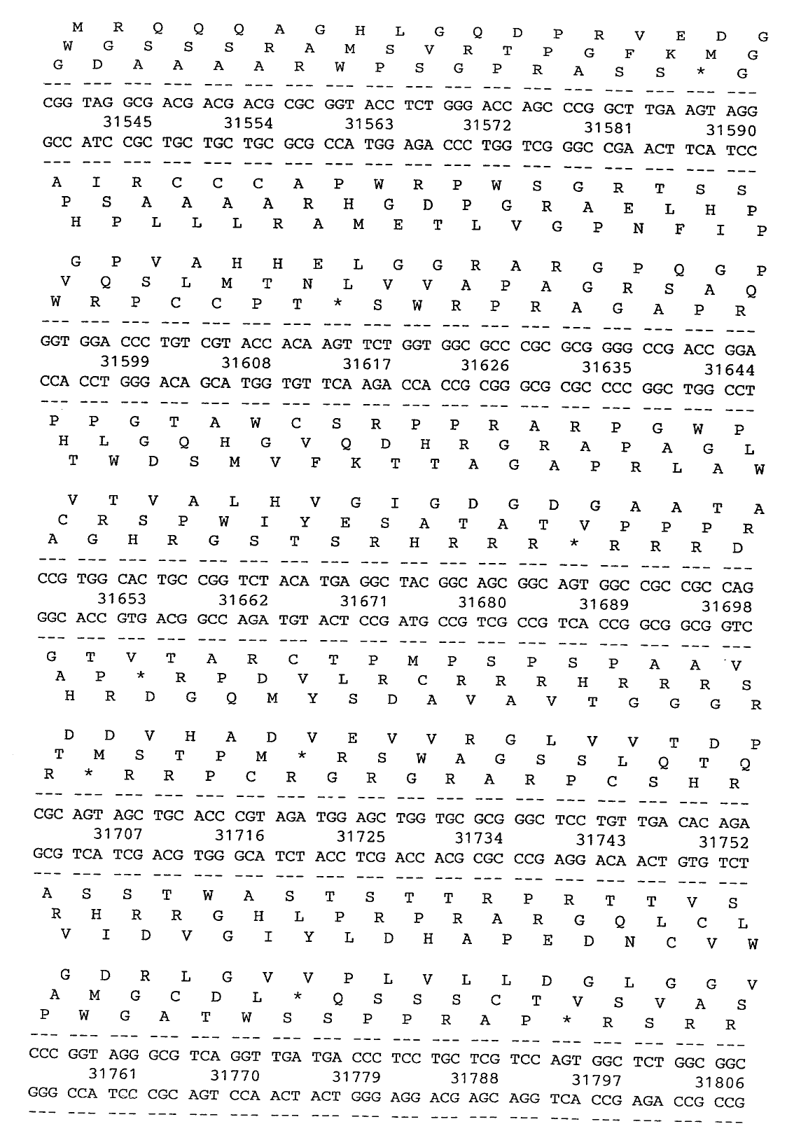

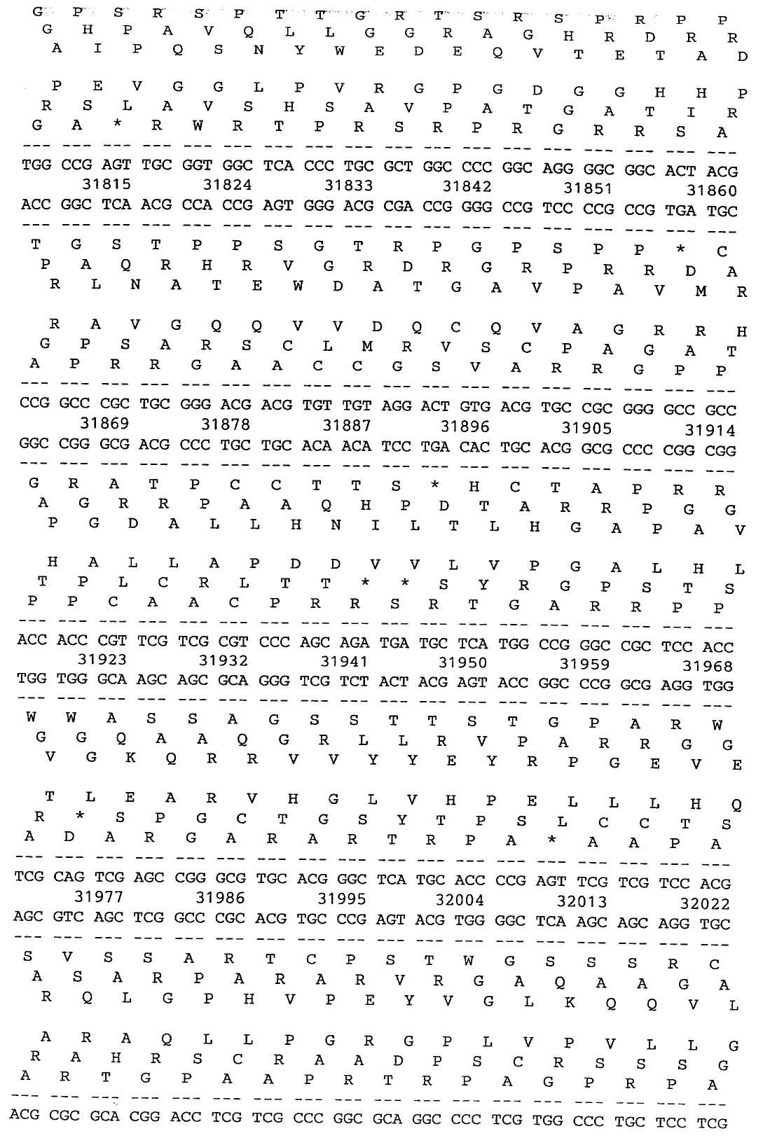

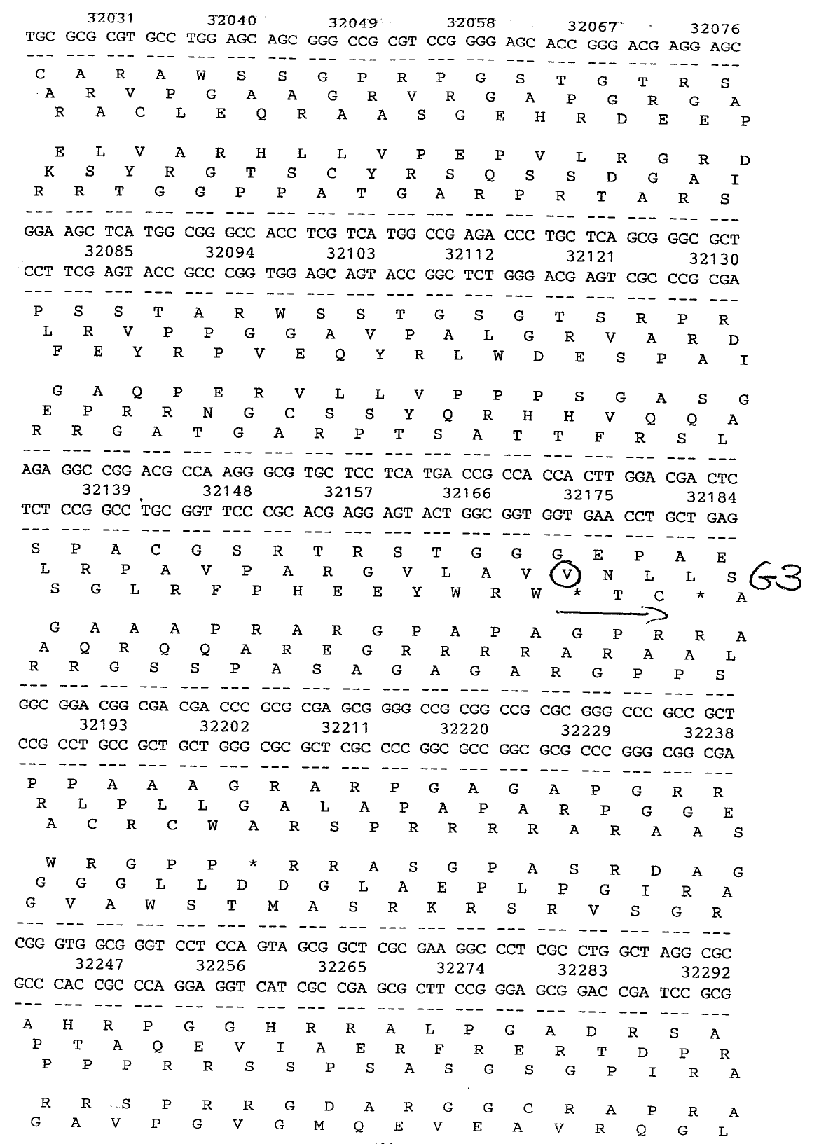

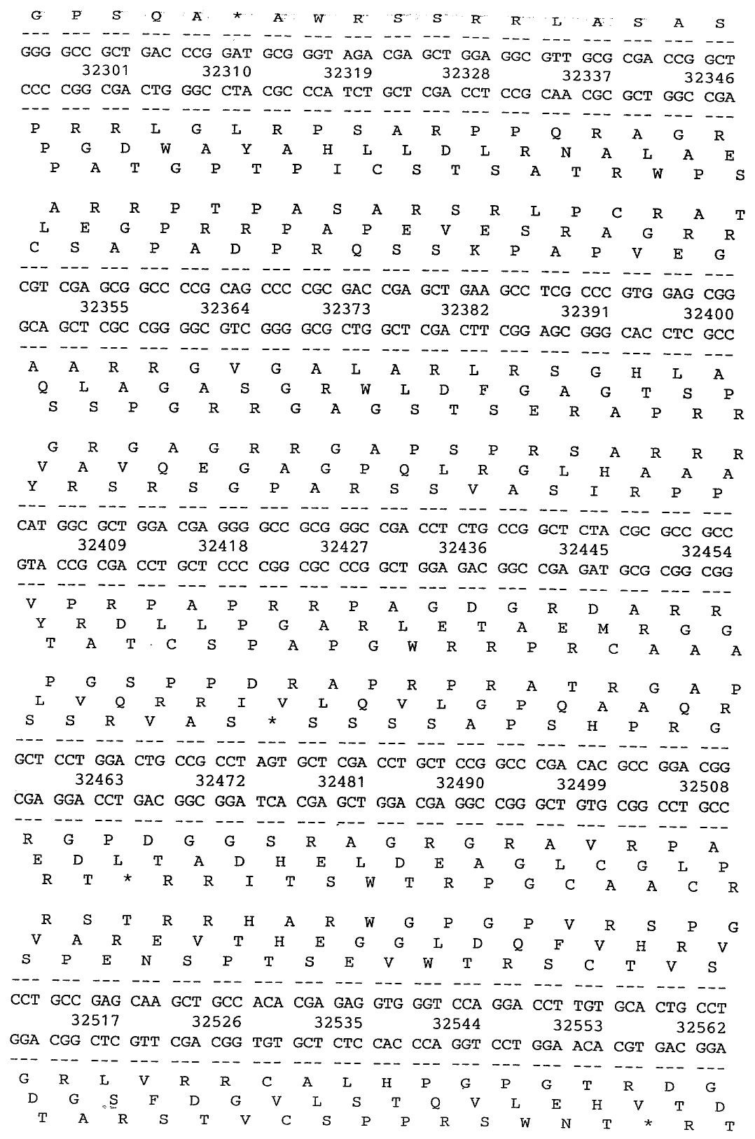

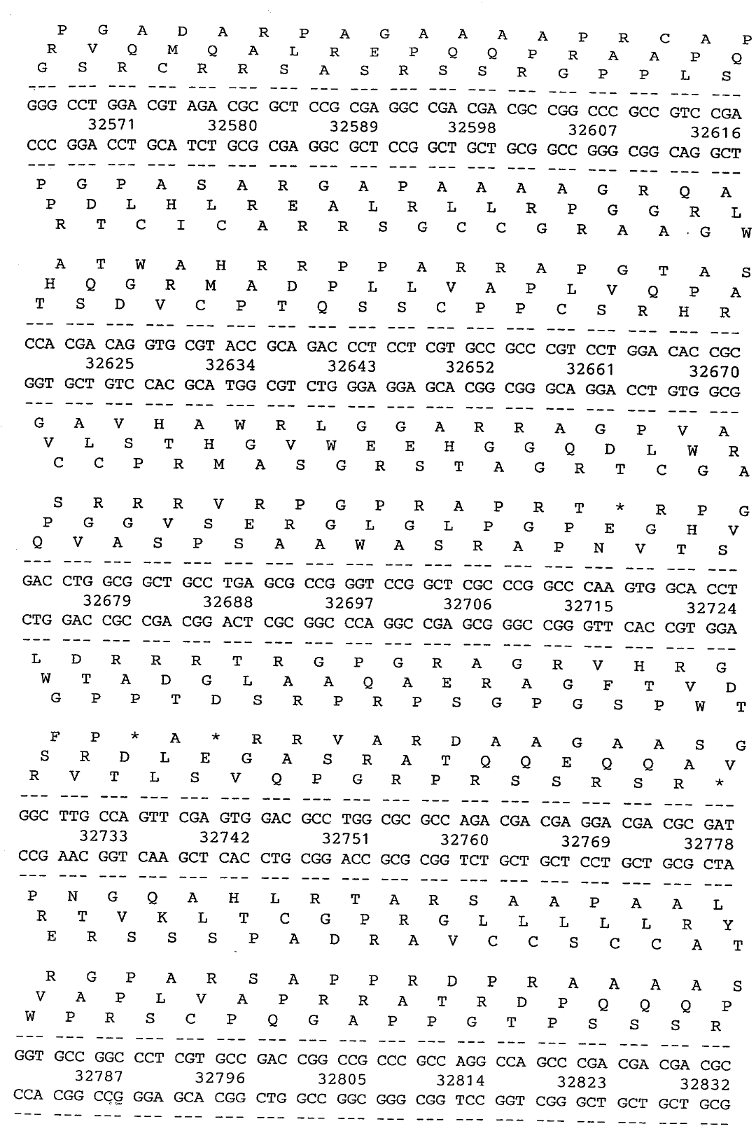

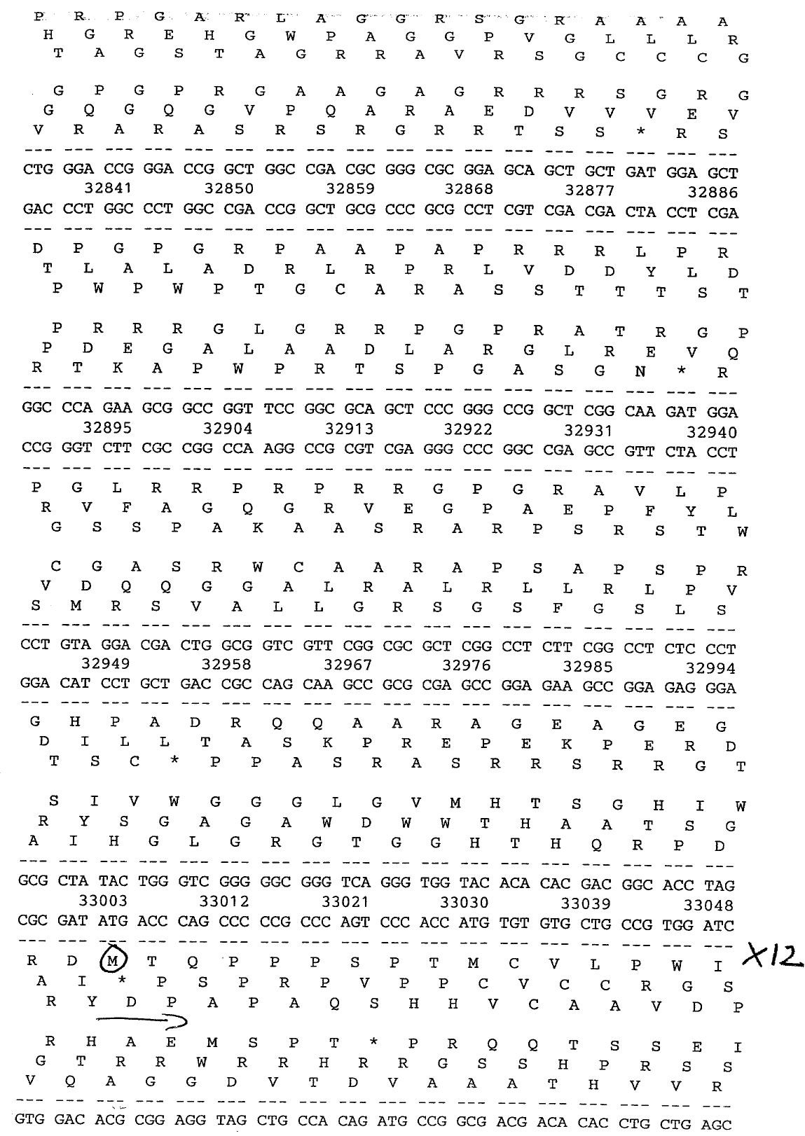

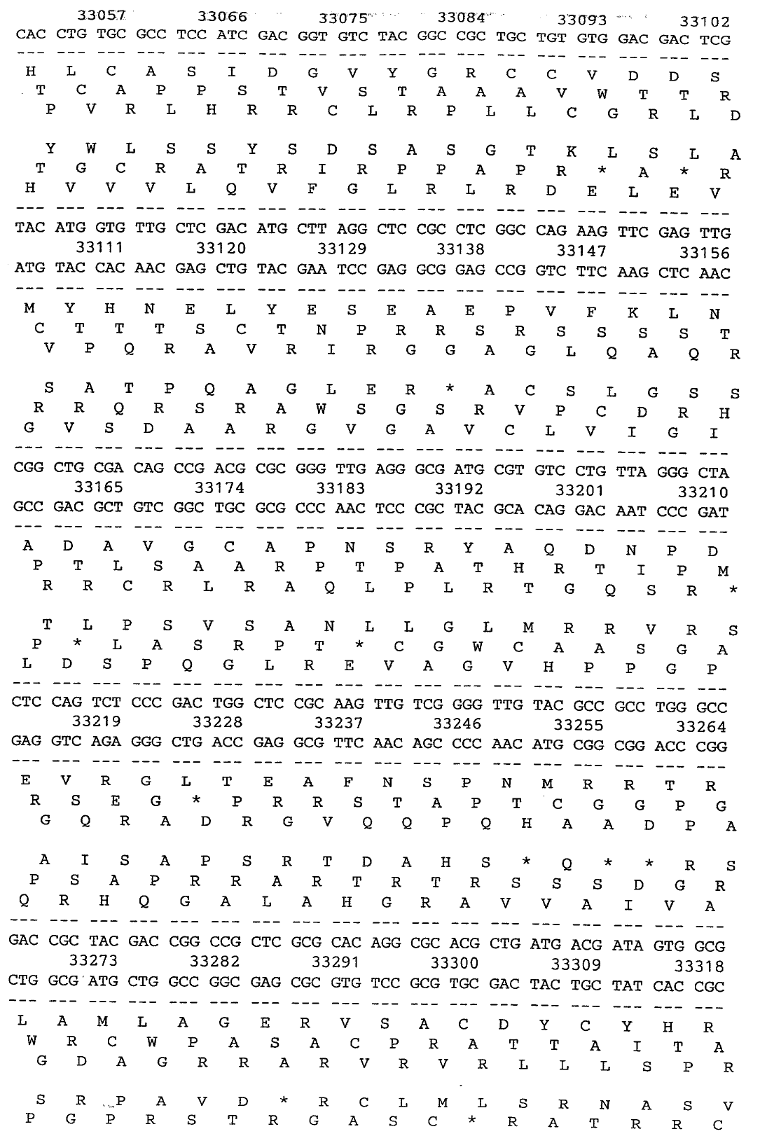

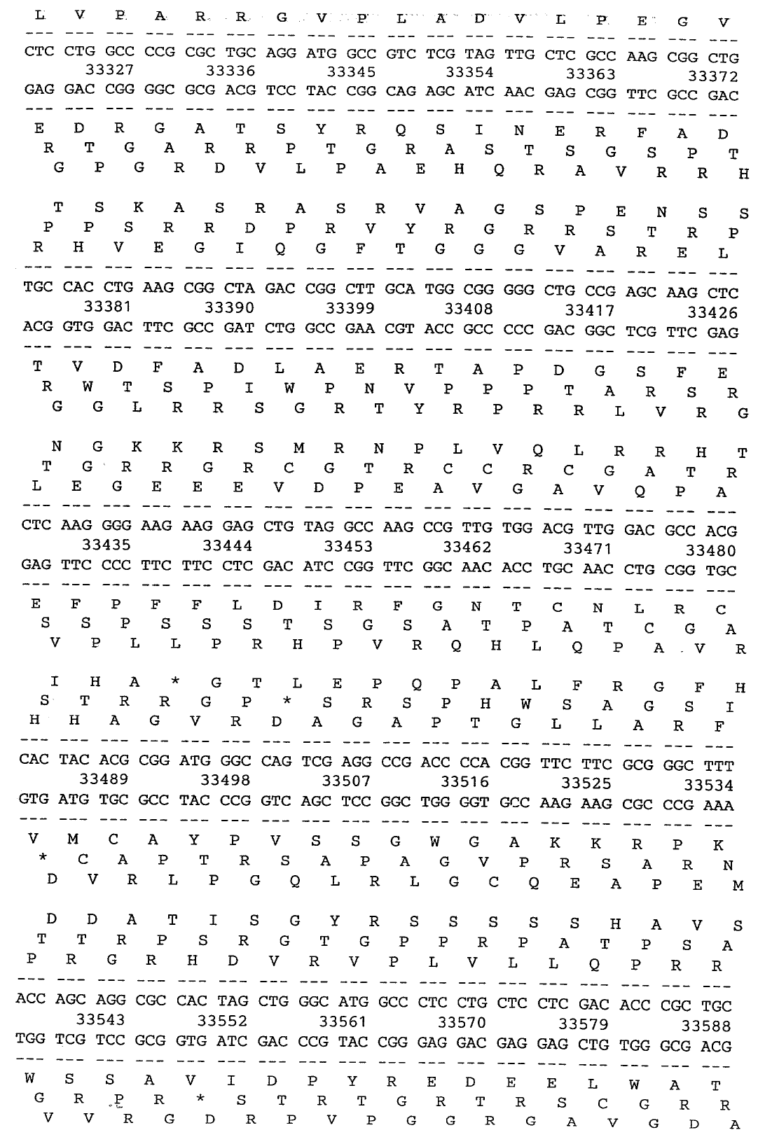

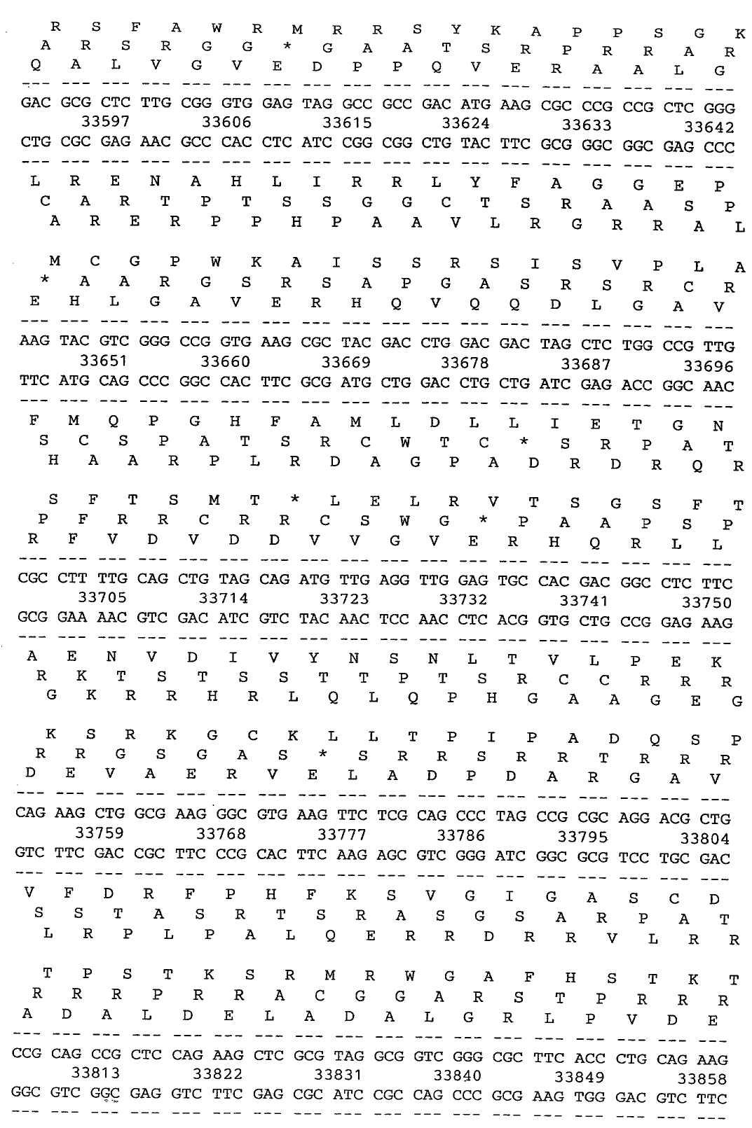

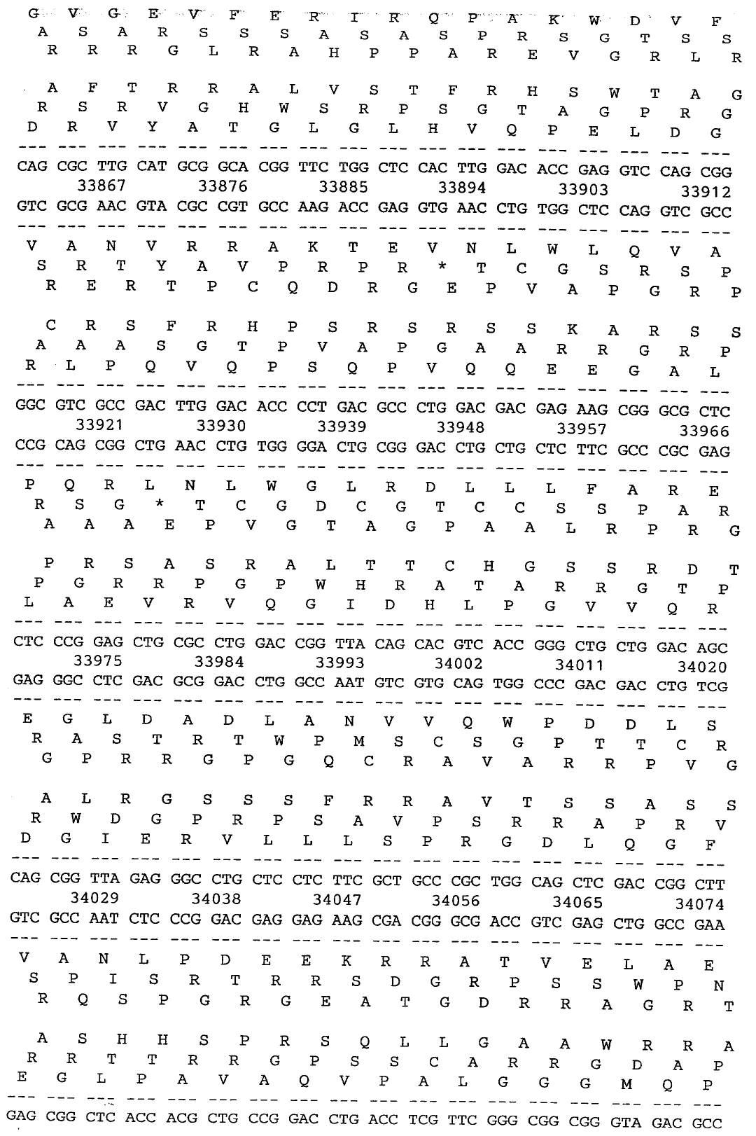

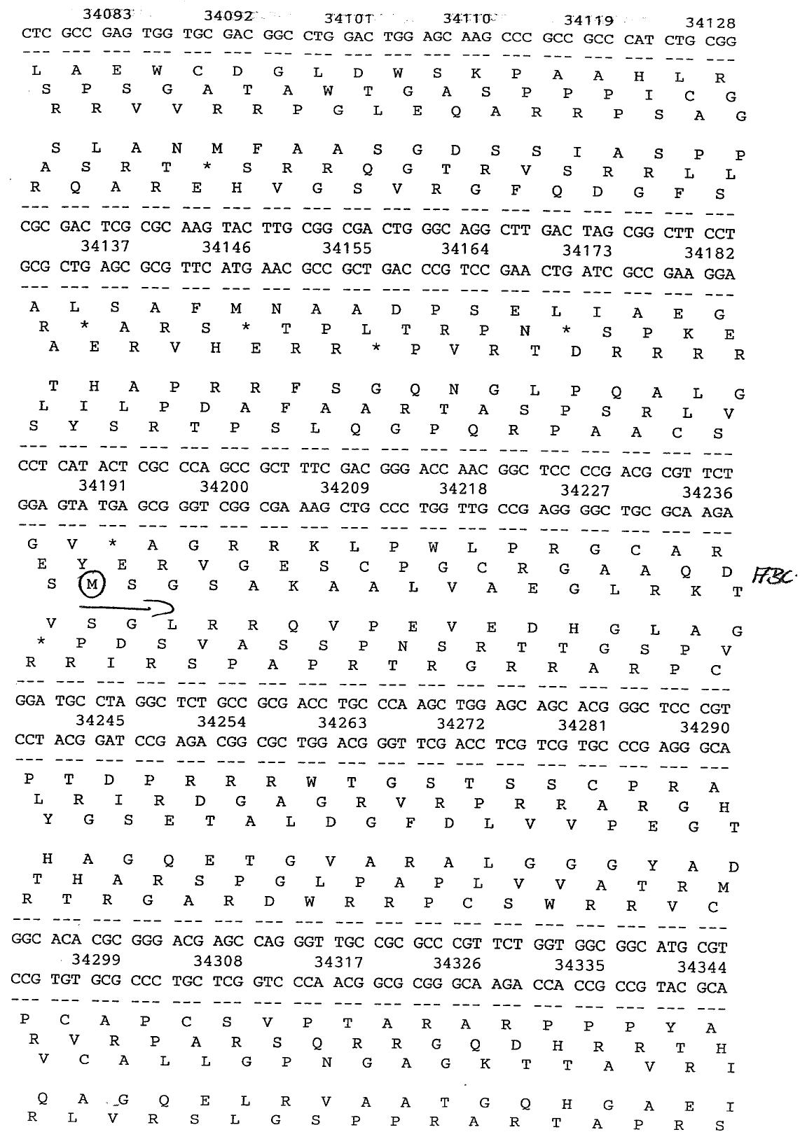

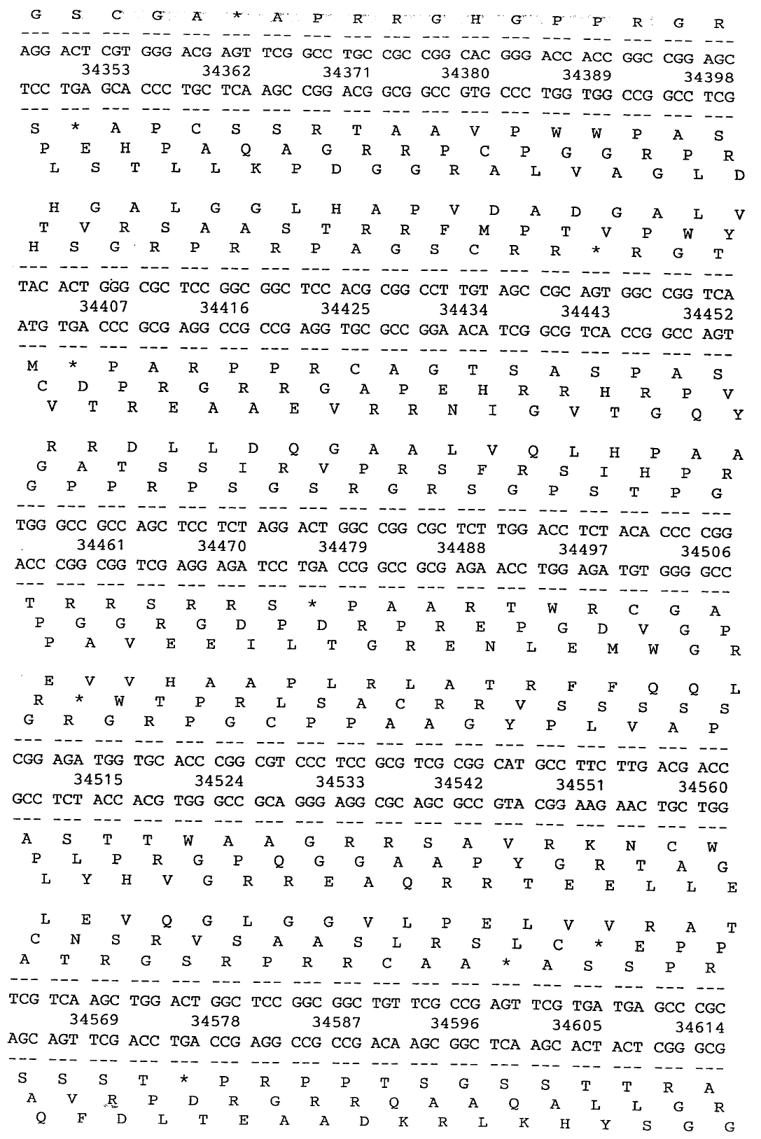

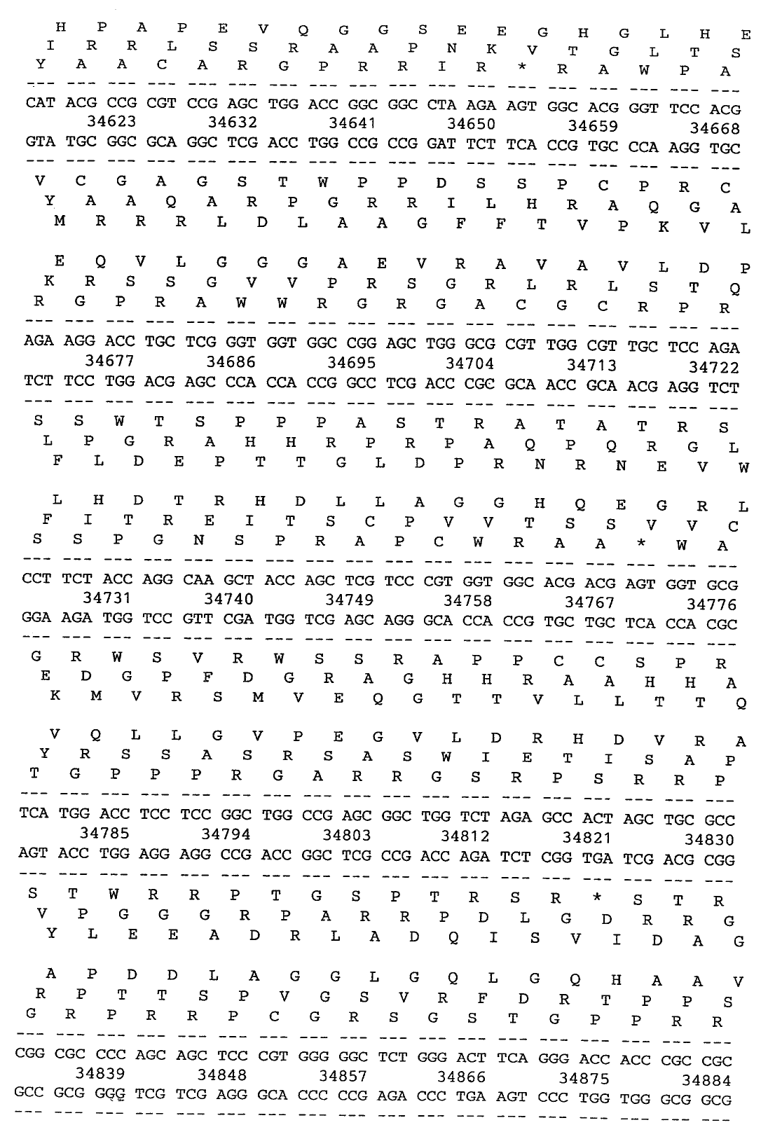

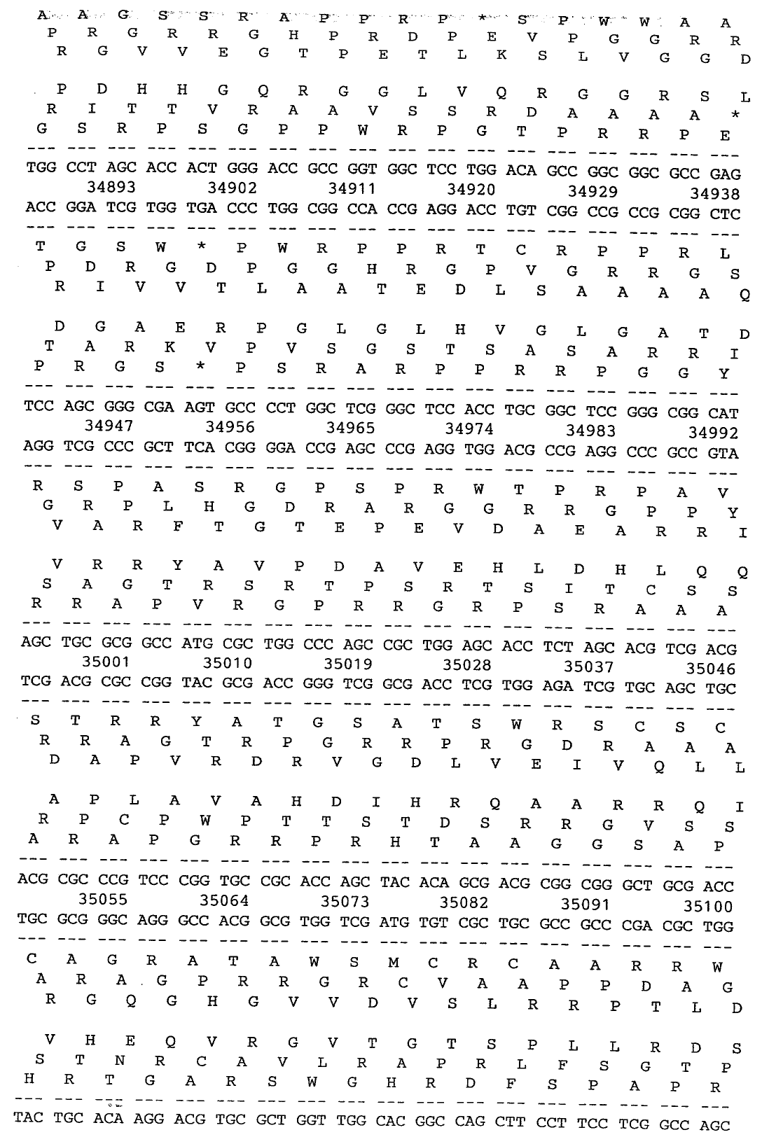

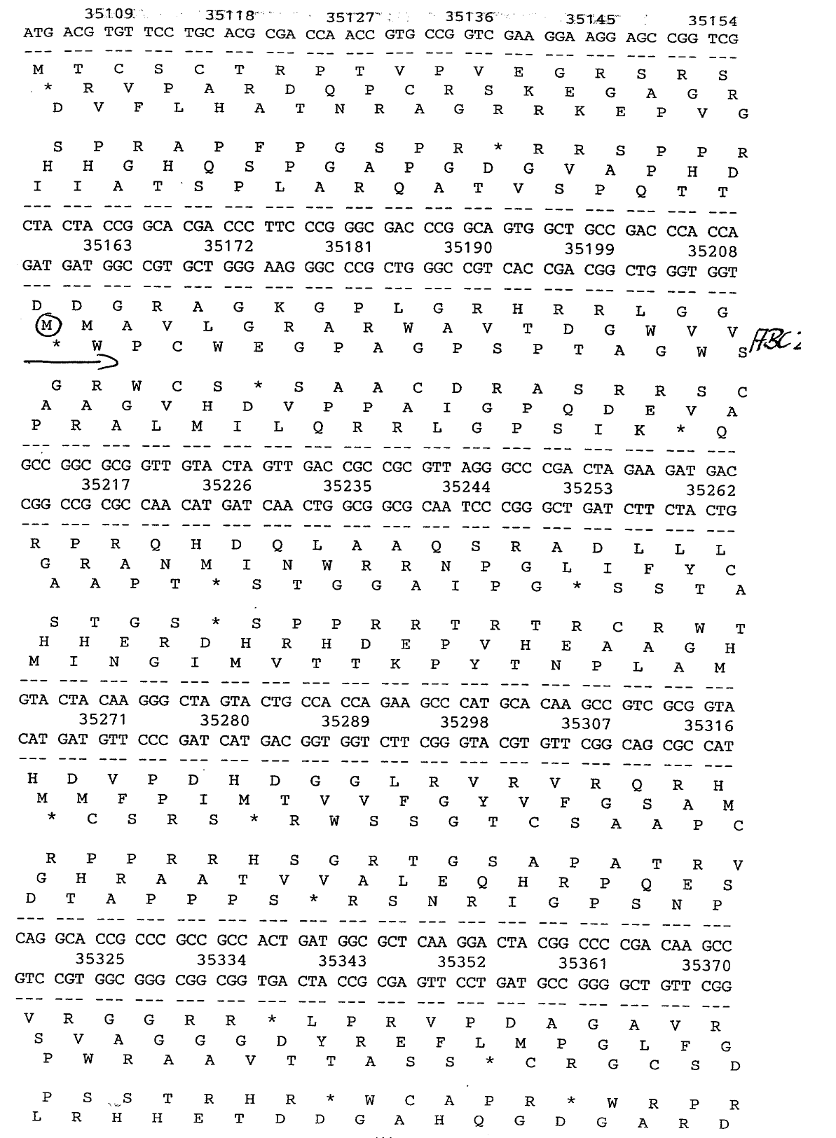

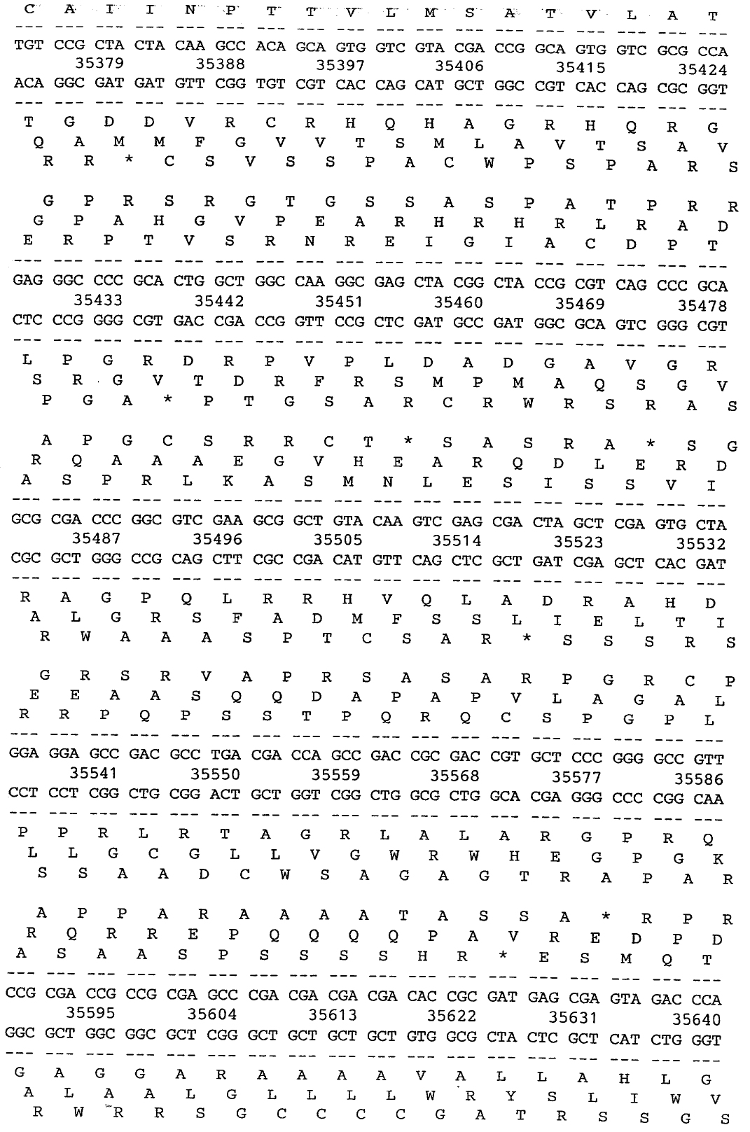

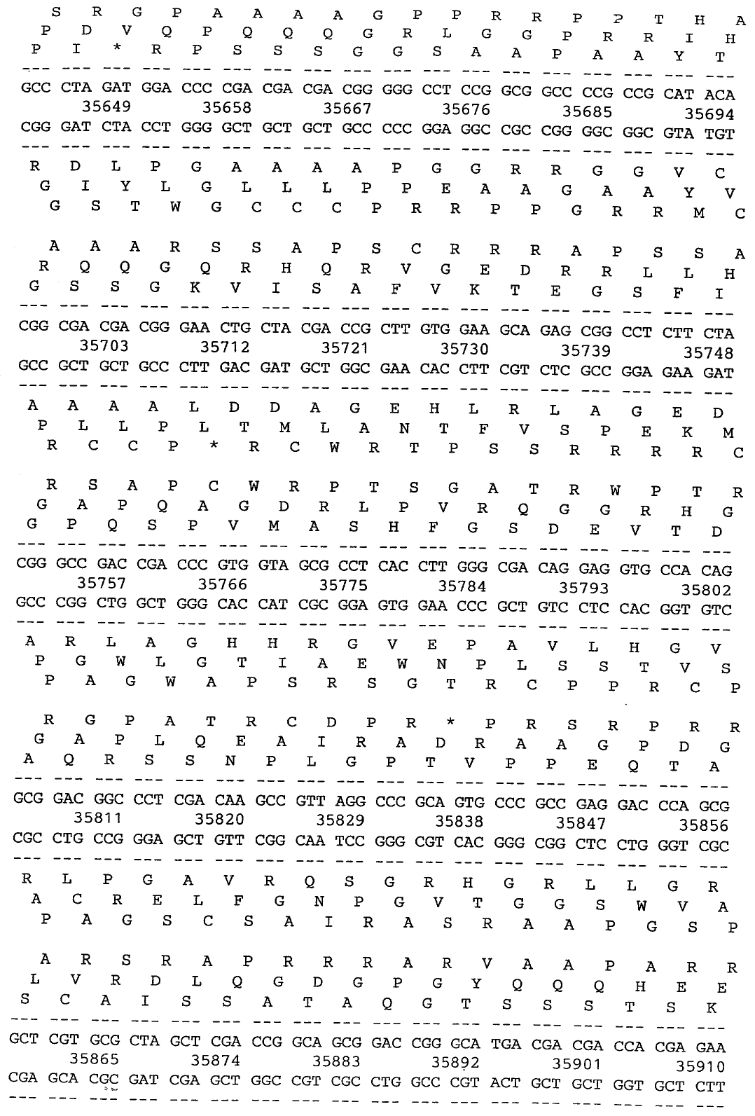

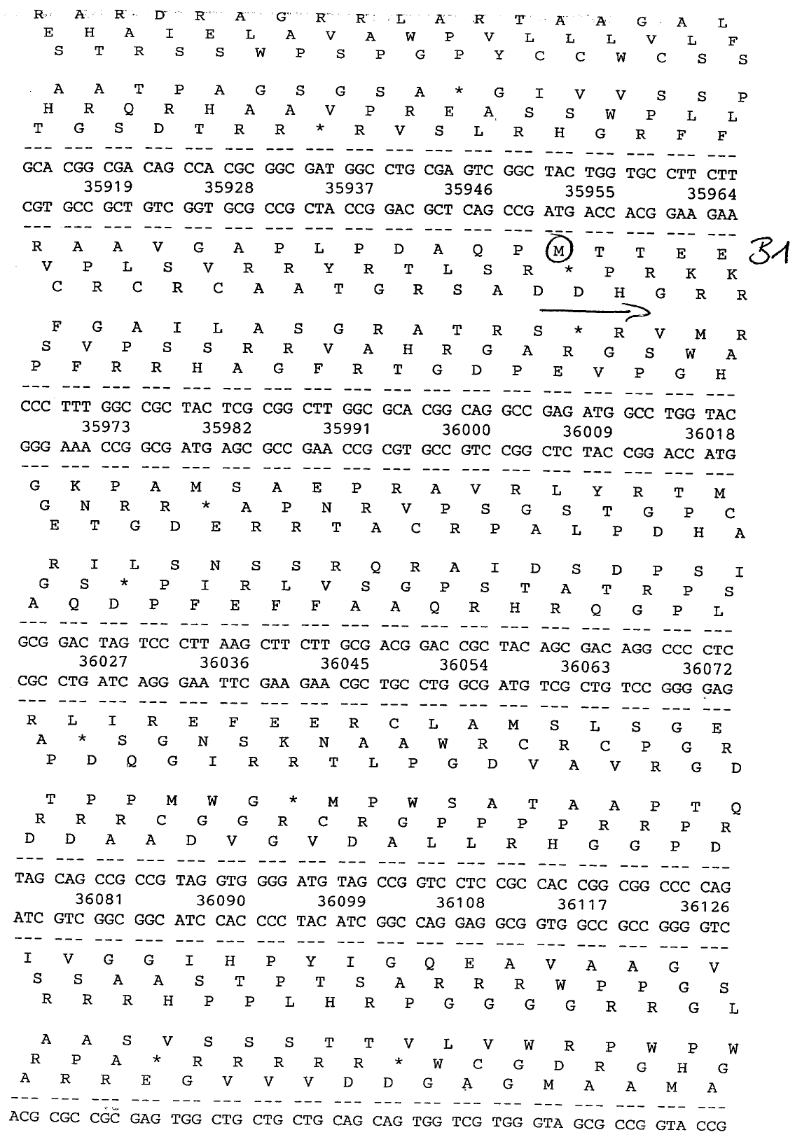

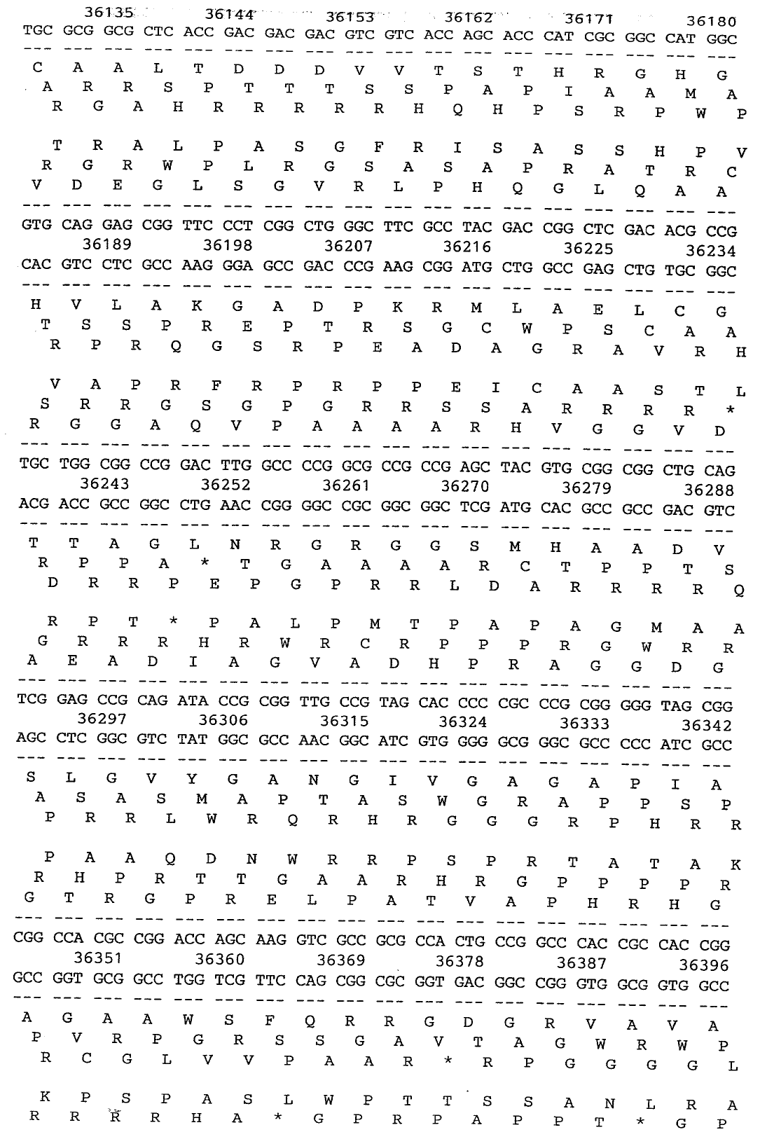

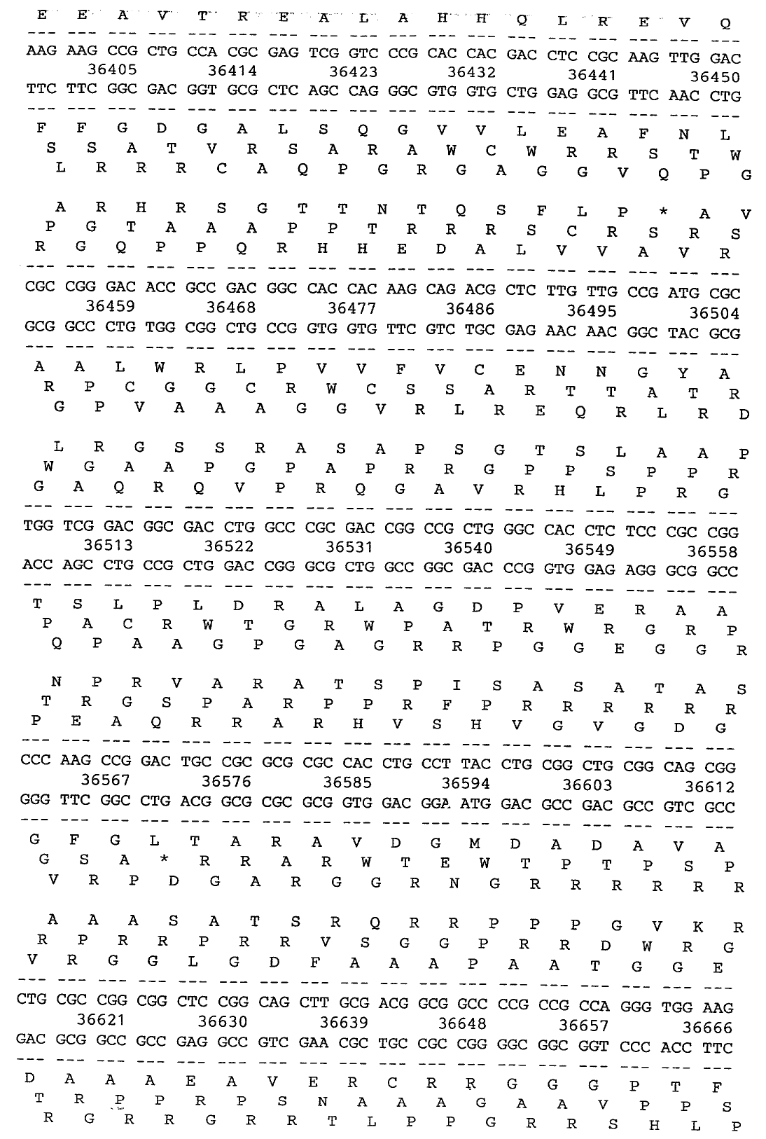

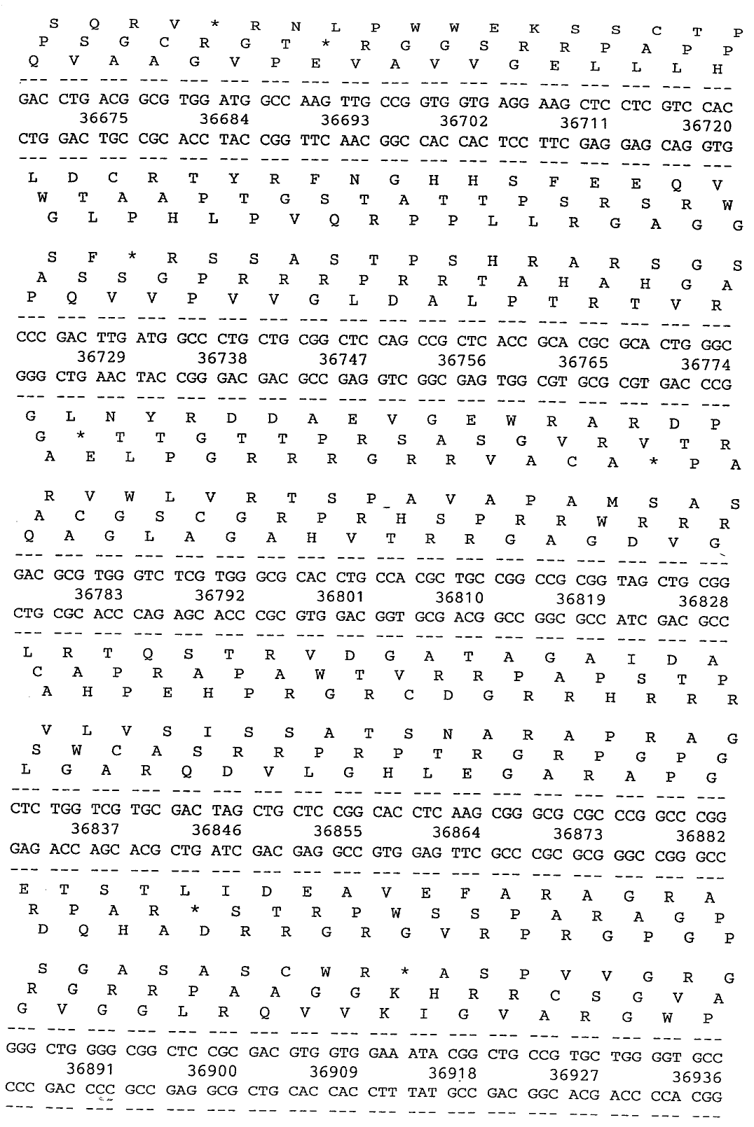

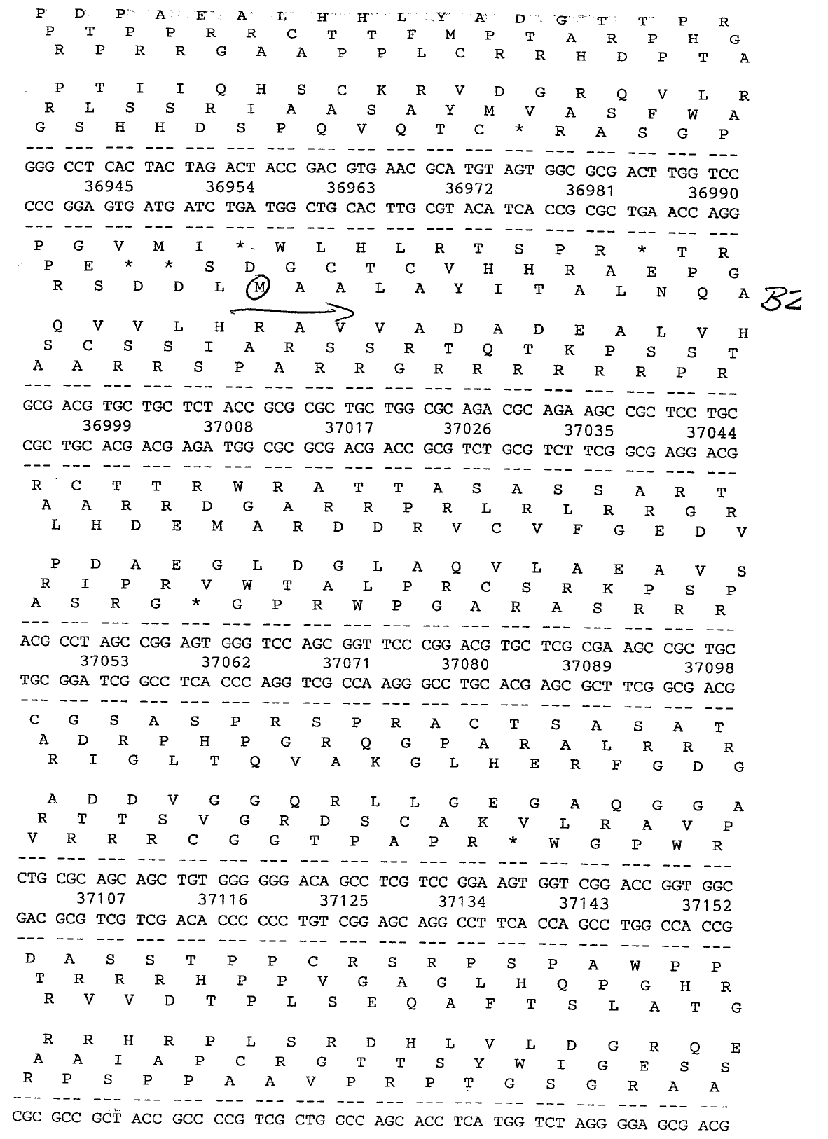

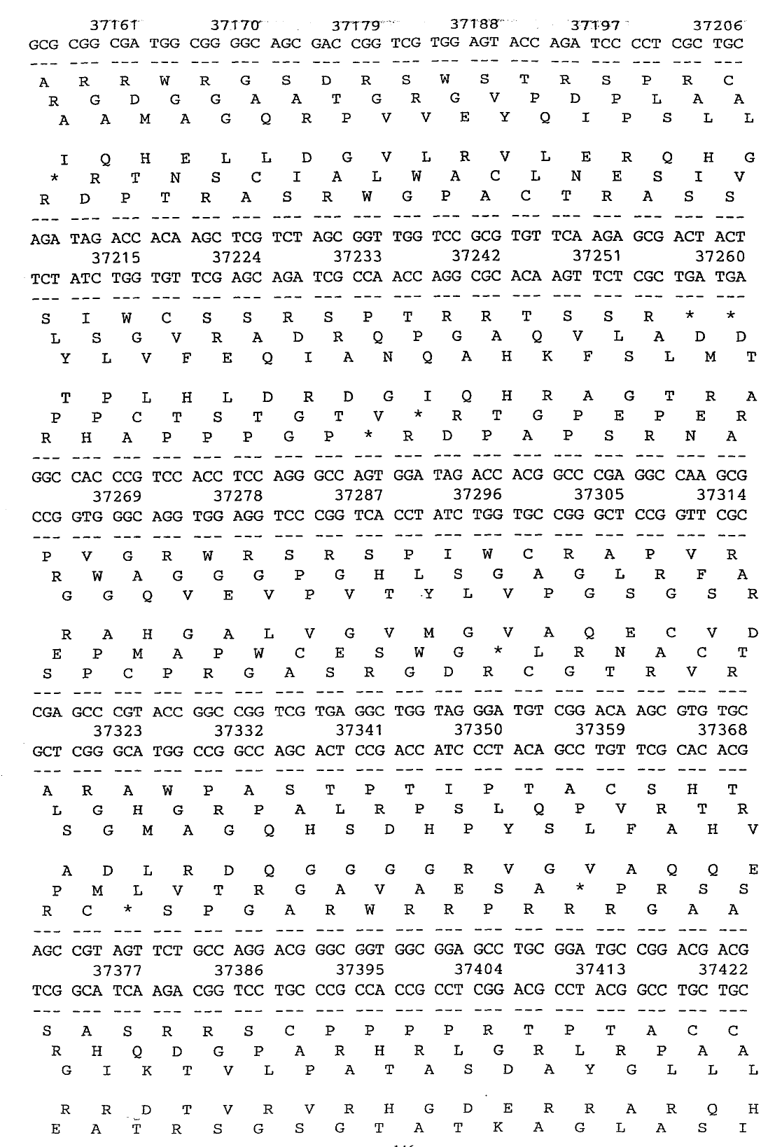

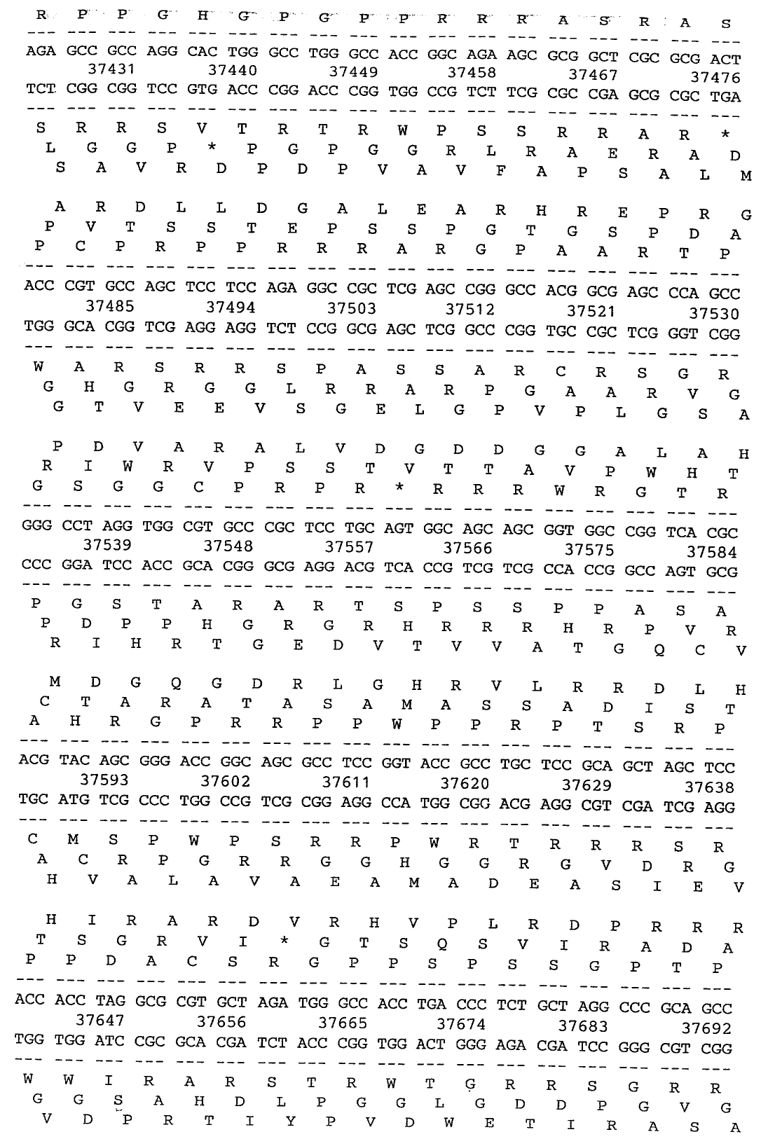

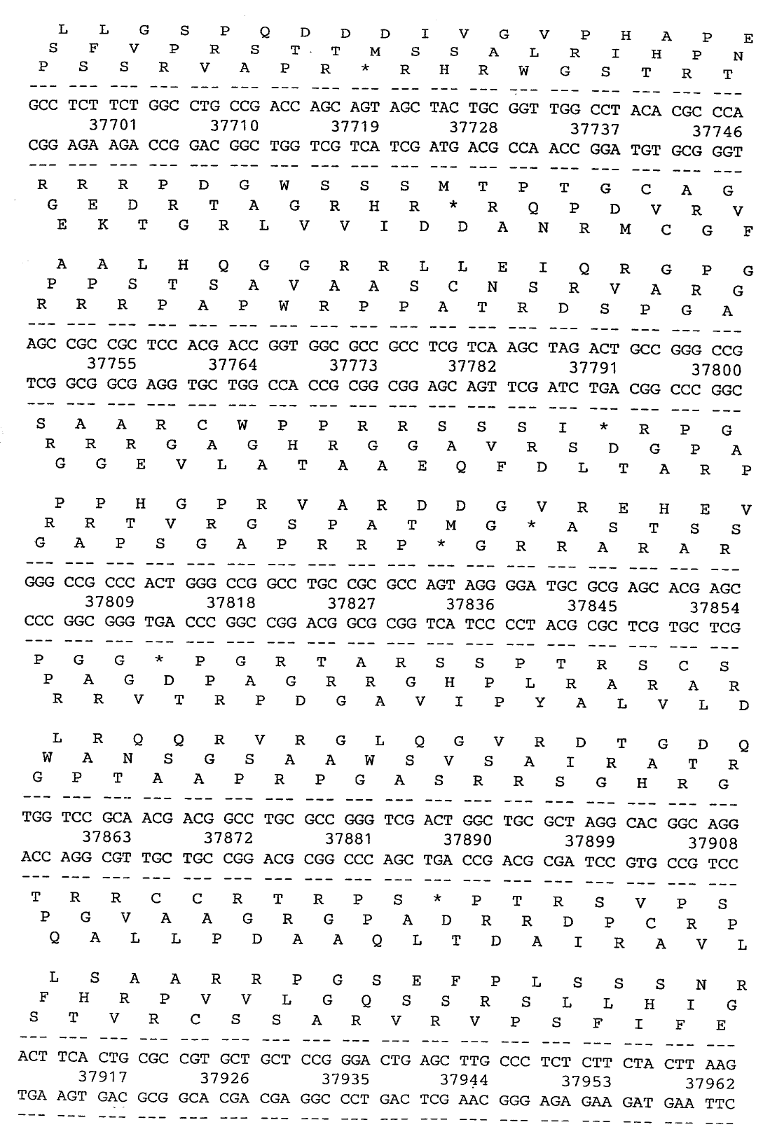

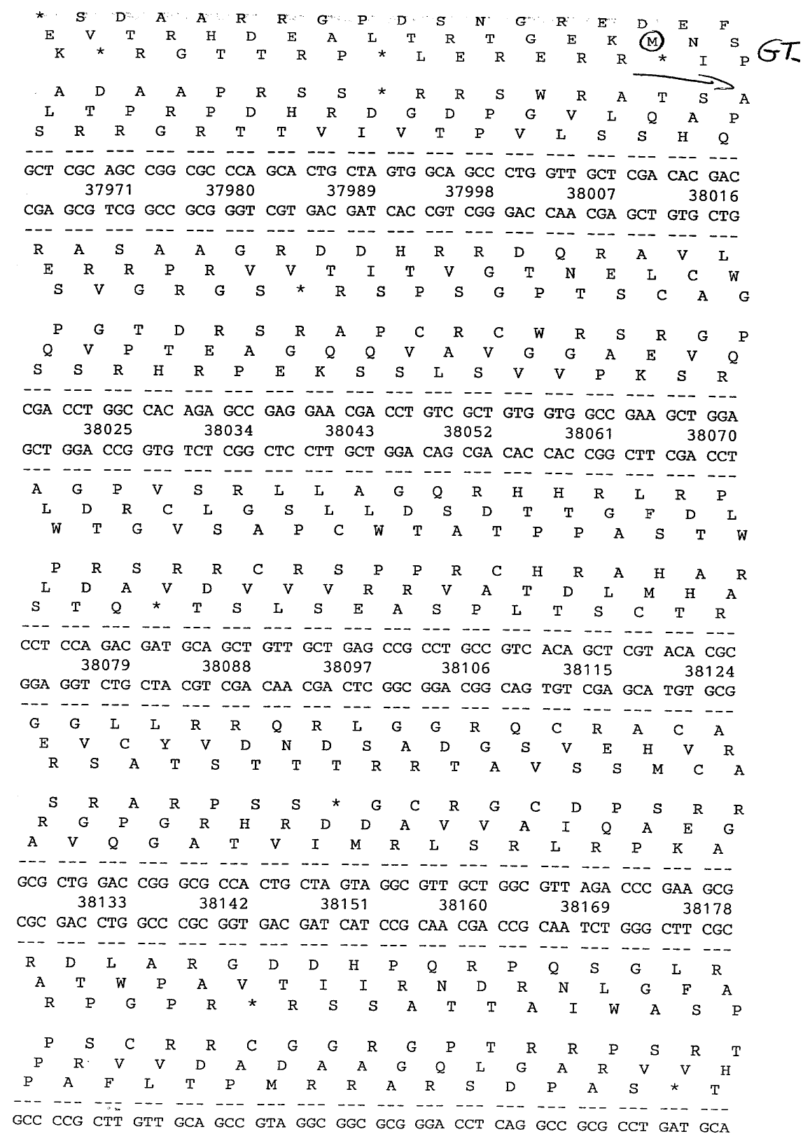

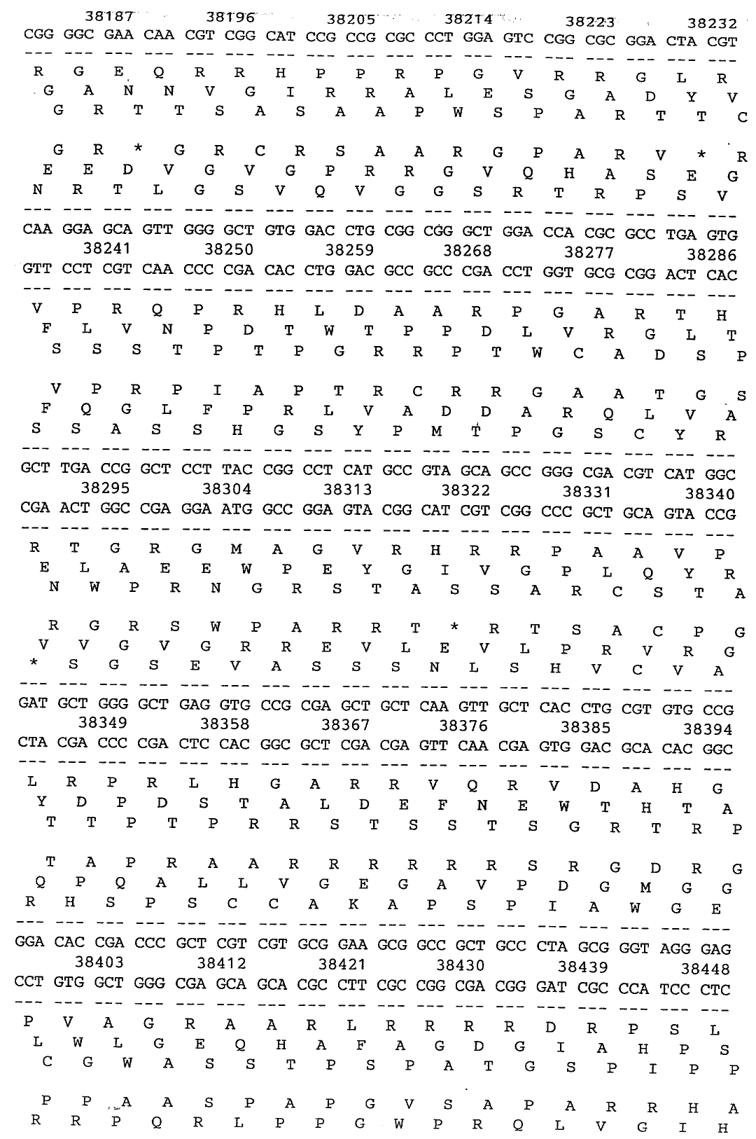

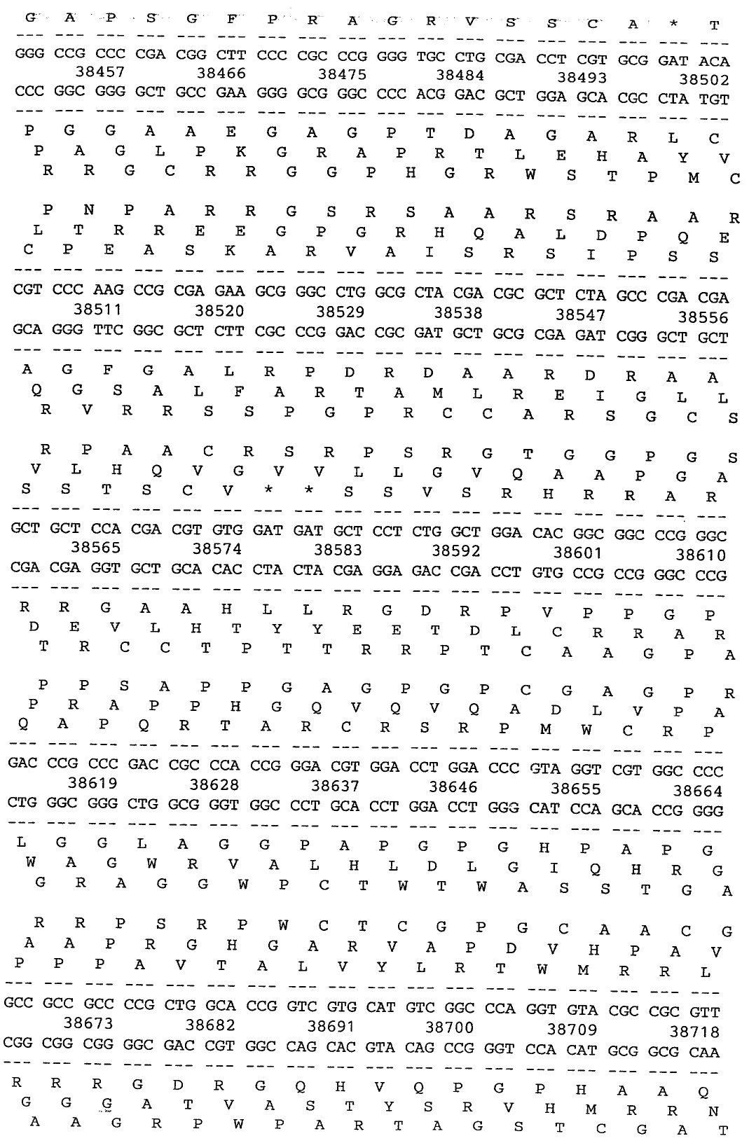

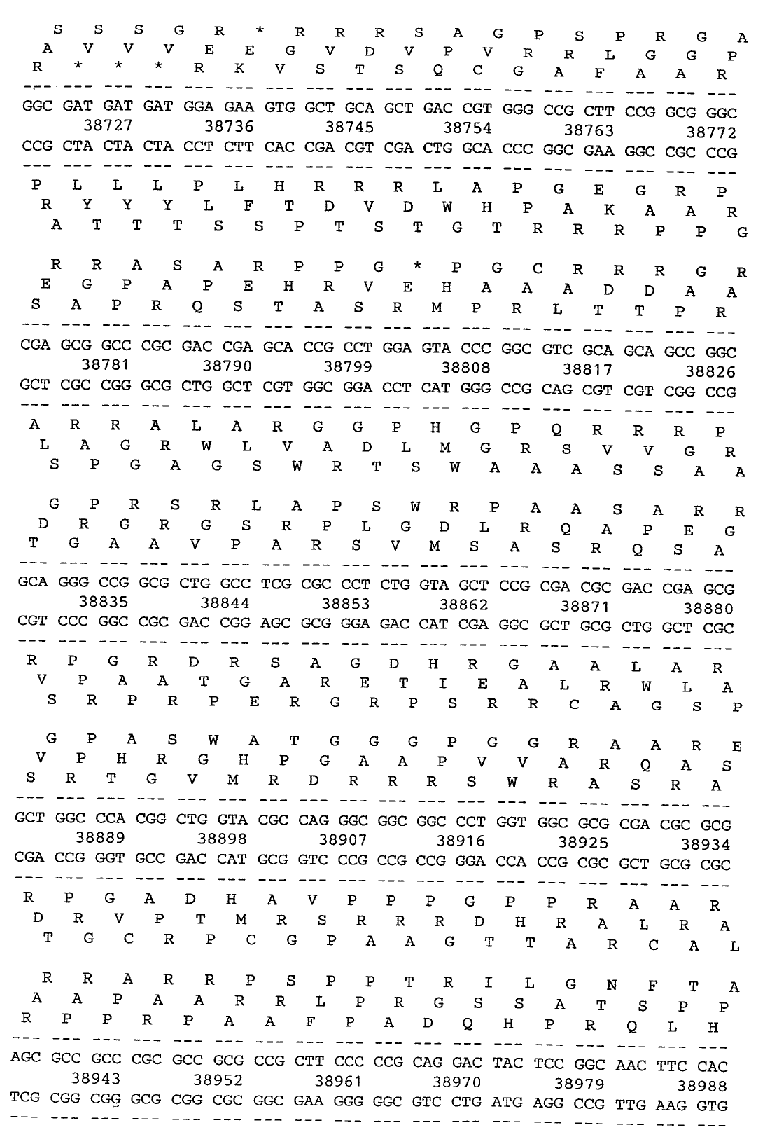

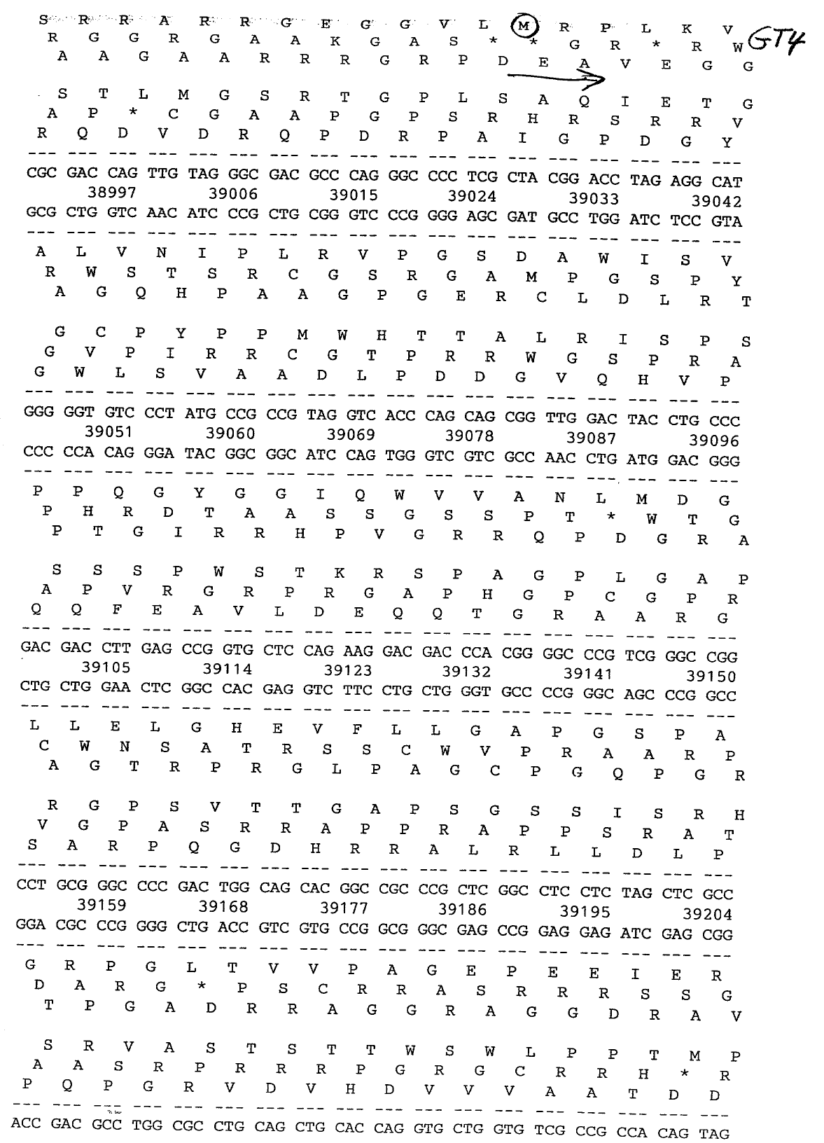

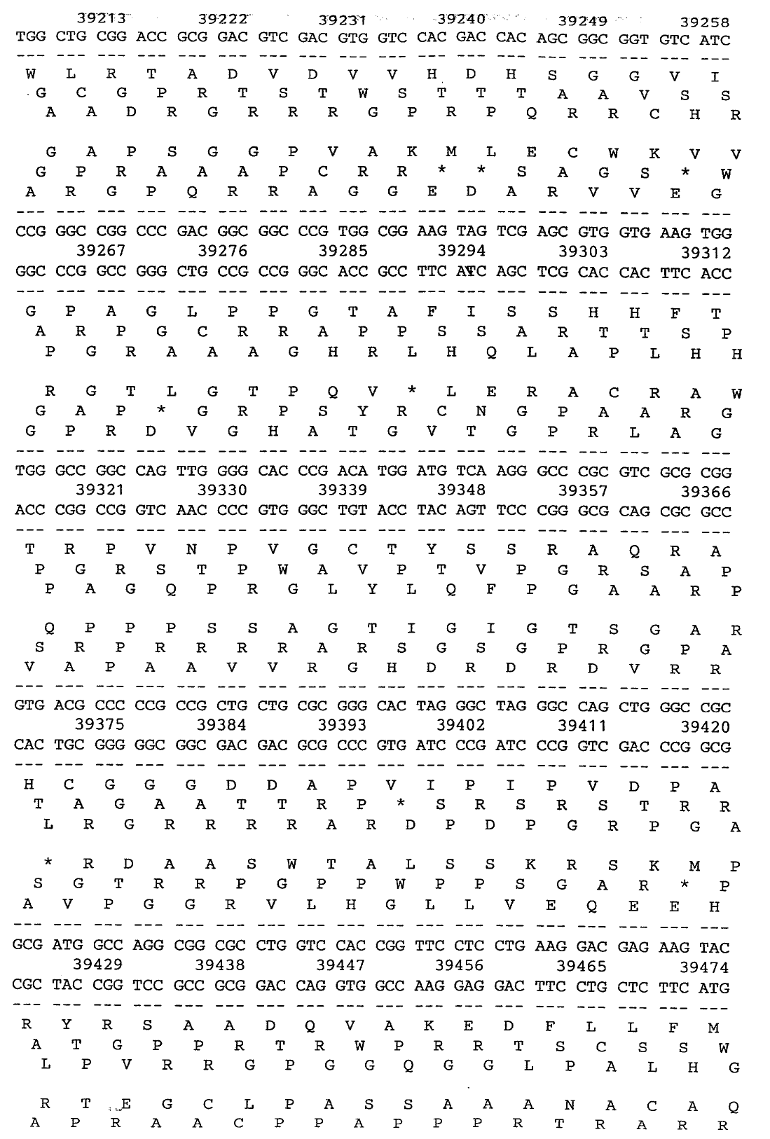

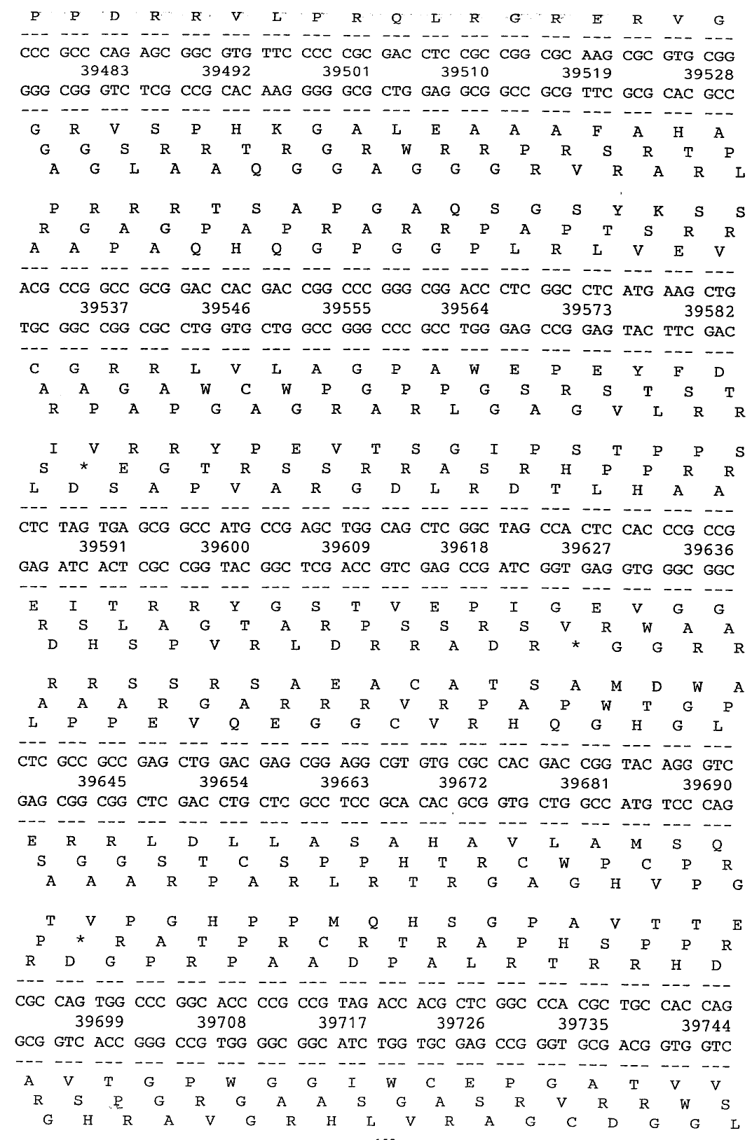

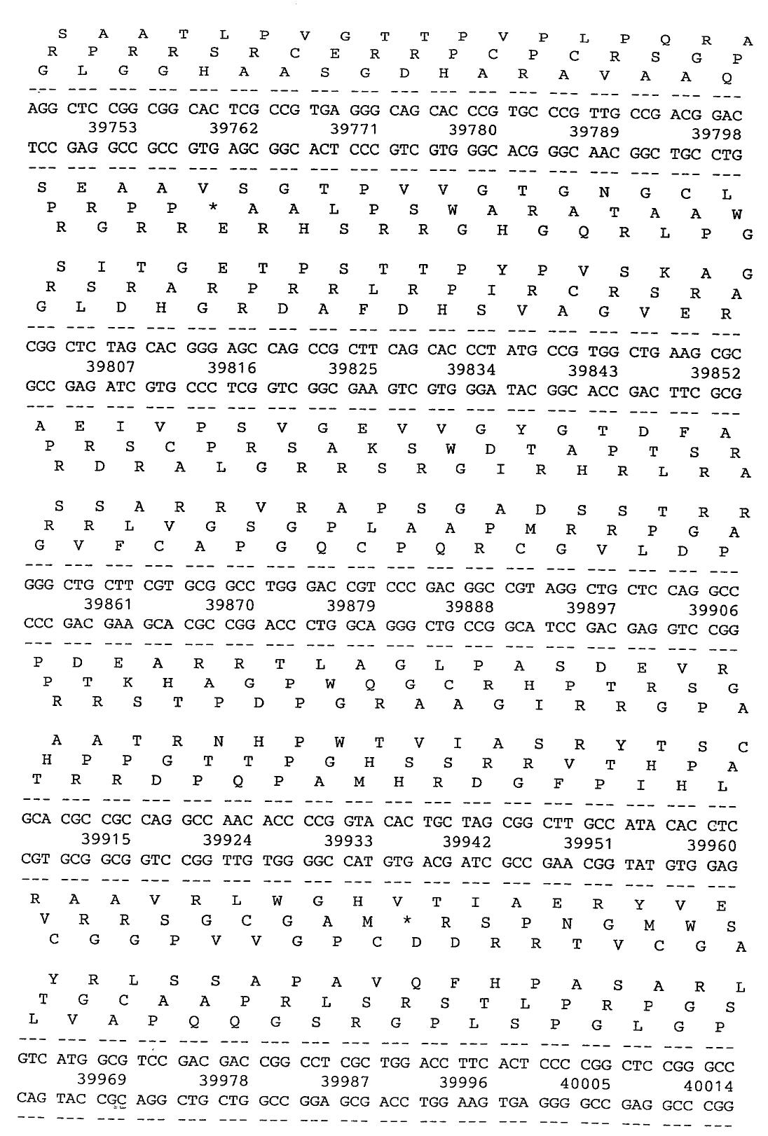

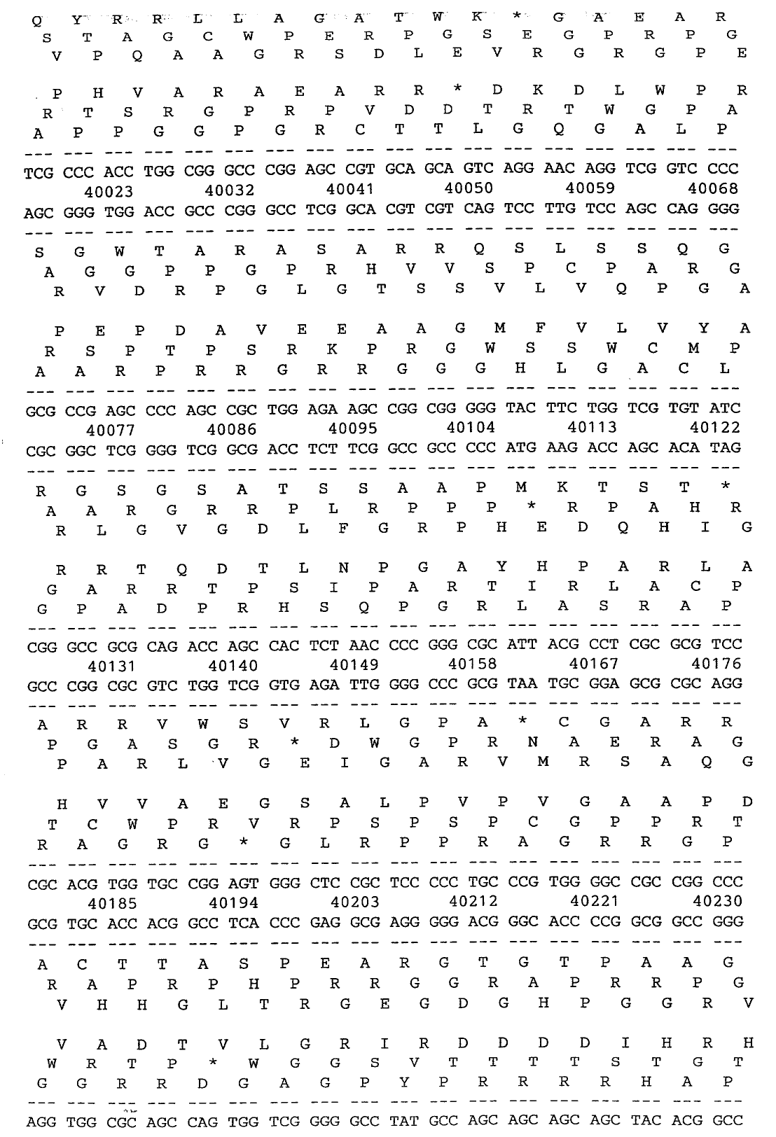

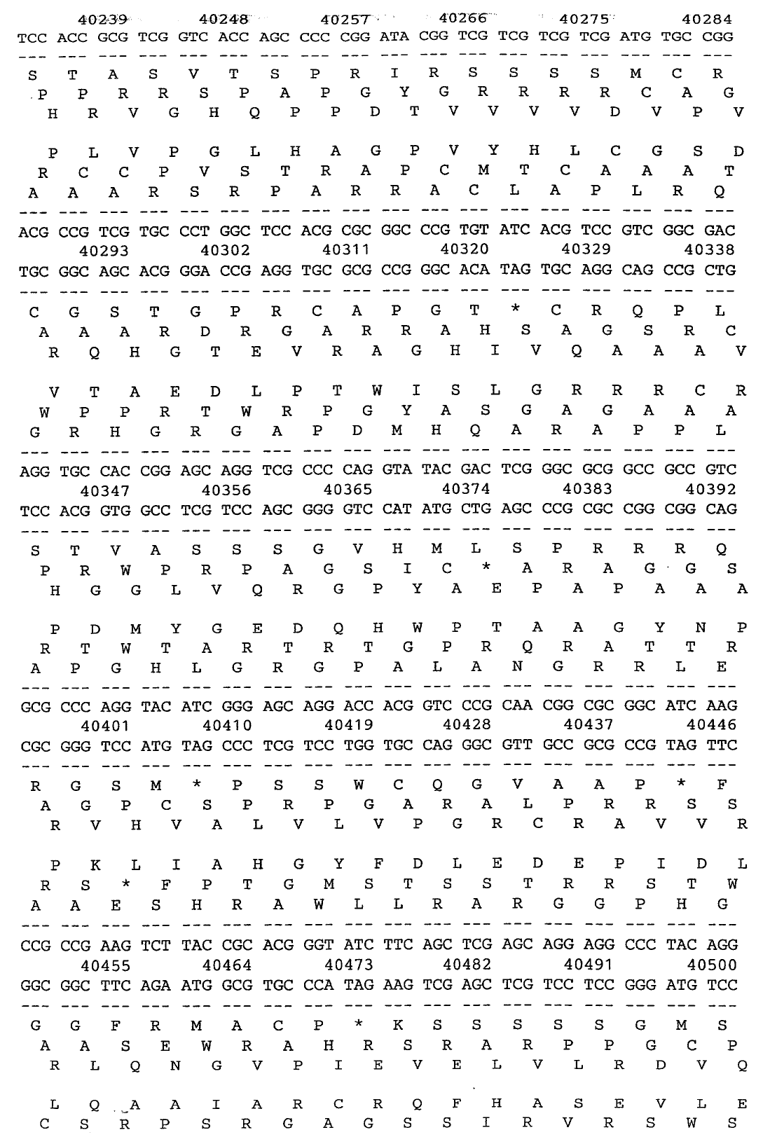

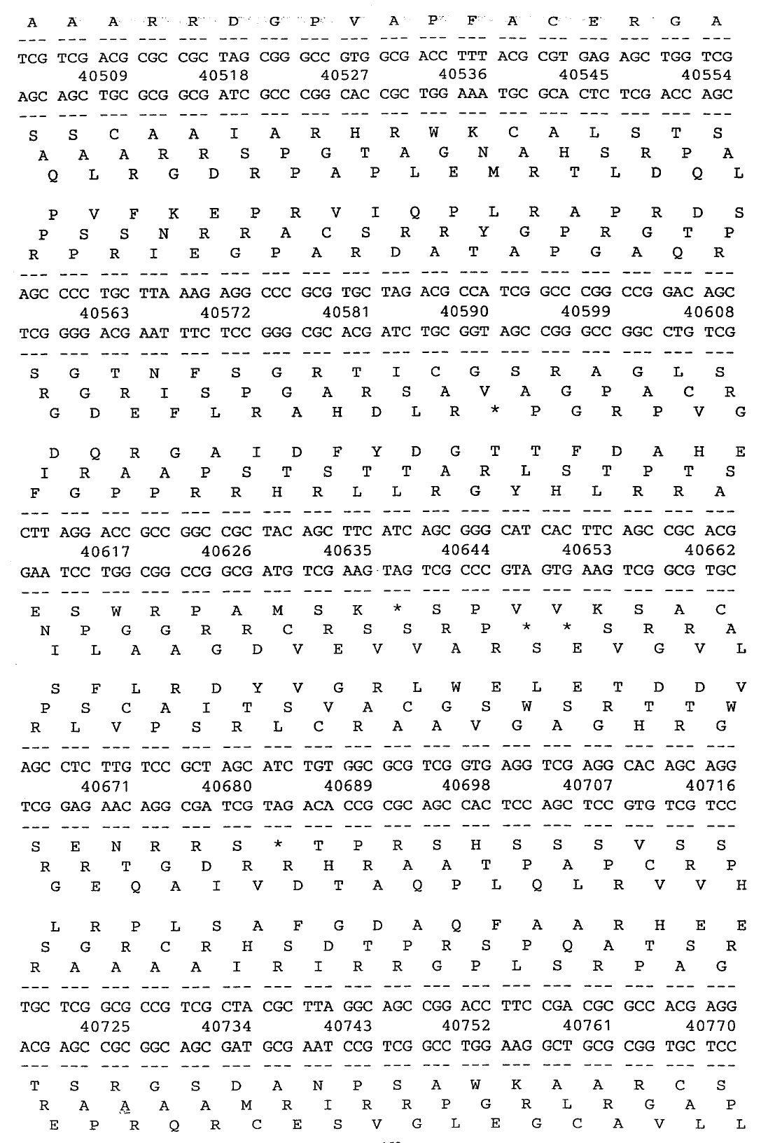

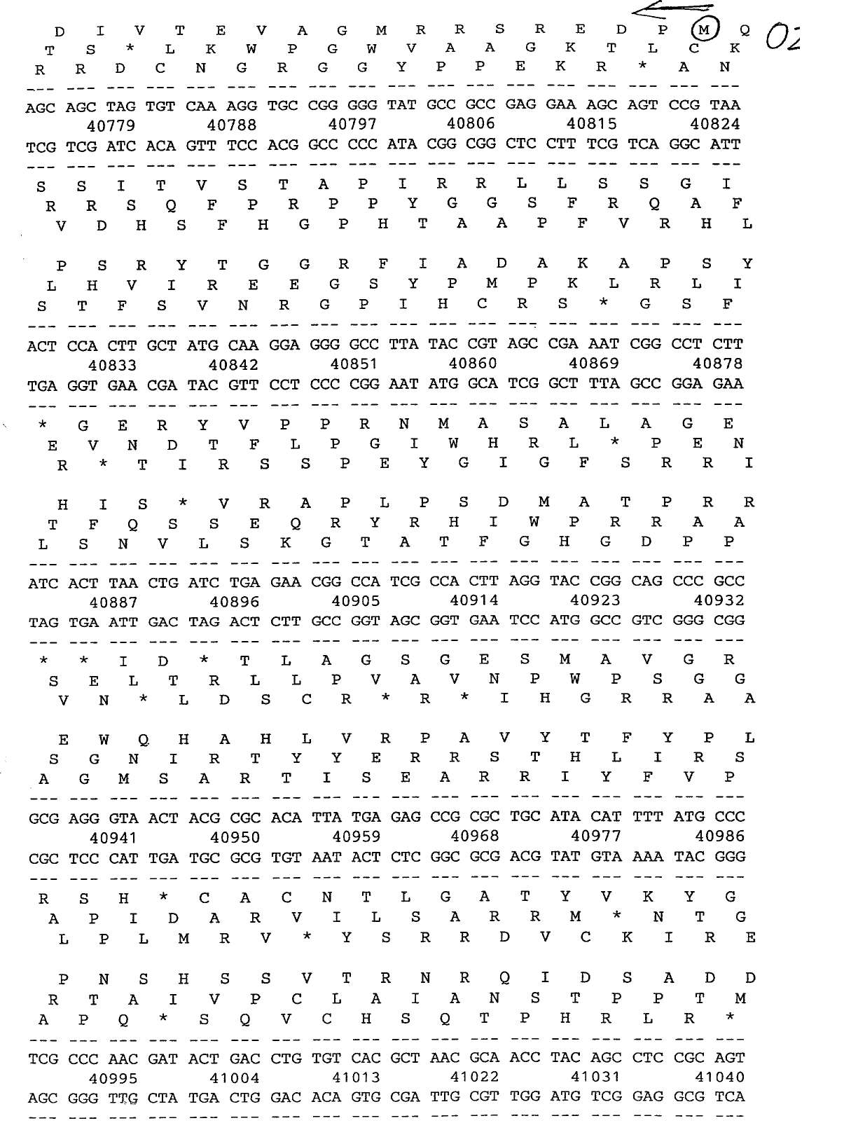

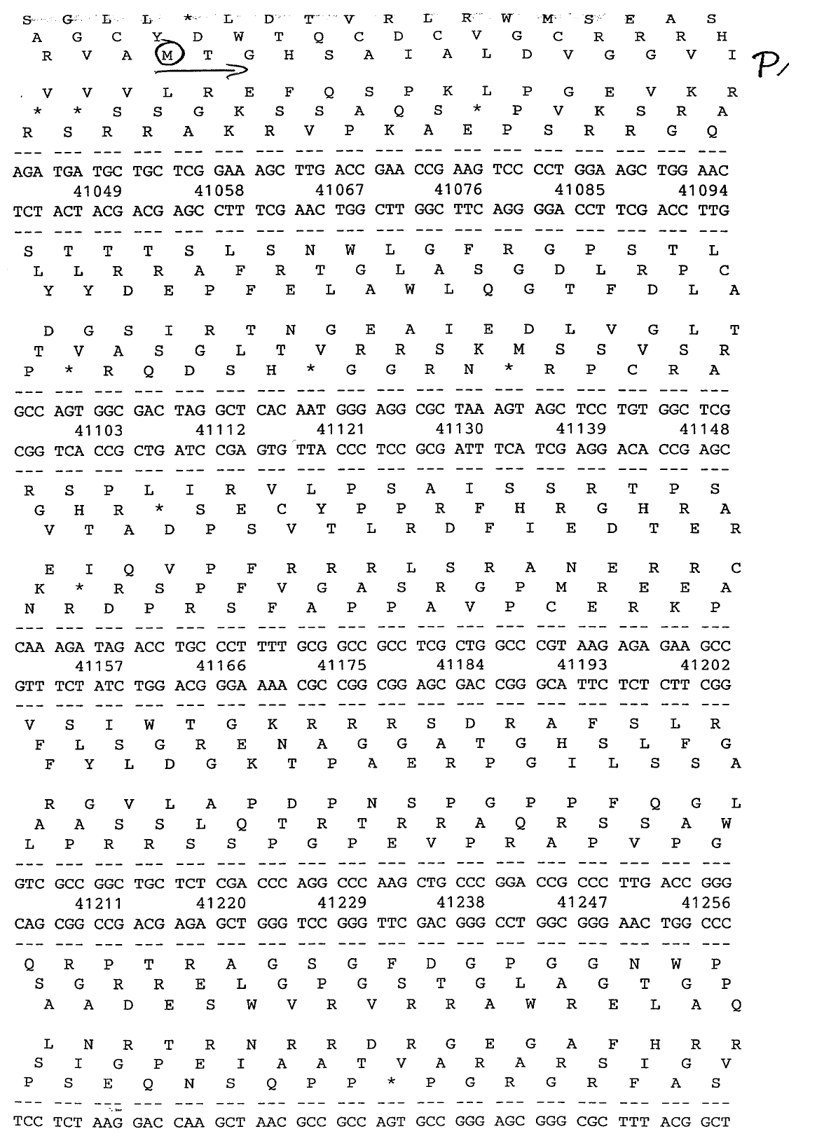

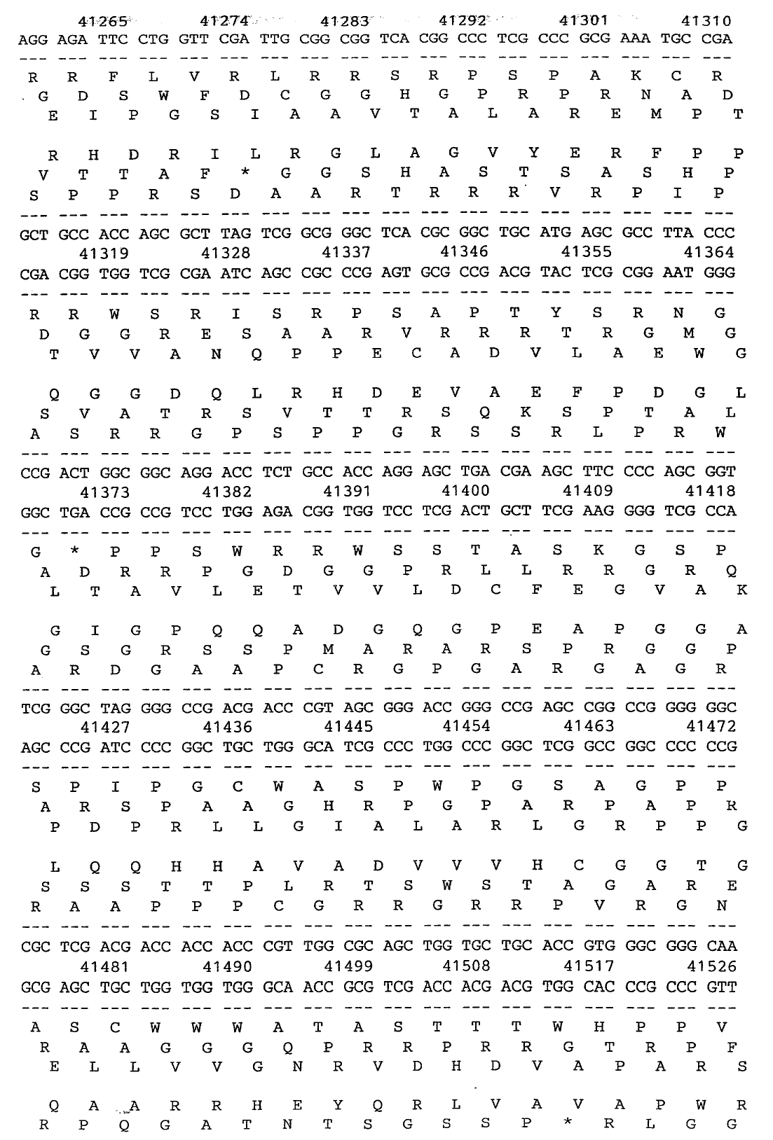

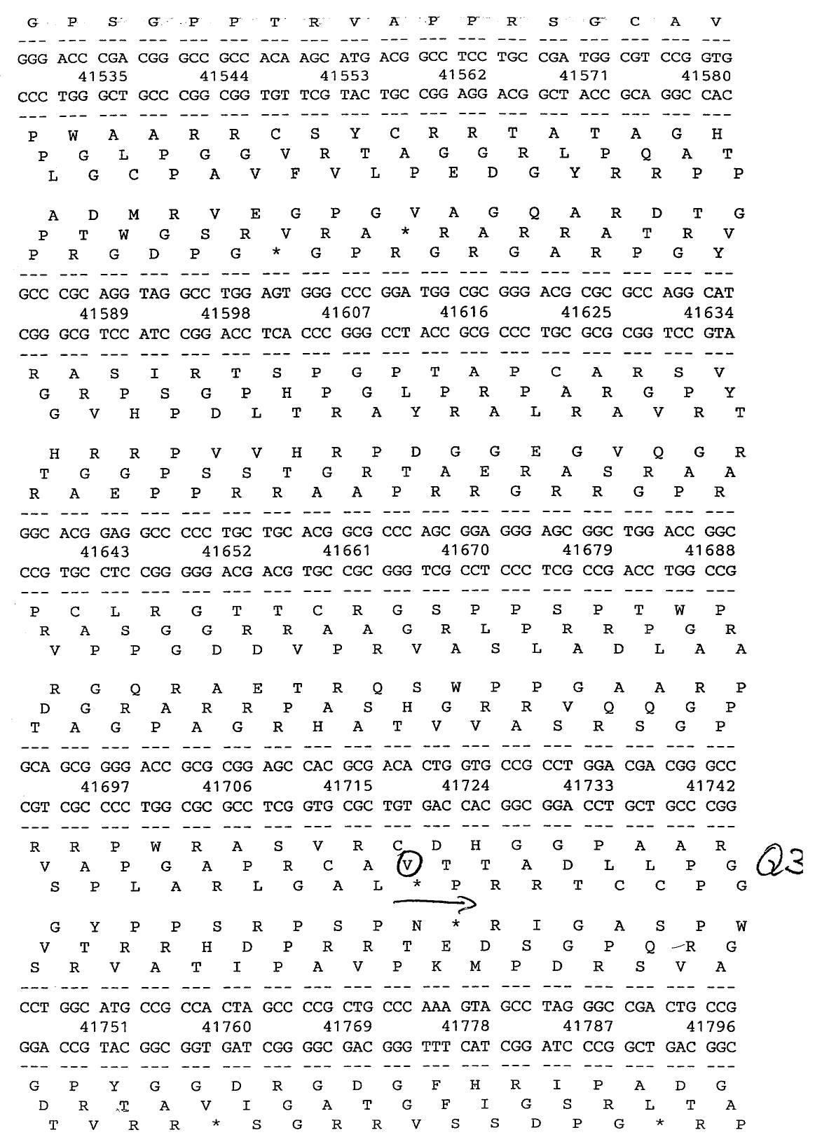

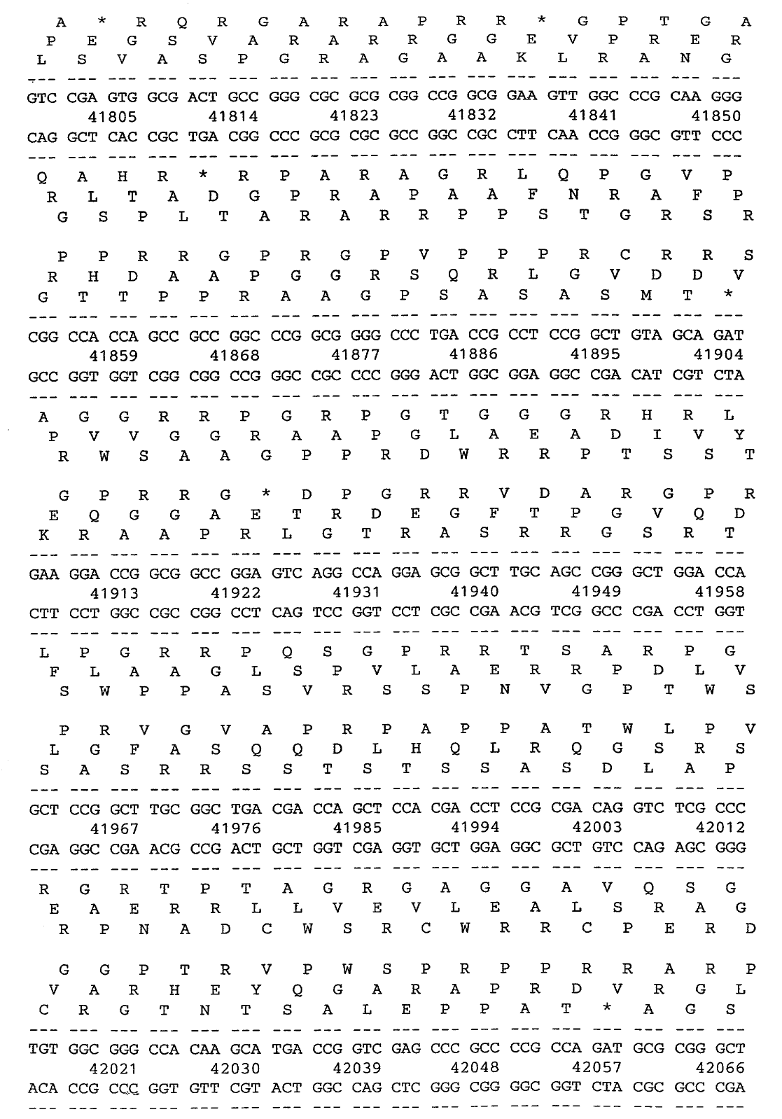

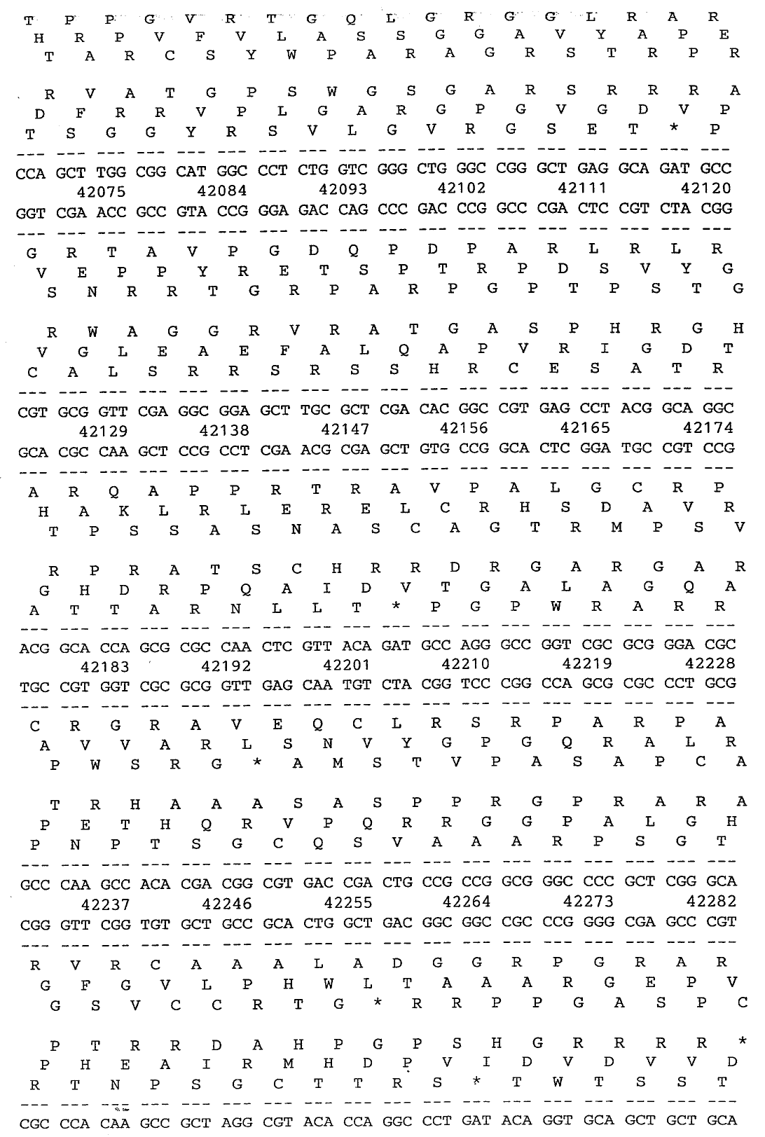

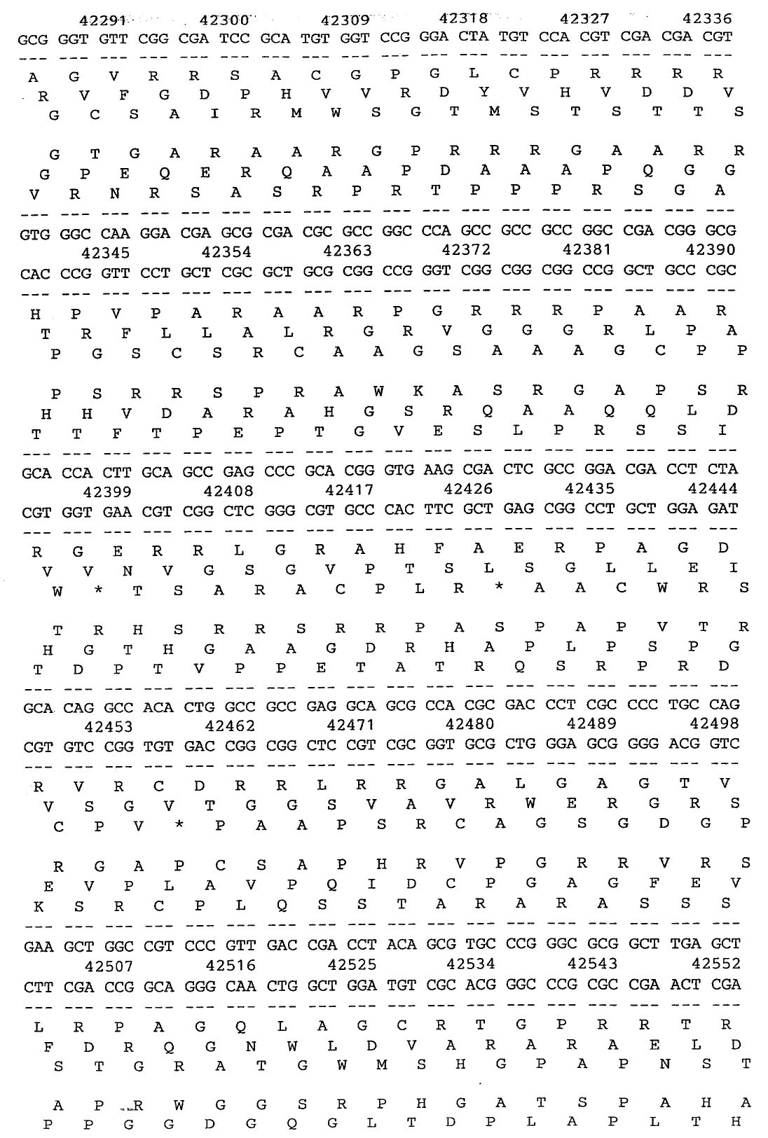

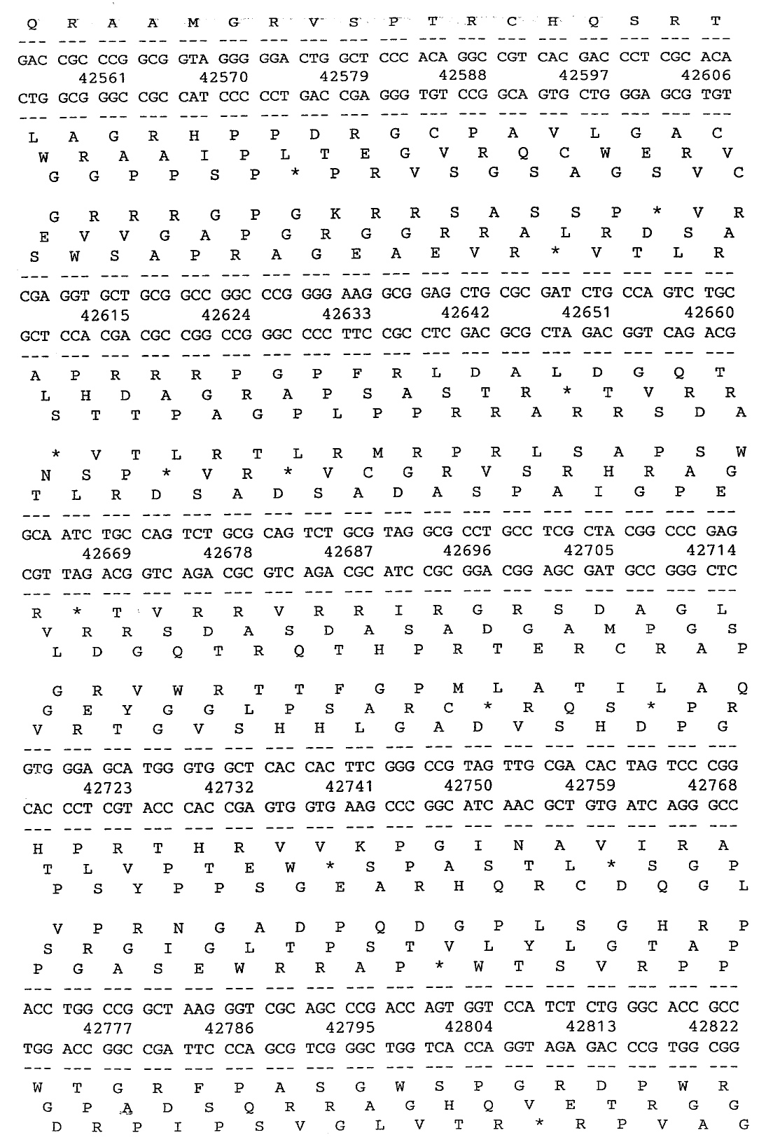

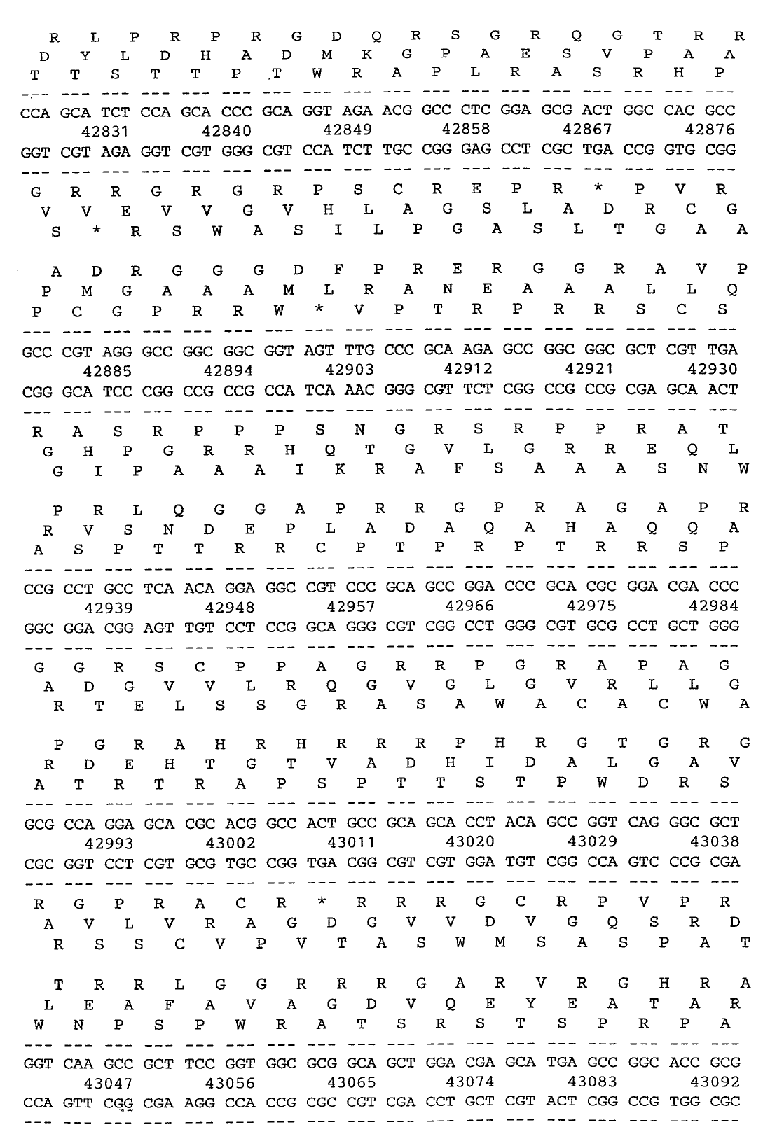

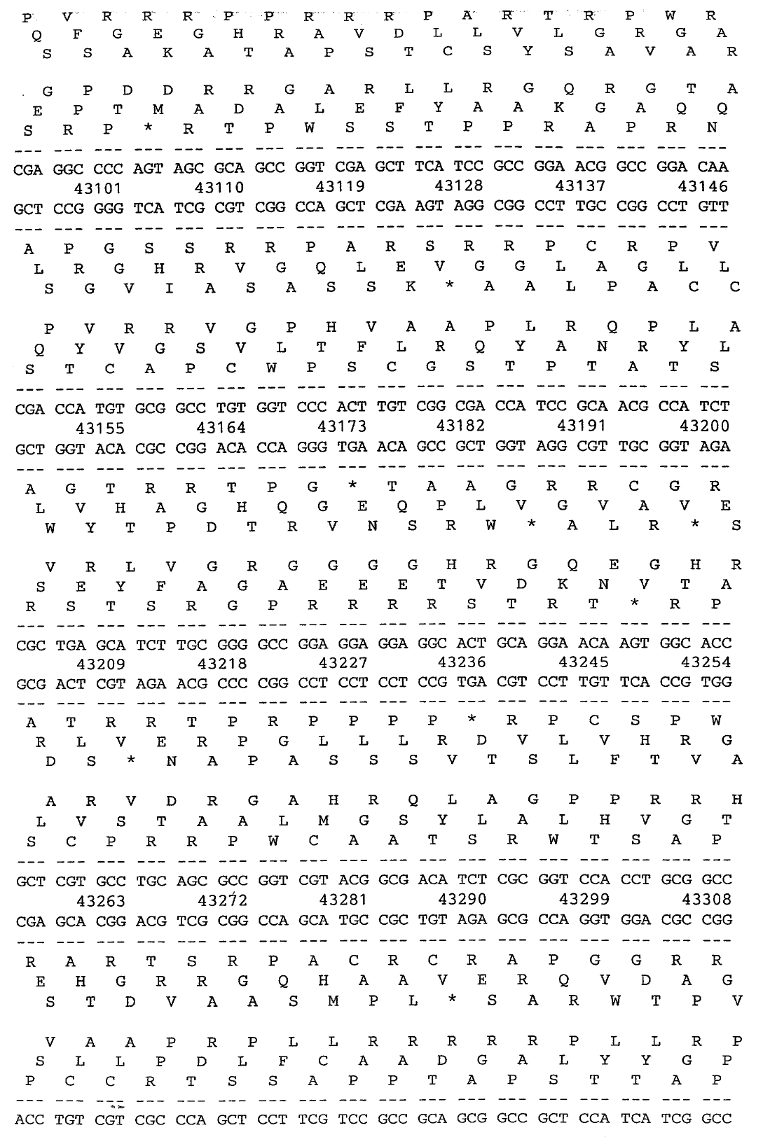

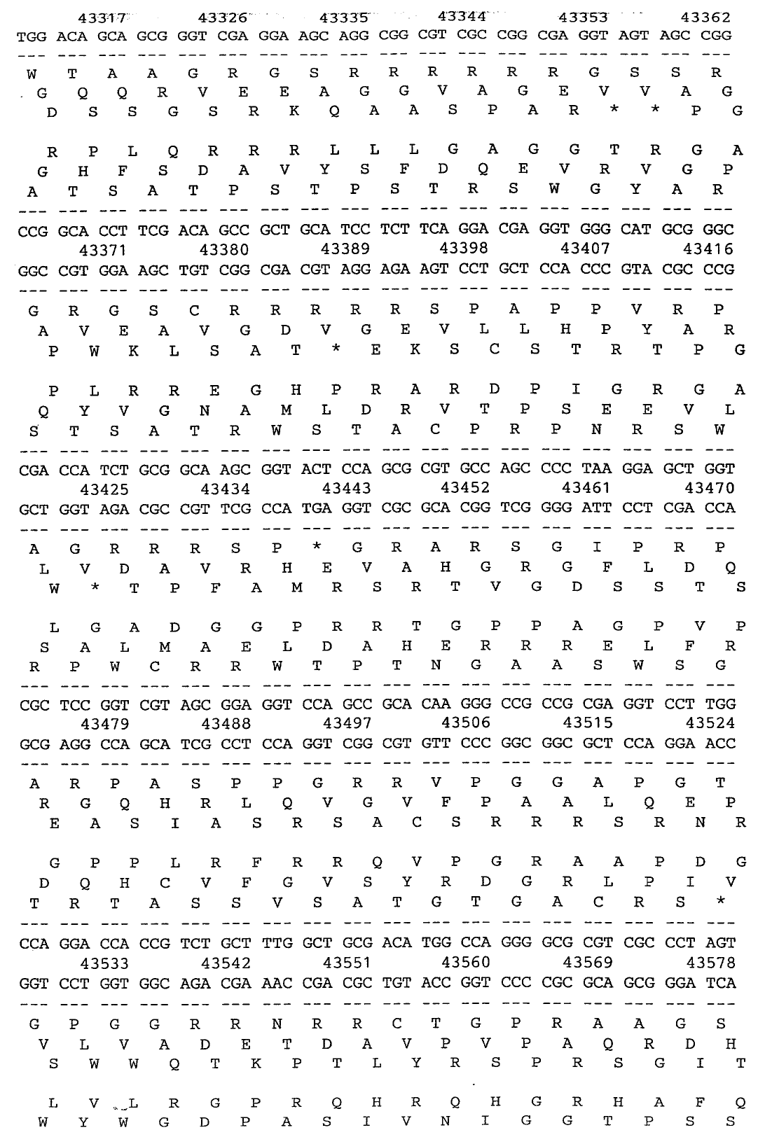

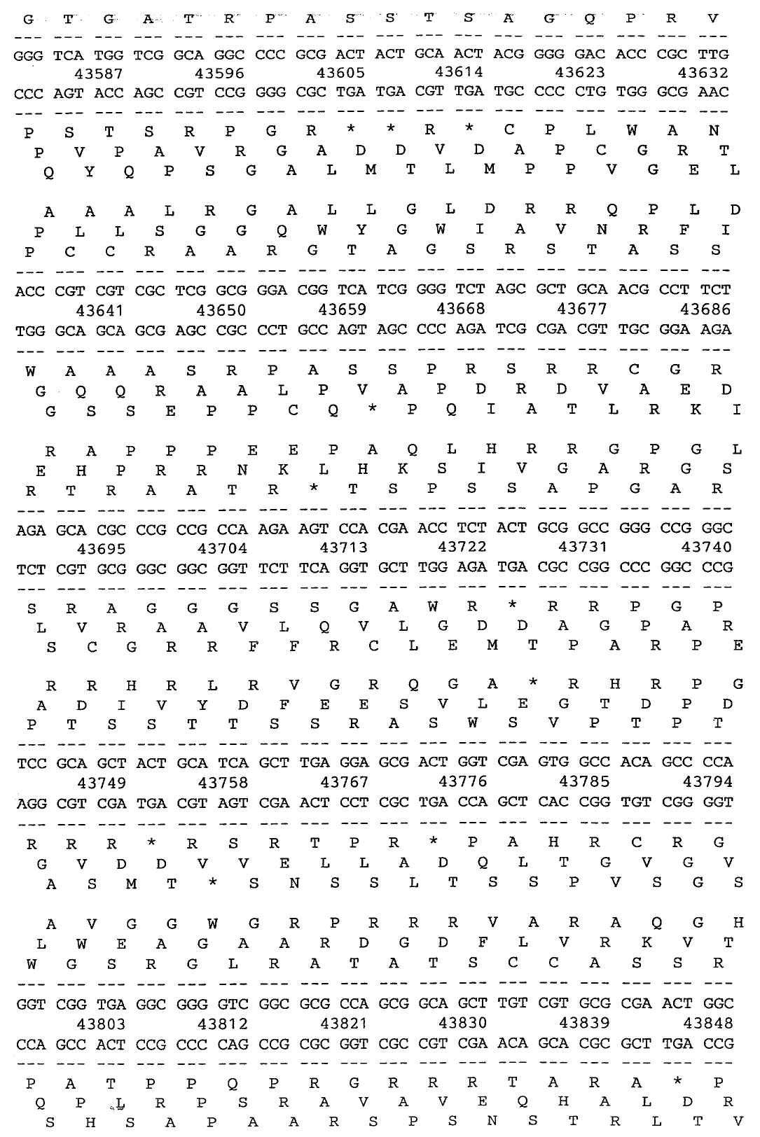

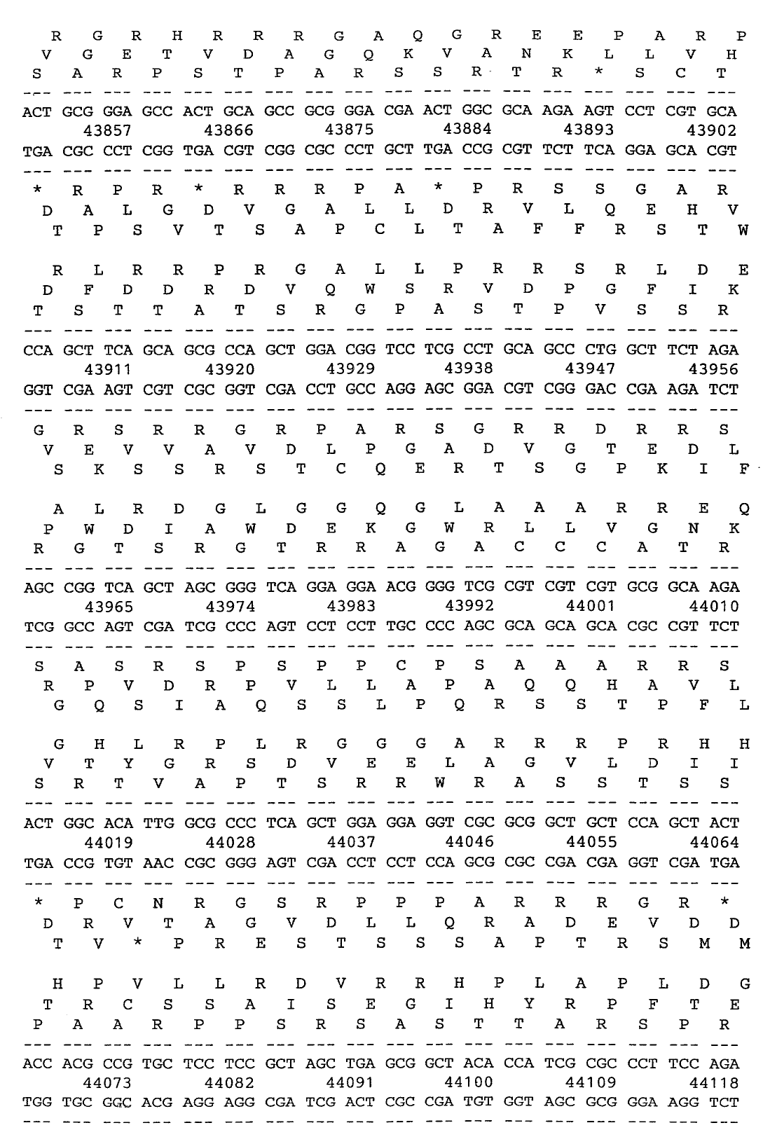

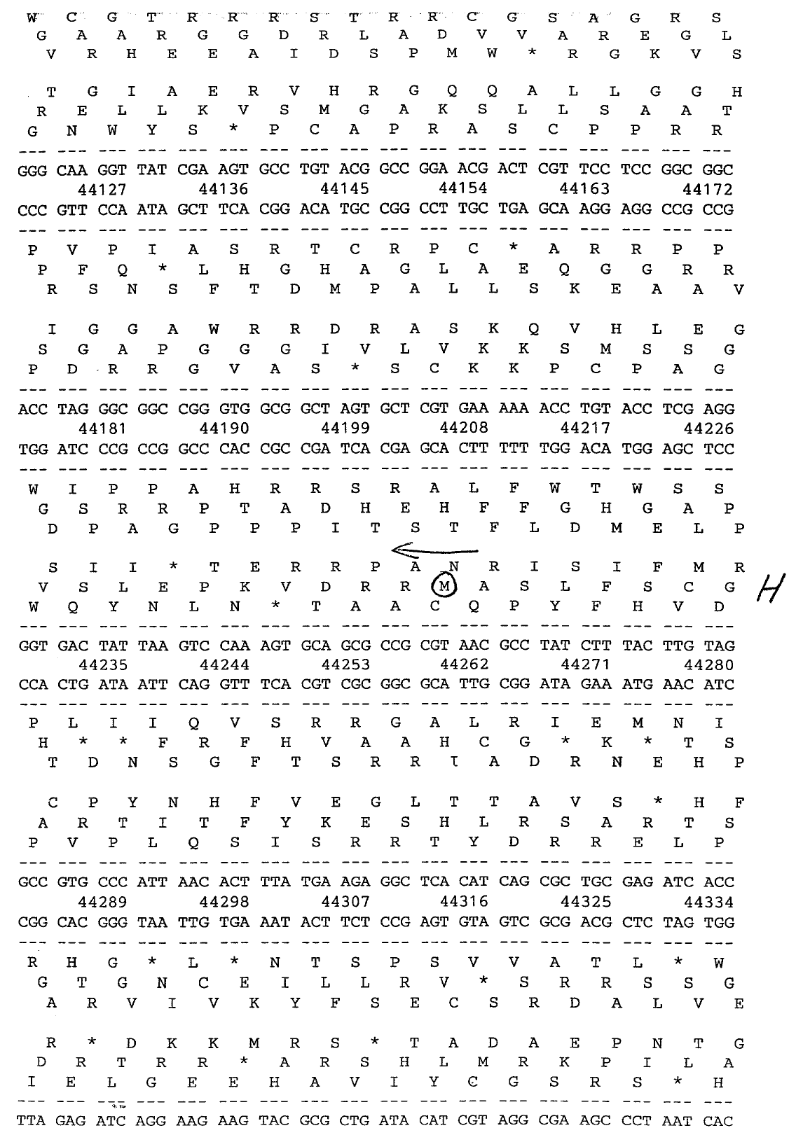

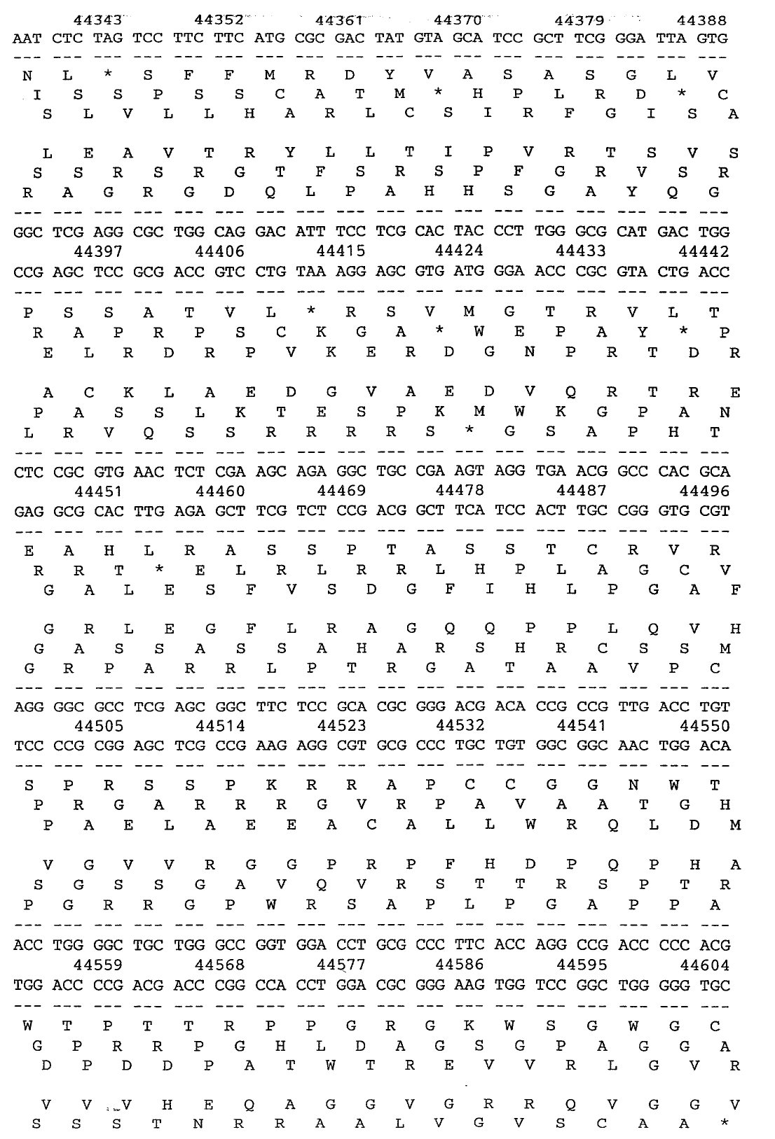

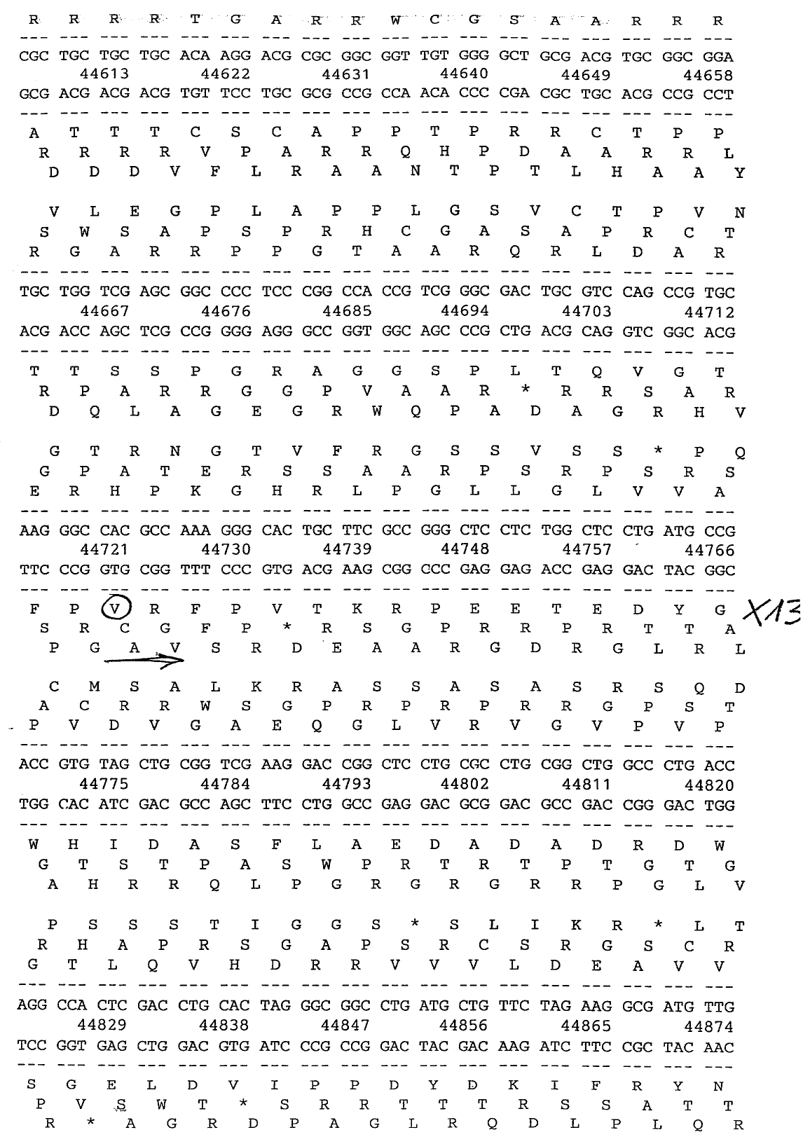

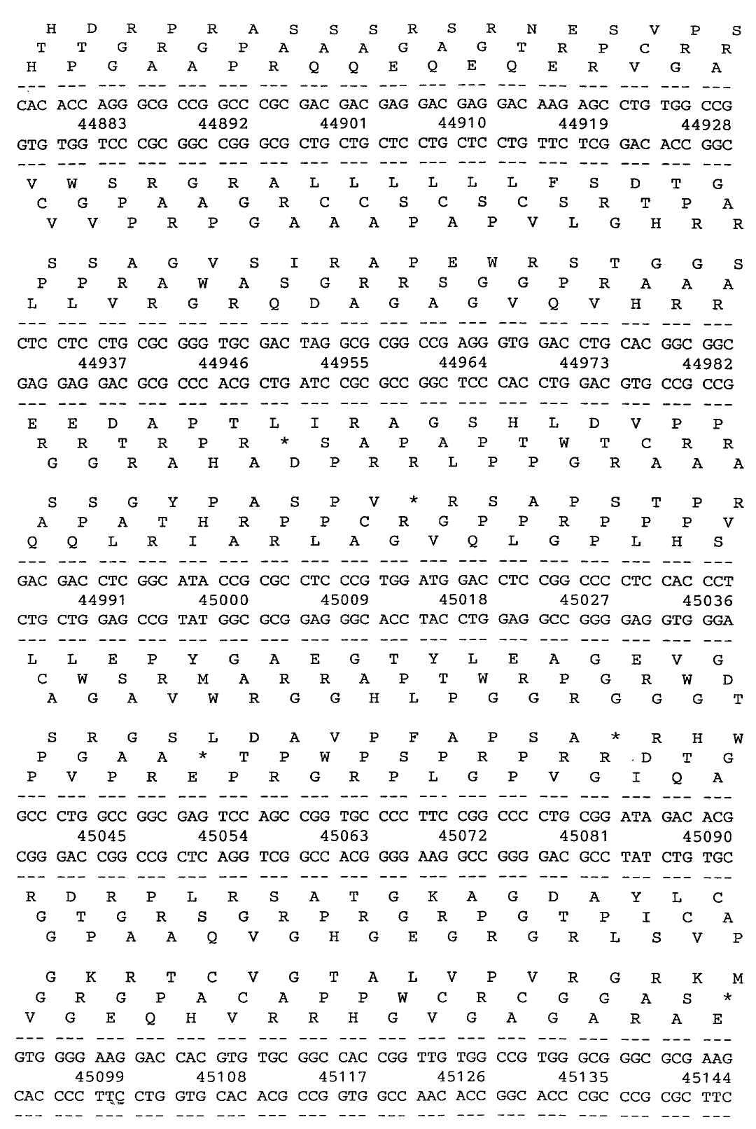

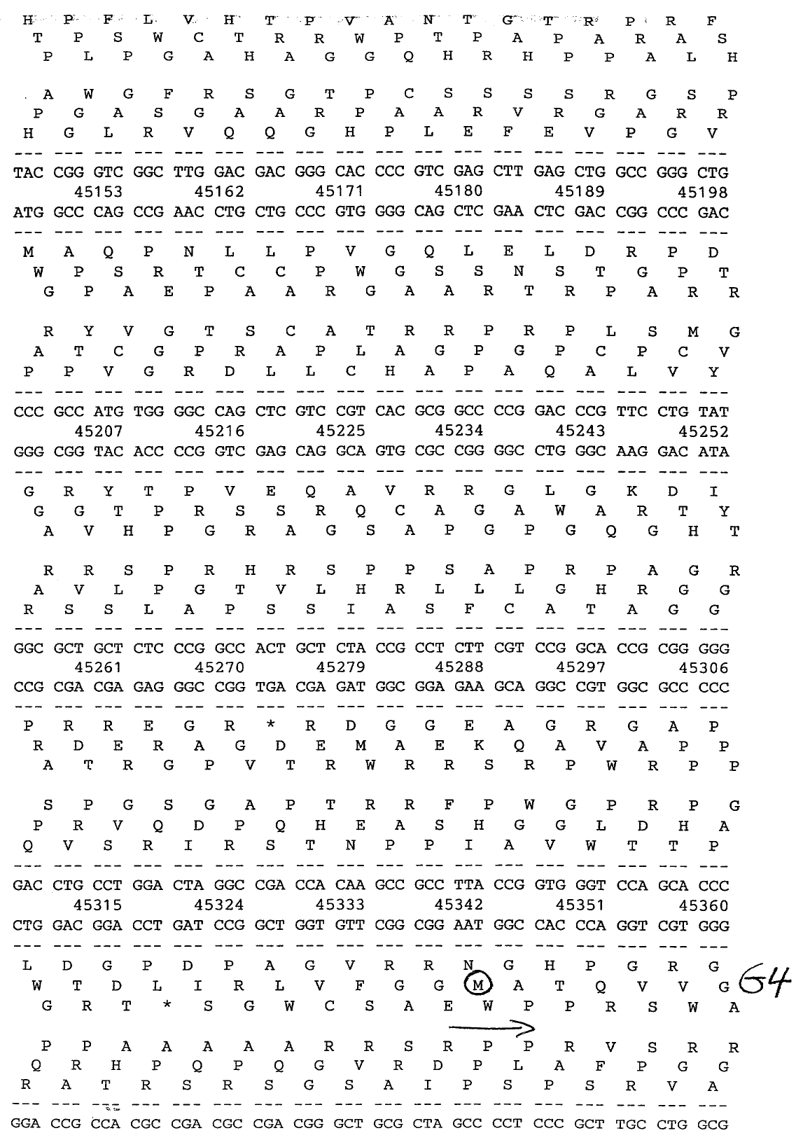

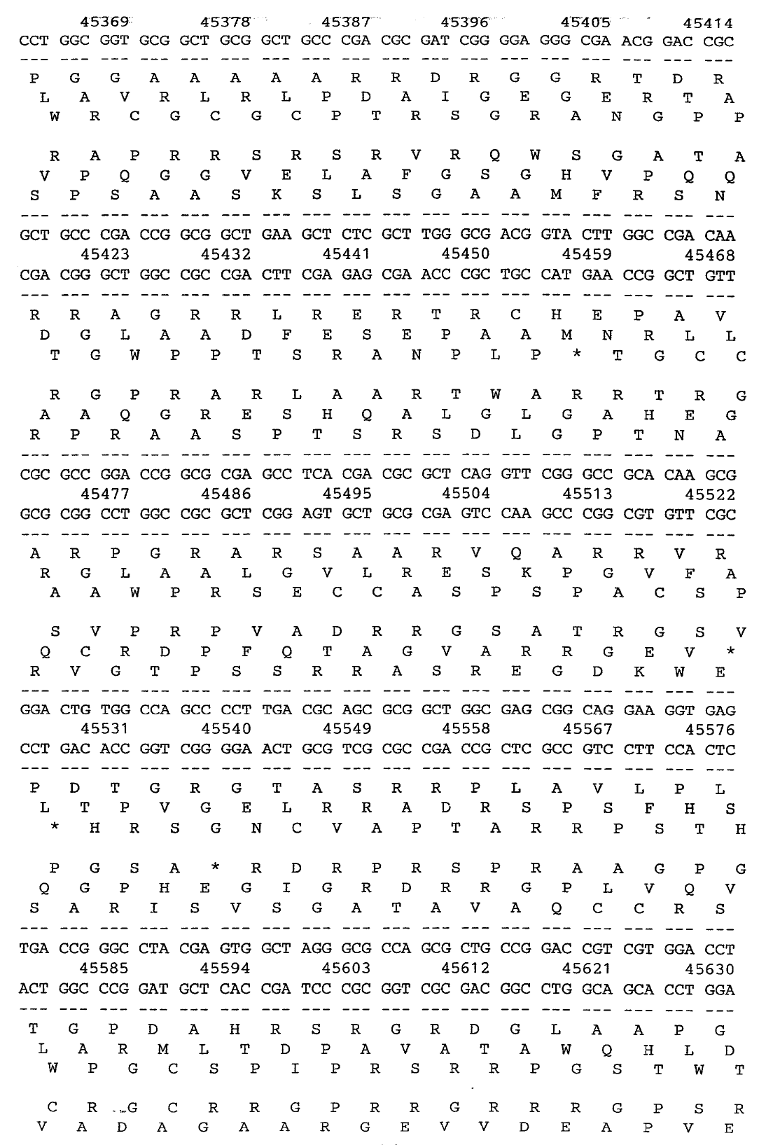

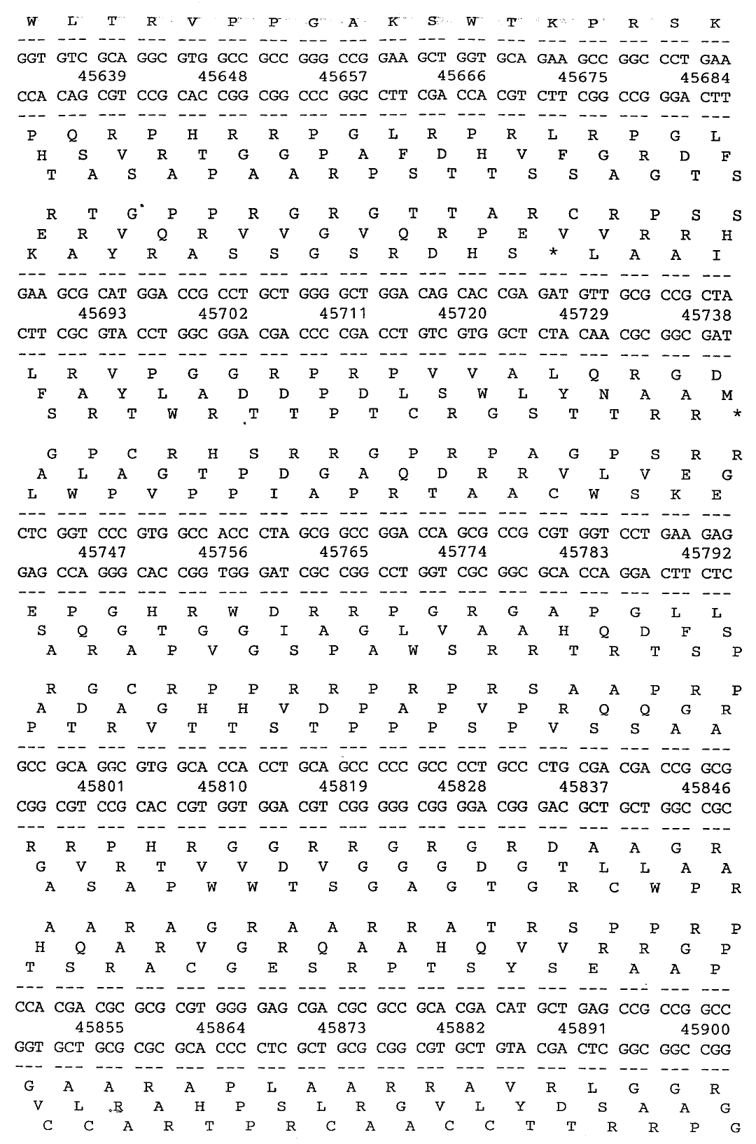

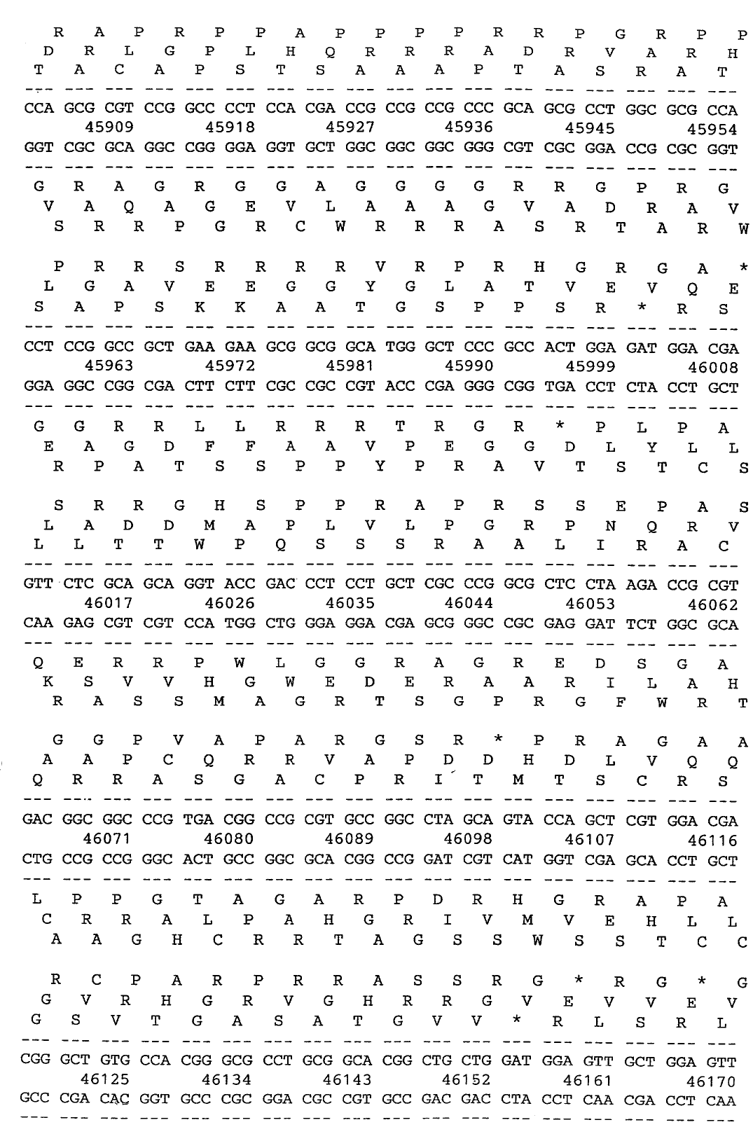

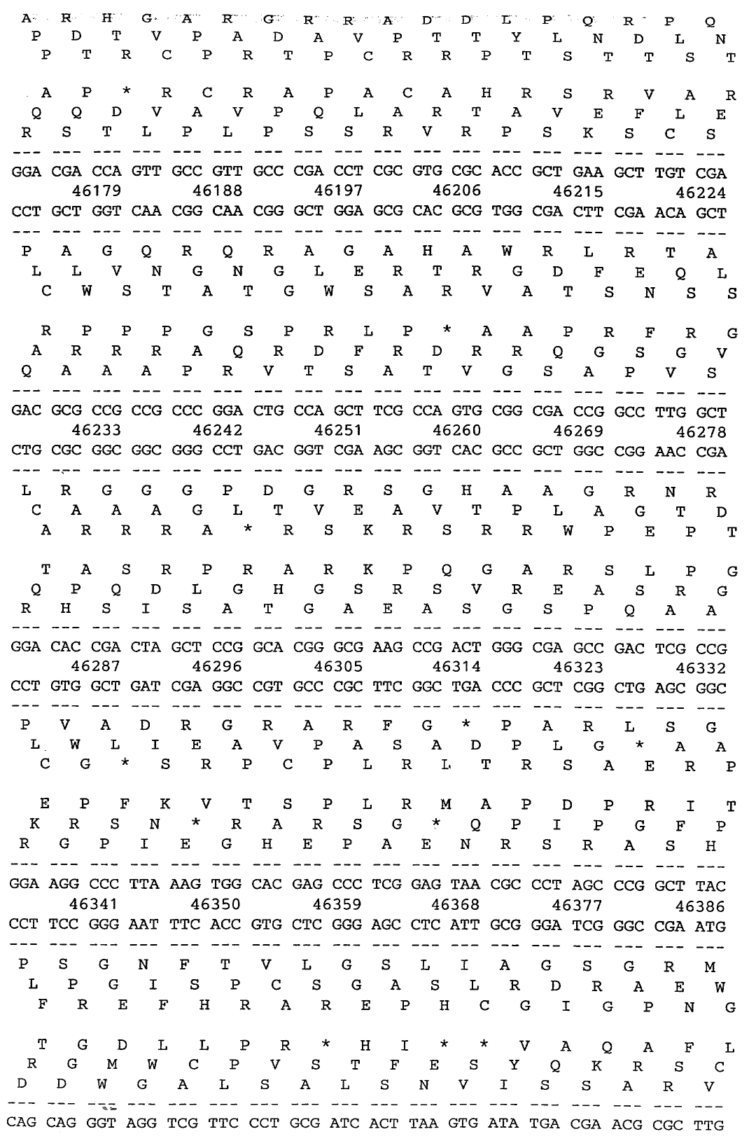

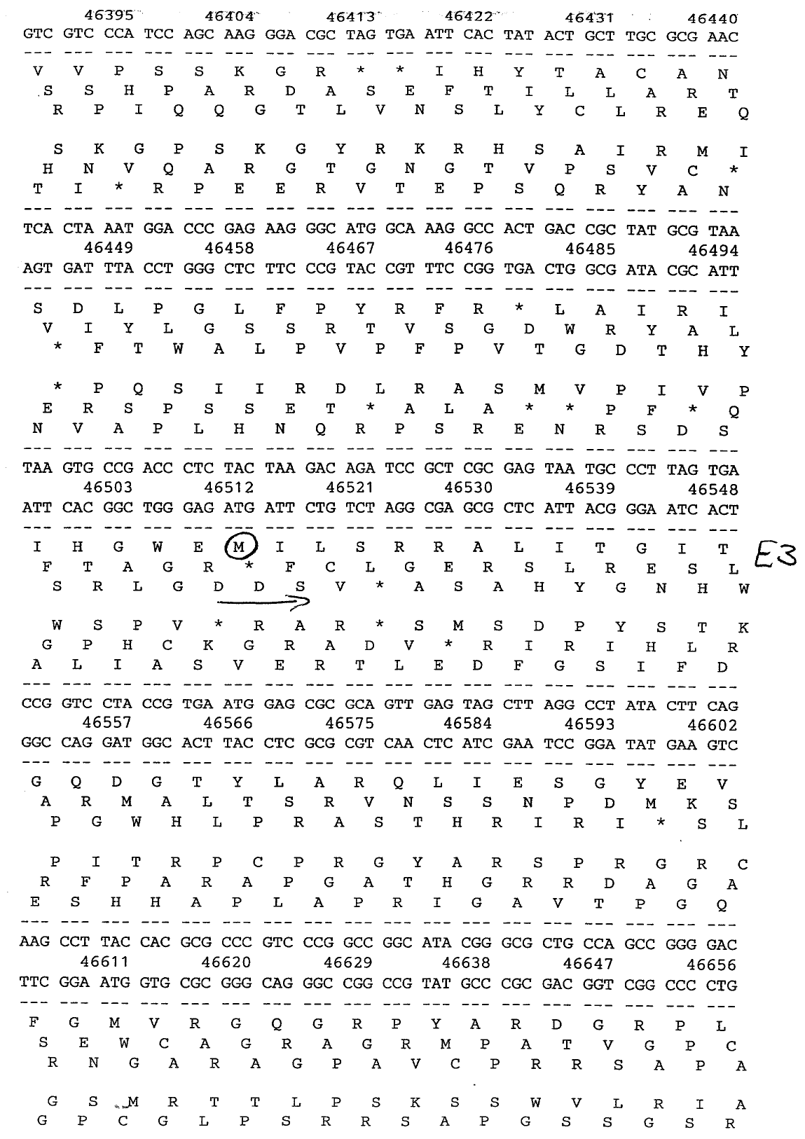

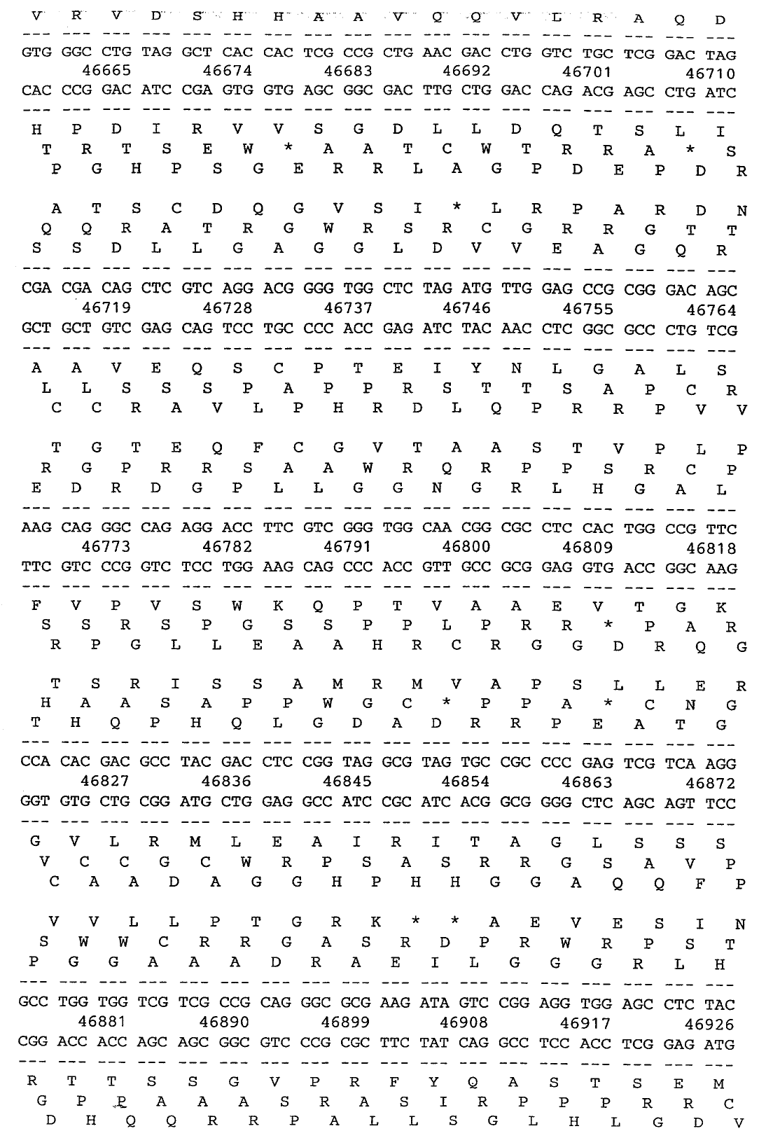

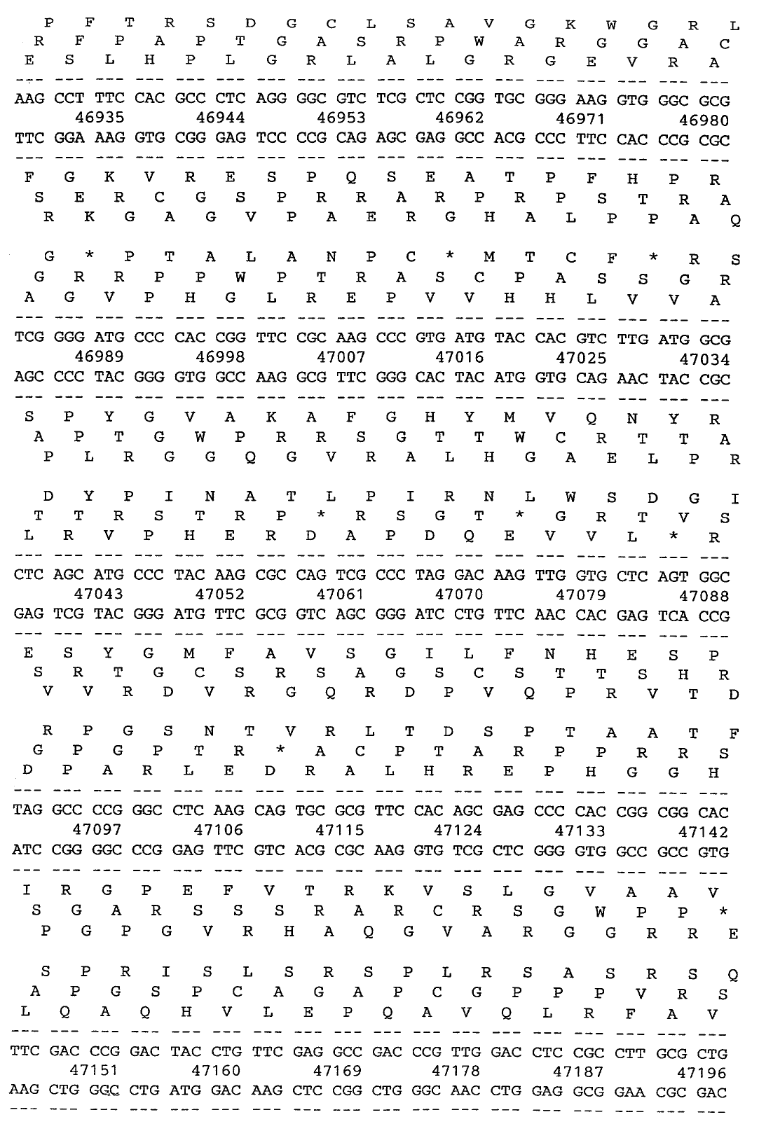

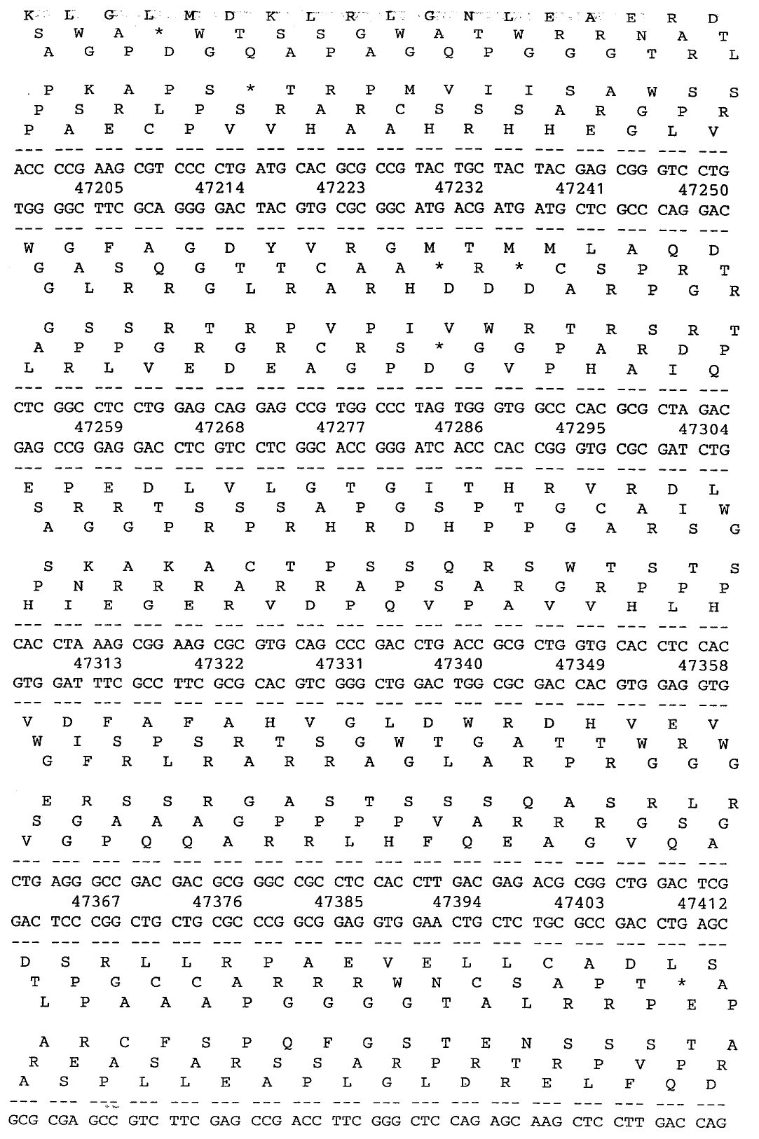

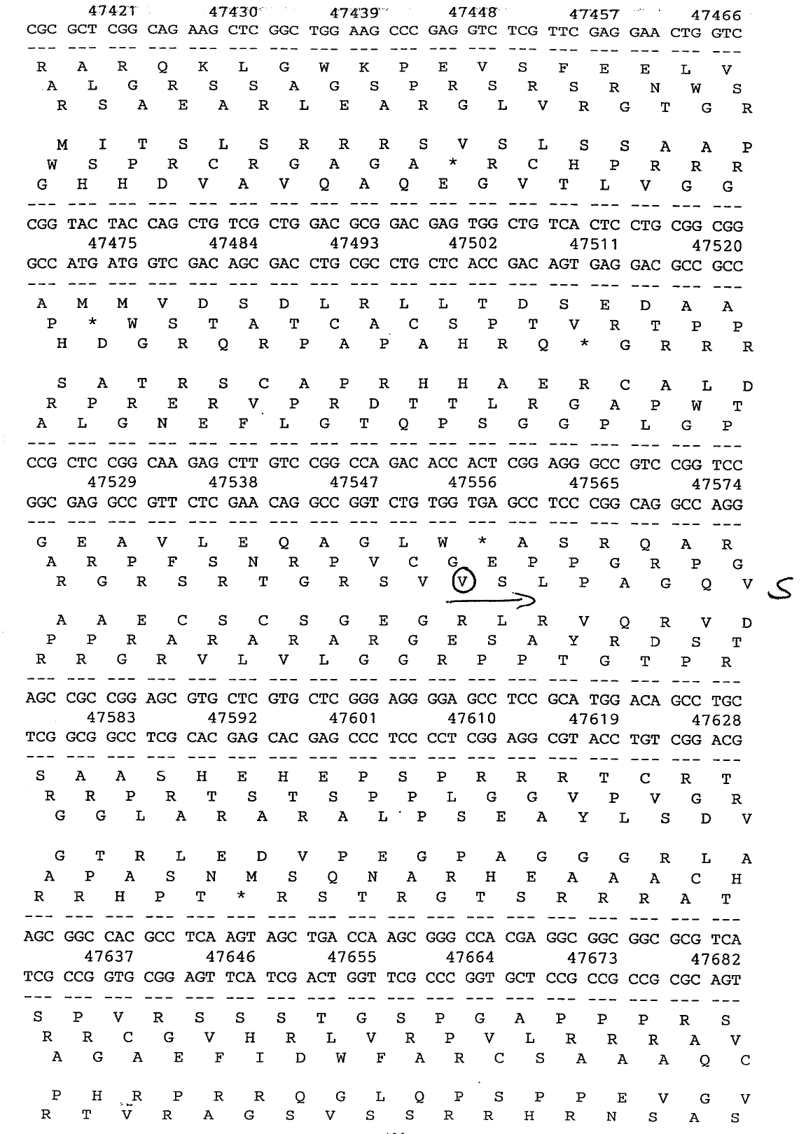

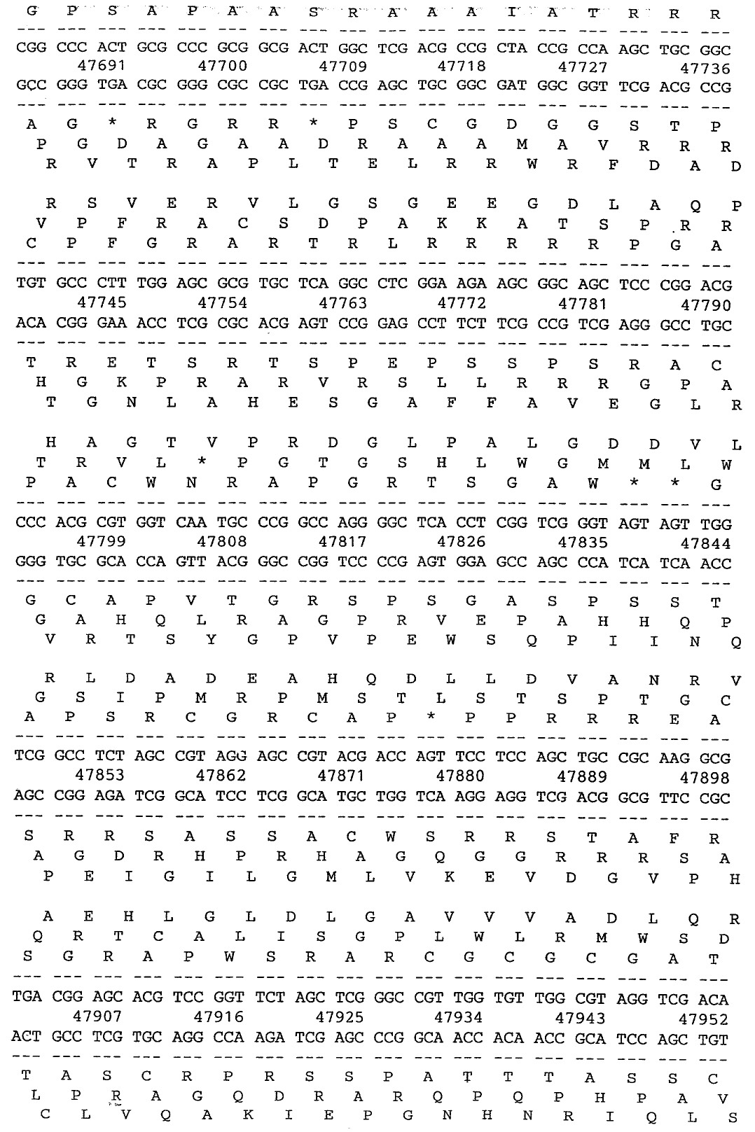

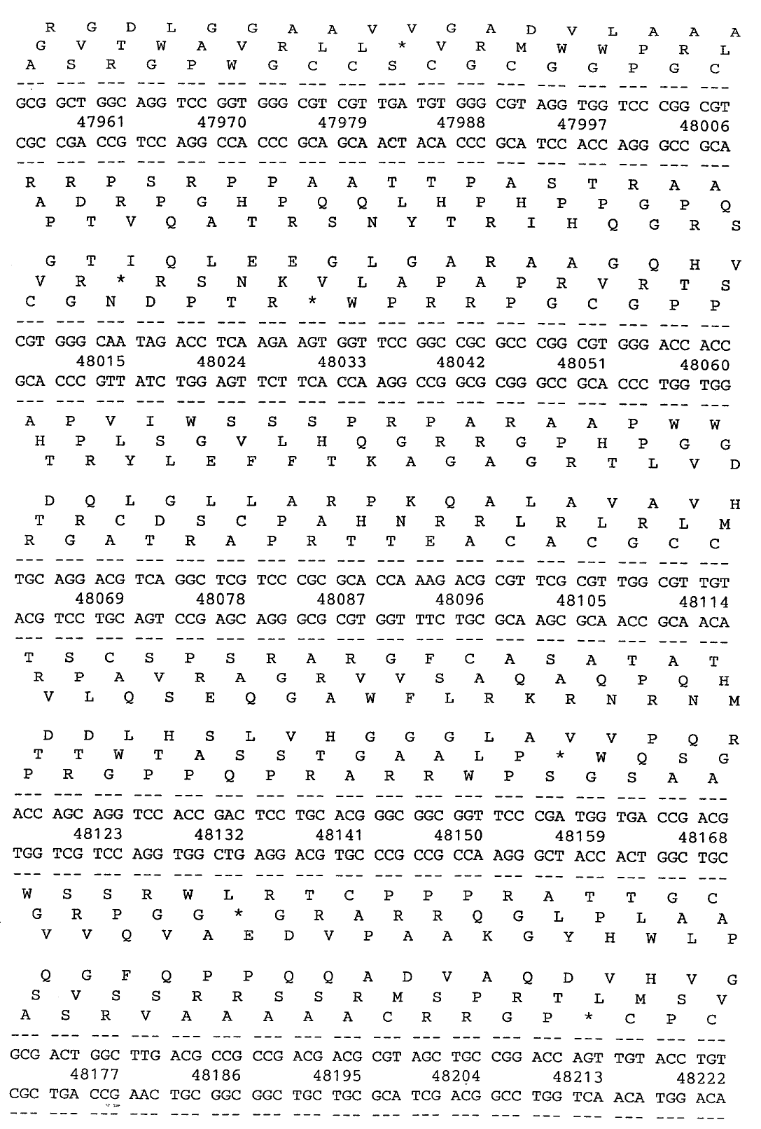

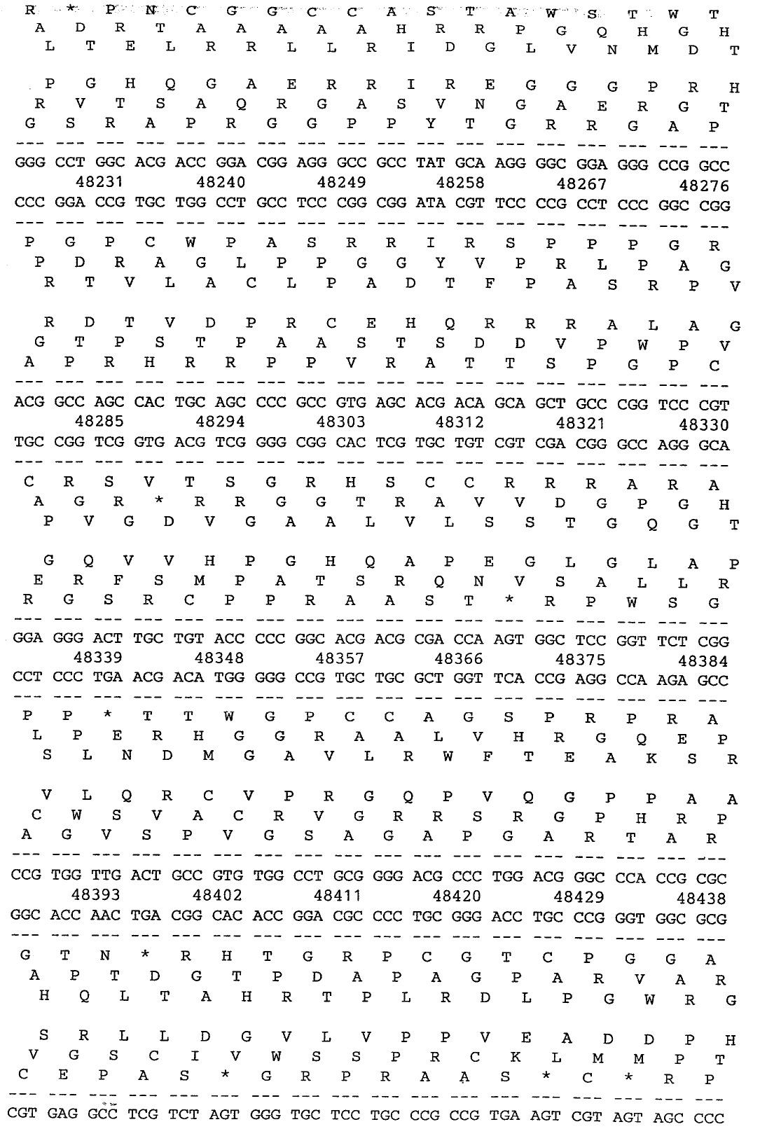

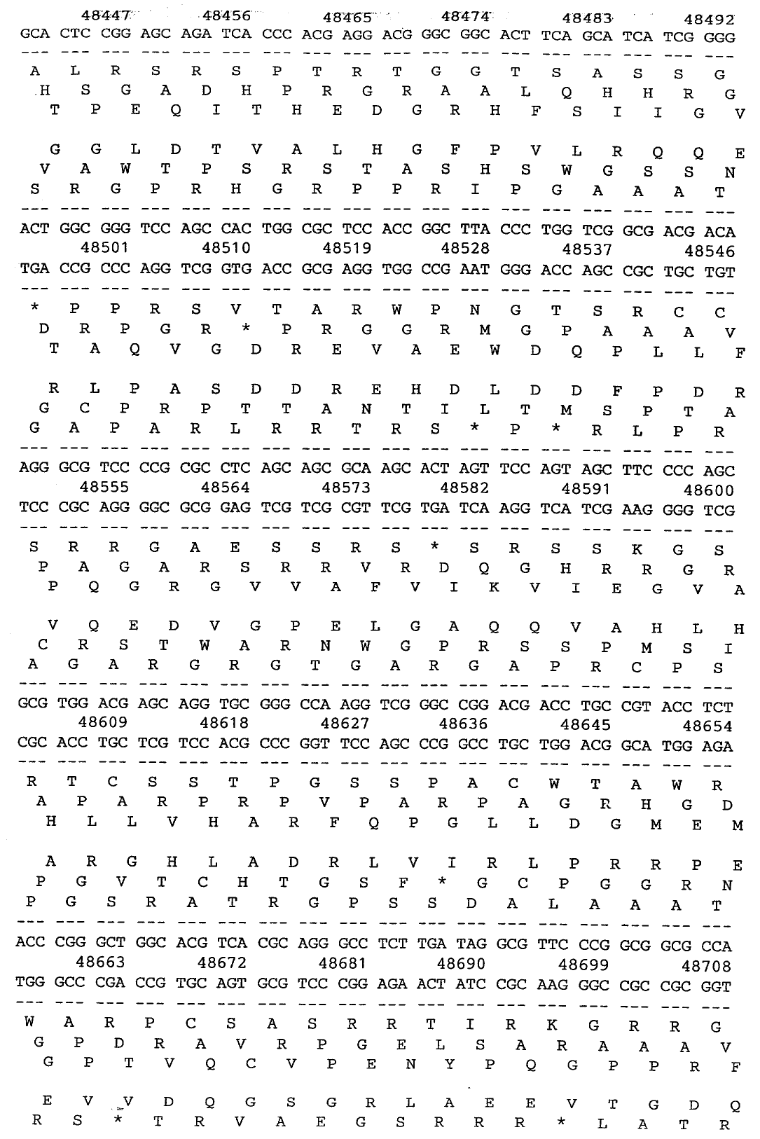

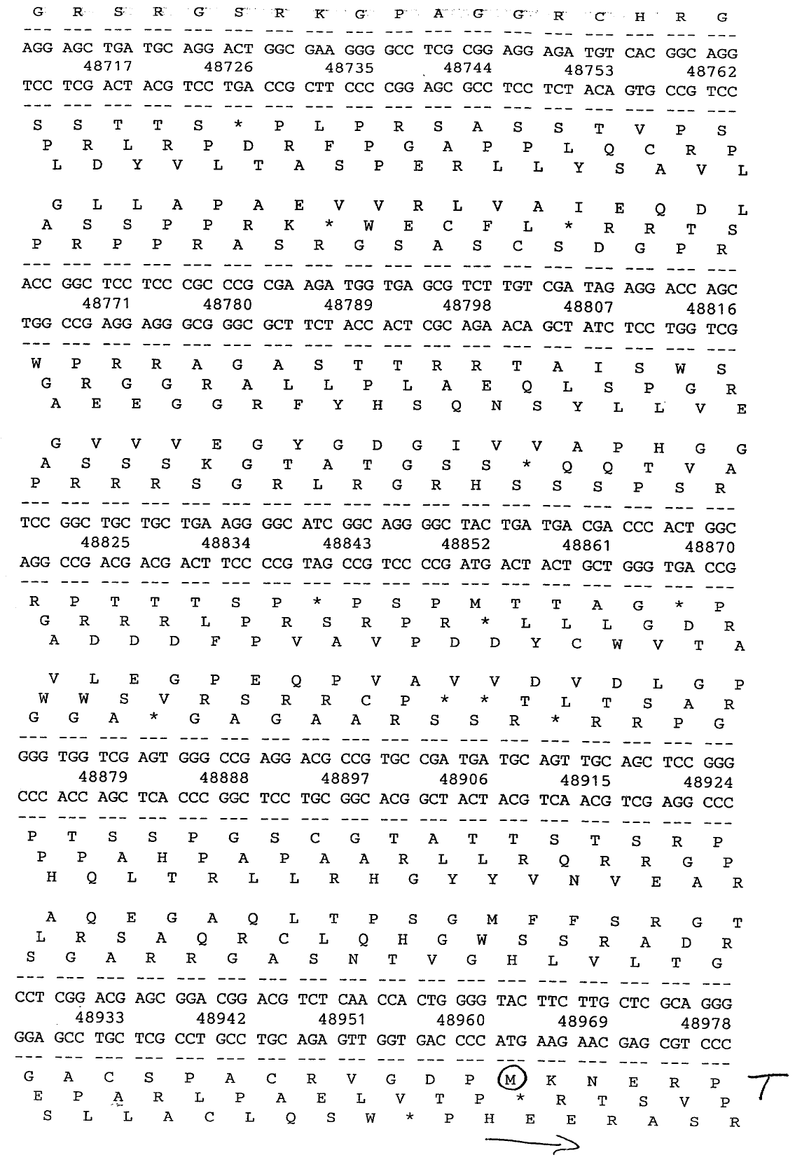

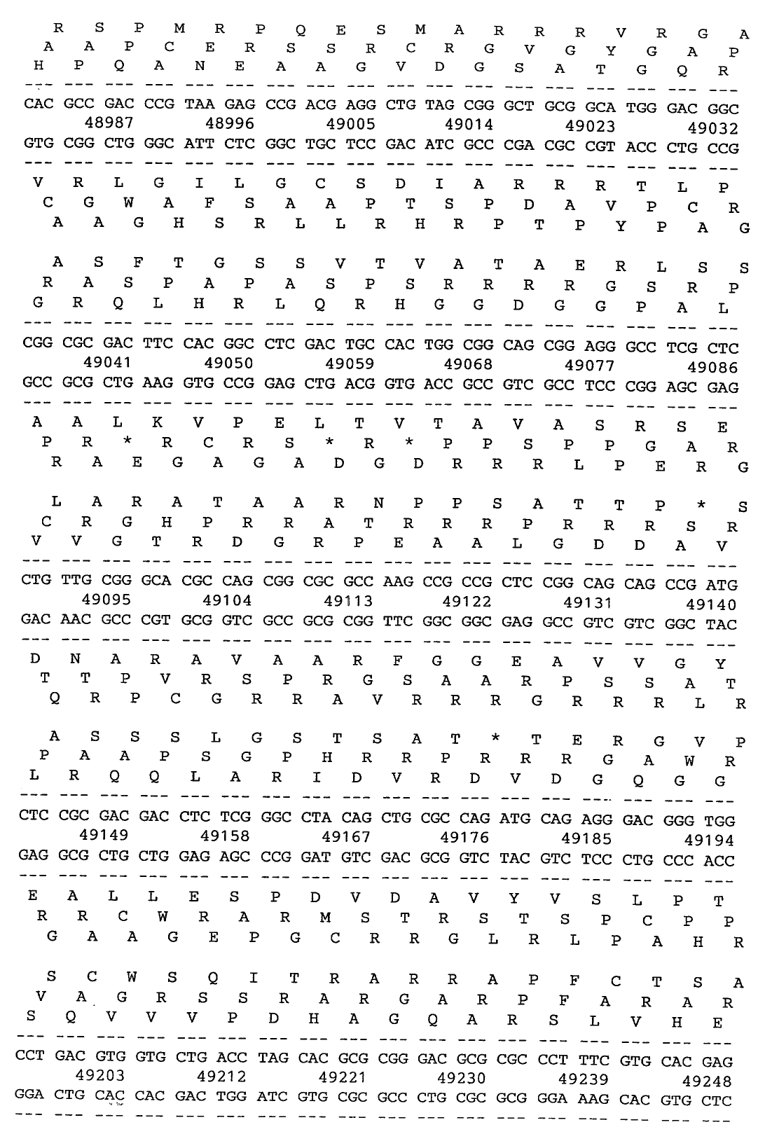

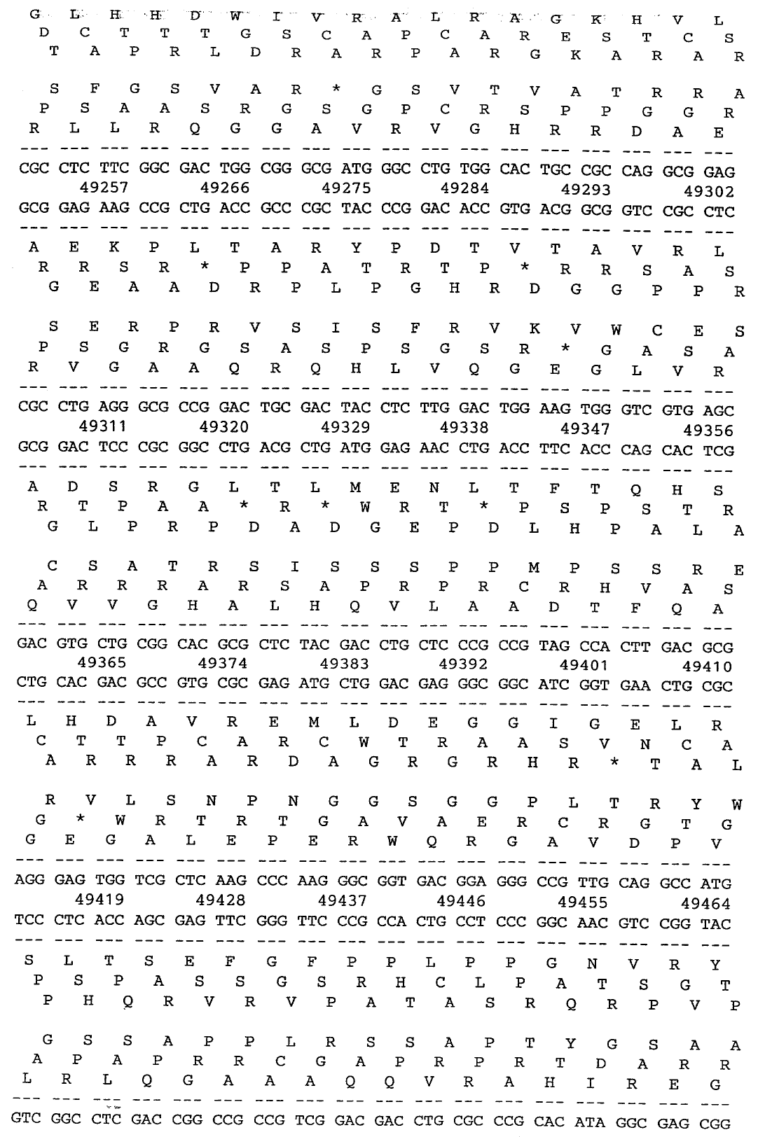

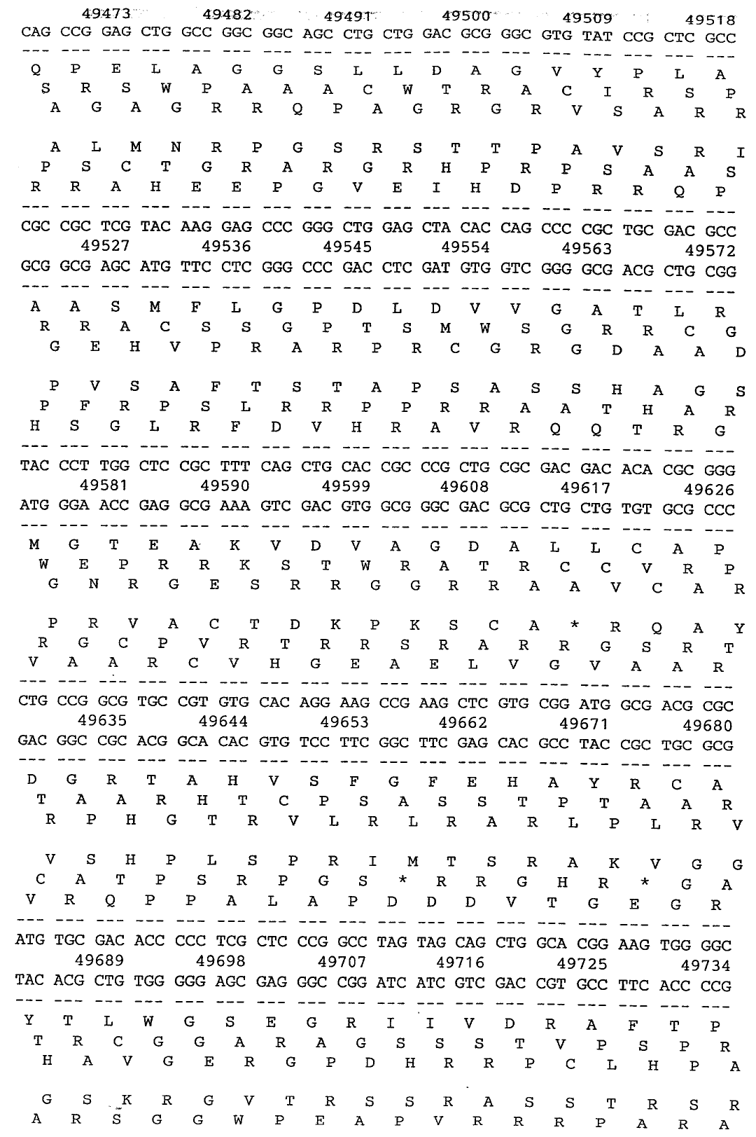

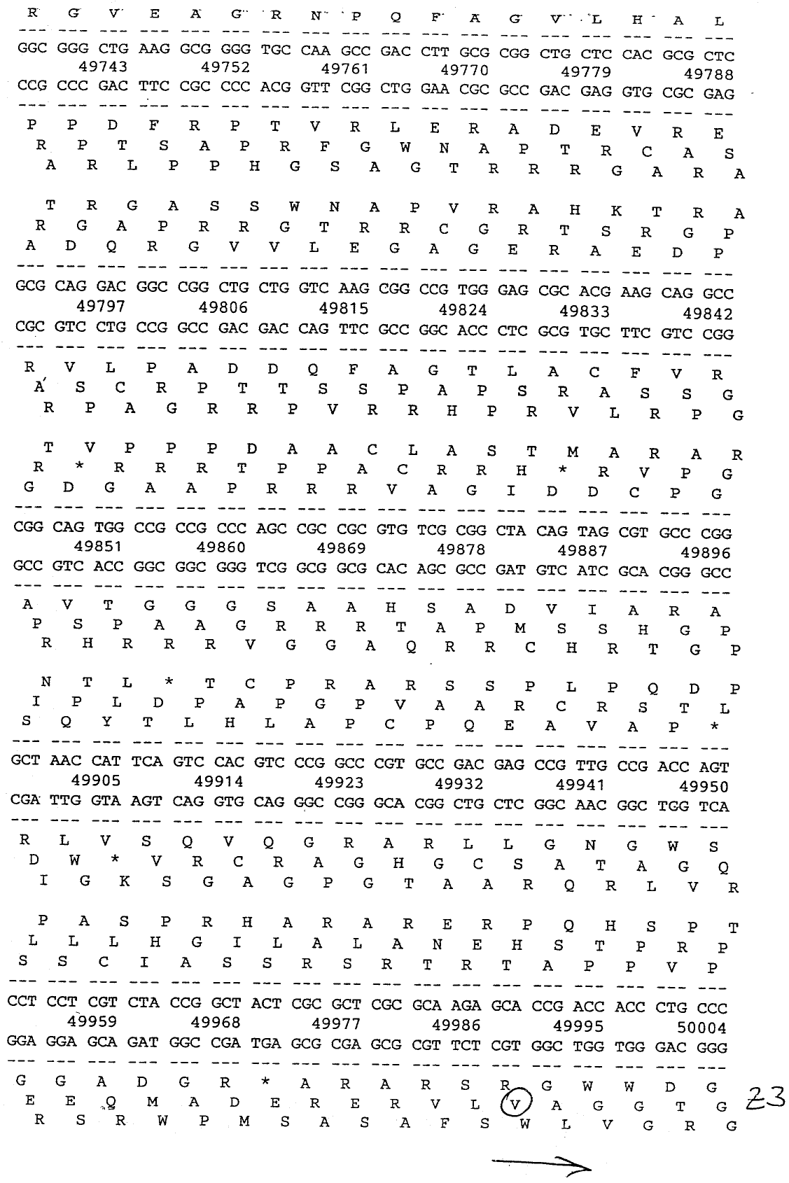

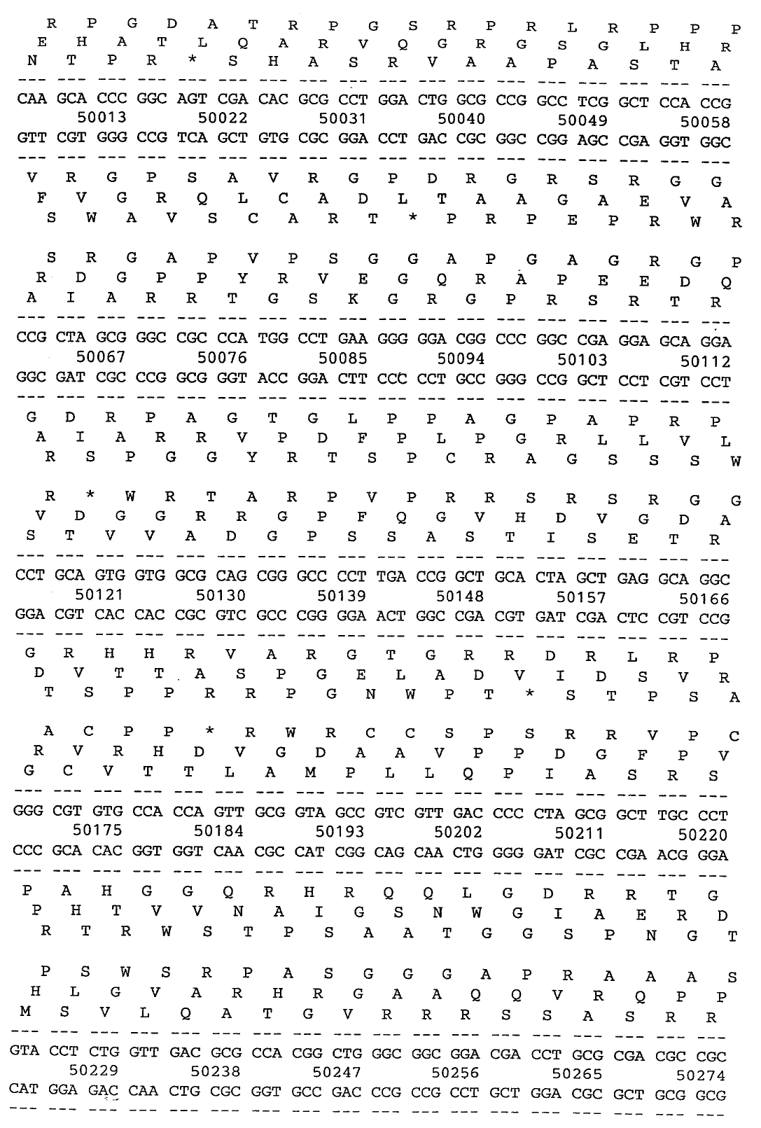

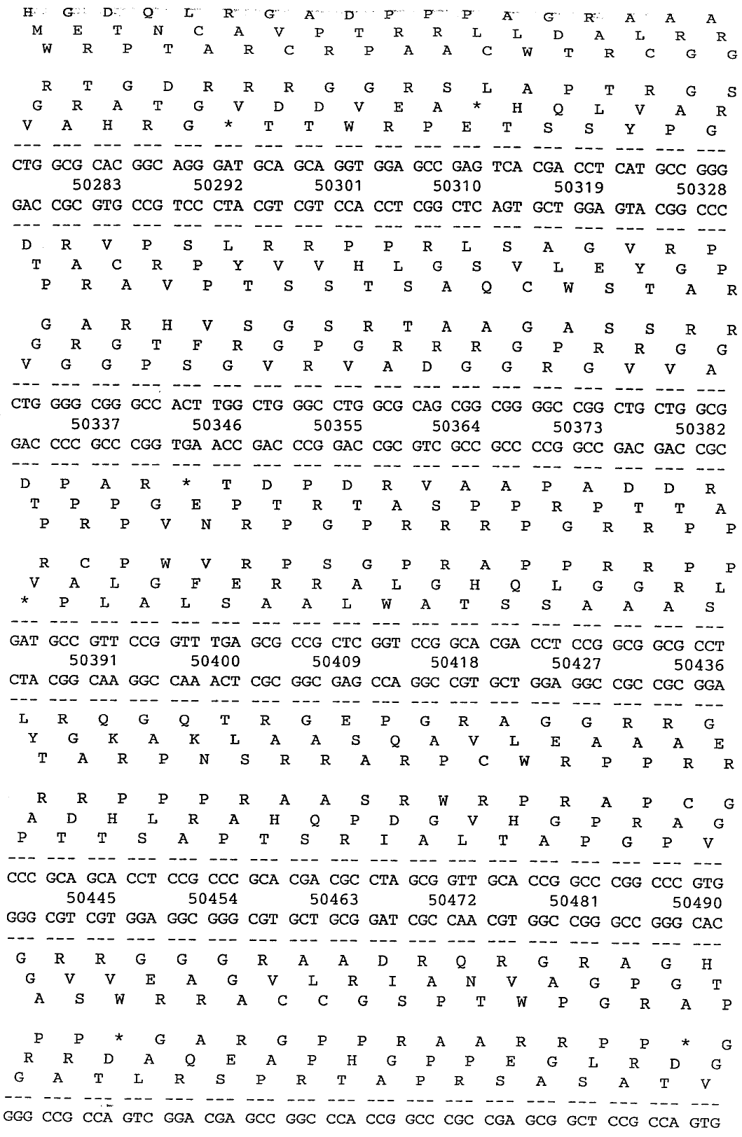

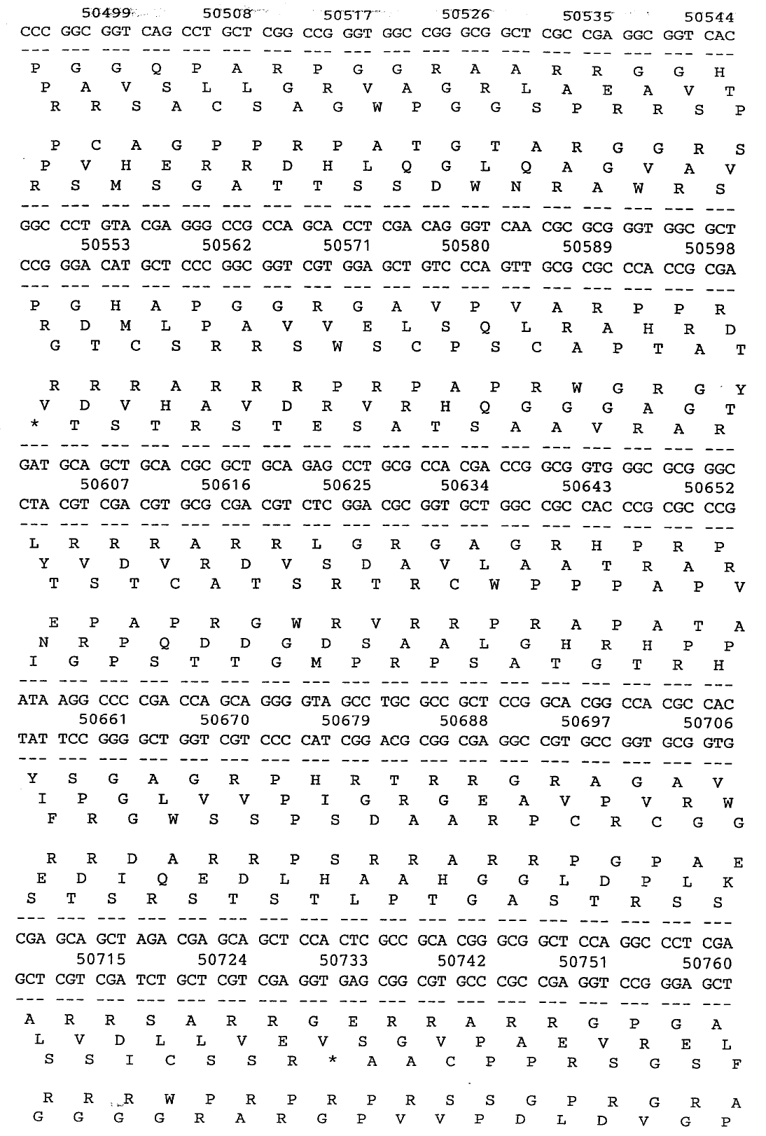

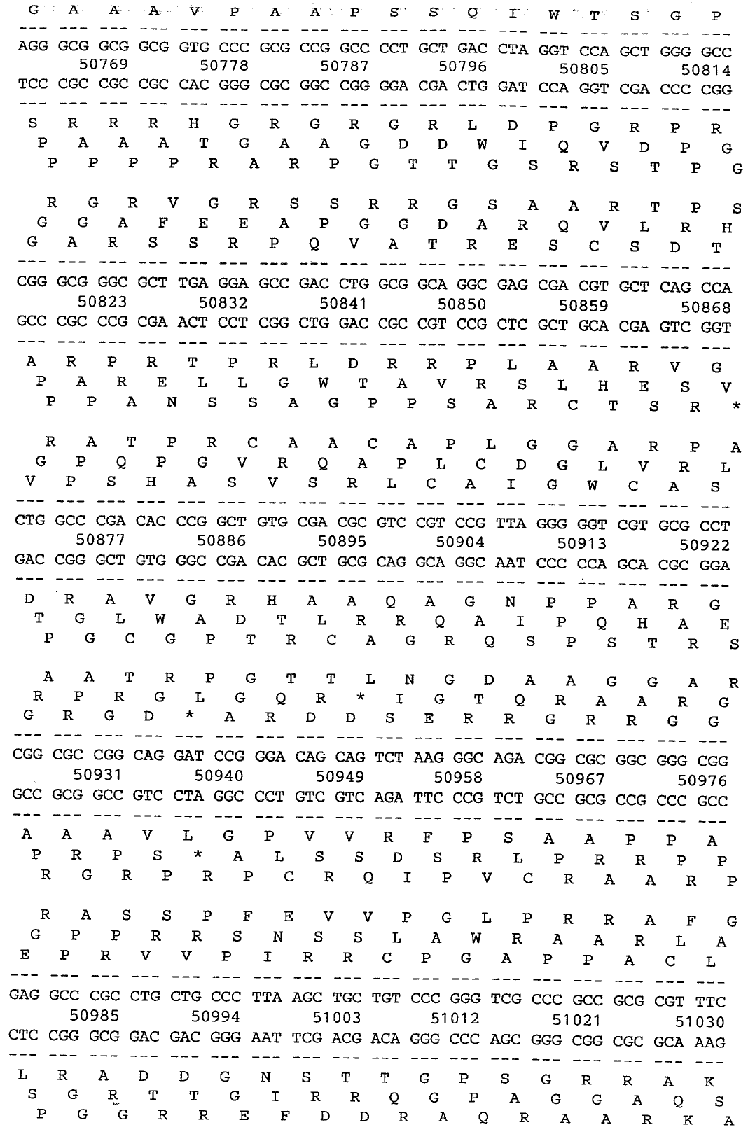

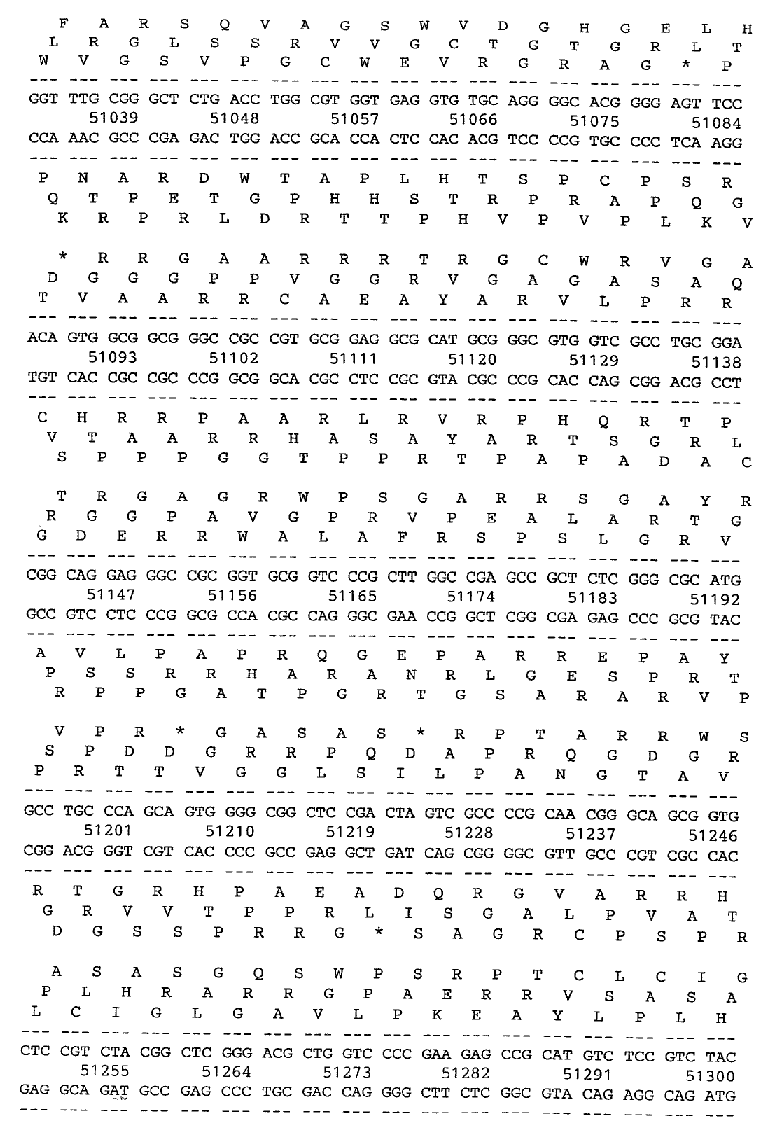

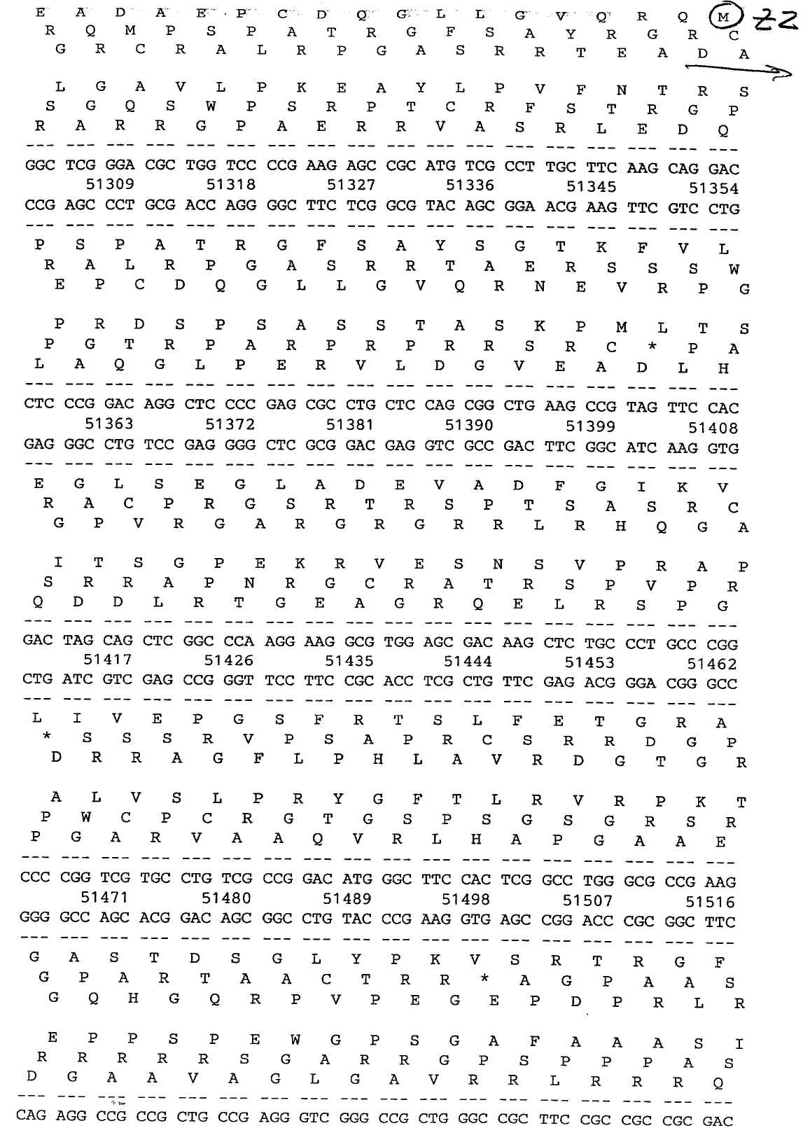

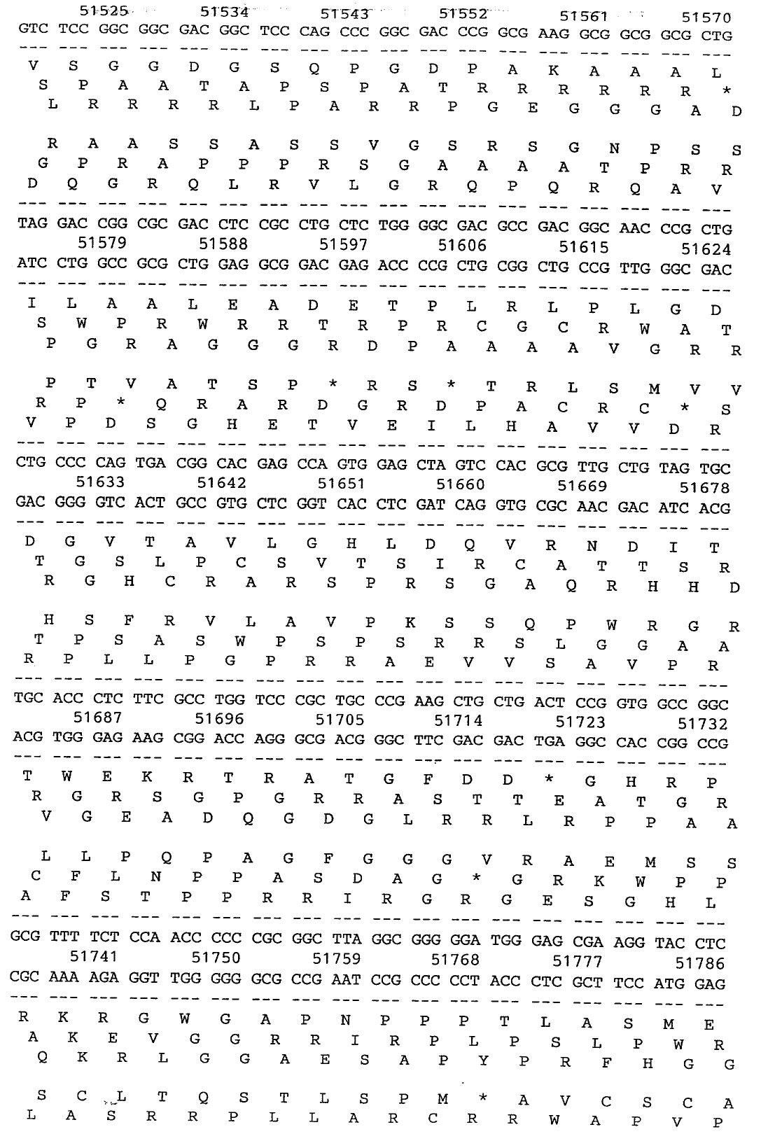

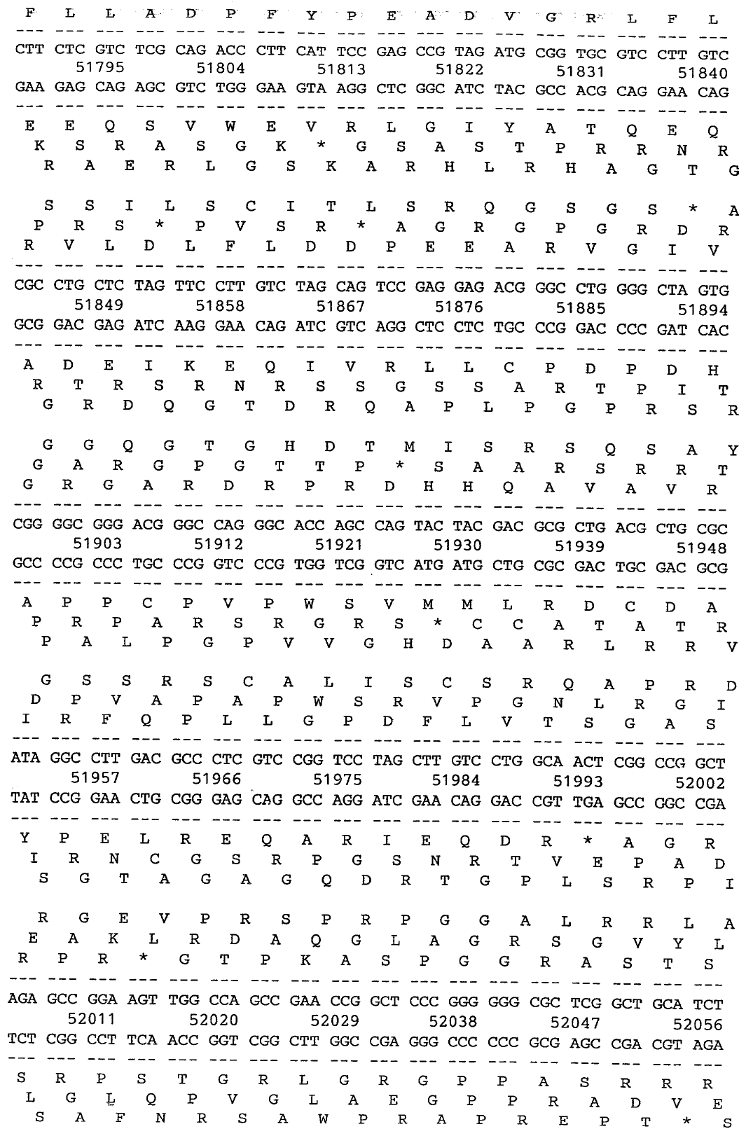

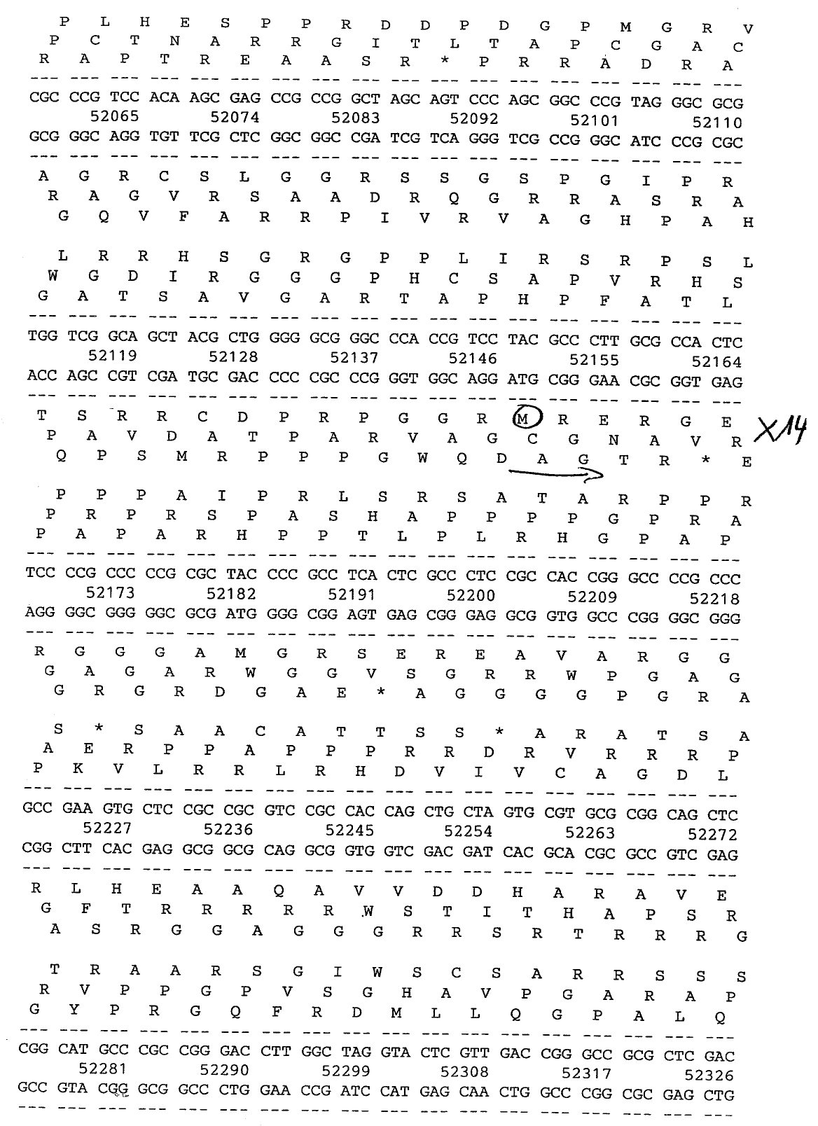

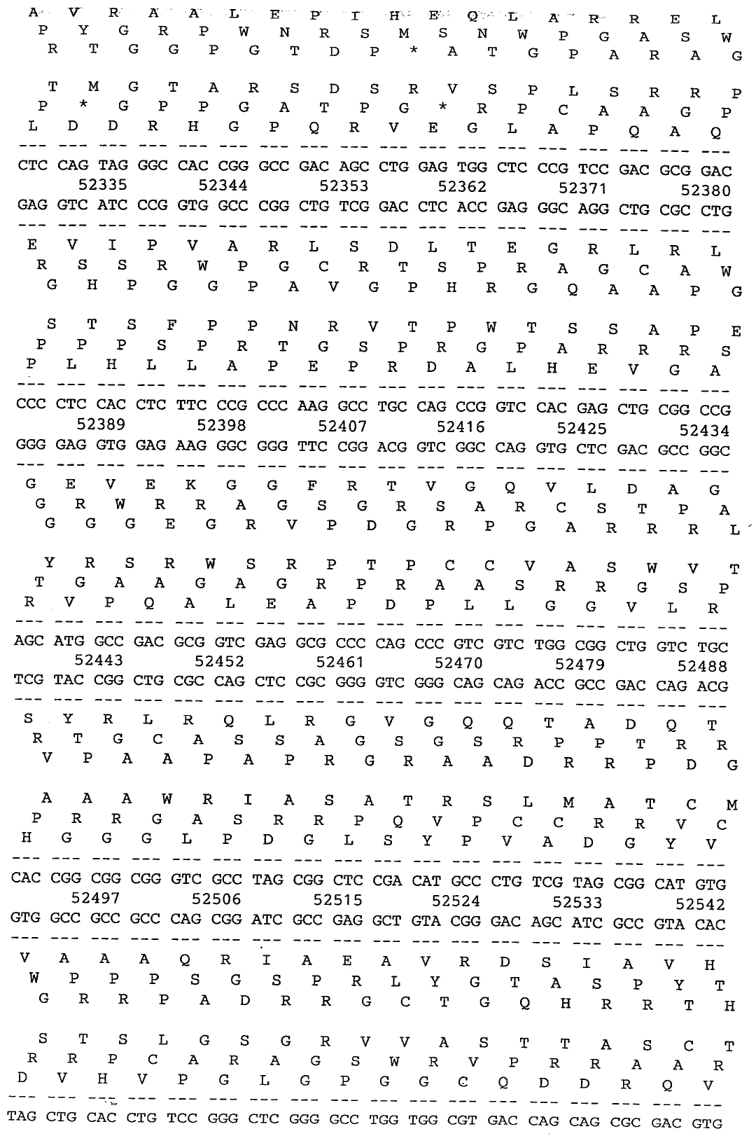

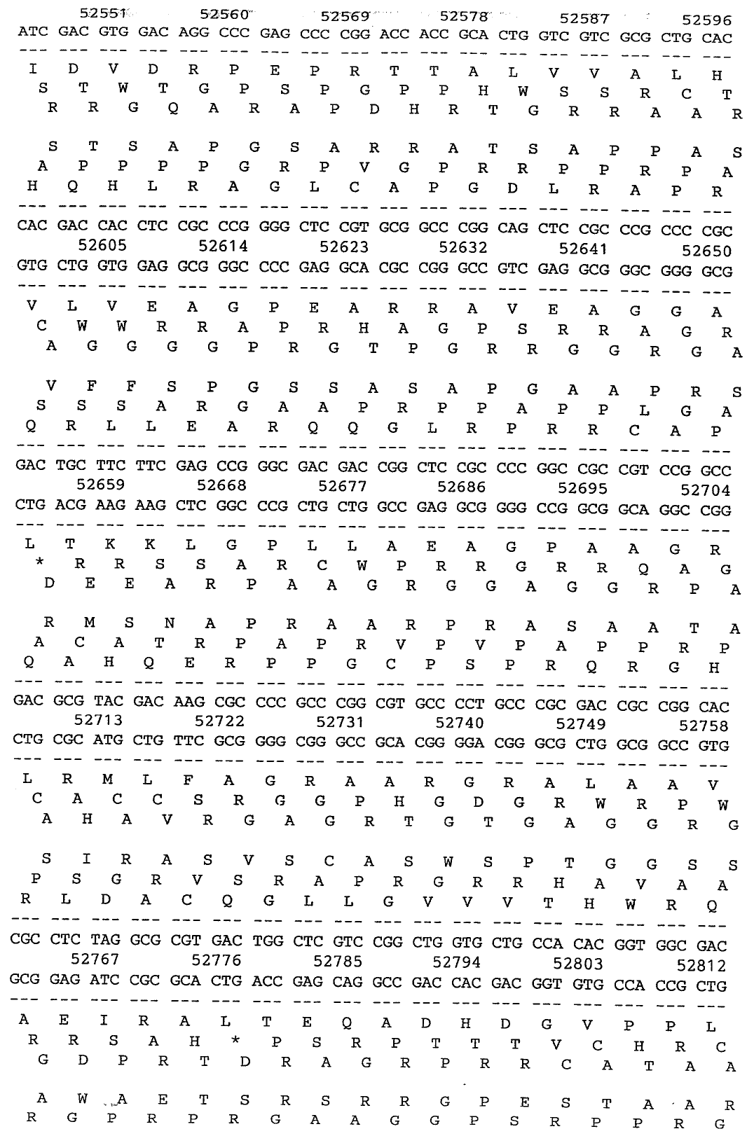

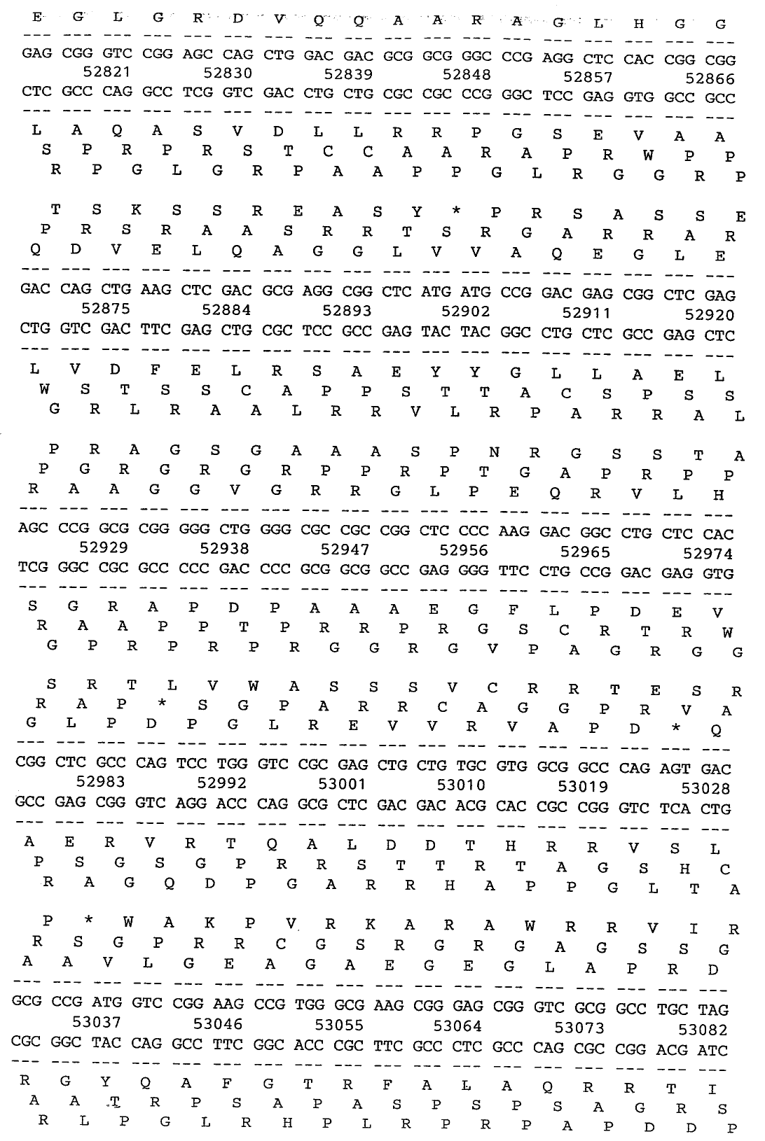

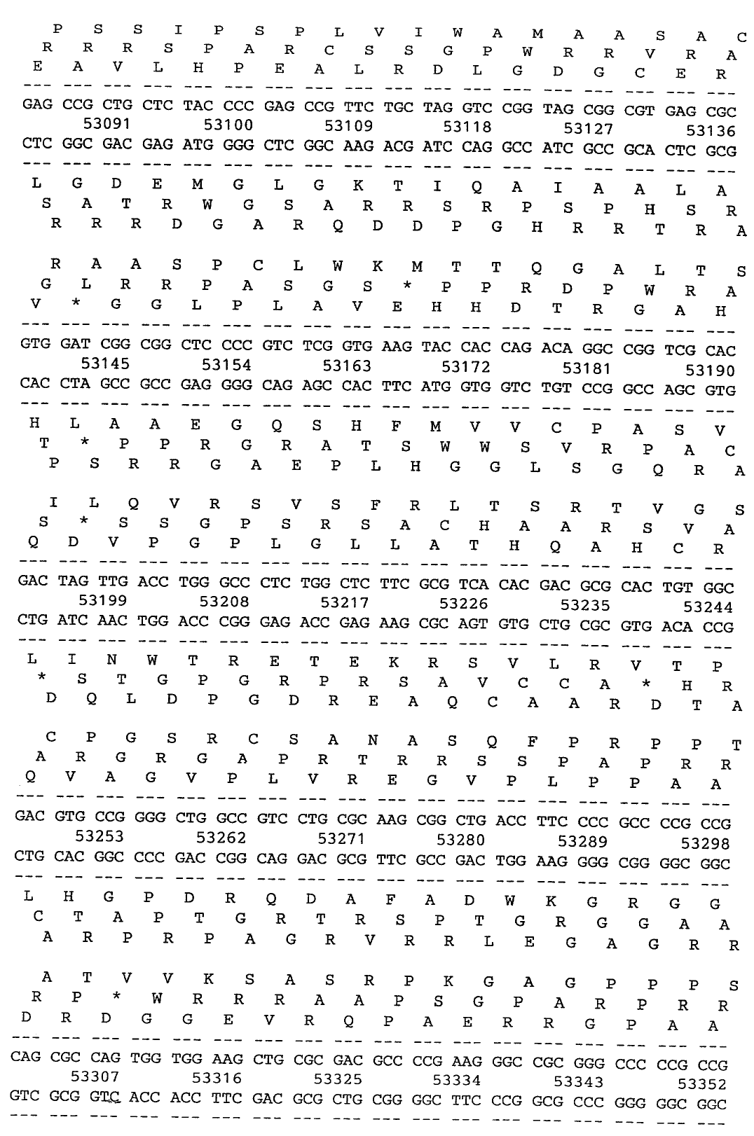

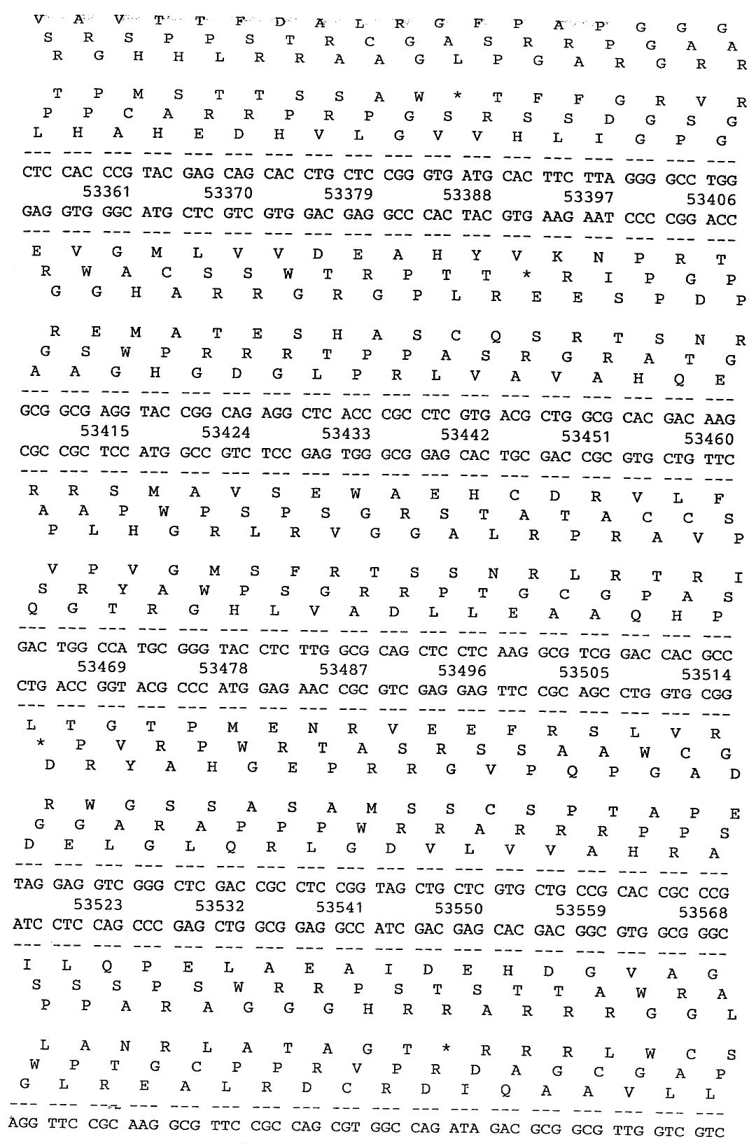

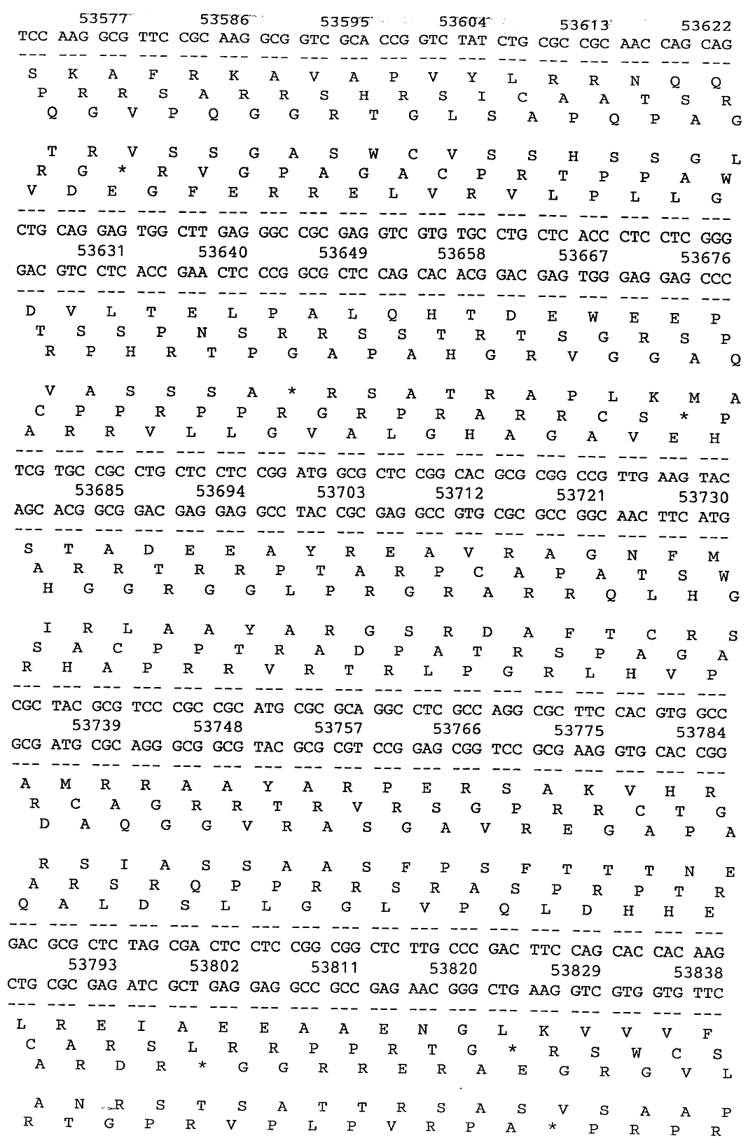

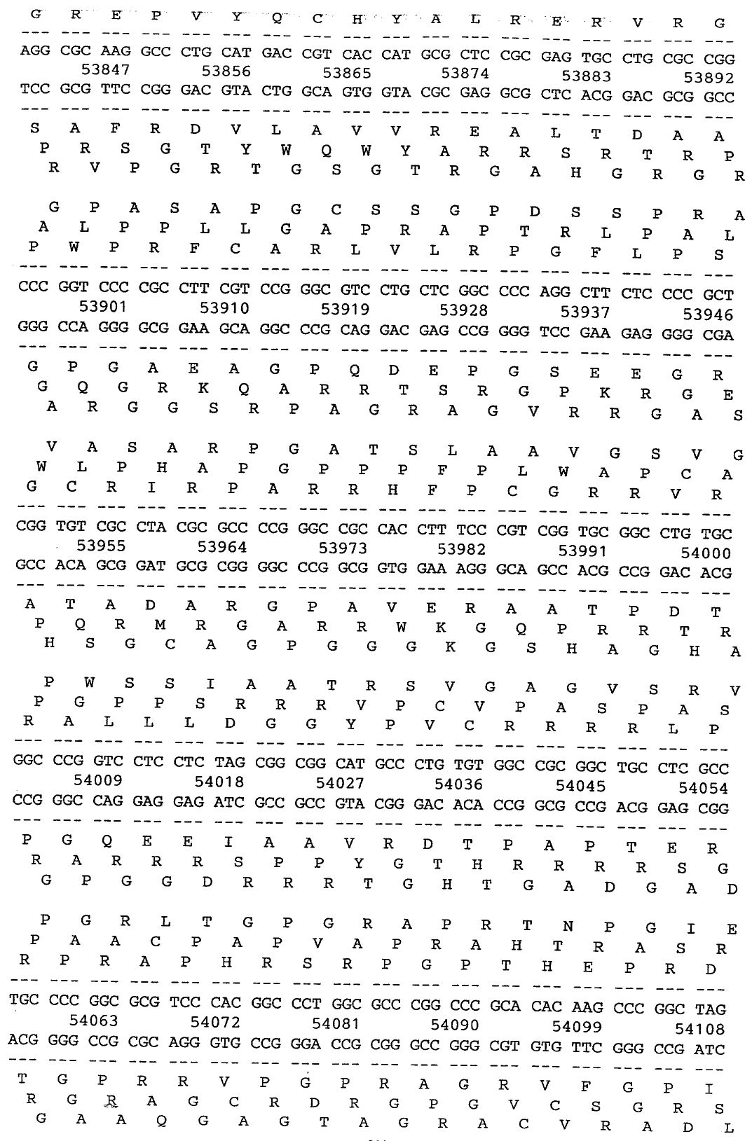

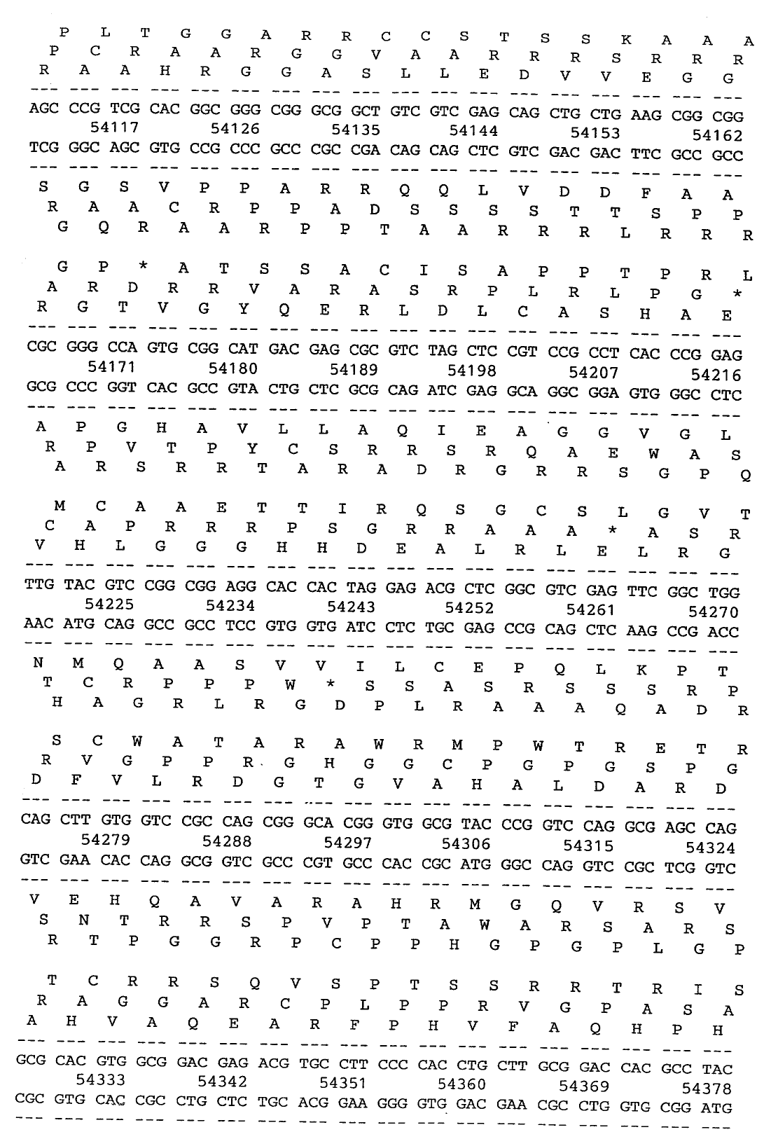

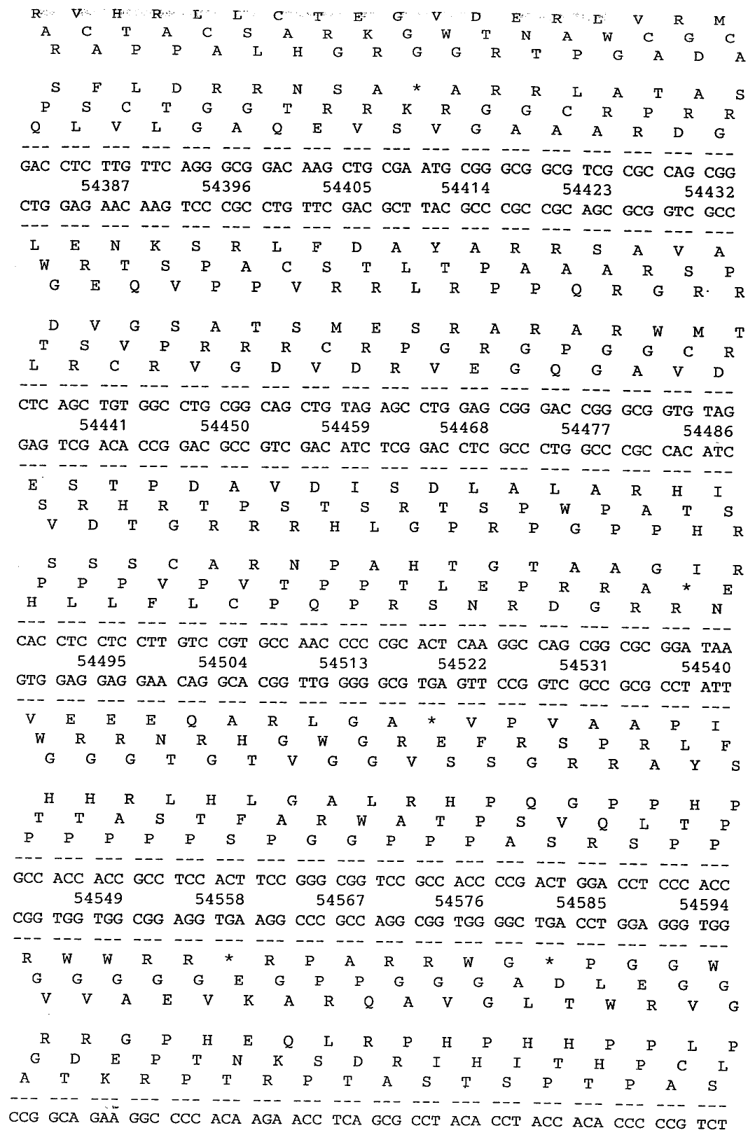

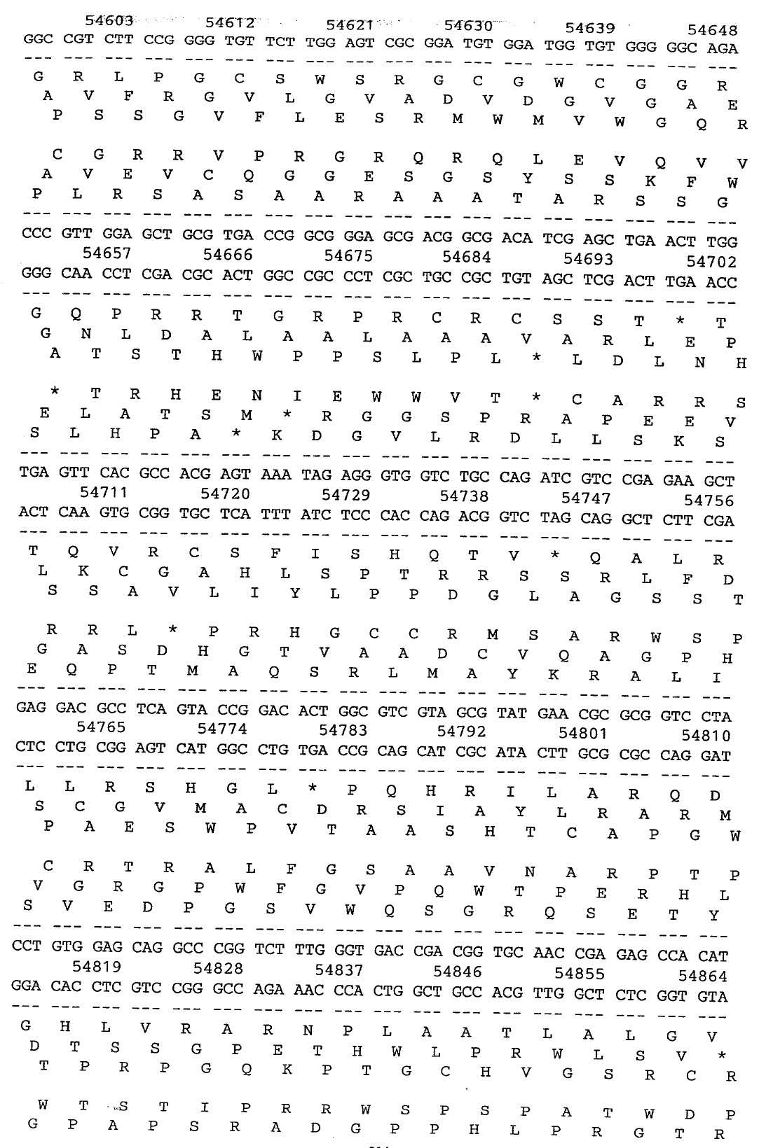

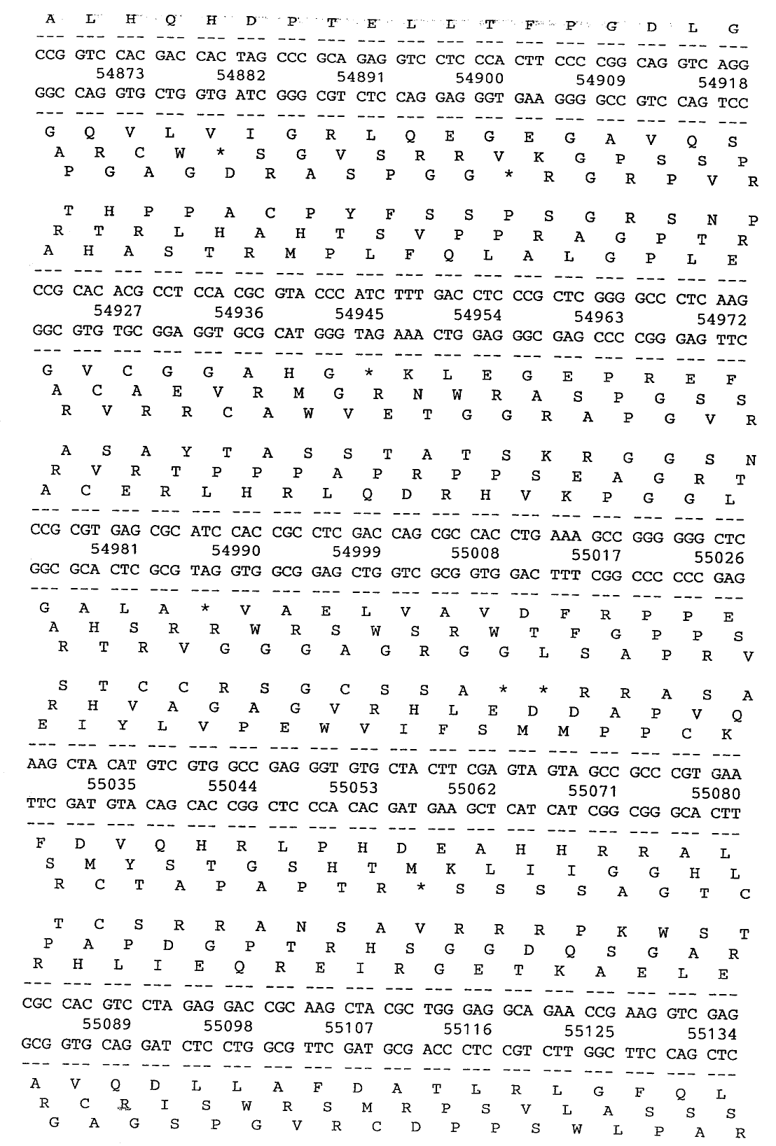

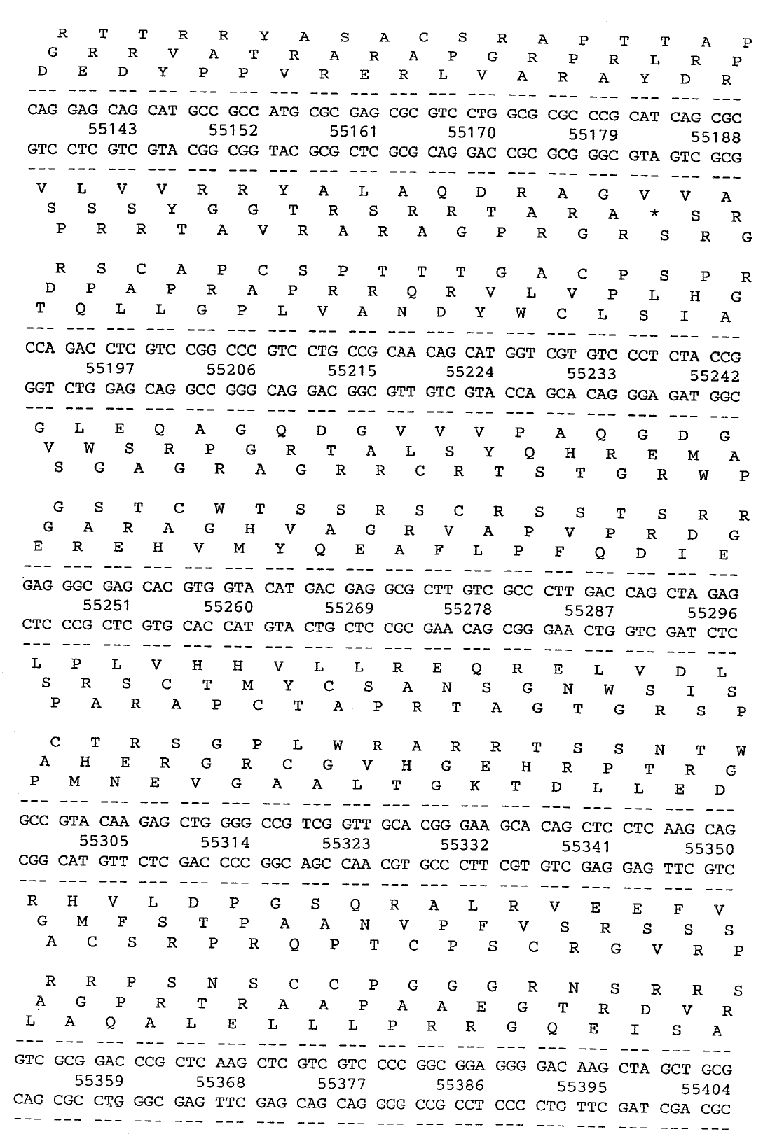

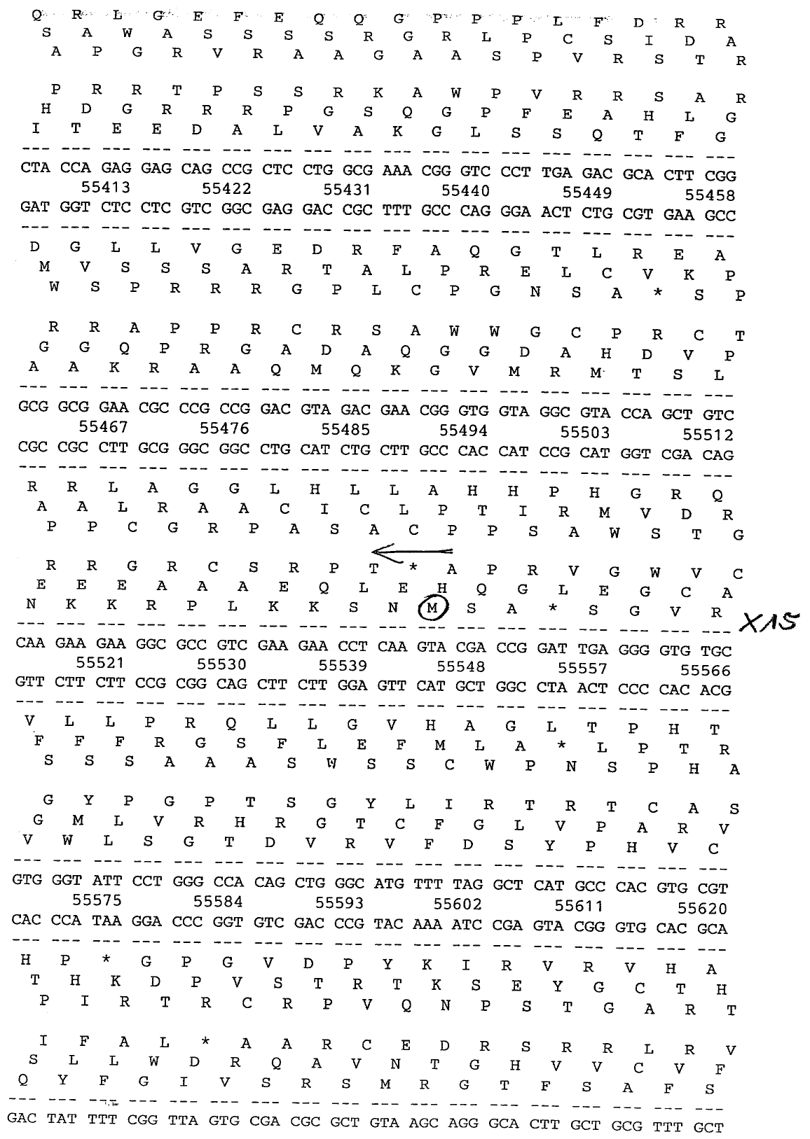

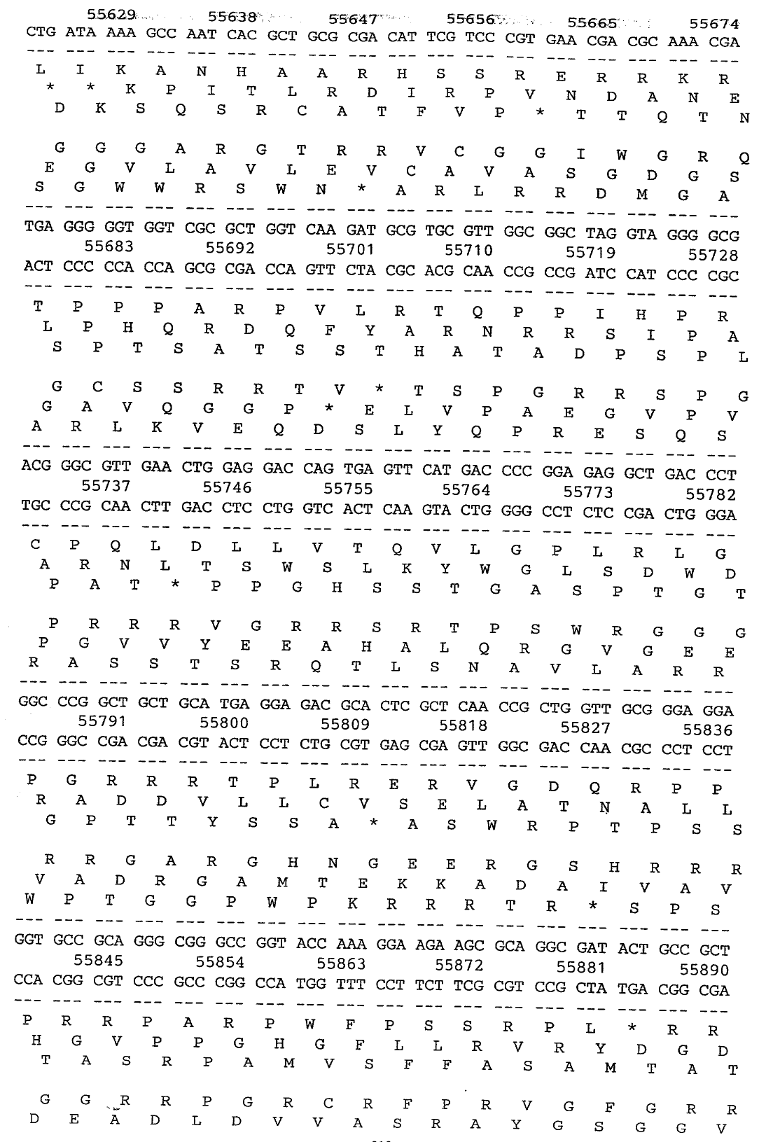

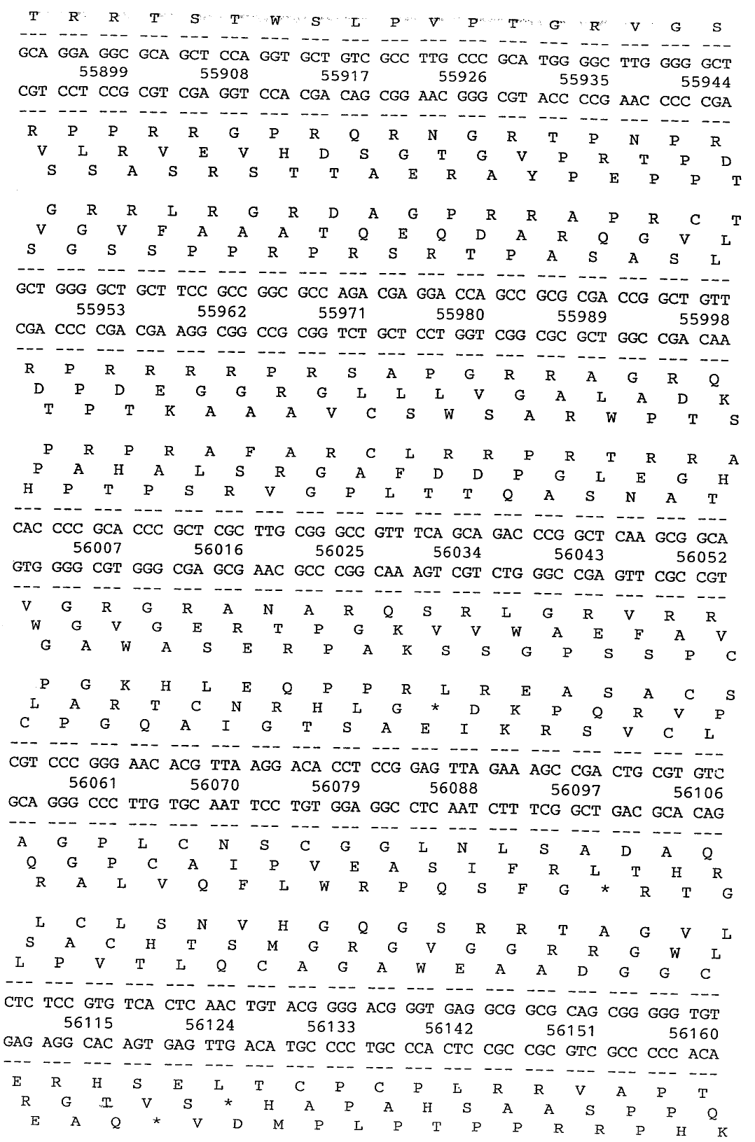

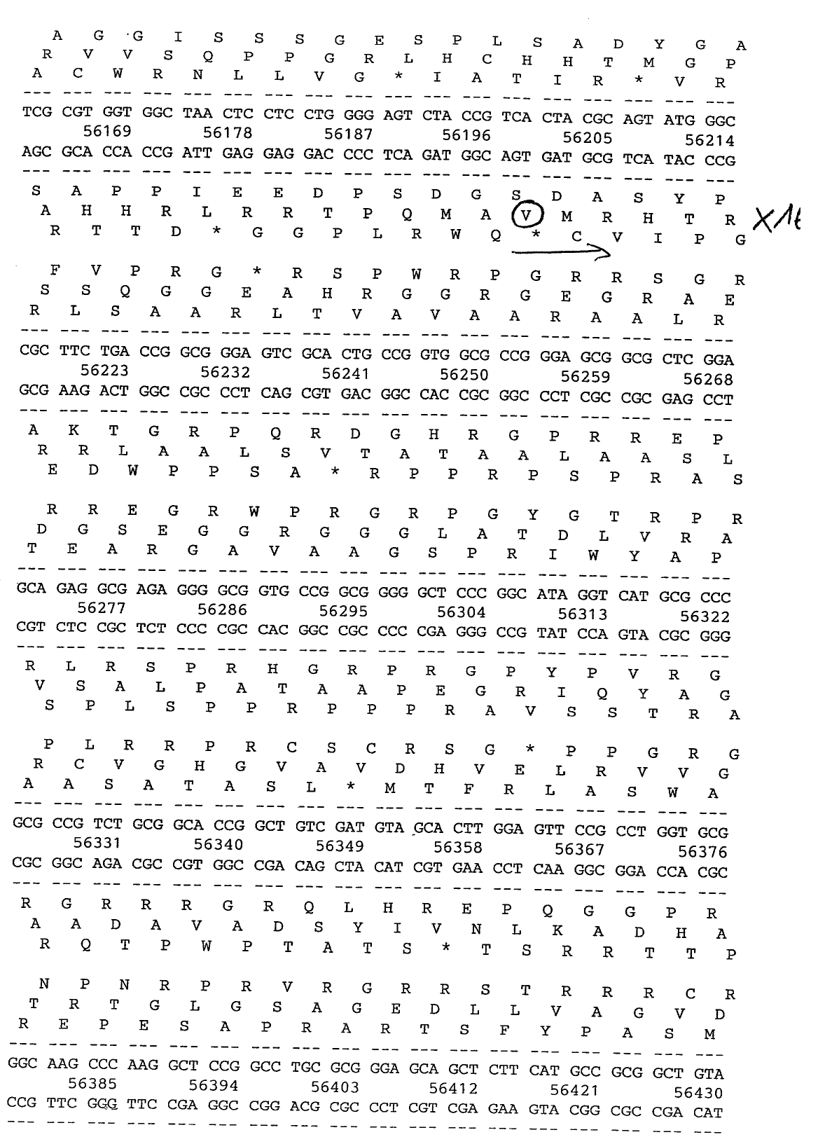

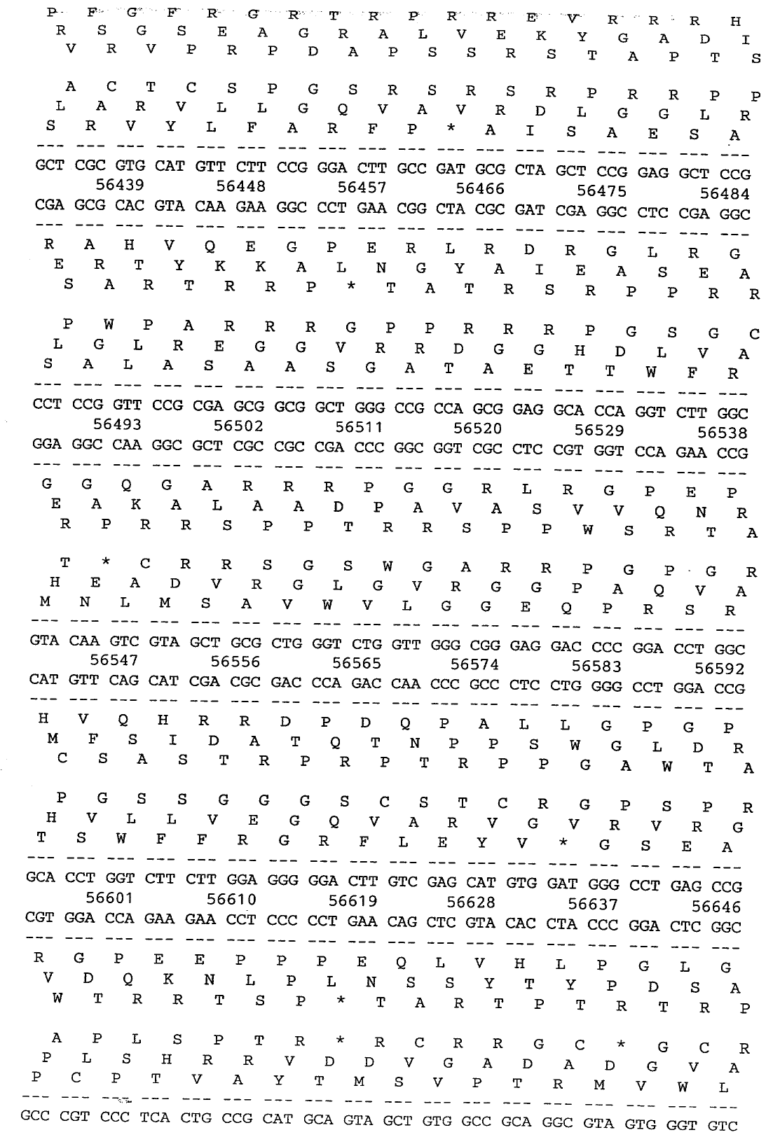

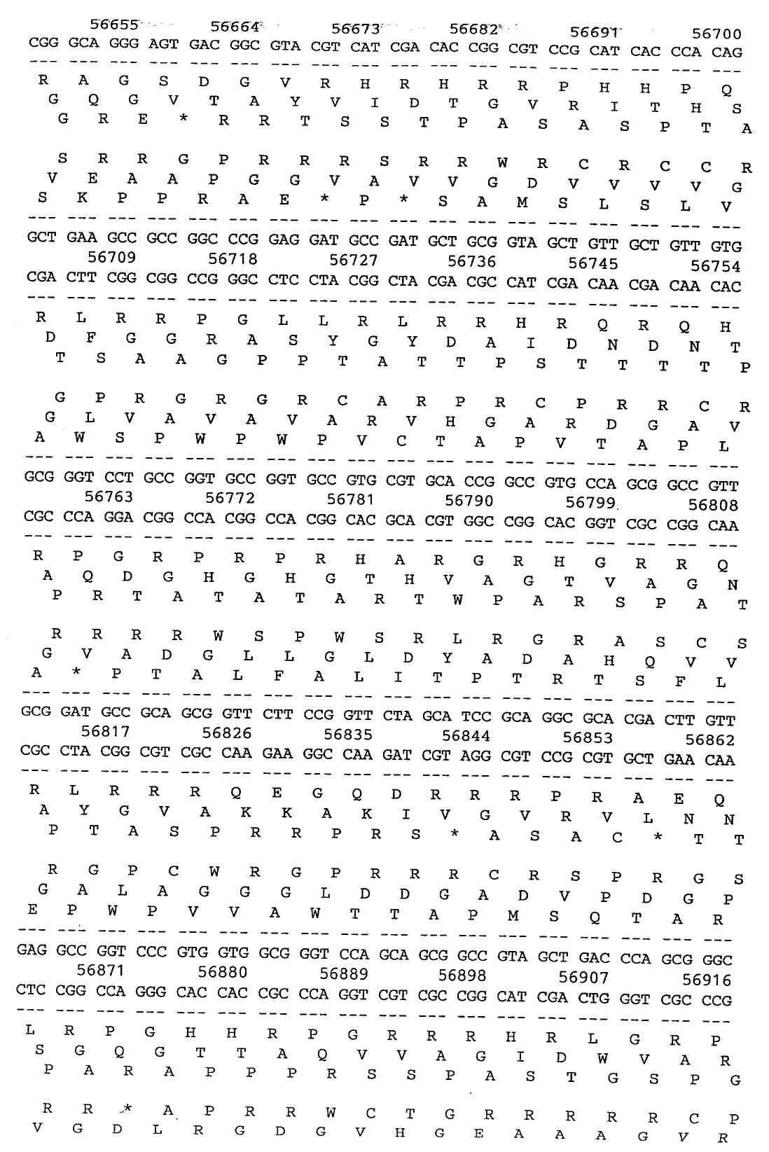

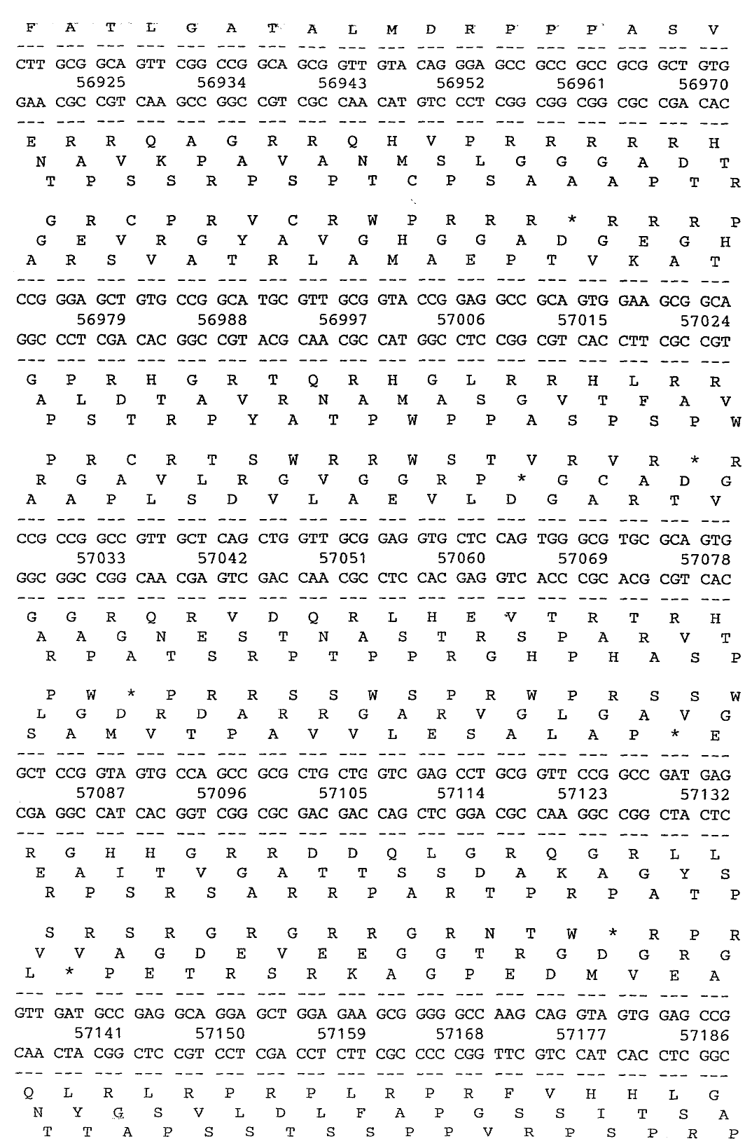

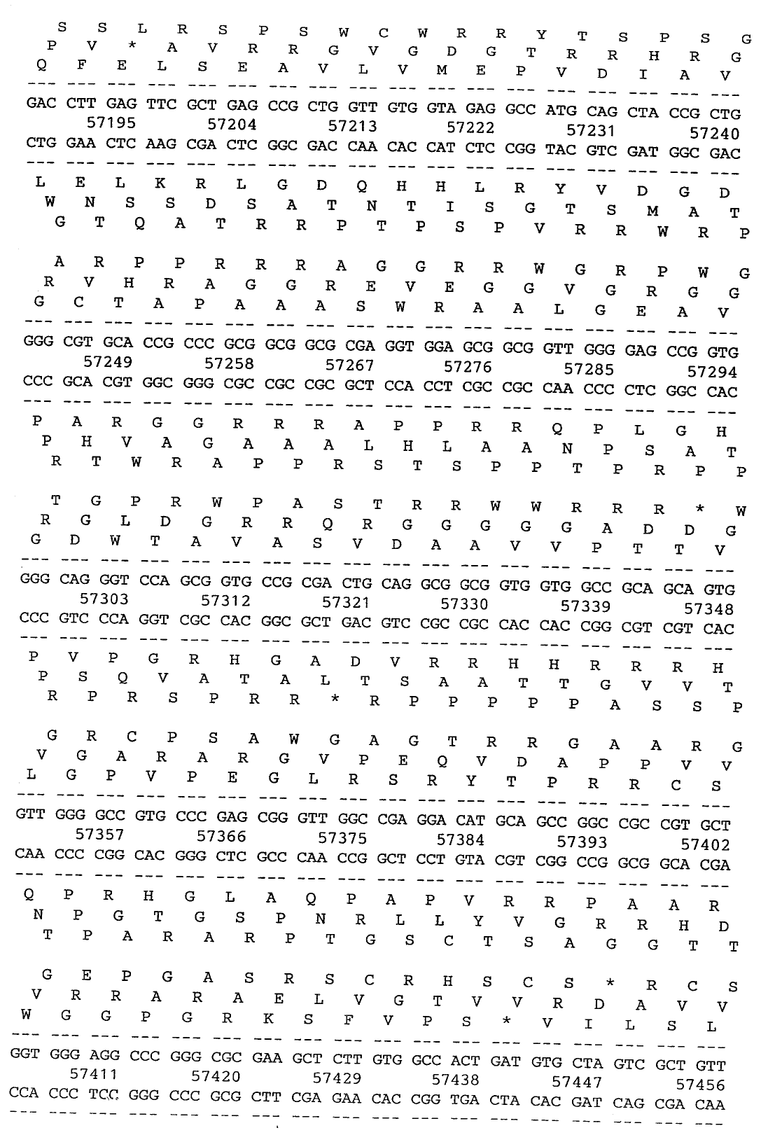

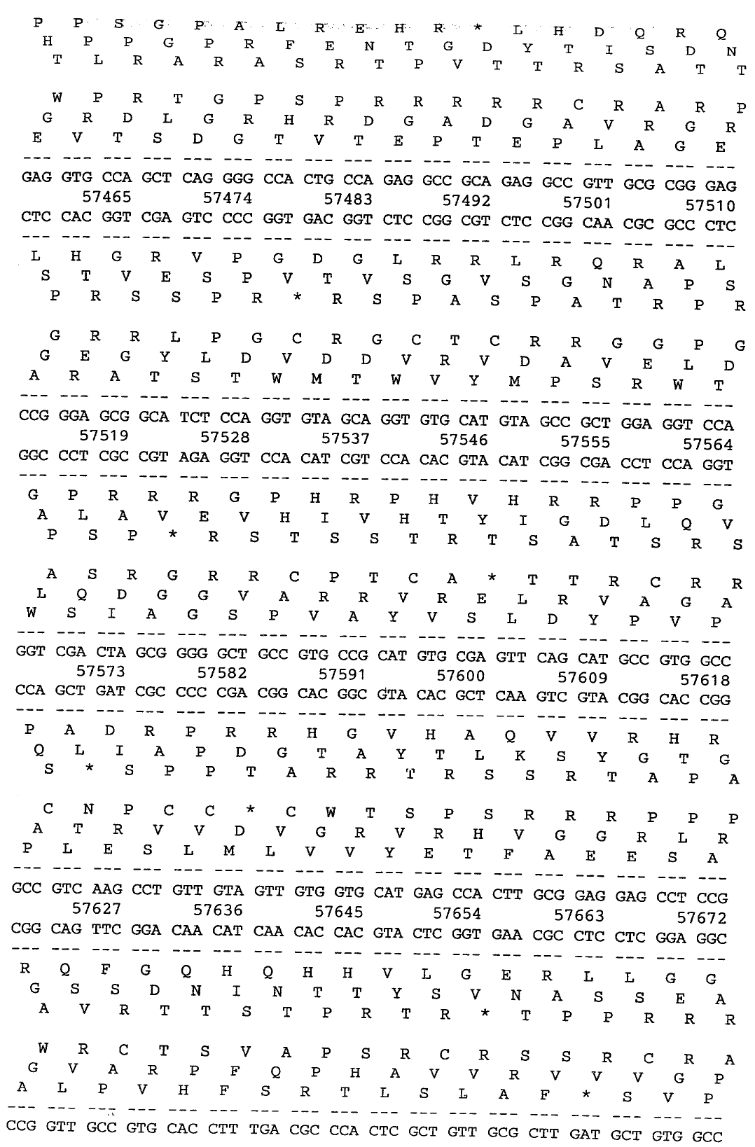

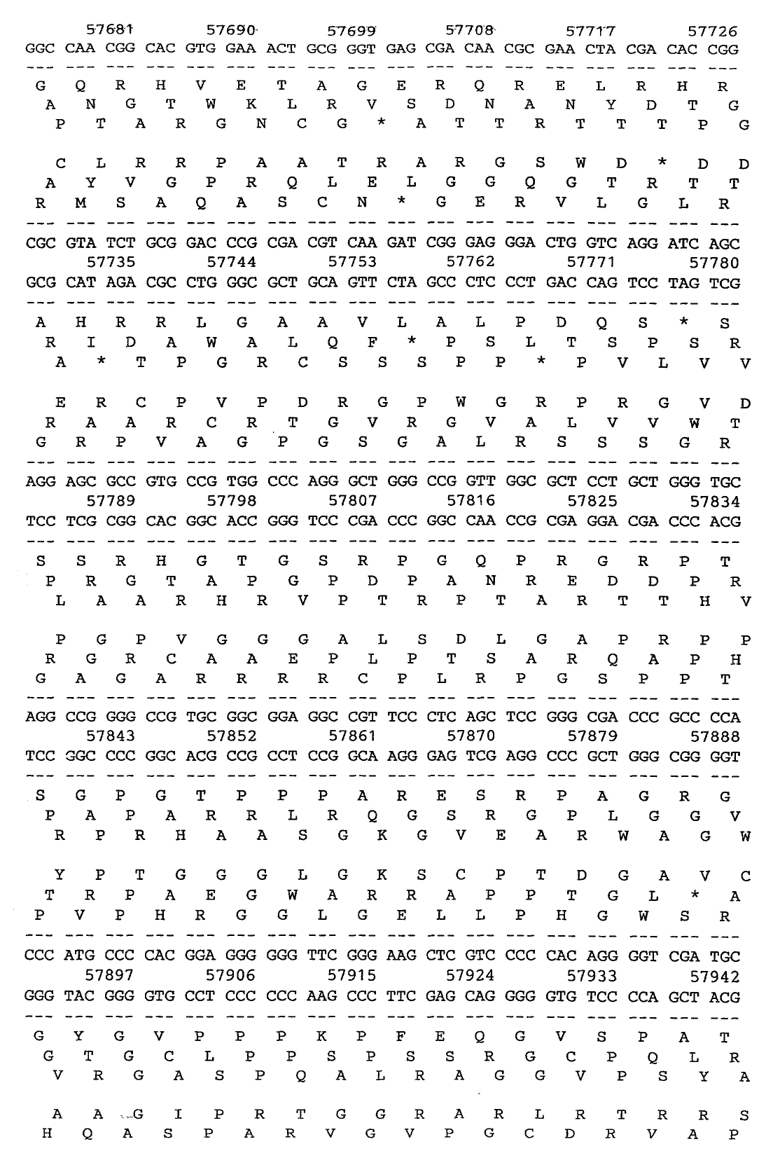

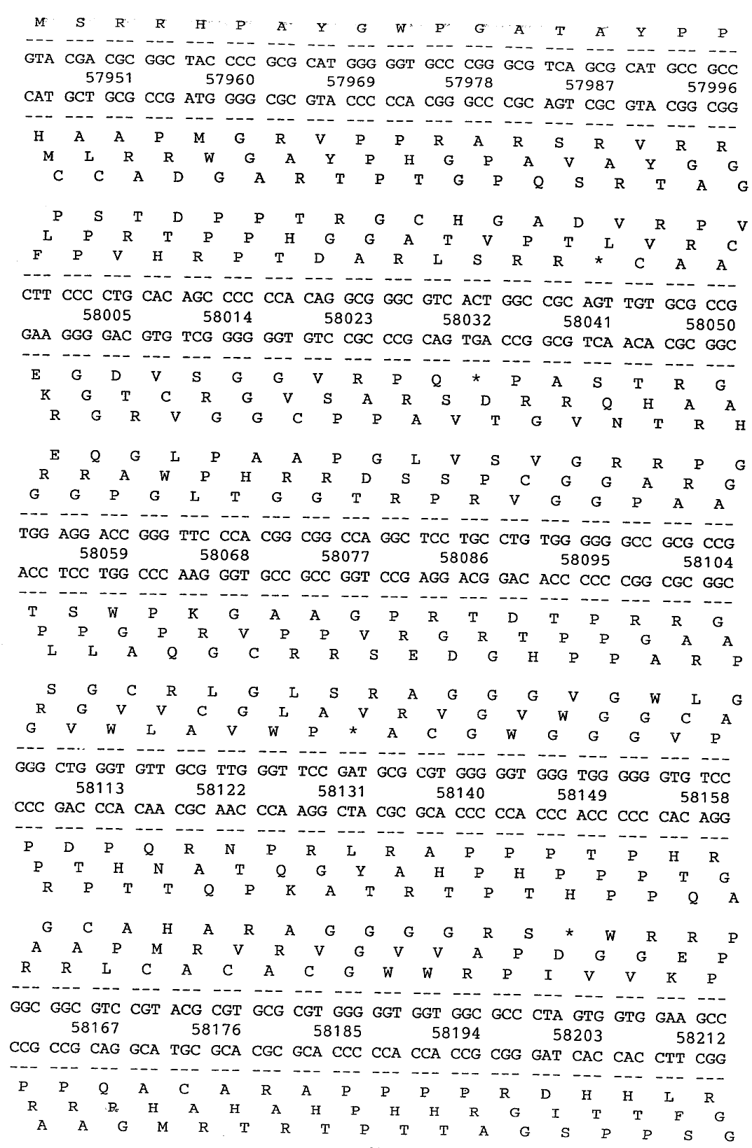

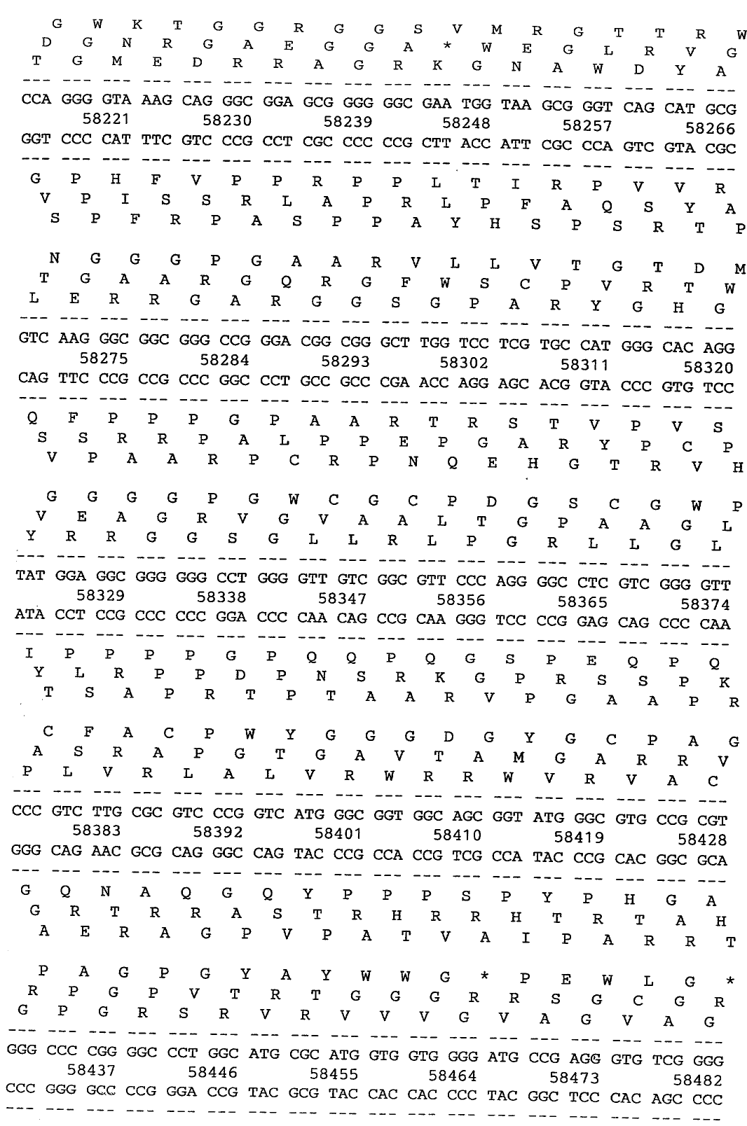

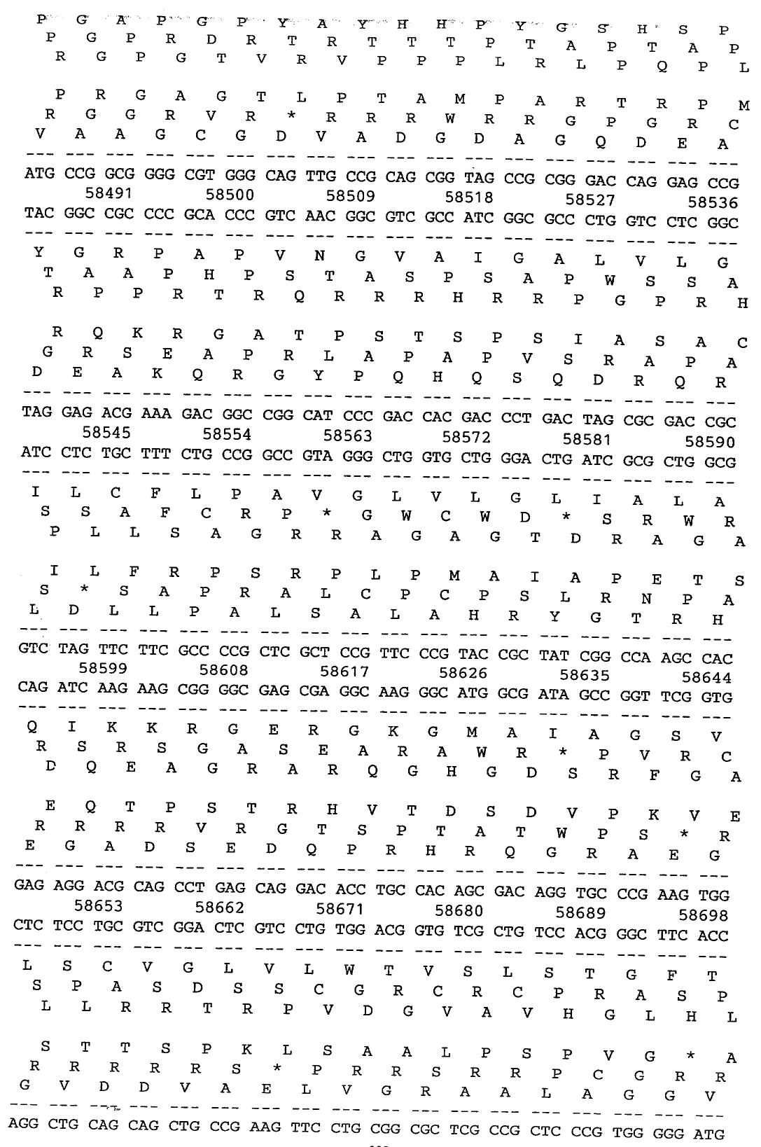

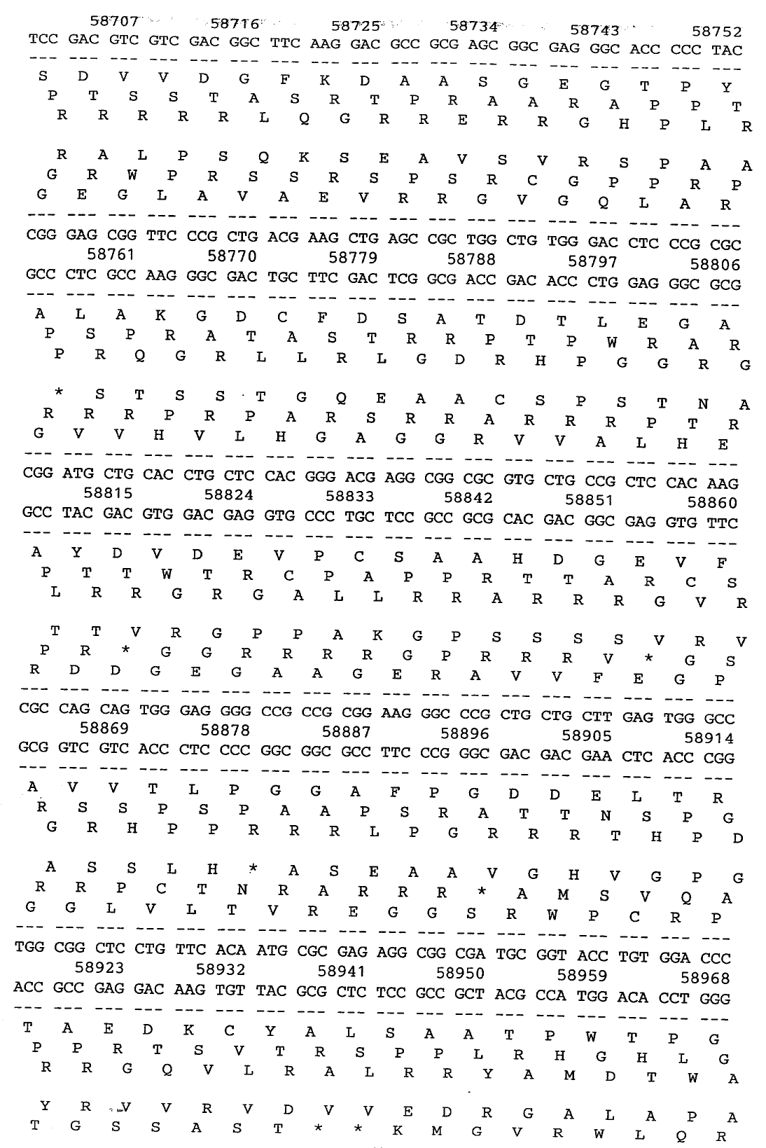

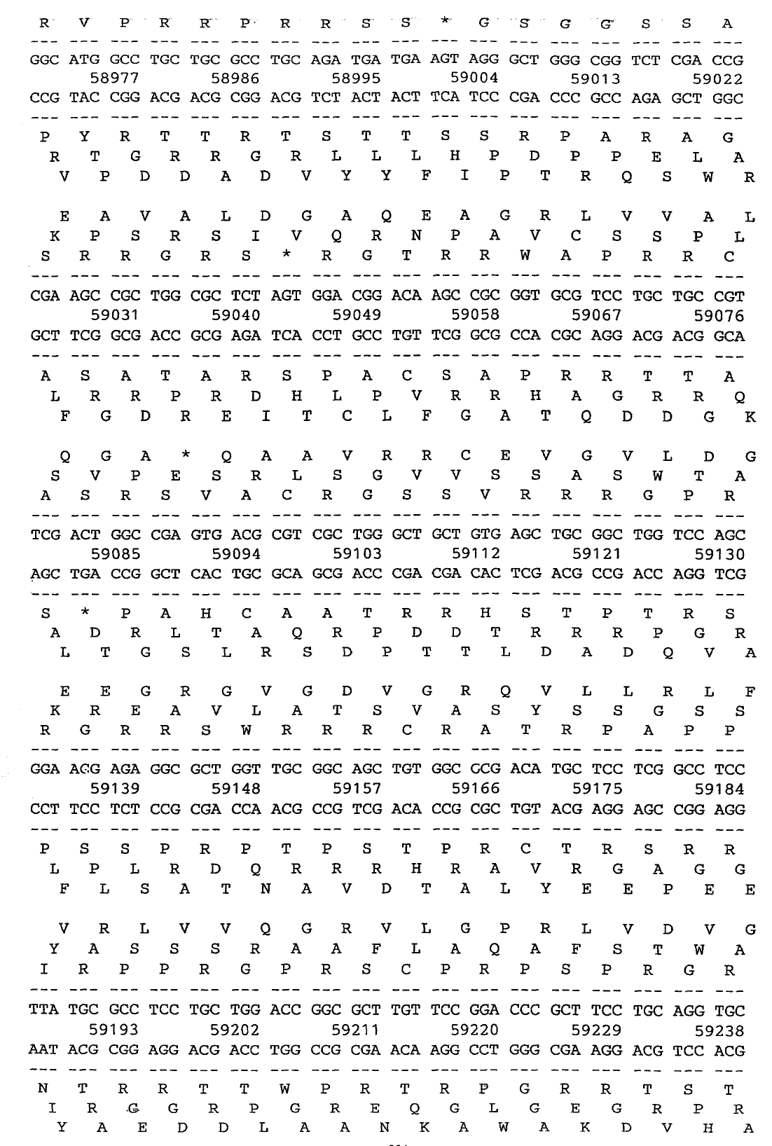

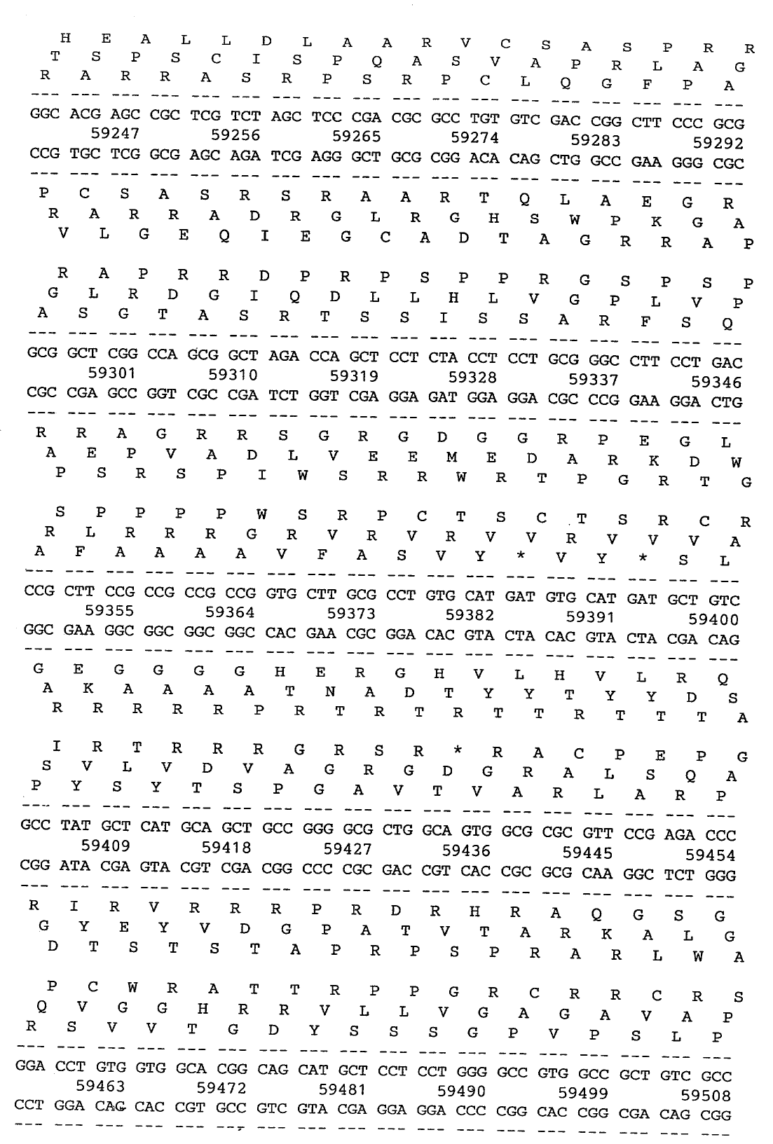

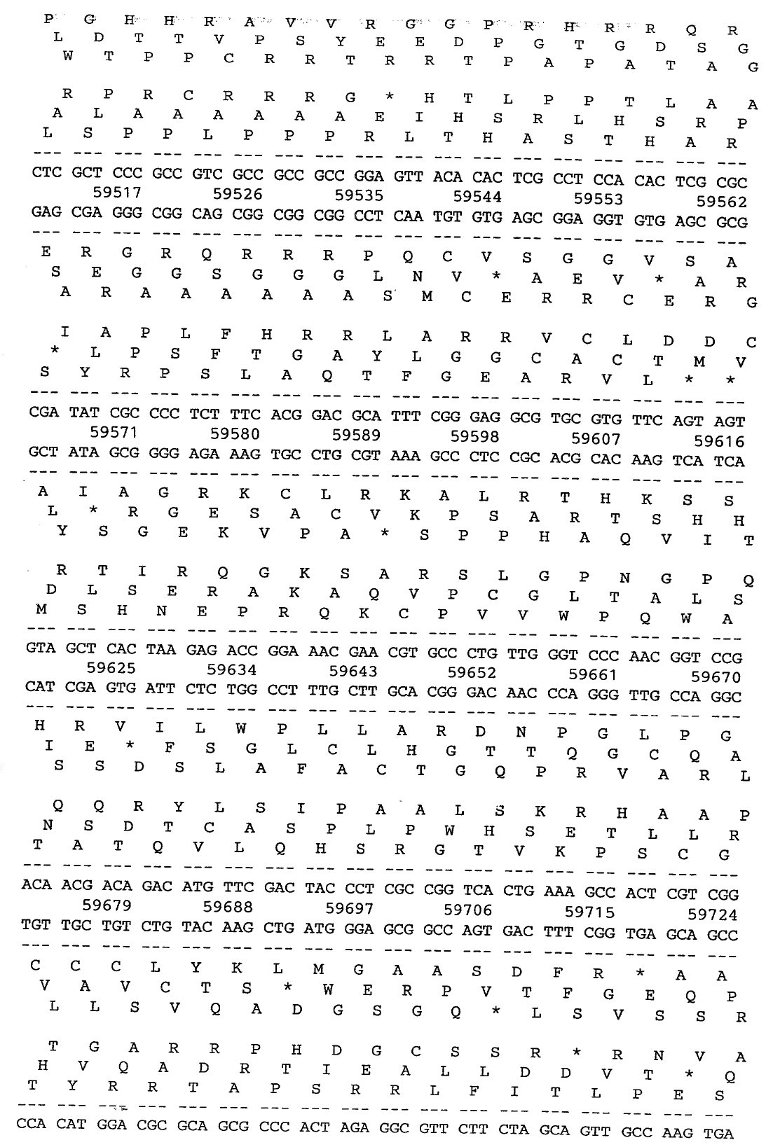

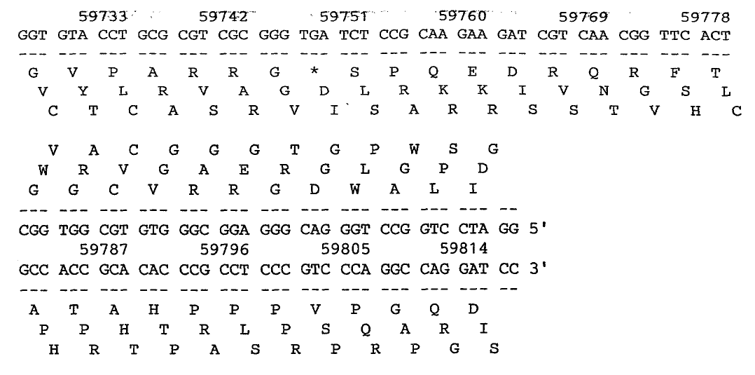

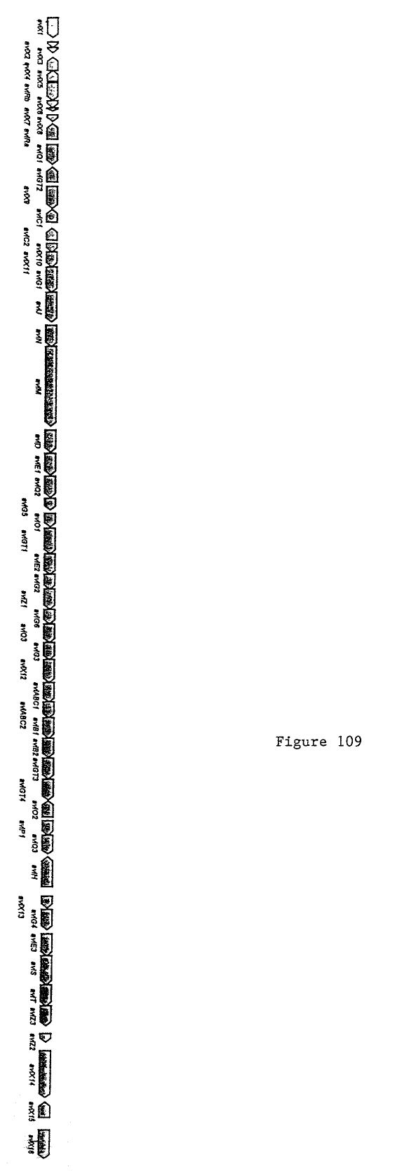

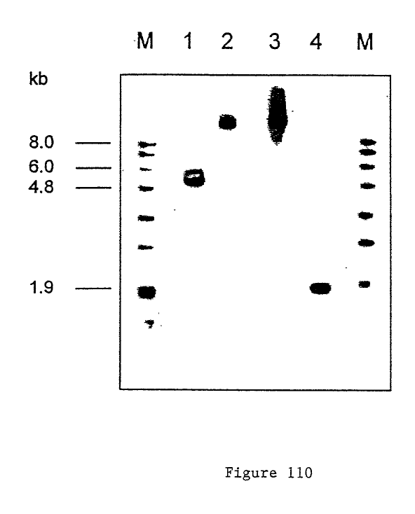

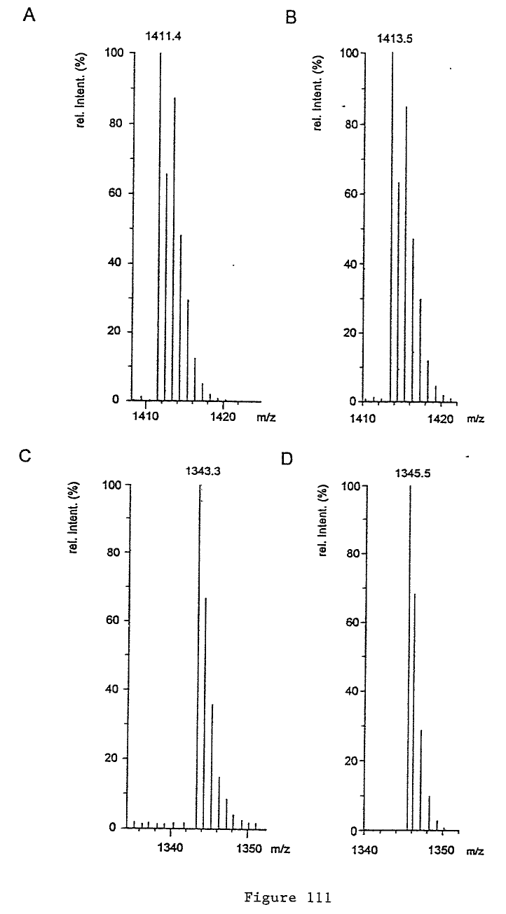

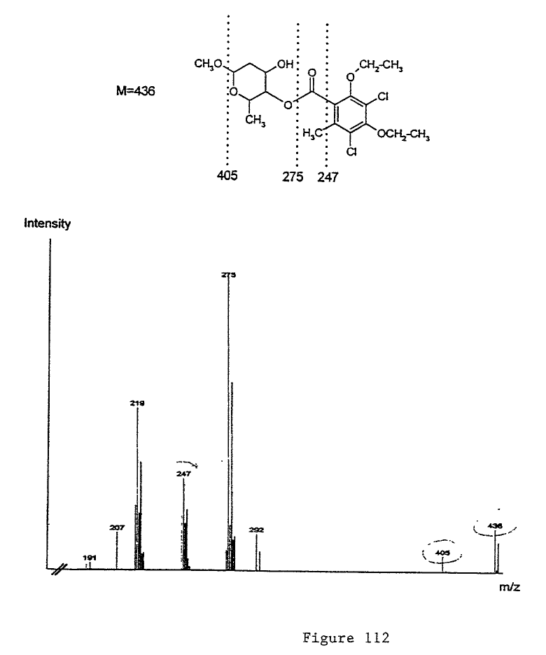

The assignment of the ORF abbreviation to its function and sequence (including the derived protein sequence) can be found in the following Table:

| Gene (ORF/ | | |
|---|---|---|
| protein or | | Consecutive number: Gene (ORF)/ |
| polypeptide | Function | protein or polypeptide in FIG. 1 |
| AviX1 | Regulation | 8/62 |
| AviX2 | | 33/87 |
| AviX3 | | 34/88 |
| AviX4 | | 35/89 |
| AviX5 | | 36/90 |
| AviRb | Resistance/methylation of the rRNA | 48/102 |
| AviX6 | | 37/91 |
| AviX7 | | 38/92 |
| AviX8 | | 39/39 |
| AviRa | Resistance/methylation of the rRNA | 49/103 |
| AviQ1 | Sugar biosynthesis | 9/63 |
| AviGT2 | Biosynthesis of the heptasaccharide chain | 10/64 |
| AviX9 | | 40/94 |
| AviC1 | Regulation | 11/65 |
| AviC2 | Regulation | 12/66 |
| AviX10 | | 41/95 |
| AviX11 | | 42/96 |
| AviG1 | Sugar biosynthesis (2-deoxy-D-evalose)/modification | 3/57 |
| | (methylation) | |
| AViJ | Antibiotic transport | 13/67 |
| AViN | Biosynthesis of orsellinic acid | 14/68 |
| AViM | Biosynthesis of orsellinic acid | 52/106 |
| AviD | Sugar biosynthesis (D-olivose, 2-deoxy-D-evalose) | 53/107 |
| AviE1 | Sugar biosynthesis (D-olivose, 2-deoxy-D-evalose) | 54/108 |
| AViQ2 | Sugar biosynthesis | 15/69 |
| AViG5 | Modification (methylation) | 6/60 |
| AviO1 | Modification | 43/97 |
| AviGT1 | Biosynthesis of the heptasaccharide chain | 16/70 |
| AviE2 | Sugar biosynthesis | 17/71 |
| AviG2 | Modification (methylation) | 4/58 |
| AviZ1 | Sugar biosynthesis | 18/72 |
| AviG6 | Modification (methylation) | 7/61 |
| AviO3 | Modification | 44/98 |
| AviG3 | Modification (methylation) | 5/59 |
| AviX12 | | 45/99 |
| AviABC1 | Antibiotic transport | 50/104 |
| AviABC2 | Antibiotic transport | 51/105 |
| AviB1 | Modification | 19/73 |
| AviB2 | Modification | 20/74 |
| AViGT3 | Biosynthesis of the heptasaccharide chain | 21/75 |
| AviGT4 | Biosynthesis of the heptasaccharide chain | 22/76 |
| AviO2 | Modification | 46/100 |
| AviP1 | Sugar biosynthesis (L-lyxose) | 23/77 |
| AviQ3 | Sugar biosynthesis | 24/78 |
| AviH | Modification (halogenation) | 1/55 |
| AviX13 | | 47/101 |
| AViG4 | Modification (methylation) | 2/56 |
| AviE3 | Sugar biosynthesis (4-O-methyl-D-fucose) | 25/79 |
| AviS | Sugar biosynthesis (D-olivose, 2-deoxy-D-evalose) | 26/80 |
| AViT | Sugar biosynthesis (D-olivose, 2-deoxy-D-evalose) | 27/81 |
| AviZ3 | Sugar biosynthesis (D-olivose, 2-deoxy-D-evalose) | 28/82 |
| AviZ2 | Sugar biosynthesis | 29/83 |
| AviX14 | Regulation | 30/84 |
| AviX15 | Regulation | 31/85 |
| AviX16 | Regulation | 32/86 |

### EXAMPLES

### Example 1

### General Methods and Materials

a) Bacteria Strains, Plasmids and Culturing Conditions

Streptomyces viridochromogenes Tü57 was cultivated with 1% malt extract, 0.4% yeast extract, 0.4% glucose, and 1 mM CaCl₂, at a pH of 7.2 (HA medium) at 37° C. For the production of avilamycin A, Streptomyces viridochromogenes Tü57 and all the mutants were cultivated in NL19+medium that contained 2% D-mannitol, 2% soybean meal, and 20 mM L-valine and was adjusted to pH 7.5. For DNA manipulation, Escherichia coli XL-1 Blue MRF′ (Stratagene) was used as the host cell. Before the transformation of S. viridochromogenes Tü57, the plasmids were raised in E. coli ET 12567 (dam-, dcm-, hsdS, Cm^{R}), in order to obtain unmethylated DNA. E. coli strains were cultivated on Luria-Bertani (LB) agar or liquid medium that contained the suitable antibiotic.

b) General Gene Technology Manipulations, PCR/DNA Sequencing/Sequence Analysis

Standard methods of molecular biology, which are known to persons skilled in the art, were carried out. The isolation of E. coli plasmid DNA, DNA restriction, DNA modification as well as “filling in sticky ends” and “Southern” hybridization were carried out in accordance with the protocols of the manufacturers of the kits, enzymes, and reagents (Amersham-Pharmacia, Boehringer Mannheim, Promega, Stratagene). Streptomyces protoplast formation, transformation, and regeneration were carried out in the usual manner. The PCR was carried out with a Perkin Elmer GeneAmp 2400 thermal cycler, where the conditions were as described and as usual. The oligonucleotide primers used were:

| | |
|---|---|
| AviG4F | (5′-GGACGCCTATCTGTGCCACCCCTTCCTGGT-3′) |
| AviG4R | (5′-TGAGCGCTCGCCTAGACAGAATCATCTCCC3′) |
| S2A | (5′-GCGTCCATCTTGCCGGGA-3′) |
| S2B | (5′-CGTGGATCCCGCCGGCCC-3′). |

Nucleotide sequencing was carried out using the dideoxynucleotide chain termination, using an automatic laser fluorescence sequencer (Perkin Elmer ABI). The sequencing reactions were carried out with a thermosequenase Cycle Sequencing Kit with 7-deaza-dGTP (Amersham) and standard primers (M 13 universal and reverse, T3, T7). A computer-supported sequence analysis was carried out using the DNASIS software package (version 2.1, 1995; Hitachi Software Engineering), and the database search was carried out using the BLAST 2.0 program, on the server of the National Center for Biotechnology Information, Bethesda, Md., USA. The sequences presented are filed in the “GenBank” database under the access number (“Accession Number”) AF333038.

c) Construction of a Gene-Inactivation Plasmid

aviG4: A unique NcoI restriction site in the gene aviG4 (consecutive number 2, FIG. 1), which is located on the 1.9 kb fragment that is ligated into the SacI and EcoRI sites of pBSK- was selected for targeted inactivation by means of a shift in the reading frame. The 1.9 kb fragment was digested with SacI and KpnI and was ligated into the gene inactivation plasmid pSP 1. After restriction digestion with NcoI, treatment with the Klenow fragment of the E. coli DNA polymerase I and religation, the intended modification was confirmed by means of DNA sequencing. The plasmid formed was referred to as pMIKG4E3.

aviH: The uniqueNarI restriction site in the aviH gene, which is present on the 3.7 kb SacI fragment ligated into pBSK-, was modified by means of NarI restriction digestion and subsequent treatment as described for aviG4. The sequencing of different plasmids showed the correct change. The 3.7 kb fragment was cloned in pSP1, in order to form the gene inactivation plasmid pSP 1S2Nar.

d) Analysis of New Avilamycin Derivatives

TLC analysis: Streptomyces viridochromogenes Tü57 and the mutants GW-4 and GW4-AM1 were incubated for three days. The cultures were filtered off, and the filtrate was applied to a solid-phase extraction cartridge (SepPakC₁₈, Waters). The cartridge was eluted with a gradient between 10% and 100% methanol in water. Avilamycin derivatives eluted with the fraction that contains 60-70% methanol. After extraction with ethyl acetate and withdrawal of the solvent, the avilamycin derivatives were dissolved in methanol once again and measured by means of TLC on silica gel plates (silica gel 60 F254, Merck), with methylene chloride/methanol (9:1, v/v) as the solvent. Avilamycin derivatives had been detected after treatment with anisaldehyde/H₂SO₄.

e) HPLC-UV Analysis

An analytical HPLC-UV was carried out on a Hewlett Packard 1090 Liquid Chromatograph with a photodiode array detector and a HP-ODS-Hypersil 5 μm, 200×2 mm column. The sequence of the solutions was as follows: Solution A, 0.04M (NH₄)₂HPO₄ pH 7.0 buffer; Solution B, 100% methanol; a non-linear gradient, with 30-62% of Solution B distributed over a period of 25 min at a flow rate of 0.2 mL/min.

f) HPLC-MS Analysis

For the HPLC-MS analysis, the avilamycin derivatives were allowed to flow on a HPLC system (HP 1110, Hewlett-Packard, Waldbronn) with a HP ODS Hypersil C₁₈ column (2.1×100 mm; 5 mm) at a flow rate of 0.1 mL/min, detection at 220 nm and the following gradient: 0-5 min from 0% to 20% B, 5.1-120 min to 90% B (Solution A, H₂O: MeOH 3:2; Solution B: MeOH). Mass spectra were recorded on a Bruker Esquire-LC 1.6 n mass spectrometer (Bruker Daltonik, Bremen) with an electrospray (ES) ion source (positive ion mode). The scan range was from 200 to 1800 m/z with nominal mass resolution.

g) GC-MS Analysis

An analysis of the new gavibamycin derivatives (avilamycin derivatives according to the invention) that were synthesized by means of the mutated cell Streptomyces viridochromogenes GW4 was carried out after ethylation, using GC-MS analysis. The derivatives were dissolved in a mixture of DMSO and acetonitrile (3:40). After addition of ethyl iodide and K₂CO₃, the reaction took place overnight. After evaporation of the solvent, the derivatives were hydrolyzed for 15 min, using HCl/methanol, at 115° C. After evaporation of the solvent, the derivatives were extracted with diethyl ether and analyzed by GC-MS. A Hewlett Packard 5973 MSD system was used, in order to obtain electron impact (EI) spectra (column: SE54,12 m×0.25 mm; d_{f}=0.125 m). The column temperature was programmed as follows: 50° C. for 2 min; 25° C./min up to 100° C.; 5° C./min up to 250° C.

### Example 2

### Cloning and Sequencing of the Avilamycin Cluster

A 60 kb segment of the chromosome of S. viridochromogenes Tü57, which contains genes involved in the biosynthesis of avilamycins, was cloned and sequenced. An analysis of the DNA sequences resulted in 54 “open reading frames.” It was already known that the NDP-glucose 4,6-dehydratase gene aviE1 and the orsellinic acid synthase gene aviM are essential for the biosynthesis of avilamycin A. The DNA that flanks the aviE1 and aviMgenes was isolated and sequenced in order to identify the biosynthetic avilamycin gene cluster. A 17.6 kb piece upstream from aviM and a 35.9 kb piece downstream from aviE1 were sequenced. The sequenced genes and their function are shown in Table 1. FIG. 109 shows the genetic arrangement of the avilamycin biosynthetic gene cluster. The cluster is flanked by an avilamycin resistance gene (aviRb) and a deoxy sugar synthesis gene (aviZ2). In the center of the sequenced segment, there are 25 genes (aviX10-aviGT4), all of which are transcribed in the same direction.

### Example 3

### Analysis of the ORFs

a) General Information

A computer analysis of the sequences of ORFs found was carried out. For the most part, the results of a sequence comparison were brought into connection with the knowledge about the biosynthesis of avilamycin A. The results of the deliberations based on the present experiments can be read off in Table 1.

In the following, the deliberations regarding function will be presented using selected examples, particularly the methyl transferases and halogenases.

b) Genes with a Function in the Biosynthesis of Dichloroisoeverninic Acid

AviM is responsible for the formation of orsellinic acid during avilamycin biosynthesis. AviN, which is located upstream from aviM, probably codes for an enzyme that controls the starter unit for orsellinic acid synthesis. The biosynthesis of dichloroisoeverninic acid (A in Formula I), proceeding from orsellinic acid, presupposes methylation and dihalogenation. Surprisingly, it was found that AviG4, resembling DmpM, an O-demethyl puromycin-O-methyl transferase from S. alboniger (44% identical amino acids), and AviH, resembling PltA, a halogenase from Pseudomonas fluorescens Pf-5, which is involved in pyoluteorin biosynthesis (39% identical amino acids), are responsible for modification of orsellinic acid.

c) Genes with a Function in the Biosynthesis of Deoxy Sugars

2-deoxy-D-evalose differs from D-olivose in a methyl group at the C3 position. It can be assumed that dNDP-4-keto-2,6-dideoxy-D-glucose is an important intermediate product in the biosynthesis of this methylated deoxy sugar. Methylation by means of AviGI, which is similar to TylCIII, a 3 C-methyl transferase from S. fradiae (54% identical amino acids) and ketoreduction by either AviZ1 or AviZ2, both of which are similar to ketoreductases and oxidoreductases, complete the biosynthesis.

d) Genes with a Function in the Modification of the Heptasaccharide Chain

In addition to aviG1, aviG4, aviRa, and aviRb, four additional methyl transferase genes were found in the cluster (aviG2, aviG3, aviG5 and aviG6). They were identified as potential methyl transferase genes because of the fact that either their product is similar to methyl transferases from other organisms, or that they contain motifs that are typically found in various methylating proteins. A cell line according to the invention produced various avilamycin derivatives that did not contain any methyl group at different positions in the molecule. This indicates that methylation takes place at a very late point in the biosynthesis. AviG2, AviG3, AviG5 and AviG6 probably methylate at the D-fucose moiety (E in Formula I), D-mannose moiety (F in Formula I), and at the methyl eurekanate moiety (H in Formula I) of avilamycin A.

### Example 4

### Production of an aviG4 Gene Substitution Mutant

The plasmid pMIKG4E3 was constructed for inactivation of aviG4 (see Example 1), in order to allow replacement of the wild type gene with a mutated allele. After formation of protoplasts and transformation of S. viridochromogenes with the plasmid pMIKG4E3, erythromycin-resistant colonies were obtained. The transformation efficiency was approximately 10 colonies per mg plasmid DNA. Several colonies were cultivated without erythromycin, on plates, in order to select for the loss of resistance. Several sensitive colonies were obtained, which indicates that this is the result of a “double cross-over.” Two mutants, G4/24/20 and G4/24/30, were studied further. PCR fragments that were amplified from G4/24/20 and G4/24/30 using the primers aviG4F-DNA and aviG4R-DNA, could not be cut by NcoI, while it was possible to cut PCR fragments from wild type DNA with this enzyme. In order to document deletion in aviG4, Southern blot analyses were carried out as follows. Chromosomal DNA cut with NcoI was obtained from G4/24/20 and G4/24/30. When this DNA was hybridized with a 1.9 kb fragment that contained the entire aviG4 gene, a 11 kb fragment was detected, while the expected 5 kb and 6 kb fragments were found in the S. viridochromogenes Tü57 line (FIG. 110). The mutant G4/24/30 was utilized for additional experiments, under the new name S. viridochromogenes GW4.

### Example 5

### Production of an aviG4-aviH Double Gene Replacement Mutant

The plasmid pSP 1 S2Nar was developed in order to delete the aviH gene (Example 1). S. viridochromogenes GW4 protoplasts were transformed with this plasmid. Approximately 20 erythromycin-resistant colonies occurred per mg DNA. Some of them were cultivated for screening as to whether or not the erythromycin resistance is lost (indicating a “double cross-over”). The mutant GW4-AM1 was selected for additional experiments. A 1.34 kb PCR fragment that was obtained from GW4-AM1 using the primers S2A and S2B could not be cut by NarI, while the PCR fragment from GW4 was digested by the enzyme. In order to prove the deletion in aviH, a Southern blot analysis was carried out. Chromosomal DNA from GW4-AM1 was cut with NarI, and hybridized with a 3.7 kb probe that contained the aviH gene. A 5.7 kb fragment was detected, while in the case of chromosomal DNA from GW4, the fragments were at 4.3 kb and 1.4 kb, as expected (not shown).

### Example 6

### Completion of S. viridochromogenes GW4 and S. viridochromogenes GW4-AM1

In order to clearly check whether the mutation relates only to the desired genes and not to any others, aviG4 and aviH were ligated behind the ermE-up promoter, cloned into the integration plasmid pSET152, and introduced into the corresponding mutants by means of protoplast transformation. The production of avilamycins, and gavibamycins, respectively, was restored. Therefore any kind of “upstream” or “downstream” effect can be precluded.

### Example 7

### Analysis of the Newly Formed Avilamycin Derivatives from S. viridochromogenes GW4 and S. viridochromogenes GW4-AM1

Avilamycin A (M+Na: 1425) and avilamycin C (M+Na: 1427) were detected in extracts of S. viridochromogenes Tü57, by means of liquid chromatography (LC) mass spectrometry analysis. Avilamycin C was the main component. Measurements at high resolution showed that both compounds contain two chlorine atoms, which can be recognized by their typical isotopic pattern. The mass of the two main compounds formed by S. viridochromogenes GW4 was 1411 (M+Na) and 1413 (M+Na) (FIG. 111), which shows that aviG4 does in fact code for a methyl transferase. The main products of the mutant GW4 were isolated, ethylated by means of treatment with ethyl iodide, and hydrolyzed using methanol and hydrochloric acid. The reaction products were analyzed by means of GC-MS. The mass spectrum of this sample showed several peaks (FIG. 112). The peak at m/z 436 corresponds to the D-olivosyl ester of dichloro-di-O-ethyl orsellinic acid and most of the other peaks (m/z 405, m/z 275, m/z 247) corresponded to fragments that proceed from the orsellinic acid moiety (FIG. 112). This makes it obvious to conclude that the difference between avilamycin A (C) and the new derivative, gavibamycin A1 (A3) results from a modification of the structure of the orsellinic acid moiety.

Gavibamycin A1 and A3 correspond to the general Formula I, with the following significance for the residues R1-R9:

| No. | R1 | R2 | R3 | R4 | R5 | R6 | R7 | R8 | R9 |
|---|---|---|---|---|---|---|---|---|---|
| A1 | COCH(CH₃)₂ | COCH₃ | OH | Cl | Cl | CH₃ | CH₃ | CH₃ | CH₃ |
| A3 | COCH(CH₃)₂ | CH(OH)CH₃ | OH | Cl | Cl | CH₃ | CH₃ | CH₃ | CH₃ |

S. viridochromogenes GW4-AM1 was also analyzed by means of HPLC-MS. The mass of the two main avilamycin derivatives was 1343 (M+Na) and 1345 (M+Na). In a comparison with the isotopic pattern of the main derivatives of the mutant GW4, the isotopic pattern of the main products of the mutant GW4-AM1 did not demonstrate any specific signals for chlorine atoms (FIG. 111), which indicates that the inactivation of aviH leads to the loss of both chlorine atoms. The new derivatives were called gavibamycin B 1 (avilamycin A analogue) and gavibamycin B3 (avilamycin C analogue).

Gavibamycin B1 and B3 correspond to the general Formula I, with the following significance for the moieties R1-R9:

| No. | R1 | R2 | R3 | R4 | R5 | R6 | R7 | R8 | R9 |
|---|---|---|---|---|---|---|---|---|---|
| B1 | COCH(CH₃)₂ | COCH₃ | OH | Cl | Cl | CH₃ | CH₃ | CH₃ | CH₃ |
| B3 | COCH(CH₃)₂ | CH(OH)CH₃ | OH | Cl | Cl | CH₃ | CH₃ | CH₃ | CH₃ |

### Example 8

### Biological Properties of Gavibamycin A3

The antimicrobial spectrum of gavibamycin A3 was determined and compared with that of avilamycin A. In this context, the “broth microdilution” method in accordance with the regulations of the national committee for clinical laboratory standards (NCCLS) was used. Both metabolites demonstrate antibiotic activity against Bacillus subtilis, Staphylococcus aureus ATCC6538, Staphylococcus aureus ATCC6538P, Staphylococcus aureus ATCC29213, Staphylococcus aureus Q48-1.2.1, Enterococcus faecalis ATCC29212, Enterococcus faecalis H-7-6, and Streptococcus pneumoniae ATCC49619.

Furthermore, preliminary tests showed that gavibamycin is somewhat more active than avilamycin A against various strains of Staphylococcus aureus, and in addition it appears to be much more hydrophilic, as is evident from the Rf values. The non-chlorinated gavibamycin derivatives also demonstrated antibiotic activity.

### Example 9

### The mutants S. viridochromogenes GW2 and GW5 as Well as the Avilamycin Derivatives According to the Invention Formed by Them

As in the case of the variants GW4 and GW4-AM1, the mutants GW2 and GW5 were produced based on S. viridochromogenes, where in the case of all the avilamycin derivatives synthesized by the two mutants, R3=OH in each instance, and in the case of the products of the mutant GW2, R6=H, and in the case of the products of the mutant GW5, R9=H. In this context, the method of procedure was completely analogous to Examples 4 to 6, particularly 5, so that in the case of GW2, a double mutant, not only aviG4 (see Example 4) but also aviG2 was genetically modified (deleted). In the case of the mutant GW5, also a double mutant, not only aviG4 but also aviG5 was genetically modified (deleted). The products according to the invention produced by these mutants GW2 and GW5 make it evident that the corresponding methyl transferases (aviG2 or aviG5, respectively, and aviG4, in each instance) were deleted.

### Example 10

### Parenteral Administration

5 g gavibamycin A3 are dissolved in 1 l water for injection purposes, if necessary using a pharmaceutically well tolerated solubility enhancer, at room temperature, and subsequently adjusted to isotonic conditions for injection purposes by adding water-free glucose.

In an average patient, with a body weight of approximately 65 kg, 0.5 mL of this solution is administered, in other words 2.5 mg or approximately 40 mg/kg. The administered dose did not demonstrate any kind of contraindications and proved to be well tolerated by the patient. Even dosage of gavibamycin A3 that was up to 20 times greater proved to be toxicologically unproblematic and was well tolerated.

### Example 11

In summary, it should be stated that according to the invention, a detailed sequence analysis of the avi gene set demonstrates several characteristics that suggest a model of a biosynthesis metabolism path to complex oligosaccharide antibiotics according to the invention. The function of the genes responsible for sugar biosynthesis can be derived from the amino acid sequences that are similar to proteins that are involved in the biosynthesis of D-olivose in other organisms. As described for the biosynthesis of D-olivose in Streptomyces violaceoruber Tü22 (granaticin producer) and Streptomyces fradiae (urdamycin producer), the biosynthesis begins with glucose-1-phosphate, which is converted to dTDP-D-olivose and dTDP-2-deoxy-D-evalose by several enzymes.

A new characteristic in this metabolism path is that three different dNDP-hexose-4,6-dehydratase genes are involved in it. On the basis of sequence homologies, AviE1 is a dTDP-glucose-4,6-dehydratase, and AviE3 is a GDP-mannose-4,6-dehydratase, which shows that the biosynthesis of some of these different sugar units starts from different nucleotide-bound hexose pools. On the basis of the structure of avilamycin A, and also indicated by the presumed function of some gene products, the biosynthesis of L-lyxose actually begins with a third sugar pool, so that this could be a product of the pentose-phosphate metabolism path.

Moiety H of avilamycin A was originally described as methyl eurekanate, derived from 2,3-di-O-methylene-4,5-dihydroxy hexanic acid. However, the sequence analysis according to the invention shows that methyl eurekanate is also the product of a biosynthetic sugar metabolism path. All this, taken together, permits the conclusion, on the basis of the number of sugar units, that the avilamycin cluster has six glycosyl transferase genes. However, only four were found in the avilamycin cluster according to the invention. A possible explanation could be the involvement of one or more glycosyl transferases in several synthesis steps, or the involvement of glycosyl transferases that are coded in regions outside this gene cluster. Three of the four glycosyl transferases are more strongly reminiscent of glycosyl transferases for the biosynthesis of O-antigen structures or cell wall polysaccharides, which can be explained by the structure of avilamycins, which is similar to that of polysaccharides.

The avi metabolism actually contains some other interesting characteristics: two ortho ester bridges and a methylene bridge. Taking into consideration the oxidative nature of these C—O—C arrangements, the a-ketoglutarate-dependent oxygenases AviO1, AviO2, and AviO3 probably catalyze the formation of this rare bond. It is therefore described, according to the invention, that such enzymes use molecular oxygen as a direct electron acceptor for oxidation, by using a-ketoglutarate as a co-substrate and thereby finally producing the C—O—C bonds, succinate, and CO₂. Avilamycin A heptasaccharide is modified by means of methylation, attachment of acetate, attachment of dichloroisoeverninic acid, and attachment of an isobutyryl unit. Six methyl transferase genes are present in the cluster, which is sufficient for avilamycin biosynthesis in terms of numbers, while the genes that are responsible for attachment of the other residues have not yet been localized.

Interestingly enough, it was found, according to the invention, that aviB1 and aviB2 encode enzymes that are similar to the alpha chain and the beta chain of Components 1 of the 2-oxo acid dehydrogenase complexes. These complexes are normally composed of three enzyme units, namely the TPP-dependent dehydrogenases (heterotetramers (a₂b₂)), dihydrolipoamide acetyl transferases (homomultimers), and dihydrolipoamide dehydrogenases (homodimers). The ORFs that code for the latter components of these complexes have either not yet been localized within the cluster, or do not lie within the cluster, or are not utilized at all for the biosynthesis of avilamycins.

Furthermore, gavibamycin A3 was tested with regard to its antibiotic effect. The first MIC experiments showed that gavibamycin A3 has somewhat stronger activity against various Staphylococcus aureus strains than avilamycin A. Furthermore, it is also somewhat more hydrophilic than avilamycin A, as was demonstrated by the retention factors from the TLC and HPLC analysis. The non-chlorinated gavibamycin derivatives also have antibiotic activity.

### Cited Literature

Buzzetti, F., Eisenberg, F., Grant, H. N., Keller-Schierlein, W., Voser, W., Zähner, H., Experientia, 24:320-323 (1968)

Foster, D. R., Rybak, M. J., Pharmacotherapy, 19:1111-1117 (1999)

Langer, E., “Comparison studies concerning the method of effect of avilamycin A and everninomycin B,” Thesis at the Faculty for Biology, Eberhard Karls University, Tübingen (1987)

Mertz, J. L., Peloso, J. S., Barker, B. J., Babbitt, G. E., Occolowitz, J. L., Simson, V. L., Kline, R. M., Antibiot, 39:877-887 (1986)

Walker, C. A., “Everninomycin B- a possible site of action,” 16^{th} Interscience conference on antimicrobial agents and chemotherapy, Abstract 116 (1976)

Weitnauer, G., Bechthold, A., PZ Prisma, 2:117-125 (1999)

Wolf, H., FEBS Lett, 36:181-186 (1973)

Wright, D., Tetrahedron Lett, 35:1207-1237 (1979)

Zähner, H., Tübingen, personal report (1999)

We claim:

1. Avilamycin derivative according to the general Formula I, also in the form of its diastereomers or enantiomers, its racemic mixtures or other mixtures or pure diastereomers and/or enantiomers, 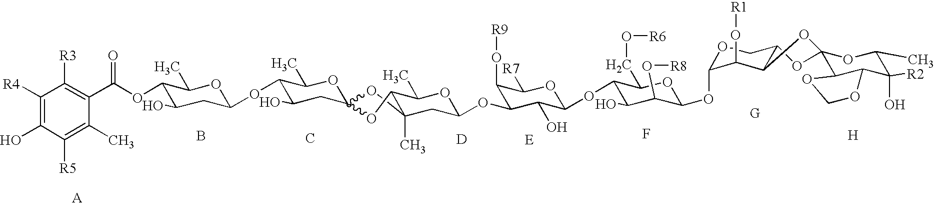
where independently of one another, with the following exception,
R1 is selected from H, COCH₃, COC₄H₉, COCH(CH₃)₂ or COCH₂CH₃,
R2 is selected from H, CHO, COCH₃ or CH(OH)CH₃,
R3 corresponds to OCH₃,
R4 corresponds to Cl,
R5 corresponds to Cl,
R6 corresponds to CH₃,
R7 corresponds to H, CH₃ or CH₂OH,
R8 corresponds to CH₃, and
R9 corresponds to CH₃,
where the following applies with reference to at least one of the residues R3-R6, R8 or R9 in Formula I, in deviation from the above definition:
R3 is to be replaced by OH,
R4 is to be replaced by H,
R5 is to be replaced by H,
R6 is to be replaced by H,
R8 is to be replaced by H, and/or
R9 is to be replaced by H,
with the proviso that R1-R9 cannot simultaneously take on the meanings in accordance with the combination, in each instance, in one of the compounds 1-4:

| No. | R1 | R2 | R3 | R4 | R5 | R6 | R7 | R8 | R9 |
|---|---|---|---|---|---|---|---|---|---|
| 1 | COCH(CH₃)₂ | COCH₃ | OH | H | Cl | CH₃ | CH₃ | CH₃ | CH₃ |
| 2 | COCH(CH₃)₂ | COCH₃ | OCH₃ | Cl | H | CH₃ | CH₃ | CH₃ | CH₃ |
| 3 | COCH(CH₃)₂ | COCH₃ | OCH₃ | Cl | CI | H | CH₃ | CH₃ | CH₃ |
| 4 | COCH(CH₃)₂ | COCH₃ | OCH₃ | Cl | CI | CH₃ | CH₃ | H | CH₃ |

2. Avilamycin derivative according to claim 1, characterized in that at least R3 is to be replaced by OH, with the proviso that R1-R9 cannot simultaneously take on the meanings in accordance with the combination in the compound 1:

| No. | R1 | R2 | R3 | R4 | R5 | R6 | R7 | R8 | R9 |
|---|---|---|---|---|---|---|---|---|---|
| 1 | COCH(CH₃)₂ | COCH₃ | OH | H | Cl | CH₃ | CH₃ | CH₃ | CH₃ |

3. Avilamycin derivative according to one of claims 1 or 2, characterized in that at least R4 and R5 in Formula I are to be replaced by H.

4. Avilamycin derivative according to one of claims 1 to 3, characterized in that at least R6, R8 and/or R9 is/are to be replaced by H, with the proviso that R1-R9 cannot simultaneously take on the meanings in accordance with the combination in the compound 3 or cannot simultaneously take on the meanings in accordance with the combination in the compound 4:

| No. | R1 | R2 | R3 | R4 | R5 | R6 | R7 | R8 | R9 |
|---|---|---|---|---|---|---|---|---|---|
| 3 | COCH(CH₃)₂ | COCH₃ | OCH₃ | Cl | Cl | H | CH₃ | CH₃ | CH₃ |
| 4 | COCH(CH₃)₂ | COCH₃ | OCH₃ | Cl | Cl | CH₃ | CH₃ | H | CH₃ |

5. Avilamycin derivative according to one of claims 1 to 4, characterized in that at least R3 is to be replaced by OH, for one thing, and at least R4 and R5 are to be replaced by H, for another thing, or at least R6, R8 and/or R9 is/are to be replaced by H.

6. Avilamycin derivative according to the general Formula I, also in the form of its diastereomers or enantiomers, its racemic mixtures or other mixtures or pure diastereomers and/or enantiomers, that is selected from among compounds in which R1-R9 are combined as follows:

| No. | R1 | R2 | R3 | R4 | R5 | R6 | R7 | R8 | R9 |
|---|---|---|---|---|---|---|---|---|---|
| 5 | COCH(CH₃)₂ | COCH₃ | OH | Cl | Cl | CH₃ | CH₃ | CH₃ | CH₃ |
| 6 | COCH₂CH₃ | H | OH | Cl | Cl | CH₃ | CH₃ | CH₃ | CH₃ |
| 7 | COCH₃ | COCH₃ | OH | Cl | Cl | CH₃ | CH₃ | CH₃ | CH₃ |
| 8 | COCH(CH₃)₂ | CH(OH)CH₃ | OH | Cl | Cl | CH₃ | CH₃ | CH₃ | CH₃ |
| 9 | H | COCH₃ | OH | Cl | Cl | CH₃ | CH₃ | CH₃ | CH₃ |
| 10 | COCH₃ | CH(OH)CH₃ | OH | Cl | Cl | CH₃ | CH₃ | CH₃ | CH₃ |
| 11 | H | CH(OH)CH₃ | OH | Cl | Cl | CH₃ | CH₃ | CH₃ | CH₃ |
| 12 | COC₄H₉ | COCH₃ | OH | Cl | Cl | CH₃ | CH₃ | CH₃ | CH₃ |
| 13 | COCH(CH₃)₂ | COCH₃ | OH | Cl | H | CH₃ | CH₃ | CH₃ | CH₃ |
| 14 | COCH₃CH₃ | COCH₃ | OH | Cl | Cl | CH₃ | CH₃ | CH₃ | CH₃ |
| 15 | COCH(CH₃)₂ | COCH₃ | OH | Cl | CL | H | CH₃ | CH₃ | CH₃ |
| 16 | COCH(CH₃)₂ | COCH₃ | OH | Cl | Cl | CH₃ | CH₂OH | CH₃ | CH₃ |
| 17 | COCH(CH₃)₂ | CHO | OH | Cl | Cl | CH₃ | CH₃ | CH₃ | CH₃ |
| 18 | COCH(CH₃)₂ | COCH₃ | OH | Cl | Cl | CH₃ | H | CH₃ | CH₃ |
| 19 | COCH(CH₃)₂ | COCH₃ | OH | Cl | Cl | CH₃ | CH₃ | H | CH₃ |
| 20 | COCH(CH₃)₂ | COCH₃ | OH | H | H | CH₃ | CH₃ | CH₃ | CH₃ |
| 21 | COCH₂CH₃ | H | OH | H | H | CH₃ | CH₃ | CH₃ | CH₃ |
| 22 | COCH₃ | COCH₃ | OH | H | H | CH₃ | CH₃ | CH₃ | CH₃ |
| 23 | COCH(CH₃)₂ | CH(OH)CH₃ | OH | H | H | CH₃ | CH₃ | CH₃ | CH₃ |
| 24 | H | COCH₃ | OH | H | H | CH₃ | CH₃ | CH₃ | CH₃ |
| 25 | COCH₃ | CH(OH)CH₃ | OH | H | H | CH₃ | CH₃ | CH₃ | CH₃ |
| 26 | H | CH(OH)CH₃ | OH | H | H | CH₃ | CH₃ | CH₃ | CH₃ |
| 27 | COCH(CH₃)₂ | COCH₃ | OH | H | H | CH₃ | CH₃ | CH₃ | CH₃ |
| 28 | COC₄H₉ | COCH₃ | OH | H | H | CH₃ | CH₃ | CH₃ | CH₃ |
| 29 | COCH(CH₃)₂ | COCH₃ | OH | H | H | CH₃ | CH₃ | CH₃ | CH₃ |
| 30 | COCH₂CH₃ | COCH₃ | OH | H | H | CH₃ | CH₃ | CH₃ | CH₃ |
| 31 | COCH(CH₃)₂ | COCH₃ | OH | H | H | H | CH₃ | CH₃ | CH₃ |
| 32 | COCH(CH₃)₂ | COCH₃ | OH | H | H | CH₃ | CH₂OH | CH₃ | CH₃ |
| 33 | COCH(CH₃)₂ | CHO | OH | H | H | CH₃ | CH₃ | CH₃ | CH₃ |
| 34 | COCH(CH₃)₂ | COCH₃ | OH | H | H | CH₃ | H | CH₃ | CH₃ |
| 35 | COCH(CH₃)₂ | COCH₃ | OH | H | H | CH₃ | CH₃ | H | CH₃ |
| 36 | COCH(CH₃)₂ | COCH₃ | OH | Cl | Cl | H | CH₃ | CH₃ | CH₃ |
| 37 | COCH₂CH₃ | H | OH | Cl | Cl | H | CH₃ | CH₃ | CH₃ |
| 38 | COCH₃ | COCH₃ | OH | Cl | Cl | H | CH₃ | CH₃ | CH₃ |
| 39 | COCH(CH₃)₂ | CH(OH)CH₃ | OH | Cl | Cl | H | CH₃ | CH₃ | CH₃ |
| 40 | H | COCH₃ | OH | Cl | Cl | H | CH₃ | CH₃ | CH₃ |
| 41 | COCH₃ | CH(OH)CH₃ | OH | Cl | Cl | H | CH₃ | CH₃ | CH₃ |
| 42 | H | CH(OH)CH₃ | OH | Cl | Cl | H | CH₃ | CH₃ | CH₃ |
| 43 | COCH(CH₃)₂ | COCH₃ | OH | H | Cl | H | CH₃ | CH₃ | CH₃ |
| 44 | COC₄H₉ | COCH₃ | OH | Cl | Cl | H | CH₃ | CH₃ | CH₃ |
| 45 | COCH(CH₃)₂ | COCH₃ | OH | Cl | H | H | CH₃ | CH₃ | CH₃ |
| 46 | COCH₂CH₃ | COCH₃ | OH | Cl | Cl | H | CH₃ | CH₃ | CH₃ |
| 47 | COCH(CH₃)₂ | COCH₃ | OH | Cl | Cl | H | CH₃ | CH₃ | CH₃ |
| 48 | COCH(CH₃)₂ | COCH₃ | OH | Cl | Cl | H | CH₂OH | CH₃ | CH₃ |
| 49 | COCH(CH₃)₂ | CHO | OH | Cl | Cl | H | CH₃ | CH₃ | CH₃ |
| 50 | COCH(CH₃)₂ | COCH₃ | OH | Cl | Cl | H | H | CH₃ | CH₃ |
| 51 | COCH(CH₃)₂ | COCH₃ | OH | Cl | Cl | H | CH₃ | H | CH₃ |
| 52 | COCH(CH₃)₂ | COCH₃ | OH | Cl | Cl | CH₃ | CH₃ | H | CH₃ |
| 53 | COCH₂CH₃ | H | OH | Cl | Cl | CH₃ | CH₃ | H | CH₃ |
| 54 | COCH₃ | COCH₃ | OH | Cl | Cl | CH₃ | CH₃ | H | CH₃ |
| 55 | COCH(CH₃)₂ | CH(OH)CH₃ | OH | Cl | Cl | CH₃ | CH₃ | H | CH₃ |
| 56 | H | COCH₃ | OH | Cl | Cl | CH₃ | CH₃ | H | CH₃ |
| 57 | COCH₃ | CH(OH)CH₃ | OH | Cl | Cl | CH₃ | CH₃ | H | CH₃ |
| 58 | H | CH(OH)CH₃ | OH | Cl | Cl | CH₃ | CH₃ | H | CH₃ |
| 59 | COCH(CH₃)₂ | COCH₃ | OH | H | Cl | CH₃ | CH₃ | H | CH₃ |
| 60 | COC₄H₉ | COCH₃ | OH | Cl | Cl | CH₃ | CH₃ | H | CH₃ |
| 61 | COCH(CH₃)₂ | COCH₃ | OH | Cl | H | CH₃ | CH₃ | H | CH₃ |
| 62 | COCH₂CH₃ | COCH₃ | OH | Cl | Cl | CH₃ | CH₃ | H | CH₃ |
| 63 | COCH(CH₃)₂ | COCH₃ | OH | Cl | Cl | H | CH₃ | H | CH₃ |
| 64 | COCH(CH₃)₂ | COCH₃ | OH | Cl | Cl | CH₃ | CH₂OH | H | CH₃ |
| 65 | COCH(CH₃)₂ | CHO | OH | Cl | Cl | CH₃ | CH₃ | H | CH₃ |
| 66 | COCH(CH₃)₂ | COCH₃ | OH | Cl | Cl | CH₃ | H | H | CH₃ |
| 67 | COCH(CH₃)₂ | COCH₃ | OH | Cl | Cl | CH₃ | CH₃ | H | CH₃ |
| 68 | COCH(CH₃)₂ | COCH₃ | OH | Cl | Cl | CH₃ | CH₃ | CH₃ | H |
| 69 | COCH₂CH₃ | H | OH | Cl | Cl | CH₃ | CH₃ | CH₃ | H |
| 70 | COCH₃ | COCH₃ | OH | Cl | Cl | CH₃ | CH₃ | CH₃ | H |
| 71 | COCH(CH₃)₂ | CH(OH)CH₃ | OH | Cl | Cl | CH₃ | CH₃ | CH₃ | H |
| 72 | H | COCH₃ | OH | Cl | Cl | CH₃ | CH₃ | CH₃ | H |
| 73 | COCH₃ | CH(OH)CH₃ | OH | Cl | Cl | CH₃ | CH₃ | CH₃ | H |
| 74 | H | CH(OH)CH₃ | OH | Cl | Cl | CH₃ | CH₃ | CH₃ | H |
| 75 | COCH(CH₃)₂ | COCH₃ | OH | H | Cl | CH₃ | CH₃ | CH₃ | H |
| 76 | COC₄H₉ | COCH₃ | OH | Cl | Cl | CH₃ | CH₃ | CH₃ | H |
| 77 | COCH(CH₃)₂ | COCH₃ | OH | Cl | H | CH₃ | CH₃ | CH₃ | H |
| 78 | COCH₂CH₃ | COCH₃ | OH | Cl | Cl | CH₃ | CH₃ | CH₃ | H |
| 79 | COCH(CH₃)₂ | COCH₃ | OH | Cl | Cl | H | CH₃ | CH₃ | H |
| 80 | COCH(CH₃)₂ | COCH₃ | OH | Cl | Cl | CH₃ | CH₂OH | CH₃ | H |
| 81 | COCH(CH₃)₂ | CHO | OH | Cl | Cl | CH₃ | CH₃ | CH₃ | H |
| 82 | COCH(CH₃)₂ | COCH₃ | OH | Cl | Cl | CH₃ | H | CH₃ | H |
| 83 | COCH(CH₃)₂ | COCH₃ | OH | Cl | Cl | CH₃ | CH₃ | H | H |
| 84 | COCH(CH₃)₂ | COCH₃ | OH | Cl | Cl | H | CH₃ | H | H |
| 85 | COCH₂CH₃ | H | OH | Cl | Cl | H | CH₃ | H | H |
| 86 | COCH₃ | COCH₃ | OH | Cl | Cl | H | CH₃ | H | H |
| 87 | COCH(CH₃)₂ | CH(OH)CH₃ | OH | Cl | Cl | H | CH₃ | H | H |
| 88 | H | COCH₃ | OH | Cl | Cl | H | CH₃ | H | H |
| 89 | COCH₃ | CH(OH)CH₃ | OH | Cl | Cl | H | CH₃ | H | H |
| 90 | H | CH(OH)CH₃ | OH | Cl | Cl | H | CH₃ | H | H |
| 91 | COCH(CH₃)₂ | COCH₃ | OH | H | Cl | H | CH₃ | H | H |
| 92 | COC₄H₉ | COCH₃ | OH | Cl | Cl | H | CH₃ | H | H |
| 93 | COCH(CH₃)₂ | COCH₃ | OH | Cl | H | H | CH₃ | H | H |
| 94 | COCH₂CH₃ | COCH₃ | OH | Cl | Cl | H | CH₃ | H | H |
| 95 | COCH(CH₃)₂ | COCH₃ | OH | Cl | Cl | H | CH₃ | H | H |
| 96 | COCH(CH₃)₂ | COCH₃ | OH | Cl | Cl | H | CH₂OH | H | H |
| 97 | COCH(CH₃)₂ | CHO | OH | Cl | Cl | H | CH₃ | H | H |
| 98 | COCH(CH₃)₂ | COCH₃ | OH | Cl | Cl | H | H | H | H |
| 99 | COCH(CH₃)₂ | COCH₃ | OH | Cl | Cl | H | CH₃ | H | H |

preferably,

| R1 | R2 | R3 | R4 | R5 | R6 | R7 | R8 | R9 |
|---|---|---|---|---|---|---|---|---|
| COCH(CH₃)₂ | COCH₃ | OH | Cl | Cl | CH₃ | CH₃ | CH₃ | CH₃ |
| COCH(CH₃)₂ | CH(OH)CH₃ | OH | Cl | Cl | CH₃ | CH₃ | CH₃ | CH₃ |
| COCH(CH₃)₂ | COCH₃ | OH | H | H | CH₃ | CH₃ | CH₃ | CH₃ |
| COCH(CH₃)₂ | CH(OH)CH₃ | OH | H | H | CH₃ | CH₃ | CH₃ | CH₃ |
| COCH(CH₃)₂ | COCH₃ | OH | Cl | Cl | H | CH₃ | CH₃ | CH₃ |
| COCH(CH₃)₂ | CH(OH)CH₃ | OH | Cl | Cl | H | CH₃ | CH₃ | CH₃ |
| COCH(CH₃)₂ | COCH₃ | OH | Cl | Cl | CH₃ | CH₃ | H | CH₃ |
| COCH(CH₃)₂ | CH(OH)CH₃ | OH | Cl | Cl | CH₃ | CH₃ | H | CH₃ |
| COCH(CH₃)₂ | COCH₃ | OH | Cl | Cl | CH₃ | CH₃ | CH₃ | H |
| COCH(CH₃)₂ | CH(OH)CH₃ | OH | Cl | Cl | CH₃ | CH₃ | CH₃ | H |

7. Avilamycin derivative that can be obtained in that in a cell that can be cultivated, which contains the necessary genes and/or enzymes for the synthesis of an orthosomycin basic body consisting of
a) a terminal dichloroisoeverninic acid moiety (A in Formula I) and
b) a heptasaccharide esterified with it, linked via normal ester bonds and ortho ester bonds (B to H in Formula I), composed of:
(i) two D-olivose moieties (B and C),
(ii) a 2-deoxy-D-evalose moiety (D),
(iii) a D-fucose moiety (E),
(iv) a D-mannose moiety (F),
(v) an L-lyxose moiety (G), and
(vi) a (methyl) eurekanate moiety (H),
by modifying with gene technology, deleting, and/or not expressing at least one nucleic acid, the sequence of which corresponds by at least 95%, preferably 97%, and particularly precisely to the nucleic acid sequence in accordance with one of FIGS. 1 to 54, by cultivating the cell modified in this way, by recovering and processing the top portion of the culture, by purifying and isolating the avilamycin derivative(s), and, if applicable, by separating different derivatives from one another,
with the proviso that R1-R9 cannot simultaneously take on the meanings in accordance with the combination, in each instance, as shown below:

| R1 | R2 | R3 | R4 | R5 | R6 | R7 | R8 | R9 |
|---|---|---|---|---|---|---|---|---|
| COCH(CH₃)₂ | COCH₃ | OH | H | Cl | CH₃ | CH₃ | CH₃ | CH₃ |
| COCH(CH₃)₂ | COCH₃ | OCH₃ | Cl | H | CH₃ | CH₃ | CH₃ | CH₃ |
| COCH(CH₃)₂ | COCH₃ | OCH₃ | Cl | Cl | H | CH₃ | CH₃ | CH₃ |
| COCH(CH₃)₂ | COCH₃ | OCH₃ | Cl | Cl | CH₃ | CH₃ | H | CH₃ |
| COCH(CH₃)₂ | CHO | OCH₃ | Cl | Cl | CH₃ | CH₃ | CH₃ | CH₃ |
| COCH(CH₃)₂ | COCH₃ | OCH₃ | Cl | Cl | CH₃ | H | CH₃ | CH₃ |
| COCH(CH₃)₂ | COCH₃ | OCH₃ | Cl | Cl | CH₃ | CH₃ | CH₃ | CH₃ |
| COCH₂CH₃ | H | OCH₃ | Cl | Cl | CH₃ | CH₃ | CH₃ | CH₃ |
| COCH₃ | COCH₃ | OCH₃ | Cl | Cl | CH₃ | CH₃ | CH₃ | CH₃ |
| COCH(CH₃)₂ | CH(OH)CH₃ | OCH₃ | Cl | Cl | CH₃ | CH₃ | CH₃ | CH₃ |
| H | COH₃ | OCH₃ | Cl | Cl | CH₃ | CH₃ | CH₃ | CH₃ |
| COCH₃ | CH(OH)CH₃ | OCH₃ | Cl | Cl | CH₃ | CH₃ | CH₃ | CH₃ |
| H | CH(OH)CH₃ | OCH₃ | Cl | Cl | CH₃ | CH₃ | CH₃ | CH₃ |
| COC₄H₉ | COCH₃ | OCH₃ | Cl | Cl | CH₃ | CH₃ | CH₃ | CH₃ |
| COCH₂CH₃ | COCH₃ | OCH₃ | Cl | Cl | CH₃ | CH₃ | CH₃ | CH₃ |
| COCH(CH₃)₂ | COCH₃ | OCH₃ | Cl | Cl | CH₃ | CH₂OH | CH₃ | CH₃ |

8. Avilamycin derivative according to claim 7, characterized in that the cell that can be cultivated is selected from a cell of the type Streptomyces viridochromogenes or a cell that with the exception of the nucleic acid(s) modified by gene technology, deleted, or not expressed, contains the nucleic acids in accordance with a sequence of one of the consecutive numbers 1-54 (in accordance with Table 1 in combination with FIG. 1), or nucleic acids that are homologous to it by at least 95%, preferably 97%, or contains the gene cluster in accordance with FIG. 109, is preferably selected from a cell of the type Streptomyces viridochromogenes, particularly a cell of the type Streptomyces viridochromogenes Tü 57.

9. Avilamycin derivative according to one of claims 7 or 8, characterized in that the modified nucleic acid(s) coded for a methyl transferase and/or for a halogenase.

10. Avilamycin derivative according to claim 9, characterized in that the sequence(s) of the modified nucleic acid(s) before being modified correspond(s) by at least 95%, preferably 97%, and particularly precisely to the nucleic acid sequence(s) of at least one of the sequences in accordance with consecutive number 1 or 2-7 (in accordance with Table 1 in combination with FIG. 1), preferably one of the sequences with consecutive number 1, 2, 4, or 6 (Table 1 in combination with FIG. 1), particularly the sequence with consecutive number 2 or the sequences with consecutive numbers 2 and 1, numbers 2 and 4, or numbers 2 and 6 (in accordance with Table 1 in combination with FIG. 1)

11. Avilamycin derivative according to one of claims 7 to 10, characterized in that the modification of the nucleic acid(s) has the result that the protein(s) or polypeptide(s) coded by the nucleic acid(s) modified by gene technology is/are no longer synthesized after the modification by gene technology.

12. Avilamycin derivative according to one of claims 1 to 5, 6 or 7 to 11, characterized in that it is more hydrophilic than avilamycin A or C or evernimycin (ziracin).

13. Nucleic acid in its sequence corresponding by at least 95%, preferably 97%, and particularly precisely to the nucleic acid sequence in accordance with one of the sequences with the consecutive number 1 to 51 (in accordance with Table 1 in combination with FIG. 1).

14. Nucleic acid according to claim 13, characterized in that the nucleic acid corresponds by at least 95%, preferably 97%, and particularly precisely to the nucleic acid sequence in accordance with one of the sequences with consecutive number 1 to 32 and 48 to 51 (in accordance with Table 1 in combination with FIG. 1), preferably 1 to 7, especially 1, 2, 4, or 6.

15. Gene clusters containing “Open reading frames,” preferably 54, which correspond, in their nucleic acid sequence, by at least 95%, preferably 97%, and particularly precisely to the nucleic acid sequences according to the sequences with the consecutive numbers 1 to 54 (in accordance with Table 1 in combination with FIG. 1) and that are arranged on a nucleic acid strand, preferably in accordance with FIG. 109.

16. Protein or polypeptide corresponding, in its amino acid sequence, by at least 95%, preferably 97%, and particularly precisely to the amino acid sequence in accordance with one of the sequences with the consecutive number 55-105 (in accordance with Table 1 in combination with FIG. 1).

17. Protein or polypeptide according to claim 16, characterized in that the protein or polypeptide corresponds by at least 95%, preferably 97%, and particularly precisely to the nucleic acid sequence in accordance with one of the sequences with the consecutive number 55 to 86 or 97, 98 or 100 or 102 to 105 (in accordance with Table 1 in combination with FIG. 1), preferably 55 to 61, particularly 55, 56, 58, or 60.

18. Protein or polypeptide coded by a nucleic acid according to one of claims 13 or 14.

19. Cell modified by gene technology, containing at least one non-endogenous nucleic acid according to one of claims 13 or 14, a non-endogenous gene cluster according to claim 15, and/or a non-endogenous protein or polypeptide according to one of claims 16-18.

20. Cell containing at least one nucleic acid modified by gene technology, the sequence of which, before modification, corresponded by at least 95%, preferably 97, and particularly precisely to the nucleic acid sequence in accordance with one of the sequences with the consecutive number 1 to 54 (in accordance with Table 1 in combination with FIG. 1).

21. Cell of the type Streptomyces viridochromogenes, preferably of the subtype Tü57, characterized in that at least one of the nucleic acids with a sequence in accordance with one of the sequences with consecutive number 1-54 (in accordance with Table 1 in combination with FIG. 1) was modified by gene technology or deleted.

22. Cell according to claim 21, characterized in that at least one of the nucleic acids with a sequence in accordance with one of the sequences with the consecutive number 1 or 2-7 (in accordance with Table 1 in combination with FIG. 1), preferably with one of the sequences with the consecutive number 1, 2, 4, or 6 (Table 1 in combination with FIG. 1), particularly with a sequence with the consecutive number 2 or with a sequence with the consecutive numbers 2 and 1, 2 and 4, or 2 and 6 (in accordance with Table 1 in combination with FIG. 1) was modified by gene technology or deleted.

23. Cell according to claim 21, of the mutant type Streptomyces viridochromogenes GW4, Streptomyces viridochromogenes GW4-AM1, Streptomyces viridochromogenes GW2 or Streptomyces viridochromogenes GW5, characterized in that avilamycin derivatives in which R3=OH are synthesized by the mutant type Streptomyces viridochromogenes GW4, avilamycin derivatives in which R3=OH, R4=H, and R5=H are synthesized by the mutant type Streptomyces viridochromogenes GW4-AM1, avilamycin derivatives in which R3=OH and R6=H are synthesized by the mutant type Streptomyces viridochromogenes GW2, and avilamycin derivatives in which R3=OH and R9=H are synthesized by the mutant type Streptomyces viridochromogenes GW5.

24. Use of a nucleic acid according to one of claims 13 or 14, of a gene cluster according to claim 15, of a protein or polypeptide according to one of claims 16 to 18, and/or of a cell according to one of claims 19 to 23, for the production of an avilamycin derivative, preferably according to one of claims 1 to 12.

25. Process for the production of avilamycin derivatives according to one of claims 1 to 6, characterized by the following steps:
(1) in a cell that can be cultivated, which contains the necessary genes, and/or enzymes for the synthesis of the orthosomycin basic body consisting of
a) a terminal dichloroisoeverninic acid moiety (A in Formula I) and
b) a heptasaccharide esterified with it, linked via normal ester bonds and ortho ester bonds (B to H in Formula I), composed of
(i) two D-olivose moieties (B and C),
(ii) a 2-deoxy-D-evalose moiety (D),
(iii) a D-fucose moiety (E),
(iv) a D-mannose moiety (F),
(v) an L-lyxose moiety (G), and
(vi) a (methyl) eurekanate moiety (H),
at least one nucleic acid, the sequence of which corresponds by at least 95%, preferably 97%, and particularly precisely to the nucleic acid sequence in accordance with one of the sequences with a consecutive number from 1 to 54 (in accordance with Table 1 in combination with FIG. 1), is modified by gene technology, deleted and/or not expressed,
(2) the cell modified by gene technology in this way is cultivated,
(3) the top portion of the culture is recovered
(4) the top portion of the culture is processed and the avilamycin derivative(s) formed in this way is/are purified and isolated,
(5) if applicable, different derivatives are separated from one another.

26. Process according to claim 25, characterized in that the cell that can be cultivated is selected from a cell of the type Streptomyces viridochromogenes or a cell that, with the exception of the nucleic acid modified by gene technology, deleted or not expressed contains the nucleic acids in accordance with a sequence with the consecutive number 1-54 (in accordance with Table 1 in combination with FIG. 1), or nucleic acids homologous to them by at least 95%, preferably 97%, or the gene cluster according to claim 15, preferably selected from a cell of the type Streptomyces viridochromogenes, especially a cell of the type Streptomyces viridochromogenes Tü 57.

27. A process according to one of claims 25 or 26, characterized in that the modified nucleic acid(s) coded for a methyl transferase and/or for a halogenase.

28. Process according to claim 27, characterized in that the sequence(s) of the modified nucleic acid(s) before modification correspond(s) by at least 95%, preferably 97%, and particularly precisely to the nucleic acid sequence(s) of at least one of the sequences with the consecutive numbers 1 or 2-7 (in accordance with Table 1 in combination with FIG. 1), preferably one of the sequences with the consecutive number 1, 2, 4, or 6 (Table 1 in combination with FIG. 1), particularly the sequence with the consecutive number 2 or the sequences with the consecutive numbers 2 and 1, 2 and 4, or 2 and 6 (in accordance with Table 1 in combination with FIG. 1).

29. Process according to one of claims 25 to 28, characterized in that modification of the nucleic acid(s) has the result that the protein(s) or polypeptide(s) coded by the nucleic acid(s) modified by gene technology is/are no longer synthesized after the gene technology modification.

30. Medication containing avilamycin derivatives according to one of claims 1 to 12, as well as any suitable additives and/or ancillary substances, if necessary.

31. Use of an avilamycin derivative according to one of claims 1 to 12 for the production of a medication with an antibiotic effect for treatment, or for the production of a medication for treatment of diseases, for example infectious diseases.
